(19) United States
(12) Patent Application Publication
(10) Pub. No.: US 2022/0227707 A1
(43) Pub. Date: Jul. 21, 2022

(54) COMPOUNDS AND COMPOSITIONS FOR TREATING CONDITIONS ASSOCIATED WITH NLRP ACTIVITY

(71) Applicant: Novartis AG, Basel (CH)

(72) Inventors: Jason KATZ, Newton, MA (US); Dong-Ming SHEN, Edison, NJ (US)

(21) Appl. No.: 17/422,644

(22) PCT Filed: Jan. 21, 2020
(86) PCT No.: PCT/US2020/014468

§ 371 Date: Jul. 13, 2021

Related U.S. Application Data
(60) Provisional application No. 62/796,361, filed on Jan. 24, 2019.
(60) Provisional application No. 62/796,356, filed on Jan. 24, 2019.
(60) Provisional application No. 62/795,894, filed on Jan. 23, 2019.
(60) Provisional application No. 62/795,395, filed on Jan. 22, 2019.

(51) Int. Cl. C07C 381/10; C07D 207/12; C07D 333/22; C07C 311/51; C07D 231/12; C07C 311/56

(52) U.S. Cl. CPC C07C 381/10; C07D 207/12; C07C 311/56; C07C 311/51; C07D 231/12; C07D 333/22

(57) ABSTRACT

In one aspect, compounds of Formula AA, or a pharmaceutically acceptable salt thereof, are featured: Formula (AA) or a pharmaceutically acceptable salt thereof, wherein the variables shown in Formula A can be as defined anywhere herein.

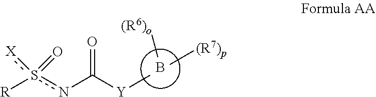

### TECHNICAL FIELD

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) contributes to the pathology and/or symptoms and/or progression of the condition, disease or disorder in a subject (e.g., a human). This disclosure also features compositions as well as other methods of using and making the same.

The present disclosure also relates to, in part, methods and compositions for treating anti-TNFα resistance in a subject with an NLRP3 antagonist. The present disclosure also relates, in part, to methods, combinations and compositions for treating TFNα related diseases and anti-TNFα resistance in a subject that include administration of an NLRP3 antagonist, an NLRP3 antagonist and an anti-TNFα agent, or a composition encompassing an NLRP3 antagonist and an anti-TNFα agent.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS) and neonatal onset multi-system inflammatory disease (NOMID) are examples of indications that have been reported to be associated with gain of function mutations in NLRP3.

NLRP3 can form a complex and has been implicated in the pathogenesis of a number of complex diseases, including but not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In light of the above, it would be desirable to provide compounds that modulate (e.g., antagonize) NLRP3.

Separately, several patients having inflammatory or autoimmune diseases are treated with anti-TNFα agents. A subpopulation of such patients develop resistance to treatment with the anti-TNFα agents. It is desirable to develop methods for reducing a patient's resistance to anti-TNFα agents. In light of the this, it would also be desirable to provide alternative therapies for treating inflammatory or autoimmune diseases (for example NLRP3 inflammasome inhibitors) to avoid or minimise the use of anti-TNFα agents.

Intestinal bowel disease (IBD), encompassing Ulcerative Colitis (UC) and Crohn's disease (CD), are chronic diseases characterized by barrier dysfunction and uncontrolled inflammation and mucosal immune reactions in the gut. A number of inflammatory pathways have been implicated in the progression of IBD, and anti-inflammatory therapy such as tumor necrosis factor-alpha (TNF-α) blockade has shown efficacy in the clinic (Rutgeerts P et al N Engl J Med 2005; 353: 2462-76). Anti-TNFα therapies, however, do not show complete efficacy, however, other cytokines such as IL-1β, IL-6, IL-12, IL-18, IL-21, and IL-23 have been shown to drive inflammatory disease pathology in IBD (Neurath M F Nat Rev Immunol 2014; 14; 329-42). IL-1β and IL-18 are produced by the NLRP3 inflammasome in response to pathogenic danger signals, and have been shown to play a role in IBD. Anti-IL-1β therapy is efficacious in patients with IBD driven by genetic mutations in CARD8 or IL-10R (Mao L et al, J Clin Invest 2018; 238:1793-1806, Shouval D S et al, Gastroenterology 2016; 151: 1100-1104), IL-18 genetic polymorphisms have been linked to UC (Kanai T et al, Curr Drug Targets 2013; 14: 1392-9), and NLRP3 inflammasome inhibitors have been shown to be efficacious in murine models of IBD (Perera A P et al, Sci Rep 2018; 8:8618). Resident gut immune cells isolated from the lamina propria of IBD patients can produce IL-1β, either spontaneously or when stimulated by LPS, and this IL-1β production can be blocked by the ex vivo addition of a NLRP3 antagonist. Based on strong clinical and preclinical evidence showing that inflammasome-driven IL-1β and IL-18 play a role in IBD pathology, it is clear that NLRP3 inflammasome inhibitors could be an efficacious treatment option for UC, Crohn's disease, or subsets of IBD patients. These subsets of patients could be defined by their peripheral or gut levels of inflammasome related cytokines including IL-1β, IL-6, and IL-18, by genetic factors that pre-dispose IBD patients to having NLRP3 inflammasome activation such as mutations in genes including ATG16L1, CARDS, IL-10R, or PTPN2 (Saitoh T et al, Nature 2008; 456: 264, Spalinger M R, Cell Rep 2018; 22:1835), or by other clinical rationale such as non-response to TNF therapy.

Though anti-TNF therapy is an effective treatment option for Crohn's disease, 40% of patients fail to respond. One-third of non-responsive CD patients fail to respond to anti-TNF therapy at the onset of treatment, while another third lose response to treatment over time (secondary non-response). Secondary non-response can be due to the generation of anti-drug antibodies, or a change in the immune compartment that desensitizes the patient to anti-TNF (Ben-Horin S et al, Autoimmun Rev 2014; 13: 24-30, Steenholdt C et al Gut 2014; 63: 919-27). Anti-TNF reduces inflammation in IBD by causing pathogenic T cell apoptosis in the intestine, therefore eliminating the T cell mediated inflammatory response (Van den Brande et al Gut 2007: 56: 509-17). There is increased NLRP3 expression and increased production of IL-1β in the gut of TNF-non-responsive CD patients (Leal R F et al Gut 2015; 64: 233-42) compared to TNF-responsive patients, suggesting NLRP3 inflammasome pathway activation. Furthermore, there is increased expression of TNF-receptor 2 (TNF-R2), which allows for TNF-mediated proliferation of T cells (Schmitt H et al Gut 2018; 0: 1-15). IL-1β signaling in the gut promotes T cell differentiation toward Th1/17 cells which can escape anti-TNF-α mediated apoptosis. It is therefore likely that NLRP3 inflammasome activation can cause non-responsiveness in CD patients to anti-TNF-α therapy by sensitizing pathogenic T cells in the gut to anti-TNF-α mediated apoptosis. Experimental data from immune cells isolated from the gut of TNF-resistant Crohn's patients show that these cells spontaneously release IL-1β, which can be inhibited by the addition of an NLRP3 antagonist. NLRP3 inflammasome antagonists—in part by blocking IL-1β secretion—would be expected to inhibit the mechanism leading to anti-TNF non-responsiveness, re-sensitizing the patient to anti-TNF therapy. In IBD patients who are naive to anti-TNF therapy, treatment with an NLRP3 antagonist would be expected to prevent primary- and secondary-non responsiveness by blocking the mechanism leading to non-response.

NLRP3 antagonists that are efficacious locally in the gut can be efficacious drugs to treat IBD; in particular in the treatment of TNF-resistant CD alone or in combination with anti-TNF therapy. Systemic inhibition of both IL-1β and TNF-α has been shown to increase the risk of opportunistic infections (Genovese M C et al, Arthritis Rheum 2004; 50: 1412), therefore, only blocking the NLRP3 inflammasome at the site of inflammation would reduce the infection risk inherent in neutralizing both IL-1β and TNF-α. NLRP3 antagonists that are potent in NLRP3-inflammasome driven cytokine secretion assays in cells, but have low permeability in vitro in a permeability assay such as an MDCK assay, have poor systemic bioavailability in a rat or mouse pharmacokinetic experiment, but high levels of compound in the colon and/or small intestine could be a useful therapeutic option for gut restricted purposes.

In light of the above, the present invention also provides alternative therapies for the treatment of inflammatory or autoimmune diseases, including IBD, that solves the above problems associated with anti-TNFα agents.

### SUMMARY

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling).

In some embodiments, provided herein is a compound of Formula AA

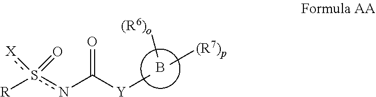

or a pharmaceutically acceptable salt thereof, wherein the variables in Formula AA can be as defined anywhere herein.

In some embodiments, provided herein is a compound of Formula AB

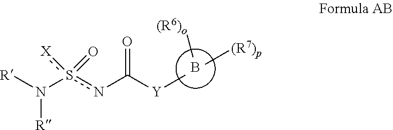

or a pharmaceutically acceptable salt thereof, wherein the variables in Formula AA can be as defined anywhere herein.

The present invention is also relates to the Applicant's discovery that inhibition of NLRP3 inflammasomes can increase a subject's sensitivity to an anti-TNFα agent or can overcome resistance to an anti-TNFα agent in a subject, or indeed provide an alternative therapy to anti-TNFα agents.

Provided herein are methods of treating a subject that include: (a) identifying a subject having a cell that has an elevated level of NLRP3 inflammasome activity and/or expression as compared to a reference level; and (b) administering to the identified subject a therapeutically effective amount of an compound of Formula I or a pharmaceutically acceptable salt, solvate, or co-crystal thereof.

Provided herein are methods for the treatment of inflammatory or autoimmune disease including IBD, such as UC and CD in a subject in need thereof, comprising administering to said subject a therapeutically effective amount a compound for Formula I or a pharmaceuticalltreat″y acceptable salt, solvate, or co-crystal thereof, wherein the NLRP3 antagonist is a gut-targeted NLRP3 antagonist.

Provided herein are methods of treating a subject in need thereof, that include: (a) identifying a subject having resistance to an anti-TNFα agent; and (b) administering a treatment comprising a therapeutically effective amount of a compound for Formula I, or a pharmaceutically acceptable salt, solvate, or co-crystal thereof to the identified subject.

Provided herein are methods of treating a subject in need thereof, that include: administering a treatment comprising a therapeutically effective amount of a compound for Formula I or a pharmaceutically acceptable salt, solvate, or co-crystal thereof to a subject identified as having resistance to an anti-TNFα agent.

Provided herein are methods of selecting a treatment for a subject in need thereof, that include: (a) identifying a subject having resistance to an anti-TNFα agent; and (b) selecting for the identified subject a treatment comprising a therapeutically effective amount of a compound for Formula I or a pharmaceutically acceptable salt, solvate, or co-crystal thereof.

Provided herein are methods of selecting a treatment for a subject in need thereof, that include selecting a treatment comprising a therapeutically effective amount of a compound for Formula I or a pharmaceutically acceptable salt, solvate, or co-crystal thereof for a subject identified as having resistance to an anti-TNFα agent.

In some embodiments of any of the methods described herein, the treatment further includes a therapeutically effective amount of an anti-TNFα agent, in addition to the NLRP3 antagonist.

This disclosure also features compositions as well as other methods of using and making the same.

An “antagonist” of NLRP3 includes compounds that inhibit the ability of NLRP3 to induce the production of IL-1β and/or IL-18 by directly binding to NLRP3, or by inactivating, destabilizing, altering distribution, of NLRP3 or otherwise.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating (e.g., agonizing, partially agonizing, antagonizing) NLRP3 activity are featured that include contacting NLRP3 with a chemical entity described herein (e.g., a compound described generically or specifically herein or a pharmaceutically acceptable salt thereof or compositions containing the same). Methods include in vitro methods, e.g., contacting a sample that includes one or more cells comprising NLRP3, as well as in vivo methods.

In a further aspect, methods of treatment of a disease in which NLRP3 signaling contributes to the pathology and/or symptoms and/or progression of the disease are featured that include administering to a subject in need of such treatment an effective amount of a chemical entity described herein (e.g., a compound described generically or specifically herein or a pharmaceutically acceptable salt thereof or compositions containing the same).

In a further aspect, methods of treatment are featured that include administering to a subject a chemical entity described herein (e.g., a compound described generically or specifically herein or a pharmaceutically acceptable salt thereof or compositions containing the same), wherein the chemical entity is administered in an amount effective to treat a disease in which NLRP3 signaling contributes to the pathology and/or symptoms and/or progression of the disease, thereby treating the disease.

Embodiments can include one or more of the following features.

The chemical entity can be administered in combination with one or more additional therapies with one or more agents suitable for the treatment of the condition, disease or disorder.

Examples of the indications that may be treated by the compounds disclosed herein include but are not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as osteoarthritis, osteoporosis and osteopetrosis disorders, eye disease, such as glaucoma and macular degeneration, diseases caused by viral infection such as HIV and AIDS, autoimmune disease such as rheumatoid arthritis, systemic Lupus erythematosus, autoimmune thyroiditis; Addison's disease, pernicious anemia, cancer and aging.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP3 molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a modulator of NLRP3, or a pharmaceutically acceptable salt and/or hydrate and/or cocrystal thereof) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, saturated or unsaturated, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six-membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocyclic rings include five-membered, six-membered, and seven-membered heterocyclic rings.

The term “cycloalkyl” as used herein includes an nonaromatic cyclic, bicylic, fused, or spiro hydrocarbon radical having 3 to 10 carbons, such as 3 to 8 carbons, such as 3 to 7 carbons, wherein the cycloalkyl group which may be optionally substituted. Examples of cycloalkyls include five-membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to an nonaromatic 5-8 membered monocyclic, 8-12 membered bicyclic, or 11-14 membered tricyclic ring, fused, or spiro system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like.

The term “aryl” is intended to mean an aromatic ring radical containing 6 to 10 ring carbons. Examples include phenyl and naphthyl.

The term “heteroaryl” is intended to mean an aromatic ring system containing 5 to 14 aromatic ring atoms that may be a single ring, two fused rings or three fused rings wherein at least one aromatic ring atom is a heteroatom selected from, but not limited to, the group consisting of O, S and N. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

The term “hydroxy” refers to an OH group.

The term “amino” refers to an NH₂ group.

The term “oxo” refers to O. By way of example, substitution of a CH₂ a group with oxo gives a C═O group.

As used herein, the terms “the ring A” or “A” are used interchangeably to denote

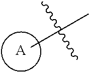

in formula AA, wherein the bond that is shown as being broken by the wavy line [symbol] connects A to Z in Formula AA.

As used herein, the terms “the ring B” or “B” are used interchangeably to denote

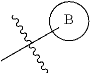

in formula AA wherein the bond that is shown as being broken by the wavy line [symbol] connects B to Y in Formula AA.

As used herein, the term “the optionally substituted ring A” is used to denote

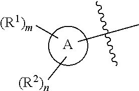

in formula AA, wherein the bond that is shown as being broken by the wavy line [symbol] connects A to Z in Formula AA.

As used herein, the term “the substituted ring B” is used to denote

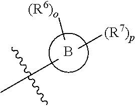

in formula AA, wherein the bond that is shown as being broken by the wavy line [symbol] connects B to Y in Formula AA.

As used herein, the recitation “S(O₂)”, alone or as part of a larger recitation, refers to the group

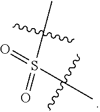 [SMILES: *S(*)(=O)=O]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The scope of the compounds disclosed herein includes tautomeric form of the compounds. Thus, by way of example, a compound that is represented as containing the moiety

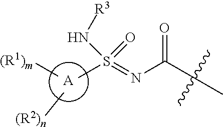

is also intended to include the tautomeric form containing the moiety

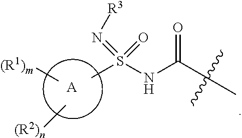

In addition, by way of example, a compound that is represented as containing the moiety

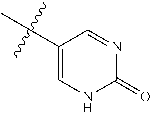 [SMILES: *c1cnc(=O)[nH]c1]

is also intended to include the tautomeric form containing the moiety

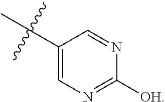 [SMILES: *c1cnc(O)nc1]

Non-limiting exemplified compounds of the formulae described herein include a stereogenic sulfur atom and optionally one or more stereogenic carbon atoms. This disclosure provides examples of stereoisomer mixtures (e.g., racemic mixture of enantiomers; mixture of diastereomers). This disclosure also describes and exemplifies methods for separating individual components of said stereoisomer mixtures (e.g., resolving the enantiomers of a racemic mixture). In cases of compounds containing only a stereogenic sulfur atom, resolved enantiomers are graphically depicted using one of the two following formats: formulas A/B (hashed and solid wedge three-dimensional representation); and formula C (“flat structures with *-labelled stereogenic sulfur).

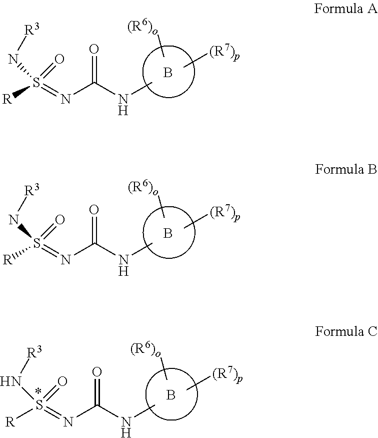

In reaction schemes showing resolution of a racemic mixture, Formulas A/B and C are intended only to convey that the constituent enantiomers were resolved in enantiopure pure form (about 98% ee or greater). The schemes that show resolution products using the formula A/B format are not intended to disclose or imply any correlation between absolute configuration and order of elution. Some of the compounds shown in the tables below are graphically represented using the formula A/B format.

The details of one or more embodiments of the invention are set forth in the accompanying drawings and the description below. Other features and advantages of the invention will be apparent from the description and drawings, and from the claims.

### DESCRIPTION OF THE DRAWINGS

FIG. 1: Expression levels of RNA encoding NLRP3 in Crohn's Disease patients who are responsive and non-responsive to infliximab.

FIG. 2: Expression levels of RNA encoding IL-1β in Crohn's Disease patients who are responsive and non-responsive to infliximab.

FIG. 3: Expression levels of RNA encoding NLRP3 in Ulcerative Colitis (UC) patients who are responsive and non-responsive to infliximab.

FIG. 4: Expression levels of RNA encoding IL-1β in Ulcerative Colitis (UC) patients who are responsive and non-responsive to infliximab.

### DETAILED DESCRIPTION

In some embodiments, provided herein is a compound of Formula AA

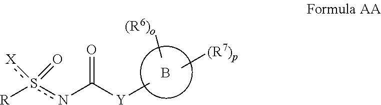

wherein

### R is: —Z-Q, or NR′R″;

### Q is:

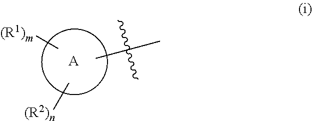

wherein ring A is selected from the group consisting of 5- to 10-membered heteroaryl, C₆-C₁₀ aryl, C₃-C₁₀ cycloalkyl, and 3-10-membered heterocycloalkyl; or

### (ii) H

### Z is:

(i) C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
(ii) 3-10-membered heterocycloalkylene optionally substituted by one or more R¹ and/or R²; or
(iii) C₃-C₁₀ cycloalkyl optionally substituted by one or more R¹ and/or R²;
R′ and R″ are each independently selected from:

### (i) Q; or

(ii) Z″-Q, wherein Z″ is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
or alternatively, wherein R′ and R″ are taken together with the N to which they are attached to form a 5-10-membered heterocycloalkyl ring optionally substituted with one or more R¹ and/or R²; [symbol]
represents a single or double bond;
wherein one of the following apply:
(i) When X is NHR³, a single bond is present between X and S, a double bond is present between S and N, and Y is selected from NH and CR⁴R⁵; or
(ii) When X is O, a double bond is present between X and S, a single bond is present between S and N, the N that is bonded to S is further substituted with an H, and Y is CR⁴R⁵;
   B is selected from the group consisting of 5-membered heteroaryl, 7-10 membered heteroaryl, and C₆-C₁₀ aryl;
   m=0, 1, or 2;
   n=0, 1, or 2;
   R¹ and R² are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
   wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R¹⁶ and R¹⁷ are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹²,
   wherein the C₁-C₆ alkyl and C₁-C₆ haloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   R³ is selected from hydrogen, hydroxy, C₁-C₆ alkoxy, C₁-C₆ alkyl, and

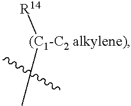

wherein the C₁-C₆ alkylene group is optionally substituted by oxo;
each of R⁴ and R⁵ is independently selected from hydrogen and C₁-C₆ alkyl;
o=1 or 2;
p=0, 1, 2, or 3;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹⁰ is C₁-C₆ alkyl;
   each of R¹¹ and R¹² at each occurrence is independently selected from hydrogen and C₁-C₆ alkyl;
   R¹³ is C₁-C₆ alkyl, C₆-C₁₀ aryl, or 5- to 10-membered heteroaryl;
   R¹⁴ is hydrogen, hydroxy, C₁-C₆ alkyl, NR⁸R⁹, 5- to 10-membered monocyclic or bicyclic heteroaryl, or C₆-C₁₀ monocyclic or bicyclic aryl, wherein each C₁-C₆ alkyl, aryl or heteroaryl is optionally independently substituted with 1 or 2 R⁶,
   each R¹⁵ at each occurrence are each independently selected from the group consisting of C₁-C₆ alkyl, C₁-C₆ haloalkyl, C₁-C₆ alkoxy, C₁-C₆ haloalkoxy, CN, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl, wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹⁵ C₃-C₇ cycloalkyl or of the R¹⁵ 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   with the proviso that the compound of Formula AA is not the following structure:

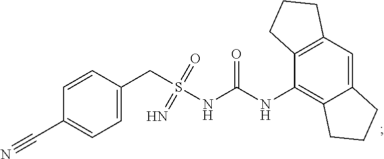 [SMILES: N#Cc1ccc(CS(=N)(=O)NC(=O)Nc2c3c(cc4c2CCC4)CCC3)cc1]

or a pharmaceutically acceptable salt thereof.

In some embodiments of Formula AA, R is Z-Q.

In some embodiments of Formula AA, R is NR′R″.

In some embodiments of Formula AA, R is NR′R″, and R′ and R″ are each independently selected from —Z″—H, wherein Z″ is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O).

In some embodiments of Formula AA, R is NR′R″, and R′ and R″ are taken together with the N to which they are attached to form a 5-10-membered heterocycloalkyl ring optionally substituted with one or more R¹ and/or R²;

In some embodiments of Formula AA, X is NHR³, a single bond is present between X and S, and a double bond is present between S and N; and the compound of Formula AA is a compound of Formula AA-1, Formula AA-2, or Formula AA-3:

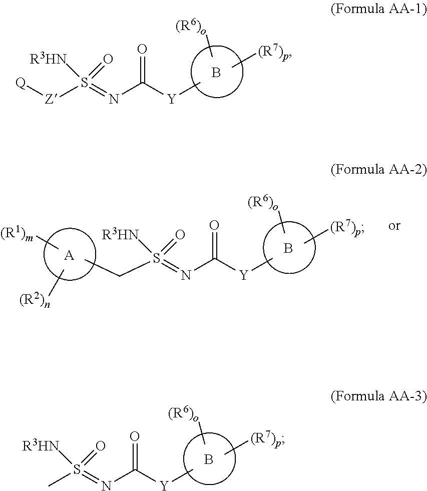

wherein

### Z′ is:

(i) C₂-C₈ alkylene having from 2-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
(ii) CHR¹⁶, CHR¹⁷, CR¹⁶R¹⁶, CR¹⁷R¹⁷, CR¹⁶R¹⁷, or C(O);
(ii) 3-10-membered heterocycloalkylene optionally substituted by one or more R¹ and/or R²; or
(iii) C₃-C₁₀ cycloalkyl optionally substituted by one or more R¹ and/or R²; and
wherein when

### (i) Formula AA is Formula AA-2,

(ii) ring A is phenyl,
(iii) the sum of m and n is 1, and
(iv) whichever of R¹ and R² that is present is CN;
then the position of the phenyl group that is para to the point of the phenyl group's connection to the sulfur of the S(O)(NHR³)═N moiety is substituted with hydrogen.

In some embodiments the variables shown in the formulae herein are as follows:

### Formula AA:

In some embodiments, the compound is a compound of Formula AA-1:

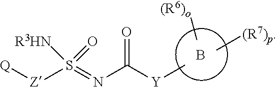

In some embodiments, the compound is a compound of Formula AA-2:

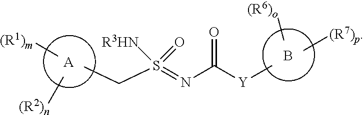

In some embodiments, the compound is a compound of Formula AA-3:

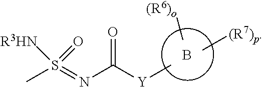

### The Variable R

In some embodiments, R is Z-Q.

### The Variables Z and Z′

### The Variable Z

In some embodiments, Z is
(i) C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
(ii) 3-10-membered heterocycloalkylene optionally substituted by one or more R¹ and/or R²; or
(iii) C₃-C₁₀ cycloalkyl optionally substituted by one or more R¹ and/or R².

In some embodiments, Z is
(i) C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O); or
(ii) 3-10-membered heterocycloalkylene optionally substituted by one or more R¹ and/or R².

In some embodiments, Z is
(i) C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O); or
(iii) C₃-C₁₀ cycloalkyl optionally substituted by one or more R¹ and/or R²;

In some embodiments, Z is
(ii) 3-10-membered heterocycloalkylene optionally substituted by one or more R¹ and/or R²; or
(iii) C₃-C₁₀ cycloalkyl optionally substituted by one or more R¹ and/or R²;

In some embodiments, Z is (i) C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O).

In some embodiments, Z is C₁₋₆alkylene having from 1-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O).

In some embodiments, Z is C₁₋₂alkylene having from 1-2 carbon atoms independently selected from the group consisting of CH₂, CHR¹⁶, CHR¹⁷, CR¹⁶R¹⁶, CR¹⁷R¹⁷, CR¹⁶R¹⁷, and C(O).

In some embodiments, Z is C₁ alkylene having 1 carbon atom selected from the group consisting of CH₂, CHR¹⁶, CHR¹⁷, CR¹⁶R¹⁶, CR¹⁷R¹⁷, CR¹⁶R¹⁷, and C(O).

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is CH₂.

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene comprises C(O).

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is C(O).

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is 1-methyl-1-propyl.

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is 2-methyl-1-propyl.

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is 2,2-dimethyl-1-propyl.

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is ethyl.

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is n-propyl.

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is n-butyl.

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is branched.

In some embodiments (when Z is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is linear.

In some embodiments, Z is (ii) 3-10-membered heterocycloalkylene optionally substituted by one or more R¹ and/or R².

In some embodiments, Z is a 5-6-membered heterocycloalkylene optionally substituted by one or more R¹ and/or R².

In some embodiments, Z is a 5-membered heterocycloalkylene optionally substituted by one or more R¹ and/or R².

In some embodiments, Z is a 6-membered heterocycloalkylene optionally substituted by one or more R¹ and/or R².

In some embodiments, Z is pyrrolidinylene (e.g., 3-pyrrolidinylene) optionally substituted by one or more R¹ and/or R².

In some embodiments, Z is piperidinylene (e.g., 4-piperidinylene) optionally substituted by one or more R¹ and/or R².

In some embodiments, Z is (iii) C₃-C₁₀ cycloalkyl optionally substituted by one or more R¹ and/or R².

In some embodiments, Z is cyclohexyl optionally substituted by one or more R¹ and/or R²

In some embodiments, Z is cyclopentyl optionally substituted by one or more R¹ and/or R².

In some embodiments, Z is cyclobutyl optionally substituted by one or more R¹ and/or R².

In some embodiments, Z is cyclopropyl optionally substituted by one or more R¹ and/or R².

### The Variable Z′ (Applicable to Formula AA-1)

In some embodiments, Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O).

In some embodiments, Z′ is C₂-C₄ alkylene having from 2-4 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O).

In some embodiments, Z′ is C₂ alkylene having 2 carbon atoms independently selected from the group consisting of CH₂, CHR¹⁶, CHR¹⁷, CR¹⁶C¹⁶, CR¹⁷R¹⁷, CR¹⁶R¹⁷, and C(O).

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene comprises C(O).

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), alkylene is C(O).

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is 1-methyl-1-propyl.

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is 2-methyl-1-propyl.

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is 2,2-dimethyl-1-propyl.

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is ethyl.

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is n-propyl.

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is n-butyl.

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is branched.

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), the alkylene is linear.

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is (ii) 3-10-membered heterocycloalkylene optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is a 5-6-membered heterocycloalkylene optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is a 5-membered heterocycloalkylene optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is a 6-membered heterocycloalkylene optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is pyrrolidinylene (e.g., 3-pyrrolidinylene) optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is piperidinylene (e.g., 4-piperidinylene) optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is (iii) C₃-C₁₀ cycloalkyl optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is cyclohexyl optionally substituted by one or more R¹ and/or R²

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is cyclopentyl optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is cyclobutyl optionally substituted by one or more R¹ and/or R².

In some embodiments (when Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O)), Z′ is cyclopropyl optionally substituted by one or more R¹ and/or R².

### The Variable Q

In some embodiments, Q is:

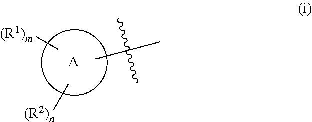

(optionally substituted ring A); or

### (ii) H

In some embodiments, Q is

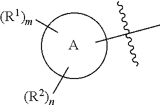

(optionally substituted ring A).

In some embodiments, Q is H.

### The Variables X and Y

In some embodiments, X is NHR³, a single bond is present between X and S, and a double bond is present between S and N.

It is understood that embodiments where X is NHR³, a single bond is present between X and S, and a double bond is present between S and N also cover the tautomeric form where X is NR³, a double bond is present between X and S, a single bond is present between S and N, and a hydrogen is bonded to the N that is single-bonded to the S.

In some embodiments (when X is NHR³, a single bond is present between X and S, and a double bond is present between S and N), Y is NH.

In some embodiments (when X is NHR³, a single bond is present between X and S, and a double bond is present between S and N), Y is CR⁴R⁵.

In some embodiments, X is O, a double bond is present between X and S, a single bond is present between S and N, the N that is bonded to S is further substituted with an H, and Y is CR⁴R⁵.

### The Variables m and n

In some embodiments m=0, 1, or 2.

In some embodiments m=0 or 1.

In some embodiments m=1 or 2.

In some embodiments m=0 or 2.

In some embodiments m=0.

In some embodiments m=1.

In some embodiments m=2.

In some embodiments n=0, 1, or 2.

In some embodiments n=0 or 1.

In some embodiments n=1 or 2.

In some embodiments n=0 or 2.

In some embodiments n=0.

In some embodiments n=1.

In some embodiments n=2.

In some embodiments, m=0 and n=0.

In some embodiments, m=1 and n=0.

In some embodiments, m=1 and n=1.

### The Ring A and Substitutions on the Ring A

In some embodiments, A is selected from the group consisting of: 5- to 10-membered heteroaryl, C₆-C₁₀ aryl, C₃-C₁₀ cycloalkyl, and 3-10-membered heterocycloalkyl.

In some embodiments, A is selected from the group consisting of 5- to 10-membered heteroaryl, C₆-C₁₀ aryl, and 3-10-membered heterocycloalkyl.

In some embodiments, A is selected from the group consisting of 5- to 10-membered heteroaryl.

In some embodiments, A is selected from the group consisting of C₆-C₁₀ aryl.

In some embodiments, A is selected from the group consisting of 3-10-membered heterocycloalkyl.

In some embodiments, A is a 5- to 10-membered (e.g., 5- to 6-membered) heteroaryl or a C₆-C₁₀ (e.g., C₆) aryl.

In some embodiments, A is a 5- to 10-membered (e.g., 5- to 6-membered) heteroaryl.

In some embodiments, A is a 5-membered heteroaryl containing a sulfur and optionally one or more nitrogens.

In some embodiments, A is a C₆-C₁₀ aryl.

In some embodiments, A is thiophenyl (e.g., 3-thiophenyl).

In some embodiments, A is thiazolyl (e.g., 5-thiazolyl).

In some embodiments, A is pyrazolyl (e.g., 4-pyrazolyl).

In some embodiments, A is isoxazolyl (e.g., 5-isoxazolyl).

In some embodiments, A is phenyl.

In some embodiments, A is pyrrolidinyl (e.g., 2-pyrrolidinyl or 3-pyrrolidinyl).

In some embodiments, A is piperidinyl (e.g., 3-piperidinyl or 4-piperidinyl).

In some embodiments, A is azetidinyl (e.g., 2-azetidinyl).

In some embodiments, A is morpholinyl (e.g., 2-morpholinyl).

In some embodiments, A is pyrrolidinyl (e.g., 2-pyrrolidinyl or 3-pyrrolidinyl).

In some embodiments, A is phenyl optionally substituted with 1 or 2 R¹ and optionally substituted with 1 or 2 R².

In some embodiments, A is naphthyl optionally substituted with 1 or 2 R¹ and optionally substituted with 1 or 2 R².

In some embodiments, A is furanyl optionally substituted with 1 or 2 R¹ and optionally substituted with 1 R².

In some embodiments, A is furanyl optionally substituted with 1 R¹ and optionally substituted with 1 or 2 R².

In some embodiments, A is thiophenyl optionally substituted with 1 or 2 R¹ and optionally substituted with 1 or 2 R².

In some embodiments, A is oxazolyl optionally substituted with 1 or 2 R¹ and optionally substituted with 1 or 2 R².

In some embodiments, A is thiazolyl optionally substituted with 1 or 2 R¹ and optionally substituted with 1 or 2 R².

In some embodiments, A is oxazolyl optionally substituted with 2 R¹ or optionally substituted with 2 R².

In some embodiments, A is thiazolyl optionally substituted with 2 R¹ or optionally substituted with 2 R².

In some embodiments, A is pyrazolyl optionally substituted with 1 or 2 R¹ and optionally substituted with 1 or 2 R².

In some embodiments, A is pyrazolyl optionally substituted with 1 R¹ and optionally substituted with 1 or 2 R².

In some embodiments, A is pyrazolyl optionally substituted with 1 or 2 R¹ and optionally substituted with 1 R².

In some embodiments, A is pyridyl optionally substituted with 1 or 2 R¹ and optionally substituted with 1 or 2 R².

In some embodiments, A is indazolyl optionally substituted with 1 or 2 R¹ and optionally substituted with 1 or 2 R².

In some embodiments, A is phenyl substituted with 1 R¹ and optionally substituted with 1 R².

In some embodiments, A is naphthyl substituted with 1 R¹ and optionally substituted with 1 R².

In some embodiments, A is furanyl substituted with 1 R¹ and optionally substituted with 1 R².

In some embodiments, A is thiophenyl substituted with 1 R¹ and optionally substituted with 1 R².

In some embodiments, A is oxazolyl substituted with 1 R¹ and optionally substituted with 1 R².

In some embodiments, A is thiazolyl substituted with 1 R¹ and optionally substituted with 1 R².

In some embodiments, A is pyrazolyl substituted with 1 R¹ and optionally substituted with 1 R².

In some embodiments, A is pyridyl substituted with 1 R¹ and optionally substituted with 1 R².

In some embodiments, A is indazolyl optionally substituted with 1 R¹ and optionally substituted with 1 R².

In some embodiments, A is phenyl substituted with 1 R¹ and substituted with 1 R².

In some embodiments, A is furanyl substituted with 1 R¹ and substituted with 1 R².

In some embodiments, A is thiophenyl substituted with 1 R¹ and substituted with 1 R².

In some embodiments, A is oxazolyl substituted with 1 R¹ and substituted with 1 R².

In some embodiments, A is thiazolyl substituted with 1 R¹ and substituted with 1 R².

In some embodiments, A is pyrazolyl substituted with 1 R¹ and substituted with 1 R².

In some embodiments, A is pyridyl substituted with 1 R¹ and substituted with 1 R².

In some embodiments, A is pyrrolidinyl (e.g., 2-pyrrolidinyl or 3-pyrrolidinyl) substituted with 1 R¹.

In some embodiments, A is piperidinyl (e.g., 3-piperidinyl or 4-piperidinyl) substituted with 1 R¹.

In some embodiments, A is azetidinyl (e.g., 2-azetidinyl) substituted with 1

In some embodiments, A is morpholinyl (e.g., 2-morpholinyl) substituted with 1

In some embodiments, A is pyrrolidinyl (e.g., 2-pyrrolidinyl or 3-pyrrolidinyl) substituted with 1 R¹ and substituted with 1 R².

In some embodiments, A is piperidinyl (e.g., 3-piperidinyl or 4-piperidinyl) substituted with 1 R¹ and substituted with 1 R².

In some embodiments, A is azetidinyl (e.g., 2-azetidinyl) substituted with 1 R¹ and substituted with 1 R².

In some embodiments, A is morpholinyl (e.g., 2-morpholinyl) substituted with 1 R¹ and substituted with 1 R².

In some embodiments, A is phenyl, m is 0 or 1, and n is 0, 1, or 2.

In some embodiments, A is furanyl, m is 0 or 1, and n is 0, 1, or 2.

In some embodiments, A is thiophenyl, m is 0 or 1, and n is 0, 1, or 2.

In some embodiments, A is oxazolyl, m is 0 or 1, and n is 0, 1, or 2.

In some embodiments, A is thiazolyl, m is 0 or 1, and n is 0, 1, or 2.

In some embodiments, A is pyrazolyl, m is 0 or 1, and n is 0, 1, or 2.

In some embodiments, A is pyridyl, m is 0 or 1, and n is 0, 1, or 2.

In some embodiments, A is indazolyl, m is 0 or 1, and n is 0, 1, or 2.

In some embodiments, A is phenyl, m is 0, and n is 0 or 1.

In some embodiments, A is furanyl, m is 0, and n is 0 or 1.

In some embodiments, A is thiophenyl, m is 0, and n is 0 or 1.

In some embodiments, A is oxazolyl, m is 0, and n is 0 or 1.

In some embodiments, A is thiazolyl, m is 0, and n is 0 or 1.

In some embodiments, A is pyrazolyl, m is 0, and n is 0 or 1.

In some embodiments, A is pyridyl, m is 0, and n is 0 or 1.

In some embodiments, A is pyrrolidinyl (e.g., 2-pyrrolidinyl or 3-pyrrolidinyl), m is 0, and n is 0 or 1.

In some embodiments, A is piperidinyl (e.g., 3-piperidinyl or 4-piperidinyl), m is 0, and n is 0 or 1.

In some embodiments, A is azetidinyl (e.g., 2-azetidinyl), m is 0, and n is 0 or 1.

In some embodiments, A is morpholinyl (e.g., 2-morpholinyl), m is 0, and n is 0 or 1.

In some embodiments, A is norbornanyl.

In some embodiments, A is one of the rings disclosed hereinbelow optionally substituted as disclosed hereinbelow, wherein in each case the bond that is shown as being broken by the wavy line [symbol] connects A to the Z variable in Formula AA.

In some embodiments, the optionally substituted ring A

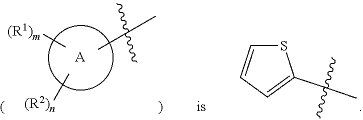

In some embodiments, the optionally substituted ring A is

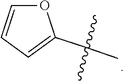 [SMILES: *c1ccco1]

In some embodiments, the optionally substituted ring A is

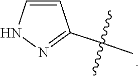 [SMILES: *c1cc[nH]n1]

In some embodiments, the optionally substituted ring A is

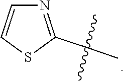 [SMILES: *c1nccs1]

In some embodiments, the optionally substituted ring A is

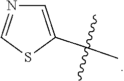 [SMILES: *c1cncs1]

In some embodiments, the optionally substituted ring A is

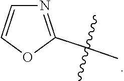 [SMILES: *c1ncco1]

In some embodiments, the optionally substituted ring A is

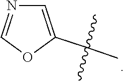 [SMILES: *c1cnco1]

In some embodiments, the optionally substituted ring A is

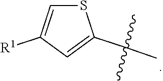

In some embodiments, the optionally substituted ring A is

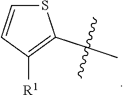

In some embodiments, the optionally substituted ring A is

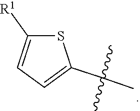

In some embodiments, the optionally substituted ring A is

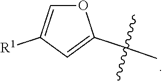

In some embodiments, the optionally substituted ring A is

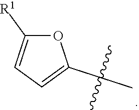

In some embodiments, the optionally substituted ring A is

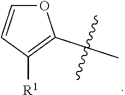

In some embodiments, the optionally substituted ring A is

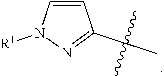

In some embodiments, the optionally substituted ring A is

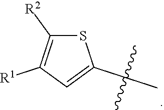

In some embodiments, the optionally substituted ring A is

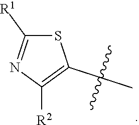

In some embodiments, the optionally substituted ring A is

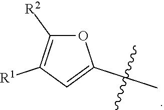

In some embodiments, the optionally substituted ring A is

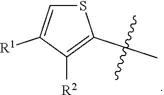

In some embodiments, the optionally substituted ring A is

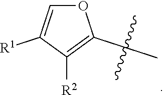

In some embodiments, the optionally substituted ring A is

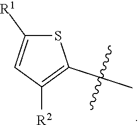

In some embodiments, the optionally substituted ring A is

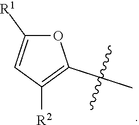

In some embodiments, the optionally substituted ring A is

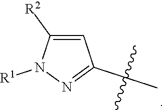

In some embodiments, the optionally substituted ring A is

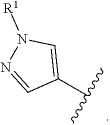

In some embodiments, the optionally substituted ring A is

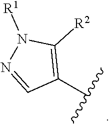

In some embodiments, the optionally substituted ring A is

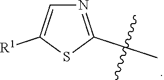

In some embodiments, the optionally substituted ring A is

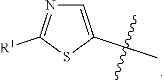

In some embodiments, the optionally substituted ring A is

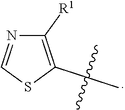

In some embodiments, the optionally substituted ring A is

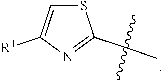

In some embodiments, the optionally substituted ring A is

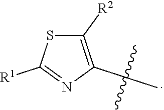

In some embodiments, the optionally substituted ring A is

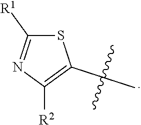

In some embodiments, the optionally substituted ring A is

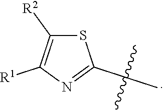

In some embodiments, the optionally substituted ring A is

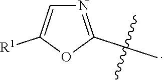

In some embodiments, the optionally substituted ring A is

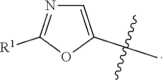

In some embodiments, the optionally substituted ring A is

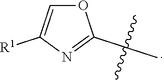

In some embodiments, the optionally substituted ring A is

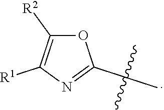

In some embodiments, the optionally substituted ring A is

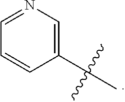 [SMILES: *c1cccnc1]

In some embodiments, the optionally substituted ring A is

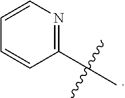 [SMILES: *c1ccccn1]

In some embodiments, the optionally substituted ring A is

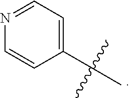 [SMILES: *c1ccncc1]

In some embodiments, the optionally substituted ring A

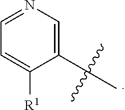

In some embodiments, the optionally substituted ring A is

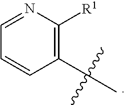

In some embodiments, the optionally substituted ring A is

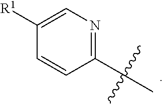

In some embodiments, the optionally substituted ring A is

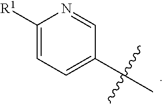

In some embodiments, the optionally substituted ring A is

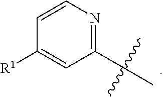

In some embodiments, the optionally substituted ring A is

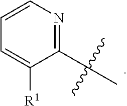

In some embodiments, the optionally substituted ring A is

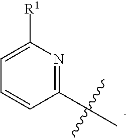

In some embodiments, the optionally substituted ring A is

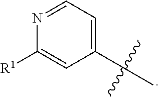

In some embodiments, the optionally substituted ring A is

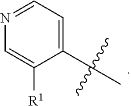

In some embodiments, the optionally substituted ring A is

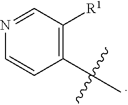

In some embodiments, the optionally substituted ring A is

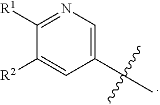

In some embodiments, the optionally substituted ring A is

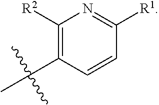

In some embodiments, the optionally substituted ring A is

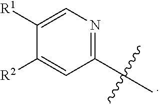

In some embodiments, the optionally substituted ring A is

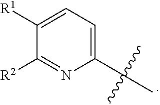

In some embodiments, the optionally substituted ring A is

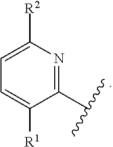

In some embodiments, the optionally substituted ring A is

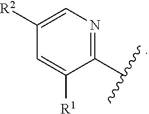

In some embodiments, the optionally substituted ring A is

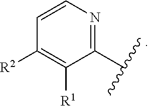

In some embodiments, the optionally substituted ring A is

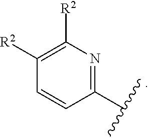

In some embodiments, the optionally substituted ring A is

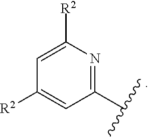

In some embodiments, the optionally substituted ring A is

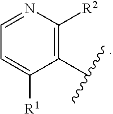

In some embodiments, the optionally substituted ring A is

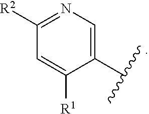

In some embodiments, the optionally substituted ring A is

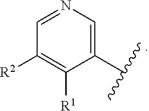

In some embodiments, the optionally substituted ring A is

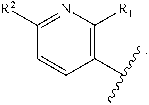

In some embodiments, the optionally substituted ring A is

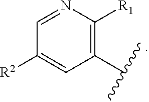

In some embodiments, the optionally substituted ring A is

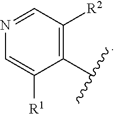

In some embodiments, the optionally substituted ring A is

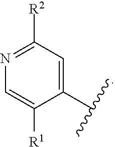

In some embodiments, the optionally substituted ring A is

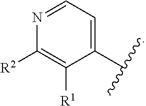

In some embodiments, the optionally substituted ring A is

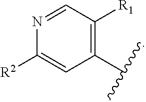

In some embodiments, the optionally substituted ring A is

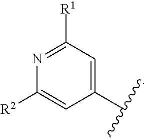

In some embodiments, the optionally substituted ring A is

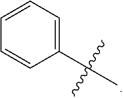 [SMILES: *c1ccccc1]

In some embodiments, the optionally substituted ring A is

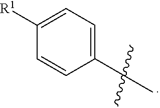

In some embodiments, the optionally substituted ring A

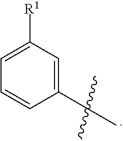

In some embodiments, the optionally substituted ring A is

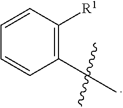

In some embodiments, the optionally substituted ring A is

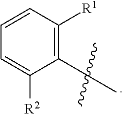

In some embodiments, the optionally substituted ring A is

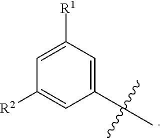

In some embodiments, the optionally substituted ring A is

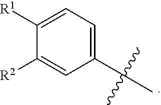

In some embodiments, the optionally substituted ring A is

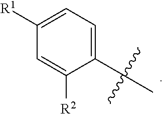

In some embodiments, the optionally substituted ring A is

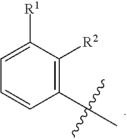

In some embodiments, the optionally substituted ring A is

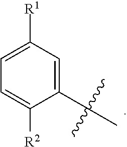

In some embodiments, the optionally substituted ring A is

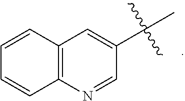 [SMILES: *c1cnc2ccccc2c1]

In some embodiments, the optionally substituted ring A is

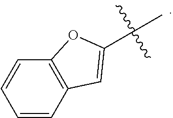 [SMILES: *c1cc2ccccc2o1]

In some embodiments, the optionally substituted ring A is

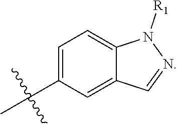

In some embodiments, the optionally substituted ring A is

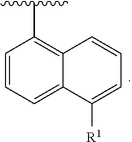

In some embodiments, the optionally substituted ring A is

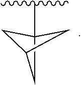 [SMILES: *C12CC(C1)C2]

In some embodiments, the optionally substituted ring A

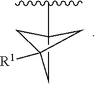

In some embodiments, the optionally substituted ring A is

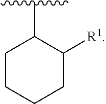

In some embodiments, the optionally substituted ring A is

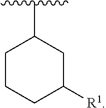

In some embodiments, the optionally substituted ring A is

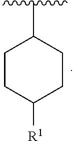

In some embodiments, the optionally substituted ring A is

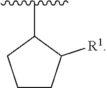

In some embodiments, the optionally substituted ring A is

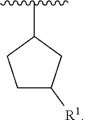

In some embodiments, the optionally substituted ring A is

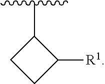

In some embodiments, the optionally substituted ring A is

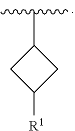

In some embodiments, the optionally substituted ring A is

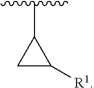

In some embodiments, the optionally substituted ring A is

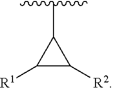

In some embodiments, the optionally substituted ring A is

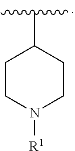

In some embodiments, the optionally substituted ring A is

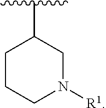

In some embodiments, the optionally substituted ring A is

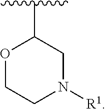

In some embodiments, the optionally substituted ring A is

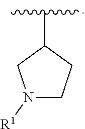

In some embodiments, the optionally substituted ring A is

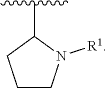

In some embodiments, the optionally substituted ring A is

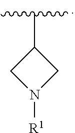

In some embodiments, the optionally substituted ring A is

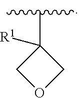

In some embodiments, the optionally substituted ring A is

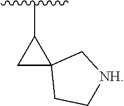 [SMILES: *C1CC12CCNC2]

In some embodiments, the optionally substituted ring A is

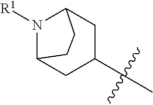

The Groups R¹ and R²

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently
   substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;

In some embodiments,

R¹ and R² are each independently selected from C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, halo, CN, NO₂, COC₁-C₆ alkyl, CO—C₆-C₁₀ aryl, CO(5- to 10-membered heteroaryl), CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl are each unsubstituted;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl, wherein the C₁-C₆ alkyl is optionally substituted with one or more substituents each independently selected from hydroxy, halo, oxo, C₁-C₆ alkoxy, and NR⁸R⁹.

In some embodiments,

R¹ and R² are each independently selected from C₁-C₆ alkyl, halo, CN, COC₁-C₆ alkyl, CO₂C₁-C₆ alkyl, C₆-C₁₀ aryl, S(O)C₁-C₆ alkyl, 5- to 10-membered heteroaryl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, m=1; n=0; and

R¹ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, m=1; n=0; and,

R¹ is selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, m=1; n=1; and

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, m=1; n=1; and,

R¹ and R² are each independently selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,
wherein the C₁-C₆ alkyl and 3- to 7-membered heterocycloalkyl is optionally substituted with one or more substituents each independently selected from hydroxy and oxo.

In some embodiments, m=1; n=1; and

R¹ and R² are on adjacent atoms, and taken together with the atoms connecting them, form a C₄-C₈ carbocyclic ring or a 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₅ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, R¹ and R² are each independently selected from methyl, ethyl, isopropyl, 2-hydroxy-2-propyl, dimethylamino, aminomethyl, methylaminomethyl, dimethylaminomethyl, methoxycarbonyl, and carboxyl.

Particular Embodiments Wherein Ring a is Substituted with One R¹ (i.e., m=1 and n=0) and/or Wherein the 3-10-Membered Heterocycloalkylene or C₃-C₁₀ Cycloalkyl of Z is Substituted with One R¹:

In some embodiments, R¹ is C₁-C₆ alkyl optionally substituted with one or more hydroxy.

In some embodiments, R¹ is 1-hydroxy-2-methylpropan-2-yl.

In some embodiments, 10 is 2-hydroxyethyl.

In some embodiments, R¹ is C₁-C₆ alkyl.

In some embodiments, R¹ is methyl.

In some embodiments, R¹ is isopropyl.

In some embodiments, R¹ is isopropyl.

In some embodiments, R¹ is C₁-C₆ alkyl substituted with hydroxy at the carbon directly connected to ring A.

In some embodiments, R¹ is 2-hydroxy-2-propyl.

In some embodiments, 10 is hydroxymethyl.

In some embodiments, 10 is 1-hydroxyethyl.

In some embodiments, R¹ is 1-hydroxy-2-propyl.

In some embodiments, R¹ is C₁-C₆ alkyl substituted with two or more hydroxy groups.

In some embodiments, R¹ is C₁-C₆ alkyl substituted with two or more hydroxy groups, wherein one of the two or more hydroxy groups is bonded to the carbon directly connected to ring A.

In some embodiments, R¹ is 1,2-dihydroxy-prop-2-yl.

In some embodiments, R¹ is C₃-C₇ cycloalkyl optionally substituted with one or more hydroxy.

In some embodiments, IV is C₃-C₇ cycloalkyl.

In some embodiments, IV is C₃-C₇ cycloalkyl substituted with hydroxy at the carbon directly connected to ring A.

In some embodiments, 10 is 1-hydroxy-1-cyclopropyl.

In some embodiments, R¹ is 1-hydroxy-1-cyclobutyl.

In some embodiments, 10 is 1-hydroxy-1-cyclopentyl.

In some embodiments, 10 is 1-hydroxy-1-cyclohexyl.

In some embodiments, IV is 3- to 7-membered heterocycloalkyl optionally substituted with one or more hydroxy.

In some embodiments, R¹ is 3- to 7-membered heterocycloalkyl.

In some embodiments, R¹ is morpholinyl (e.g., 1-morpholinyl).

In some embodiments, R¹ is 1,3-dioxolan-2-yl.

In some embodiments, IV is 3- to 7-membered heterocycloalkyl optionally substituted with one or more C₁-C₆ alkyl.

In some embodiments, R¹ is 1-methylpyrrolidin-2-yl.

In some embodiments, IV is 3- to 7-membered heterocycloalkyl substituted with hydroxy at the carbon directly connected to ring A.

In some embodiments, R¹ is C₁-C₆ alkyl optionally substituted with one or more oxo.

In some embodiments, IV is COCH₃.

In some embodiments, R¹ is COCH₂CH₃.

In some embodiments, R¹ is C₃-C₇ cycloalkyl optionally substituted with one or more oxo.

In some embodiments, R¹ is 3- to 7-membered heterocycloalkyl optionally substituted with one or more oxo.

In some embodiments, R¹ is C₁-C₆ alkyl optionally substituted with one or more C₁-C₆ alkoxy.

In some embodiments, R¹ is 2-methoxy-2-propyl.

In some embodiments, R¹ is methoxymethyl.

In some embodiments, IV is C₃-C₇ cycloalkyl optionally substituted with one or more C₁-C₆ alkoxy.

In some embodiments, R¹ is 3- to 7-membered heterocycloalkyl optionally substituted with one or more C₁-C₆ alkoxy.

In some embodiments, R¹ is C₁-C₆ alkyl optionally substituted with one or more NR⁸R⁹.

In some embodiments, R¹ is C₁-C₆ alkyl substituted with NR⁸R⁹ at the carbon directly connected to ring A.

In some embodiments, R¹ is (methylamino)methyl.

In some embodiments, R¹ is (dimethylamino)methyl.

In some embodiments, R¹ is aminomethyl.

In some embodiments, R¹ is N-methylacetamidomethyl.

In some embodiments, R¹ is 1-(dimethylamino)eth-1-yl.

In some embodiments, R¹ is 2-(dimethylamino)prop-2-yl.

In some embodiments, 10 is (2-methoxy-eth-1-yl)(methyl)aminomethyl.

In some embodiments, 10 is (methyl)(acetyl)aminomethyl.

In some embodiments, R¹ is (methyl)(cyclopropylmethyl)aminomethyl.

In some embodiments, R¹ is (methyl)(2,2-difluoroeth-1-yl)aminomethyl.

In some embodiments, R¹ is C₃-C₇ cycloalkyl optionally substituted with one or more NR⁸R⁹.

In some embodiments, R¹ is 3- to 7-membered heterocycloalkyl optionally substituted with one or more NR⁸R⁹.

In some embodiments, R¹ is C₁-C₆ haloalkyl optionally substituted with one or more hydroxy.

In some embodiments, R¹ is C₁-C₆ alkoxy.

In some embodiments, R¹ is C₁-C₆ haloalkoxy.

In some embodiments, R¹ is C₁-C₆ alkyl optionally substituted with 3- to 7-membered heterocycloalkyl, wherein the 3- to 7-membered heterocycloalkyl is further optionally substituted as defined elsewhere herein.

In some embodiments, R¹ is pyrrolidinylmethyl (e.g., pyrrolidin-1-ylmethyl).

In some embodiments, R¹ is optionally substituted pyrrolidinylmethyl (e.g., 3,3-difluoropyrrolidin-1-ylmethyl).

In some embodiments, R¹ is azetidinylmethyl (e.g., azetidin-1-ylmethyl).

In some embodiments, R¹ is optionally substituted azetidinylmethyl (e.g., 3-methoxyazetidin-1-ylmethyl).

In some embodiments, R¹ is morpholinylmethyl (e.g., morpholin-4-ylmethyl).

In some embodiments, R¹ is halo.

In some embodiments, R¹ is fluoro.

In some embodiments, R¹ is chloro.

In some embodiments, R¹ is CN.

In some embodiments, R¹ is NO₂

In some embodiments, R¹ is COC₁-C₆ alkyl.

In some embodiments, R¹ is CO—C₆-C₁₀ aryl.

In some embodiments, R¹ is CO(5- to 10-membered heteroaryl).

In some embodiments, R¹ is CO₂C₁-C₆ alkyl.

In some embodiments, R¹ is CO₂C₃-C₈ cycloalkyl.

In some embodiments, R¹ is OCOC₁-C₆ alkyl.

In some embodiments, R¹ is OCOC₆-C₁₀ aryl.

In some embodiments, R¹ is OCO(5- to 10-membered heteroaryl).

In some embodiments, R¹ is OCO(3- to 7-membered heterocycloalkyl).

In some embodiments, R¹ is C₆-C₁₀ aryl.

In some embodiments, R¹ is phenyl.

In some embodiments, R¹ is 5- to 10-membered heteroaryl.

In some embodiments, R¹ is pyridyl (e.g., 4-pyridyl).

In some embodiments, R₁ is pyrazolyl (e.g., 1-pyrazolyl).

In some embodiments, R¹ is NH₂.

In some embodiments, R¹ is NHC₁-C₆ alkyl.

In some embodiments, R¹ is N(C₁-C₆ alkyl)₂.

In some embodiments, R¹ is CONR⁸R⁹.

In some embodiments, R¹ is SF₅.

In some embodiments, R¹ is SC₁-C₆ alkyl,

In some embodiments, R¹ is S(O₂)C₁-C₆ alkyl.

In some embodiments, R¹ is S(O₂)CH₃.

In some embodiments, R¹ is S(O₂)NR¹¹R¹².

In some embodiments, R¹ is S(O₂)N(CH₃)₂.

In some embodiments, R¹ is S(O)C₁-C₆ alkyl.

In some embodiments, R¹ is S(O)CH₃.

In some embodiments, R¹ is attached to a carbon of an aryl ring A.

In some embodiments, R¹ is attached to a carbon of a heteroaryl ring A.

In some embodiments, R¹ is attached to a nitrogen of a heteroaryl ring A.

Particular Embodiments Wherein Ring a is Substituted with One R¹ and One R² (i.e., m=1 and n=1) and/or Wherein the 3-10-Membered Heterocycloalkylene or C₃-C₁₀ Cycloalkyl of Z is Substituted with One R¹ and One R²:

In some embodiments, R¹ is C₁-C₆ alkyl optionally substituted with one or more hydroxy, and R² is C₁-C₆ alkyl optionally substituted with one or more hydroxy.

In some embodiments, 10 is 1-hydroxy-2-methylpropan-2-yl, and R² is methyl.

In some embodiments, 10 is 2-hydroxy-2-propyl and R² is methyl.

In some embodiments, 10 is 2-hydroxy-2-propyl and R² is isopropyl.

In some embodiments, 10 is 2-hydroxy-2-propyl and R² is 2-hydroxy-2-propyl.

In some embodiments, 10 is 2-hydroxy-2-propyl and R² is 1-hydroxyethyl.

In some embodiments, 10 is hydroxymethyl and R² is methyl.

In some embodiments, 10 is 1-hydroxyethyl and R² is methyl.

In some embodiments, 10 is 2-hydroxyethyl and R² is methyl.

In some embodiments, 10 is 1-hydroxy-2-propyl and R² is methyl.

In some embodiments, R¹ is C₁-C₆ alkyl optionally substituted with one or more hydroxy, and R² is C₆-C₁₀ aryl.

In some embodiments, 10 is 2-hydroxy-2-propyl and R² is phenyl.

In some embodiments, R¹ is C₁-C₆ alkyl optionally substituted with one or more hydroxy, and R² is 5- to 10-membered heteroaryl.

In some embodiments, 10 is 2-hydroxy-2-propyl and R² is pyridyl.

In some embodiments, 10 is 2-hydroxy-2-propyl and R² is pyrazolyl.

In some embodiments, R¹ is C₁-C₆ alkyl optionally substituted with one or more hydroxy, and R² is SF₅.

In some embodiments, R¹ is C₁-C₆ alkyl optionally substituted with one or more hydroxy, and R² is SC₁-C₆ alkyl,

In some embodiments, R¹ is C₁-C₆ alkyl optionally substituted with one or more hydroxy, and R² is S(O₂)C₁-C₆ alkyl.

In some embodiments, R¹ is C₁-C₆ alkyl optionally substituted with one or more hydroxy, and R² is S(O₂)CH₃.

In some embodiments, R¹ is C₁-C₆ alkyl optionally substituted with one or more hydroxy, and R² is halo.

In some embodiments, 10 is 2-hydroxy-2-propyl and R² is chloro.

In some embodiments, 10 is 2-hydroxy-2-propyl and R² is fluoro.

In some embodiments, R¹ is C₃-C₇ cycloalkyl optionally substituted with one or more hydroxy, and R² is C₁-C₆ alkyl.

In some embodiments, 10 is 1-hydroxy-1-cyclopropyl, and R² is methyl.

In some embodiments, 10 is 1-hydroxy-1-cyclobutyl, and R² is methyl.

In some embodiments, 10 is 1-hydroxy-1-cyclopentyl, and R² is methyl.

In some embodiments, 10 is 1-hydroxy-1-cyclohexyl, and R² is methyl.

In some embodiments, R¹ is 3- to 7-membered heterocycloalkyl optionally substituted with one or more hydroxy, and R² is C₁-C₆ alkyl.

In some embodiments, R¹ is morpholinyl, and R² is methyl.

In some embodiments, R¹ is 1,3-dioxolan-2-yl, and R² is methyl.

In some embodiments, R¹ is 3- to 7-membered heterocycloalkyl optionally substituted with one or more hydroxy, and R² is halo.

In some embodiments, 10 is 1,3-dioxolan-2-yl, and R² is fluoro.

In some embodiments, R¹ is 1,3-dioxolan-2-yl, and R² is chloro.

In some embodiments, R¹ is C₁-C₆ alkyl optionally substituted with one or more oxo, and R² is methyl.

In some embodiments, R¹ is COCH₃, and R² is methyl.

In some embodiments, R¹ is C₁-C₆ alkyl optionally substituted with one or more C₁-C₆ alkoxy, and R² is C₁-C₆ alkyl.

In some embodiments, 10 is 2-methoxy-2-propyl, and R² is methyl.

In some embodiments, R¹ is C₁-C₆ alkyl optionally substituted with one or more NR⁸R⁹, and R² is C₁-C₆ alkyl.

In some embodiments, R¹ is (dimethylamino)methyl, and R² is methyl.

In some embodiments, R¹ is C₁-C₆ alkyl optionally substituted with one or more NR⁸R⁹, and R² is halo.

In some embodiments, R¹ is (dimethylamino)methyl, and R² is fluoro.

In some embodiments, R¹ is (dimethylamino)methyl, and R² is fluoro.

In some embodiments, R¹ is (methylamino)methyl, and R² is fluoro.

In some embodiments, R¹ is aminomethyl, and R² is fluoro.

In some embodiments, R¹ is C₁-C₆ alkyl, and R² is C₁-C₆ alkyl.

In some embodiments, R¹ is methyl, and R² is methyl.

In some embodiments, R² is 1-hydroxy-2-methylpropan-2-yl, and R¹ is methyl.

In some embodiments, R² is 2-hydroxy-2-propyl and R¹ is methyl.

In some embodiments, R² is 2-hydroxy-2-propyl and 10 is isopropyl.

In some embodiments, R² is 2-hydroxy-2-propyl and 10 is 1-hydroxyethyl.

In some embodiments, R² is hydroxymethyl and 10 is methyl.

In some embodiments, R² is 1-hydroxyethyl and 10 is methyl.

In some embodiments, R² is 2-hydroxyethyl and 10 is methyl.

In some embodiments, R² is 1-hydroxy-2-propyl and R¹ is methyl.

In some embodiments, R² is C₁-C₆ alkyl optionally substituted with one or more hydroxy, and R¹ is C₆-C₁₀ aryl.

In some embodiments, R² is 2-hydroxy-2-propyl and R¹ is phenyl.

In some embodiments, R² is C₁-C₆ alkyl optionally substituted with one or more hydroxy, and R¹ is 5- to 10-membered heteroaryl.

In some embodiments, R² is 2-hydroxy-2-propyl and R¹ is pyridyl.

In some embodiments, R² is 2-hydroxy-2-propyl and 10 is pyrazolyl.

In some embodiments, R² is C₁-C₆ alkyl optionally substituted with one or more hydroxy, and R¹ is SF₅.

In some embodiments, R² is C₁-C₆ alkyl optionally substituted with one or more hydroxy, and R¹ is SC₁-C₆ alkyl.

In some embodiments, R² is C₁-C₆ alkyl optionally substituted with one or more hydroxy, and 10 is S(O₂)C₁-C₆ alkyl.

In some embodiments, R² is C₁-C₆ alkyl optionally substituted with one or more hydroxy, and 10 is S(O₂)CH₃.

In some embodiments, R² is C₁-C₆ alkyl optionally substituted with one or more hydroxy, and R¹ is halo.

In some embodiments, R² is 2-hydroxy-2-propyl and 10 is chloro.

In some embodiments, R² is 2-hydroxy-2-propyl and 10 is fluoro.

In some embodiments, R² is C₃-C₇ cycloalkyl optionally substituted with one or more hydroxy, and R¹ is C₁-C₆ alkyl.

In some embodiments, R² is 1-hydroxy-1-cyclopropyl, and 10 is methyl.

In some embodiments, R² is 1-hydroxy-1-cyclobutyl, and 10 is methyl.

In some embodiments, R² is 1-hydroxy-1-cyclopentyl, and 10 is methyl.

In some embodiments, R² is 1-hydroxy-1-cyclohexyl, and 10 is methyl.

In some embodiments, R² is 3- to 7-membered heterocycloalkyl optionally substituted with one or more hydroxy, and R¹ is C₁-C₆ alkyl.

In some embodiments, R² is morpholinyl, and R¹ is methyl.

In some embodiments, R² is 1,3-dioxolan-2-yl, and R¹ is methyl.

In some embodiments, R² is 3- to 7-membered heterocycloalkyl optionally substituted with one or more hydroxy, and 10 is halo.

In some embodiments, R² is 1,3-dioxolan-2-yl, and 10 is fluoro.

In some embodiments, R² is 1,3-dioxolan-2-yl, and R¹ is chloro.

In some embodiments, R² is C₁-C₆ alkyl optionally substituted with one or more oxo, and R¹ is methyl.

In some embodiments, R² is COCH₃, and R¹ is methyl.

In some embodiments, R² is C₁-C₆ alkyl optionally substituted with one or more C₁-C₆ alkoxy, and R¹ is C₁-C₆ alkyl.

In some embodiments, R² is 2-methoxy-2-propyl, and R¹ is methyl.

In some embodiments, R² is C₁-C₆ alkyl optionally substituted with one or more NR⁸R⁹, and

R¹ is C₁-C₆ alkyl.

In some embodiments, R² is (dimethylamino)methyl, and R¹ is methyl.

In some embodiments, R² is C₁-C₆ alkyl optionally substituted with one or more NR⁸R⁹, and R¹ is halo.

In some embodiments, R² is (dimethylamino)methyl, and R¹ is fluoro.

In some embodiments, R² is (methylamino)methyl, and 10 is fluoro.

In some embodiments, R² is aminomethyl, and R¹ is fluoro.

In some embodiments, R² is C₁-C₆ alkoxy, and R¹ is C₁-C₆ alkyl optionally substituted with one or more NR⁸R⁹.

In some embodiments, R² is methoxy, and R¹ is (dimethylamino)methyl.

In some embodiments, R¹ and R² are each attached to a carbon of an aryl ring A.

In some embodiments, R¹ and R² are each attached to a carbon of a heteroaryl ring A.

In some embodiments, R¹ is attached to a carbon and R² is attached to a nitrogen of a heteroaryl ring A.

In some embodiments, R² is attached to a carbon and R¹ is attached to a nitrogen of a heteroaryl ring A.

In some embodiments, R¹ and R² are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a C₅ aliphatic carbocyclic ring.

In some embodiments, R¹ and R² are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a C₆ aliphatic carbocyclic ring.

In some embodiments, R¹ and R² are on adjacent atoms, and taken together with the atoms connecting them, form a C₆ aromatic carbocyclic ring.

In some embodiments, R¹ and R² are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a 5-membered aliphatic heterocyclic ring containing 1 or 2 heteroatoms independently selected from O, N, and S.

In some embodiments, R¹ and R² are on adjacent atoms, and taken together with the atoms connecting them, form a 5-membered heteroaromatic ring containing 1 or 2 heteroatoms independently selected from O, N, and S.

In some embodiments, R¹ and R² are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a 6-membered aliphatic heterocyclic ring containing 1 or 2 heteroatoms independently selected from O, N, and S.

In some embodiments, R¹ and R² are on adjacent atoms, and taken together with the atoms connecting them, form a 6-membered heteroaromatic ring containing 1 or 2 heteroatoms independently selected from O, N, and S.

In some embodiments, R¹ and R² are different.

In some embodiments, R¹ and R² are different, and R² comprises a carbonyl group.

In some embodiments, R¹ and R² are different, and R² comprises 1 or 2 (e.g., 1) nitrogen atoms.

In some embodiments, R¹ and R² are different, and R² comprises 1 or 2 (e.g., 1) oxygen atoms.

In some embodiments, R¹ and R² are different, and R² comprises a sulfur atom.

In some embodiments, R² and R¹ are different, and R² comprises a carbonyl group.

In some embodiments, R² and R¹ are different, and R² comprises 1 or 2 (e.g., 1) nitrogen atoms.

In some embodiments, R² and R¹ are different, and R² comprises 1 or 2 (e.g., 1) oxygen atoms.

In some embodiments, R² and R¹ are different, and R² comprises a sulfur atom. In some embodiments, R¹ and R² are the same.

In some embodiments, R¹ is para or meta to R².

In some embodiments, R¹ is para or ortho to R².

In some embodiments, R¹ is ortho or meta to R². In some embodiments, R¹ is para to R².

In some embodiments, R¹ is meta to R².

In some embodiments, R¹ is ortho to R².

The Groups R¹⁶ and R¹⁷

In some embodiments, R¹⁶ and R¹⁷ are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹²,
wherein the C₁-C₆ alkyl and C₁-C₆ haloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl.

In some embodiments, R¹⁶ and R¹⁷ are each independently selected from C₁-C₆ alkyl, C₁-C₆ alkoxy, C₁-C₆ haloalkoxy, halo, CN, CO₂H, COC₁-C₆ alkyl, CO—C₆-C₁₀ aryl; CO(5- to 10-membered heteroaryl); CO₂C₁-C₆ alkyl, OCOC₁-C₆ alkyl, NH₂, NHC₁-C₆ alkyl, N(C₁-C₆ alkyl)₂, NHCOC₁-C₆ alkyl, NHCOC₆-C₁₀ aryl, NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹²,
wherein the C₁-C₆ alkyl is optionally substituted with one or more substituents each independently selected from hydroxy, halo, CN, oxo, C₁-C₆ alkoxy, NR⁸R⁹, ═NR¹⁰, COOC₁-C₆ alkyl, CONR⁸R⁹, OCOC₁-C₆ alkyl, NHCOC₁-C₆ alkyl, NHCOC₆-C₁₀ aryl, and NHCOC₂-C₆ alkynyl.

In some embodiments, R¹⁶ and R¹⁷ are each independently selected from C₁-C₆ alkyl, C₁-C₆ alkoxy, halo, CN, CO₂H, COC₁-C₆ alkyl, CO₂C₁-C₆ alkyl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, and S(O₂)NR¹¹R¹²,
wherein the C₁-C₆ alkyl is optionally substituted with one or more substituents each independently selected from hydroxy, halo, CN, oxo, C₁-C₆ alkoxy, NR⁸R⁹, CONR⁸R⁹, and NHCOC₁-C₆ alkyl.

In some embodiments, R¹⁶ and R¹⁷ are each independently selected from C₁-C₆ alkoxy, halo, CN, COC₁-C₆ alkyl, CO₂C₁-C₆ alkyl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, and S(O₂)NR¹¹R¹².

In some embodiments, R¹⁶ and R¹⁷ are each independently selected from C₁-C₆ alkoxy, halo, CN, COC₁-C₆ alkyl, CO₂C₁-C₆ alkyl, N(C₁-C₆ alkyl)₂, CONR⁸R⁹, S(O₂)C₁-C₆ alkyl, S(O)C₁-C₆ alkyl, and S(O₂)NR¹¹R¹².

In some embodiments, R¹⁶ and R¹⁷ are each independently selected from C₁-C₆ alkoxy, halo, CN, COC₁-C₆ alkyl, CO₂C₁-C₆ alkyl, N(C₁-C₆ alkyl)₂, CONR⁸R⁹, S(O₂)C₁-C₆ alkyl, S(O)C₁-C₆ alkyl, and S(O₂)NR¹¹R¹².

In some embodiments, R¹⁶ and R¹⁷ are each independently selected from N(C₁-C₆ alkyl)₂.

### The Variables o and p

In some embodiments, o=1 or 2.

In some embodiments, o=1.

In some embodiments, o=2.

In some embodiments, p=0, 1, 2, or 3.

In some embodiments, p=0.

In some embodiments, p=1.

In some embodiments, p=2.

In some embodiments, o=1 and p=0.

In some embodiments, o=2 and p=0.

In some embodiments, o=1 and p=1.

In some embodiments, o=1 and p=2.

In some embodiments, o=2 and p=1.

In some embodiments, o=2 and p=2.

In some embodiments, o=2 and p=3.

### The Ring B and Substitutions on the Ring B

In some embodiments, B is a 5- to 10-membered monocyclic or bicyclic heteroaryl or a C₆-C₁₀ monocyclic or bicyclic aryl, such as phenyl.

In some embodiments, B is a 5- to 6-membered monocyclic heteroaryl or a C₆ monocyclic aryl.

In some embodiments, B is a 5- to 10-membered monocyclic or bicyclic heteroaryl.

In some embodiments, B is a C₆-C₁₀ monocyclic or bicyclic aryl.

In some embodiments, B is a 5-membered heteroaryl.

In some embodiments, B is a 7-10 membered monocyclic or bicyclic heteroaryl.

In some embodiments, B is phenyl substituted with 1 or 2 R⁶ and optionally substituted with

$$1 , 2 , or 3 {R}^{7} .$$

In some embodiments, B is pyridyl substituted with 1 or 2 R⁶ and optionally substituted with 1, 2, or 3 R⁷.

In some embodiments, B is indazolyl substituted with 1 or 2 R⁶ and optionally substituted with 1, 2, or 3 R⁷.

In some embodiments, B is pyrazolyl substituted with 1 or 2 R⁶ and optionally substituted with 1 or 2 R⁷.

In some embodiments, B is phenyl, o is 1 or 2, and p is 0, 1, 2, or 3.

In some embodiments, B is phenyl, o is 1, and p is 0, 1, 2, or 3.

In some embodiments, B is phenyl, o is 2, and p is 0, 1, 2, or 3.

In some embodiments, B is one of the rings disclosed hereinbelow, substituted as disclosed hereinbelow, wherein in each case the bond that is shown as being broken by the wavy line [symbol] connects B to the NH(CO) group of Formula AA.

In some embodiments, the substituted ring B

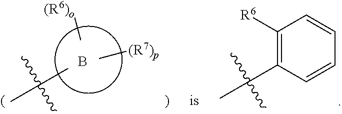

In some embodiments, the substituted ring B

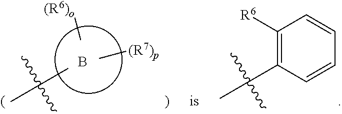

In some embodiments, the substituted ring B is

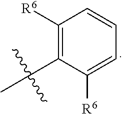

In some embodiments, the substituted ring B is

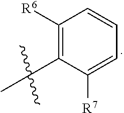

In some embodiments, the substituted ring B is

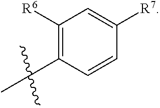

In some embodiments, the substituted ring B is

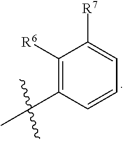

In some embodiments, the substituted ring B is

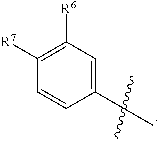

In some embodiments, the substituted ring B is

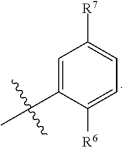

In some embodiments, the substituted ring B is

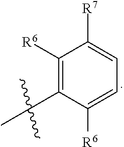

In some embodiments, the substituted ring B is

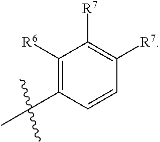

In some embodiments, the substituted ring B is

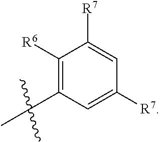

In some embodiments, the substituted ring B is

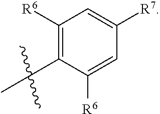

In some embodiments, the substituted ring B is

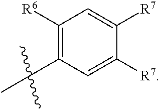

In some embodiments, the substituted ring B is

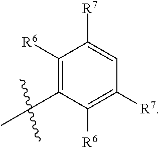

In some embodiments, the substituted ring B is

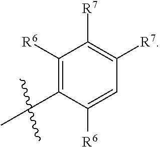

In some embodiments, the substituted ring B is

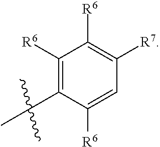

In some embodiments, the substituted ring B is

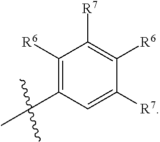

In some embodiments, the substituted ring B is

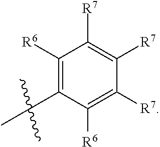

In some embodiments, the substituted ring B is

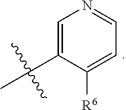

In some embodiments, the substituted ring B is

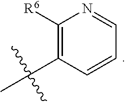

In some embodiments, the substituted ring B is

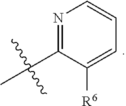

In some embodiments, the substituted ring B is

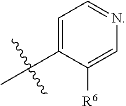

In some embodiments, the substituted ring B is

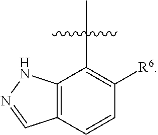

In some embodiments, the substituted ring B is

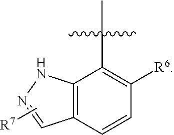

In some embodiments, the substituted ring B is

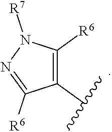

In some embodiments, the substituted ring B is

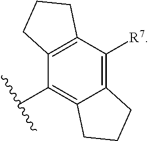

In some embodiments, the substituted ring B is

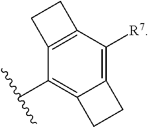

In some embodiments, the substituted ring B is

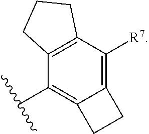

In some embodiments, the substituted ring B is

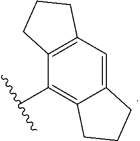 [SMILES: *c1c2c(cc3c1CCC3)CCC2]

In some embodiments, the substituted ring B is

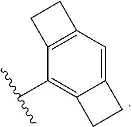 [SMILES: *c1c2c(cc3c1CC3)CC2]

In some embodiments, the substituted ring B is

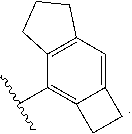 [SMILES: *c1c2c(cc3c1CC3)CCC2]

In some embodiments, the substituted ring B is

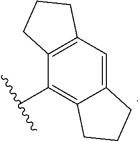 [SMILES: *c1c2c(cc3c1CCC3)CCC2]

In some embodiments, the substituted ring B is

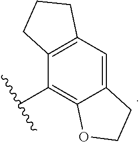 [SMILES: *c1c2c(cc3c1OCC3)CCC2]

In some embodiments, the substituted ring B is

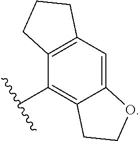 [SMILES: *c1c2c(cc3c1CCO3)CCC2]

In some embodiments, the substituted ring B is

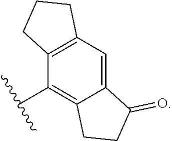 [SMILES: *c1c2c(cc3c1CCC3=O)CCC2]

In some embodiments, the substituted ring B is

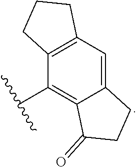 [SMILES: *c1c2c(cc3c1C(=O)CC3)CCC2]

In some embodiments, the substituted ring B is

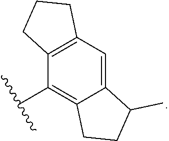 [SMILES: *c1c2c(cc3c1CCC3C)CCC2]

In some embodiments, the substituted ring B is

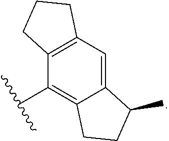 [SMILES: *c1c2c(cc3c1CC[C@@H]3C)CCC2]

In some embodiments, the substituted ring B is

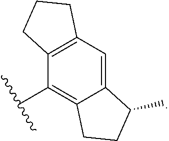 [SMILES: *c1c2c(cc3c1CC[C@H]3C)CCC2]

In some embodiments, the substituted ring B is

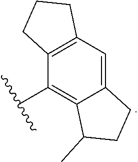 [SMILES: *c1c2c(cc3c1C(C)CC3)CCC2]

In some embodiments, the substituted ring B is

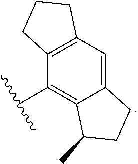 [SMILES: *c1c2c(cc3c1[C@H](C)CC3)CCC2]

In some embodiments, the substituted ring B is

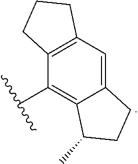 [SMILES: *c1c2c(cc3c1[C@@H](C)CC3)CCC2]

In some embodiments, the substituted ring B is

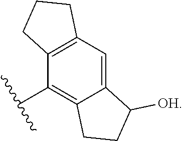 [SMILES: *c1c2c(cc3c1CCC3O)CCC2]

In some embodiments, the substituted ring B is

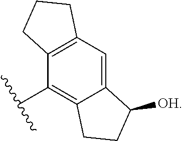 [SMILES: *c1c2c(cc3c1CC[C@@H]3O)CCC2]

In some embodiments, the substituted ring B is

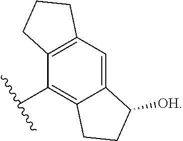 [SMILES: *c1c2c(cc3c1CC[C@H]3O)CCC2]

In some embodiments, the substituted ring B is

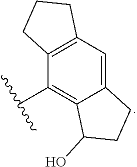 [SMILES: *c1c2c(cc3c1C(O)CC3)CCC2]

In some embodiments, the substituted ring B is

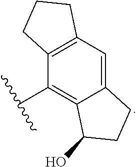 [SMILES: *c1c2c(cc3c1[C@H](O)CC3)CCC2]

In some embodiments, the substituted ring B is

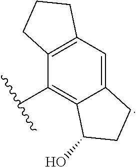 [SMILES: *c1c2c(cc3c1[C@@H](O)CC3)CCC2]

In some embodiments, the substituted ring B is

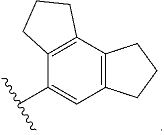 [SMILES: *c1cc2c(c3c1CCC3)CCC2]

In some embodiments, the substituted ring B is

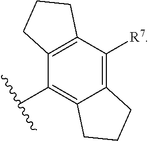

In some embodiments, the substituted ring B is selected from the group consisting of:

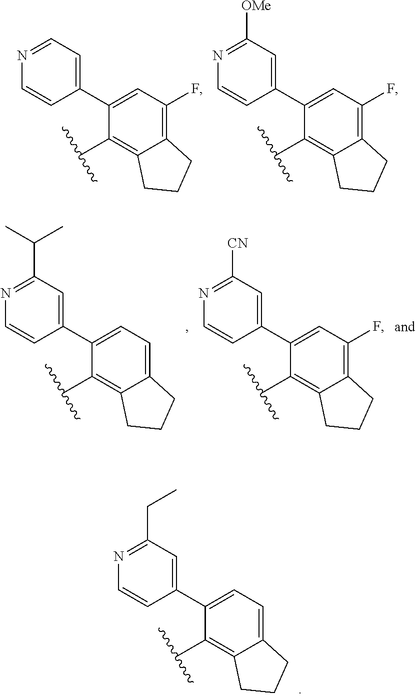

The Groups R⁶ and R⁷

In some embodiments of any of the formulas disclosed herein, at least one R⁶ is ortho to the bond connecting the B ring to the Y group of Formula AA.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from
hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₆ aliphatic carbocyclic ring or at least one 5- to 6-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₃-C₇ cycloalkyl and 3- to 7-membered heterocycloalkyl are each unsubstituted;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one 5- to 8-membered heterocyclic ring containing 1 or 2 heteroatoms independently selected from O, N, and S, wherein the heterocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₄ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₅ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₆ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one 5- to 6-membered heterocyclic ring containing 1 heteroatom independently selected from O, N, and S, wherein the heterocyclic ring is optionally independently substituted with one or more hydroxy or oxo.

In some embodiments,

at least one pair of R⁶ and R⁷ on adjacent atoms, taken together with the atoms connecting them, independently form at least one 5-membered heterocyclic ring containing 1 heteroatom independently selected from O, N, and S, wherein the heterocyclic ring is optionally independently substituted with one or more hydroxy or oxo.

In some embodiments,

at least one pair of R⁶ and R⁷ on adjacent atoms, taken together with the atoms connecting them, independently form at least one 6-membered heterocyclic ring containing 1 heteroatom independently selected from O, N, and S, wherein the heterocyclic ring is optionally independently substituted with one or more hydroxy or oxo.

In some embodiments,

at least one pair of R⁶ and R⁷ on adjacent atoms, taken together with the atoms connecting them, independently form at least one C₄ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one C₅ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one C₆ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one 5- to 6-membered heterocyclic ring containing 1 heteroatom independently selected from O, N, and S, wherein the heterocyclic ring is optionally independently substituted with one or more C₁-C₆ alkyl.

In some embodiments,

at least one pair of R⁶ and R⁷ on adjacent atoms, taken together with the atoms connecting them, independently form at least one 5-membered heterocyclic ring containing 1 heteroatom independently selected from O, N, and S, wherein the heterocyclic ring is optionally independently substituted with one or more C₁-C₆ alkyl.

In some embodiments,

at least one pair of R⁶ and R⁷ on adjacent atoms, taken together with the atoms connecting them, independently form at least one 6-membered heterocyclic ring containing 1 heteroatom independently selected from O, N, and S, wherein the heterocyclic ring is optionally independently substituted with one or more C₁-C₆ alkyl.

In some embodiments, 0=1; p=0; and

R⁶ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl, wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=1; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2; p=1, 2, or 3; and

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,
wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ (e.g., C₄-C₆) carbocyclic ring (e.g., aliphatic carbocyclic ring) or at least one 5-to-7-membered (e.g., 5-to-6-membered) heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo, or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,
wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R⁷ are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₅ carbocyclic ring, wherein the carbocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄ carbocyclic ring, wherein the carbocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, one pair of one R⁶ and one R⁷ taken together with the atoms connecting them independently form a C₄ carbocyclic ring, and the other pair of one R⁶ and one R⁷ taken together with the atoms connecting them independently form a C₅ carbocyclic ring, wherein each of C₄ and C₅ carbocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, one pair of one R⁶ and one R⁷ taken together with the atoms connecting them independently form a C₅ carbocyclic ring, and the other pair of one R⁶ and one R⁷ taken together with the atoms connecting them independently form a 5- to 8-membered heterocyclic ring containing 1 or 2 heteroatoms independently selected from O, N, and S (e.g., a 5-membered heteorocyclic ring, e.g., 5-membered heterocyclic ring containing 1 heteroatom), wherein each of carbocyclic and heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.
Particular Embodiments Wherein o=1; p=0:

In some embodiments, R⁶ is C₁-C₆ alkyl.

In some embodiments, R⁶ is isopropyl.

In some embodiments, R⁶ is ethyl.

In some embodiments, R⁶ is methyl.

In some embodiments, R⁶ is C₁-C₆ alkyl substituted with one or more halo.

In some embodiments, R⁶ is trifluoromethyl.

In some embodiments, R⁶ is trifluoromethoxy.

In some embodiments, R⁶ is C₃-C₇ cycloalkyl.

In some embodiments, R⁶ is cyclopropyl.

In some embodiments, R⁶ is halo.

In some embodiments, R⁶ is chloro.

In some embodiments, R⁶ is fluoro.

In some embodiments, R⁶ is cyano.

In some embodiments, R⁶ is attached to a carbon of an aryl ring B.

In some embodiments, R⁶ is attached to a carbon of a heteroaryl ring B.

In some embodiments, R⁶ is attached to a nitrogen of a heteroaryl ring B.

Particular Embodiments Wherein o=1 or 2; p=1, 2, or 3:

In some embodiments, at least one R⁶ is C₁-C₆ alkyl, and at least one R⁷ is C₁-C₆ alkyl optionally substituted with one or more halo.

In some embodiments, at least one R⁶ is C₁-C₆ alkyl and at least one R⁷ is C₁-C₆ alkyl.

In some embodiments, at least one R⁶ is isopropyl and at least one R⁷ is methyl.

In some embodiments, at least one R⁶ is isopropyl and at least one R⁷ is isopropyl.

In some embodiments, o=1; p=1; R⁶ is isopropyl; and R⁷ is isopropyl.

In some embodiments, at least one R⁶ is C₁-C₆ alkyl, and at least one R⁷ is C₁-C₆ alkyl substituted with one or more halo.

In some embodiments, at least one R⁶ is isopropyl and at least one R⁷ is trifluoromethyl.

In some embodiments, at least one R⁶ is C₁-C₆ alkyl, and at least one R⁷ is C₃-C₇ cycloalkyl.

In some embodiments, at least one R⁶ is isopropyl and at least one R⁷ is cyclopropyl.

In some embodiments, o=1; p=1; R⁶ is isopropyl; and R⁷ is cyclopropyl.

In some embodiments, at least one R⁶ is C₁-C₆ alkyl, and at least one R⁷ is halo.

In some embodiments, at least one R⁶ is isopropyl and at least one R⁷ is halo.

In some embodiments, at least one R⁶ is isopropyl and at least one R⁷ is chloro.

In some embodiments, at least one R⁶ is isopropyl and at least one R⁷ is fluoro.

In some embodiments, 0=1; p=1; R⁶ is isopropyl; and R⁷ is chloro.

In some embodiments, o=2; p=1; at least one R⁶ is isopropyl; and R⁷ is chloro.

In some embodiments, 0=1; p=1; R⁶ is isopropyl; and R⁷ is fluoro.

In some embodiments, o=2; p=1; at least one R⁶ is isopropyl; and R⁷ is fluoro.

In some embodiments, o=2; p=2; at least one R⁶ is isopropyl; and at least one R⁷ is fluoro.

In some embodiments, o=2; p=2; at least one R⁶ is isopropyl; one R⁷ is fluoro; and the other R⁷ is cyano.

In some embodiments, o=2; p=3; at least one R⁶ is isopropyl; two R⁷ are fluoro; and one R⁷ is chloro.

In some embodiments, o=2; p=1; at least one R⁶ is ethyl; and R⁷ is fluoro.

In some embodiments, o=2; p=1; one R⁶ is isopropyl; the other R⁶ is trifluoromethyl; and R⁷ is chloro.

In some embodiments, at least one R⁶ is C₁-C₆ alkyl, and at least one R⁷ is cyano.

In some embodiments, at least one R⁶ is isopropyl and at least one R⁷ is cyano.

In some embodiments, o=1; p=1; R⁶ is isopropyl; and R⁷ is cyano.

In some embodiments, o=2; p=1; at least one R⁶ is isopropyl; and R⁷ is cyano.

In some embodiments, at least one R⁶ is C₃-C₇ cycloalkyl, and at least one R⁷ is C₃-C₇ cycloalkyl.

In some embodiments, at least one R⁶ is cyclopropyl, and at least one R⁷ is cyclopropyl.

In some embodiments, at least one R⁶ is C₃-C₇ cycloalkyl, and at least one R⁷ is halo.

In some embodiments, at least one R⁶ is cyclopropyl and at least one R⁷ is halo.

In some embodiments, at least one R⁶ is cyclopropyl and at least one R⁷ is chloro.

In some embodiments, at least one R⁶ is cyclopropyl and at least one R⁷ is fluoro.

In some embodiments, o=1; p=1; R⁶ is cyclopropyl; and R⁷ is chloro.

In some embodiments, o=1; p=1; R⁶ is cyclopropyl; and R⁷ is fluoro.

In some embodiments, at least one R⁶ is C₁-C₆ alkyl, and at least one R⁷ is C₁-C₆ alkoxy optionally substituted with one or more halo.

In some embodiments, at least one R⁶ is isopropyl, and at least one R⁷ is C₁-C₆ alkoxy.

In some embodiments, at least one R⁶ is isopropyl, and at least one R⁷ is methoxy.

In some embodiments, o=1; p=1; R⁶ is isopropyl, and R⁷ is methoxy.

In some embodiments, o=2; p=1; at least one R⁶ is isopropyl, and R⁷ is methoxy.

In some embodiments, at least one R⁶ is C₁-C₆ alkyl, and at least one R⁷ is C₁-C₆ alkoxy substituted with one or more halo.

In some embodiments, at least one R⁶ is isopropyl, and at least one R⁷ is trifluoromethoxy.

In some embodiments, at least one R⁶ is isopropyl, and at least one R⁷ is difluoromethoxy.

In some embodiments, at least one R⁶ is halo, and at least one R⁷ is C₁-C₆ haloalkyl optionally substituted with hydroxy.

In some embodiments, 0=1; p=1; R⁶ is chloro, and R⁷ is trifluoromethyl.

In some embodiments, at least one R⁶ is halo, and at least one R⁷ is C₁-C₆ haloalkoxy.

In some embodiments, at least one R⁶ is chloro, and at least one R⁷ is trifluoromethoxy.

In some embodiments, 0=1; p=1; R⁶ is chloro, and R⁷ is trifluoromethoxy.

In some embodiments, at least one R⁶ is C₁-C₆ alkoxy; and at least one R⁷ is halo.

In some embodiments, 0=1; p=2; R⁶ is C₁-C₆ alkoxy; and at least one R⁷ is chloro.

In some embodiments, at least one R⁷ is C₁-C₆ alkyl, and at least one R⁶ is C₁-C₆ alkyl optionally substituted with one or more halo.

In some embodiments, at least one R⁷ is isopropyl and at least one R⁶ is methyl.

In some embodiments, at least one R⁷ is C₁-C₆ alkyl, and at least one R⁶ is C₁-C₆ alkyl substituted with one or more halo.

In some embodiments, at least one R⁷ is isopropyl and at least one R⁶ is trifluoromethyl.

In some embodiments, at least one R⁷ is C₁-C₆ alkyl, and at least one R⁶ is C₃-C₇ cycloalkyl.

In some embodiments, at least one R⁷ is isopropyl and at least one R⁶ is cyclopropyl.

In some embodiments, o=1; p=1; R⁷ is isopropyl; and R⁶ is cyclopropyl.

In some embodiments, at least one R⁷ is C₁-C₆ alkyl, and at least one R⁶ is halo.

In some embodiments, at least one R⁷ is isopropyl and at least one R⁶ is halo.

In some embodiments, at least one R⁷ is isopropyl and at least one R⁶ is chloro.

In some embodiments, at least one R⁷ is isopropyl and at least one R⁶ is fluoro.

In some embodiments, o=1; p=1; R⁷ is isopropyl; and R⁶ is chloro.

In some embodiments, o=2; p=1; R⁷ is isopropyl; and at least one R⁶ is chloro.

In some embodiments, o=1; p=1; R⁷ is isopropyl; and R⁶ is fluoro.

In some embodiments, o=2; p=1; R⁷ is isopropyl; and at least one R⁶ is fluoro.

In some embodiments, o=2; p=2; R⁷ is isopropyl; and at least one R⁶ is fluoro.

In some embodiments, o=2; p=2; at least one R⁷ is isopropyl; one R⁶ is fluoro; and the other R⁶ is cyano.

In some embodiments, o=2; p=1; R⁷ is ethyl; and at least one R⁶ is fluoro.

In some embodiments, o=1; p=2; one R⁷ is isopropyl; the other R⁷ is trifluoromethyl; and R⁶ is chloro.

In some embodiments, at least one R⁷ is C₁-C₆ alkyl, and at least one R⁶ is cyano.

In some embodiments, at least one R⁷ is isopropyl and at least one R⁶ is cyano.

In some embodiments, 0=1; p=1; R⁷ is isopropyl; and R⁶ is cyano.

In some embodiments, o=2; p=1; R⁷ is isopropyl; and at least one R⁶ is cyano.

In some embodiments, at least one R⁷ is C₃-C₇ cycloalkyl, and at least one R⁶ is C₃-C₇ cycloalkyl.

In some embodiments, at least one R⁷ is cyclopropyl, and at least one R⁶ is cyclopropyl.

In some embodiments, at least one R⁷ is C₃-C₇ cycloalkyl, and at least one R⁶ is halo.

In some embodiments, at least one R⁷ is cyclopropyl and at least one R⁶ is halo.

In some embodiments, at least one R⁷ is cyclopropyl and at least one R⁶ is chloro.

In some embodiments, at least one R⁷ is cyclopropyl and at least one R⁶ is fluoro.

In some embodiments, o=1; p=1; R⁷ is cyclopropyl; and R⁶ is chloro.

In some embodiments, o=1; p=1; R⁷ is cyclopropyl; and R⁶ is fluoro.

In some embodiments, at least one R⁷ is C₁-C₆ alkyl, and at least one R⁶ is C₁-C₆ alkoxy optionally substituted with one or more halo.

In some embodiments, at least one R⁷ is isopropyl, and at least one R⁶ is C₁-C₆ alkoxy.

In some embodiments, at least one R⁷ is isopropyl, and at least one R⁶ is methoxy.

In some embodiments, o=1; p=1; R⁷ is isopropyl, and R⁶ is methoxy.

In some embodiments, o=2; p=1; R⁷ is isopropyl, and at least one R⁶ is methoxy.

In some embodiments, at least one R⁷ is C₁-C₆ alkyl, and at least one R⁶ is C₁-C₆ alkoxy substituted with one or more halo.

In some embodiments, at least one R⁷ is isopropyl, and at least one R⁶ is trifluoromethoxy.

In some embodiments, at least one R⁷ is halo, and at least one R⁶ is C₁-C₆ haloalkyl optionally substituted with one or more hydroxy.

In some embodiments, o=1; p=1; R⁷ is chloro, and R⁶ is trifluoromethyl.

In some embodiments, at least one R⁷ is halo, and at least one R⁶ is C₁-C₆ haloalkoxy.

In some embodiments, at least one R⁷ is chloro, and at least one R⁶ is trifluoromethoxy.

In some embodiments, o=1; p=1; R⁷ is chloro, and R⁶ is trifluoromethoxy.

In some embodiments, at least one R⁷ is C₁-C₆ alkoxy; and at least one R⁶ is halo.

In some embodiments, o=1; p=2; at least one R⁷ is C₁-C₆ alkoxy; and R⁶ is chloro.

In some embodiments, R⁶ and R⁷ are each attached to a carbon of an aryl ring B.

In some embodiments, R⁶ and R⁷ are each attached to a carbon of a heteroaryl ring B.

In some embodiments, R⁶ is attached to a carbon and R⁷ is attached to a nitrogen of a heteroaryl ring B.

In some embodiments, R⁷ is attached to a carbon and R⁶ is attached to a nitrogen of a heteroaryl ring B.

In some embodiments, one R⁶ and one R⁷ are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₅ aliphatic carbocyclic ring.

In some embodiments, R⁶ and R⁷ are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₆ aliphatic carbocyclic ring.

In some embodiments, R⁶ and R⁷ are on adjacent atoms, and taken together with the atoms connecting them, form a C₆ aromatic carbocyclic ring.

In some embodiments, R⁶ and R⁷ are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 5-membered aliphatic heterocyclic ring containing 1 or 2 heteroatoms independently selected from O, N, and S.

In some embodiments, R⁶ and R⁷ are on adjacent atoms, and taken together with the atoms connecting them, form a 5-membered heteroaromatic ring containing 1 or 2 heteroatoms independently selected from O, N, and S.

In some embodiments, R⁶ and R⁷ are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 6-membered aliphatic heterocyclic ring containing 1 or 2 heteroatoms independently selected from O, N, and S.

In some embodiments, R⁶ and R⁷ are on adjacent atoms, and taken together with the atoms connecting them, form a 6-membered heteroaromatic ring containing 1 or 2 heteroatoms independently selected from O, N, and S.

In some embodiments, one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the ring is fused to the B ring at the 2- and 3-positions relative to the bond connecting the B ring to the NH(CO) group.

In some embodiments, o=1; p=2; and

one pair of one R⁶ and one R⁷, are on adjacent atoms; and said pair of one R⁶ and one R⁷ taken together with the atoms connecting them form form a C₄-C₈ carbocyclic ring or a 5- to 8-membered heterocyclic ring containing 1 or 2 heteroatoms independently selected from 0, N, and S, wherein the ring is fused to the B ring at the 2- and 3-positions relative to the bond connecting the B ring to the NH(CO) group.

In some embodiments, o=1; p=2; and

one pair of one R⁶ and one R⁷, are on adjacent atoms; and said pair of one R⁶ and one R⁷ taken together with the atoms connecting them form form a C₄-C₈ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1; p=2; and

one pair of one R⁶ and one R⁷, are on adjacent atoms; and said pair of one R⁶ and one R⁷ taken together with the atoms connecting them form form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1; p=2; and

one pair of one R⁶ and one R⁷, are on adjacent atoms; and said pair of one R⁶ and one R⁷ taken together with the atoms connecting them form form a C₅ aliphatic carbocyclic ring.

In some embodiments, o=2; p=2; and

one pair of one R⁶ and one R⁷, are on adjacent atoms; and said pair of one R⁶ and one R⁷ taken together with the atoms connecting them form form a C₄-C₈ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2; and

one pair of one R⁶ and one R⁷, are on adjacent atoms; and said pair of one R⁶ and one R⁷ taken together with the atoms connecting them form form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, 0=1; p=2; and

one pair of one R⁶ and one R⁷, are on adjacent atoms; and said pair of one R⁶ and one R⁷ taken together with the atoms connecting them form form a C₅ aliphatic carbocyclic ring.

In some embodiments, o=2; p=2 or 3; and

two pairs, each of one R⁶ and one R⁷, are on adjacent atoms; one pair of one R⁶ and one R⁷ taken together with the atoms connecting them form a C₄ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and the other pair of one R⁶ and one R⁷ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms; one pair of one R⁶ and one R⁷ taken together with the atoms connecting them form a C₄ aliphatic carbocyclic ring and the other pair of one R⁶ and one R⁷ taken together with the atoms connecting them form a C₅ aliphatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a C₅ aliphatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a C₆ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a C₆ aliphatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a C₆ aromatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered heteroaromatic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered heteroaromatic ring containing 1 or 2 heteroatoms independently selected from O, N, and S.

In some embodiments, o=2; p=2 or 3; and

two pairs, each of one R⁶ and one R⁷, are on adjacent atoms; one pair of one R⁶ and one R⁷ taken together with the atoms connecting them form a C₄₋₈carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and the other pair of one R⁶ and one R⁷ taken together with the atoms connecting them form a 5- to 8-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms; one pair of one R⁶ and one R⁷ taken together with the atoms connecting them form a C₅ aliphatic carbocyclic ring and the other pair of one R⁶ and one R⁷ taken together with the atoms connecting them form a 5-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms; one pair of one R⁶ and one R⁷ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and the other pair of one R⁶ and one R⁷ taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and two pairs, each of one R⁶ and one R⁷, are on adjacent atoms; one pair of one R⁶ and one R⁷ taken together with the atoms connecting them form a C₅ aliphatic carbocyclic ring and the other pair of one R⁶ and one R⁷ taken together with the atoms connecting them form a 5-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S,
wherein one of the two rings is fused to the B ring at the 2- and 3-positions relative to the bond connecting the B ring to the NH(CO) group, and the other of the two rings is fused to the B ring at the 5- and 6-positions relative to the bond connecting the B ring to the NH(CO) group.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S,
wherein one of the two rings is fused to the B ring at the 2- and 3-positions relative to the bond connecting the B ring to the NH(CO) group, and the other of the two rings is fused to the B ring at the 4- and 5-positions relative to the bond connecting the B ring to the NH(CO) group.

In some embodiments, o=2; p=2; and

two pairs, each of one R⁶ and one R⁷, are on adjacent atoms, and each pair of one R⁶ and one R⁷ taken together with the atoms connecting them form a C₅ aliphatic carbocyclic ring.

In some embodiments, o=2; p=2; and

two pairs, each of one R⁶ and one R⁷, are on adjacent atoms, one pair of one R⁶ and one R⁷ taken together with the atoms connecting them form a C₄ aliphatic carbocyclic ring, and the other pair of one R⁶ and one R⁷ taken together with the atoms connecting them form a C₅ aliphatic carbocyclic ring.

In some embodiments, o=2; p=2; and

two pairs, each of one R⁶ and one R⁷, are on adjacent atoms, and each pair of one R⁶ and one R⁷ taken together with the atoms connecting them form a C₄ aliphatic carbocyclic ring.

In some embodiments, o=2; p=2; and

two pairs, each of one R⁶ and one R⁷, are on adjacent atoms, one pair of one R⁶ and one R⁷ taken together with the atoms connecting them form a C₅ aliphatic carbocyclic ring, and the other pair of one R⁶ and one R⁷ taken together with the atoms connecting them form a 5-membered heterocyclic ring containing 1 or 2 heteroatoms independently selected from O, N, and S.

In some embodiments, o=2; p=3; and

two pairs, each of one R⁶ and one R⁷, are on adjacent atoms, and each pair of one R⁶ and one R⁷ taken together with the atoms connecting them form a C₅ aliphatic carbocyclic ring; and one R⁷ is halo (e.g., C₁ or F).

In some embodiments, o=2; p=3; and

two pairs, each of one R⁶ and one R⁷, are on adjacent atoms, and each pair of one R⁶ and one R⁷ taken together with the atoms connecting them form a C₅ aliphatic carbocyclic ring; and one R⁷ is CN.

In some embodiments, one R⁷ is pyrazolyl and is para to the bond connecting the B ring to the NH(CO) group of Formula AA.

In some embodiments, one R⁷ is 3-pyrazolyl and is para to the bond connecting the B ring to the NH(CO) group of Formula AA.

In some embodiments, one R⁷ is 4-pyrazolyl and is para to the bond connecting the B ring to the NH(CO) group of Formula AA.

In some embodiments, one R⁷ is 5-pyrazolyl and is para to the bond connecting the B ring to the NH(CO) group of Formula AA.

In some embodiments, one R⁷ is thiazolyl and is para to the bond connecting the B ring to the NH(CO) group of Formula AA.

In some embodiments, one R⁷ is 4-thiazolyl and is para to the bond connecting the B ring to the NH(CO) group of Formula AA.

In some embodiments, one R⁷ is 5-thiazolyl and is para to the bond connecting the B ring to the NH(CO) group of Formula AA.

In some embodiments, one R⁷ is furyl and is para to the bond connecting the B ring to the NH(CO) group of Formula AA.

In some embodiments, one R⁷ is 2-furyl and is para to the bond connecting the B ring to the NH(CO) group of Formula AA.

In some embodiments, one R⁷ is thiophenyl and is para to the bond connecting the B ring to the NH(CO) group of Formula AA.

In some embodiments, one R⁷ is 2-thiophenyl and is para to the bond connecting the B ring to the NH(CO) group of Formula AA.

In some embodiments, one R⁷ is phenyl and is para to the bond connecting the B ring to the NH(CO) group of Formula AA.

In some embodiments, one R⁷ is cycloalkenyl (e.g., cyclopentenyl, e.g., 1-cyclopentenyl) and is para to the bond connecting the B ring to the NH(CO) group of Formula AA.

In some embodiments, one R⁷ is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl and is para to the bond connecting the B ring to the NH(CO) group of Formula AA.

In some embodiments, one R⁷ is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl and is para to the bond connecting the B ring to the NH(CO) group of Formula AA.

In some embodiments, one R⁷ is phenyl optionally substituted with one or more C₆-C₁₀ aryloxy (e.g., phenoxy) and is para to the bond connecting the B ring to the NH(CO) group of Formula AA.

In some embodiments, one R⁷ is phenyl optionally substituted with one or more CN and is para to the bond connecting the B ring to the NH(CO) group of Formula AA.

In some embodiments, one R⁷ is phenyl optionally substituted with one or more halo (e.g., F, CO and is para to the bond connecting the B ring to the NH(CO) group of Formula AA and is para to the bond connecting the B ring to the NH(CO) group of Formula AA.

In some embodiments, one R⁷ is phenyl optionally substituted with one or more COOC₁-C₆ alkyl (e.g., CO₂ₜ-Bu) and is para to the bond connecting the B ring to the NH(CO) group of Formula AA.

In some embodiments, one R⁷ is phenyl optionally substituted with one or more S(O₂)C₁-C₆ alkyl (e.g., S(O₂)methyl) and is para to the bond connecting the B ring to the NH(CO) group of Formula AA.

In some embodiments, one R⁷ is phenyl optionally substituted with one or more 3- to 7-membered heterocycloalkyl (e.g., morpholinyl) and is para to the bond connecting the B ring to the NH(CO) group of Formula AA.

In some embodiments, one R⁷ is phenyl optionally substituted with one or more CONR⁸R⁹ (e.g., unsubstituted amido) and is para to the bond connecting the B ring to the NH(CO) group of Formula AA.

In some embodiments, one R⁷ is phenyl optionally substituted with one or more C₁-C₆ alkyl (e.g., methyl or propyl, e.g., 2-propyl) and with one or more halo (e.g., F, C₁) and is para to the bond connecting the B ring to the NH(CO) group of Formula AA and is para to the bond connecting the B ring to the NH(CO) group of Formula AA.

In some embodiments, R⁶ and R⁷ are each attached to a carbon of an aryl ring B.

In some embodiments, R⁶ and R⁷ are each attached to a carbon of a heteroaryl ring B.

In some embodiments, R⁶ is attached to a carbon and R⁷ is attached to a nitrogen of a heteroaryl ring B.

In some embodiments, R⁷ is attached to a carbon and R⁶ is attached to a nitrogen of a heteroaryl ring B.
In some embodiments, the substituted ring B is

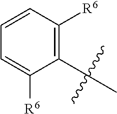

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
In some embodiments, the substituted ring B is

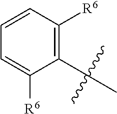

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.
In some embodiments, the substituted ring B is

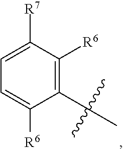

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁹, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments,

the R⁶ and R⁷ on adjacent atoms, taken together with the atoms connecting them, independently form a C₄-C₈ carbocyclic ring optionally substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; and

the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the R⁶ and R⁷ on adjacent atoms, taken together with the atoms connecting them, independently form a C₅-6 carbocyclic ring; and the other R⁶ is 5- to 6-membered heteroaryl optionally substituted with one or more substituents each independently selected from: hydroxy, halo, C₁-C₆ alkoxy, CN, and C₁-C₆ alkyl.

For example, the R⁶ and R⁷ on adjacent atoms, taken together with the atoms connecting them, independently form a C₅ carbocyclic ring; and the other R⁶ is pyridyl (e.g., 4-pyridyl) optionally substituted with one or more substituents each independently selected from: CN, OMe, isopropyl, and ethyl.

As a non-limiting example of the foregoing embodiments, substituted ring B is:

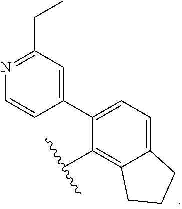 [SMILES: *c1c(-c2ccnc(CC)c2)ccc2c1CCC2]

In some embodiments, the substituted ring B is

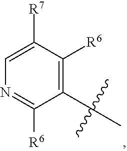

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy,

C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the substituted ring B is

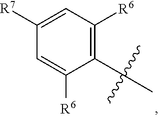

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In certain embodiments (when the substituted ring B is

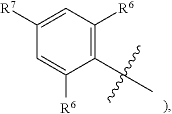

one R⁶ is C₁-C₆ alkyl; and the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₁-C₆ alkyl; and the other R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 (e.g., 6) membered heteroaryl (e.g., pyridinyl (e.g., pyridin-4-yl), pyrimidinyl, or thiazolyl) optionally substituted with a substituent selected from hydroxyl, halo, CN, C₁-C₆ alkyl, and C₁-C₆ alkoxy.

As a non-limiting example of the foregoing embodiments, substituted ring B is selected from:

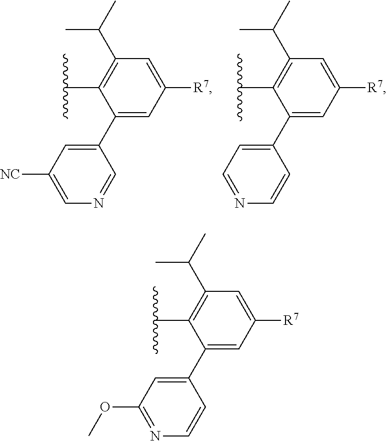

wherein R⁷ is halo (e.g., fluoro).
In some embodiments, the substituted ring B is

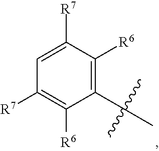

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the substituted ring B is

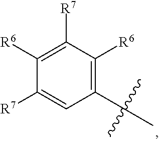

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the substituted ring B is

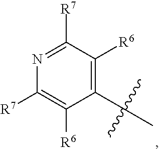

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
In some embodiments, the substituted ring B is

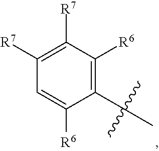

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form a

C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments,

the R⁶ and R⁷ on adjacent atoms, taken together with the atoms connecting them, independently form a C₄-C₈ carbocyclic ring optionally substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl; and

the other R⁷ is selected from C₁-C₆ alkoxy, C₁-C₆ haloalkoxy, halo, and CN.

In certain of these embodiments, the R⁶ and R⁷ on adjacent atoms, taken together with the atoms connecting them, independently form a C₅₋₆ carbocyclic ring; the other R⁶ is 5- to 6-membered heteroaryl optionally substituted with one or more substituents each independently selected from: hydroxy, halo, C₁-C₆ alkoxy, CN, and C₁-C₆ alkyl; and the other R⁷ is halo.

For example, the R⁶ and R⁷ on adjacent atoms, taken together with the atoms connecting them, independently form a C₅ carbocyclic ring; the other R⁶ is pyridyl (e.g., 4-pyridyl) optionally substituted with one or more substituents each independently selected from: CN, OMe, isopropyl, and ethyl; and the other R⁷ is F.

As non-limiting examples of the foregoing embodiments, substituted ring B is:

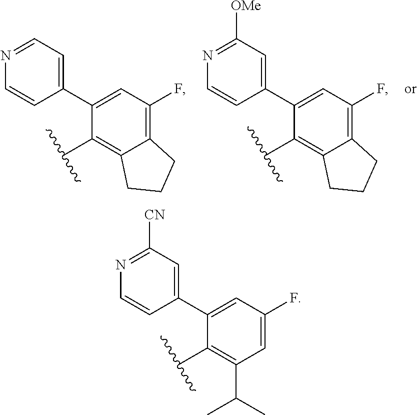

In certain of these embodiments,

the R⁶ and R⁷ on adjacent atoms, taken together with the atoms connecting them, independently form a C₄-C₈ carbocyclic ring optionally substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl; and

the other R⁷ is selected from C₁-C₆ alkoxy, C₁-C₆ haloalkoxy, halo, and CN.

In certain of these embodiments, the R⁶ and R⁷ on adjacent atoms, taken together with the atoms connecting them, independently form a C₅₋₆ carbocyclic ring; the other R⁶ is 5- to 6-membered heteroaryl optionally substituted with one or more substituents each independently selected from: hydroxy, halo, C₁-C₆ alkoxy, CN, and C₁-C₆ alkyl; and the other R⁷ is halo.

For example, the R⁶ and R⁷ on adjacent atoms, taken together with the atoms connecting them, independently form a C₅ carbocyclic ring; the other R⁶ is pyridyl (e.g., 4-pyridyl) optionally substituted with one or more substituents each independently selected from: CN, OMe, isopropyl, and ethyl; and the other R⁷ is F.

As non-limiting examples of the foregoing embodiments, substituted ring B is:

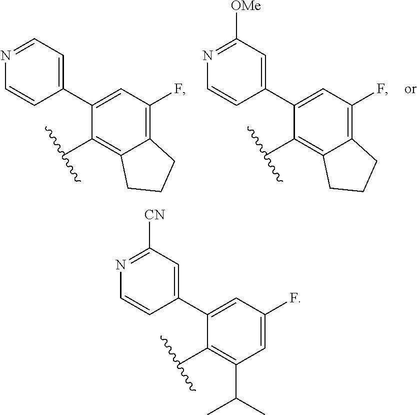

In some embodiments, the substituted ring B is

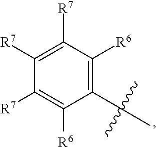

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

### The Group R³

In some embodiments, R³ is selected from hydrogen, C₁-C₆ alkyl, and

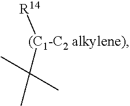

wherein the C₁-C₂ alkylene group is optionally substituted with oxo.

In some embodiments, R³ is hydrogen.

In some embodiments, R³ is hydroxy.

In some embodiments, R³ is C₁-C₆ alkoxy.

In some embodiments, R³ is C₁-C₆ alkyl.

In some embodiments, R³ is methyl.

In some embodiments, R³ is

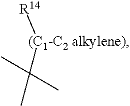

wherein the C₁-C₂ alkylene group is optionally substituted with oxo.

In some embodiments, R³ is CH₂R¹⁴.

In some embodiments, R³ is C(O)R¹⁴.

In some embodiments, R³ is CH₂CH₂R¹⁴.

In some embodiments, R³ is CHR¹⁴CH₃.

In some embodiments, R³ is CH₂C(O)R¹⁴.

In some embodiments, R³ is C(O)CH₂R¹⁴.

In some embodiments, R³ is CO₂C₁-C₆ alkyl.

### The Group R³⁴

In some embodiments, R¹⁴ is hydrogen, C₁-C₆ alkyl, 5- to 10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶.

In some embodiments, R¹⁴ is hydrogen or C₁-C₆ alkyl.

In some embodiments, R¹⁴ is hydrogen, 5- to 10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶.

In some embodiments, R¹⁴ is hydrogen.

In some embodiments, R¹⁴ is NR⁸R⁹.

In some embodiments, R¹⁴ is C₁-C₆ alkyl.

In some embodiments, R¹⁴ is methyl.

In some embodiments, R¹⁴ is 5- to 10-membered monocyclic or bicyclic heteroaryl optionally independently substituted with 1 or 2 R⁶.

In some embodiments, R¹⁴ is C₆-C₁₀ monocyclic or bicyclic aryl optionally independently substituted with 1 or 2 R⁶.

The moiety S(X)(O)N(S(═O)(NHR³)═N—)

In some embodiments, the compound is enantioenriched at the sulfur in the moiety S(X)(O)N (e.g., S(═O)(NHR³)═N—).

In certain embodiments (when the compound is enantioenriched at the sulfur in the moiety S(X)(O)N(e.g., S(═O)(NHR³)═N—)), the ee is greater than about 60% (e.g., greater than about 70%, greater than about 80%, greater than about 90%, greater than about 95%, greater than about 98%, or greater than about 99).

In some embodiments, the sulfur in the moiety S(X)(O)N(e.g., S(═O)(NHR³)═N—) has (S) stereochemistry.

In some embodiments, the sulfur in the moiety S(X)(O)N(e.g., S(═O)(NHR³)═N—) has (R) stereochemistry.

### The Group R¹⁰

In some embodiments, R¹⁰ is C₁-C₆ alkyl.

In some embodiments, R¹⁰ is methyl.

In some embodiments, R¹⁰ is ethyl.

The Groups R⁸ and R⁹

In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to.

In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to.

In some embodiments, each of R⁸ and R⁹ at each occurrence is hydrogen,

In some embodiments, each R⁸ at each occurrence is hydrogen and each R⁹ at each occurrence is C₁-C₆ alkyl.

In some embodiments, each R⁸ at each occurrence is hydrogen and each R⁹ at each occurrence is methyl.

In some embodiments, each R⁸ at each occurrence is hydrogen and each R⁹ at each occurrence is ethyl.

In some embodiments, each of R⁸ and R⁹ at each occurrence is methyl.

In some embodiments, each of R⁸ and R⁹ at each occurrence is ethyl.

In some embodiments, R⁸ and R⁹ taken together with the nitrogen they are attached to form a 3-membered ring.

In some embodiments, R⁸ and R⁹ taken together with the nitrogen they are attached to form a 4-membered ring.

In some embodiments, R⁸ and R⁹ taken together with the nitrogen they are attached to form a 5-membered ring.

In some embodiments, R⁸ and R⁹ taken together with the nitrogen they are attached to form a 6-membered ring optionally containing one or more oxygen atoms in addition to the nitrogen they are attached to.

In some embodiments, R⁸ and R⁹ taken together with the nitrogen they are attached to form a 6-membered ring optionally containing one or more nitrogen atoms in addition to the nitrogen they are attached to.

In some embodiments, R⁸ and R⁹ taken together with the nitrogen they are attached to form a 7-membered ring.

### The Group R¹³

In some embodiments, R¹³ is C₁-C₆ alkyl.

In some embodiments, R¹³ is methyl.

In some embodiments, R¹³ is ethyl.

In some embodiments, R¹³ is C₆-C₁₀ aryl.

In some embodiments, R¹³ is phenyl.

In some embodiments, R¹³ is 5- to 10-membered heteroaryl.

The Groups R¹¹ and R¹²

In some embodiments, each of R¹¹ and R¹² at each occurrence is independently selected from hydrogen and C₁-C₆ alkyl.

In some embodiments, each of R¹¹ and R¹² at each occurrence is hydrogen,

In some embodiments, each R¹¹ at each occurrence is hydrogen and each R¹² at each occurrence is C₁-C₆ alkyl.

In some embodiments, each R¹¹ at each occurrence is hydrogen and each R¹² at each occurrence is methyl.

In some embodiments, each R¹¹ at each occurrence is hydrogen and each R¹² at each occurrence is ethyl.

In some embodiments, each of R¹¹ and R¹² at each occurrence is methyl.

In some embodiments, each of R¹¹ and R¹² at each occurrence is ethyl.

Combinations of Ring A, R¹, and R²

In some embodiments of the compound of formula AA,

the substituted ring A is

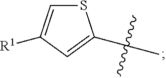

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

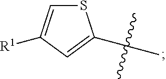

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

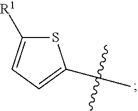

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

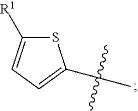

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

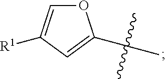

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

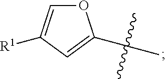

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

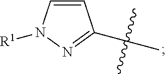

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

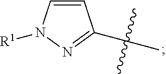

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

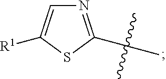

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

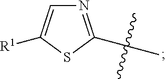

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

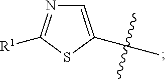

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

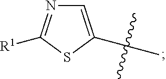

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

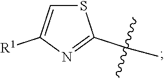

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

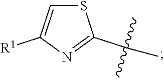

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

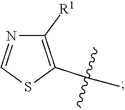

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

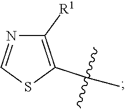

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

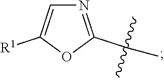

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

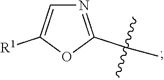

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

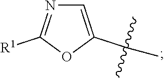

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.
      In some embodiments of the compound of formula AA,
      the substituted ring A is

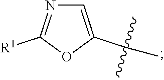

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

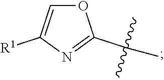

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

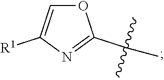

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

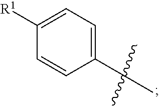

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

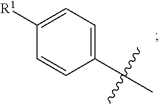

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

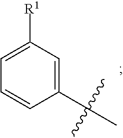

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

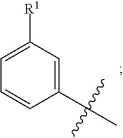

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

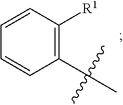

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

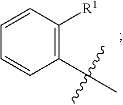

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

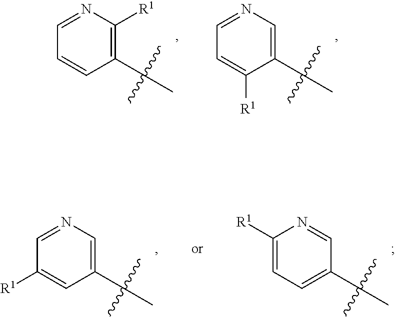

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl, wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

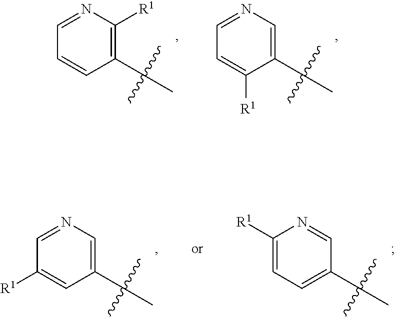

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

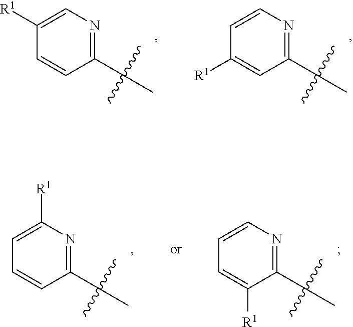

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

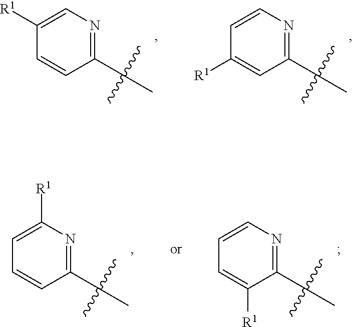

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

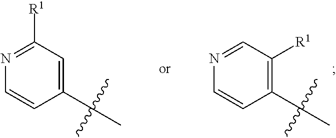

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

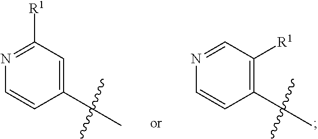

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

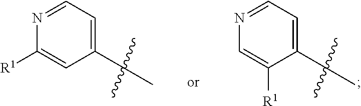

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents
   each independently selected from hydroxy, halo, oxo, C₁-C₆ alkoxy, and NR⁸R⁹.

In some embodiments of the compound of formula AA,

the substituted ring A is

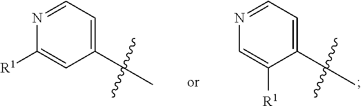

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

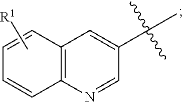

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

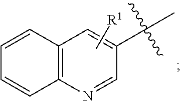

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

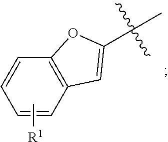

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

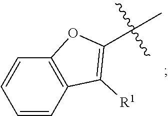

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

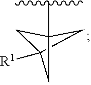

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

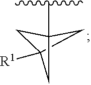

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

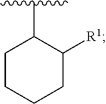

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

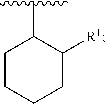

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

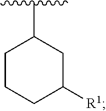

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

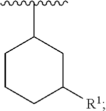

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

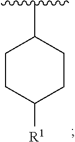

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

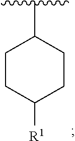

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

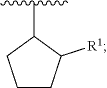

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

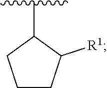

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

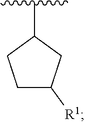

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

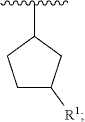

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

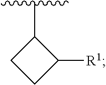

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

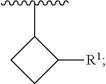

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

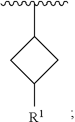

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

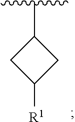

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

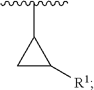

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

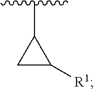

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

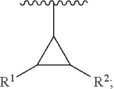

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

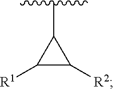

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

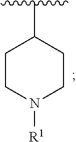

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

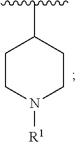

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

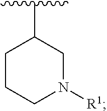

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

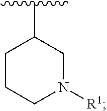

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

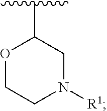

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

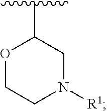

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

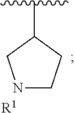

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

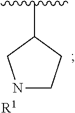

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

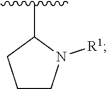

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

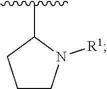

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

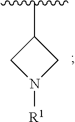

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

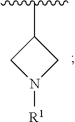

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

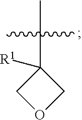

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

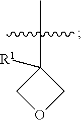

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

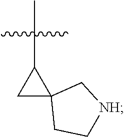 [SMILES: *C1CC12CCNC2]

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

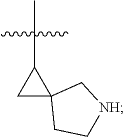 [SMILES: *C1CC12CCNC2]

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

In some embodiments of the compound of formula AA,

the substituted ring A is

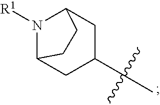

and R¹ is selected from:
C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.

In some embodiments of the compound of formula AA,

the substituted ring A is

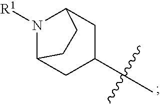

and R¹ is selected from:
methyl, ethyl, isopropyl, 2-hydroxy-2-propyl, dimethylamino, aminomethyl, methylaminomethyl, dimethylaminomethyl, methoxycarbonyl, and carboxyl.

### Subgenera of Formula AA

In some embodiments, the compound of Formula AA is a compound of Formula BB:

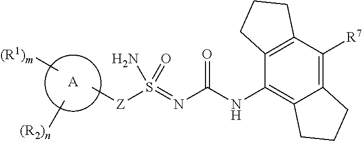

wherein:
Z is C₁₋₄alkylene having from 1-4 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O); and
when
   (i) ring A is phenyl,
   (ii) the sum of m and n is 1, and
   (iii) whichever of R¹ and R² that is present is CN;
then the position of the phenyl group that is para to the point of the phenyl group's connection to the sulfur of the S(O)(NHR³)═N moiety is substituted with hydrogen.

In some embodiments, the compound of Formula AA is a compound of Formula CC:

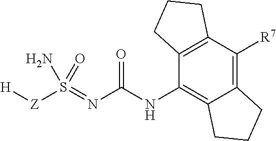

wherein Z is:
(ii) 3-10-membered heterocycloalkylene optionally substituted by one or more R¹ and/or R²; or
(iii) C₃-C₁₀ cycloalkyl optionally substituted by one or more R¹ and/or R².

### Additional Features of the Embodiments Herein

In some embodiments the compound of any of the formulae herein does not have the following structure:

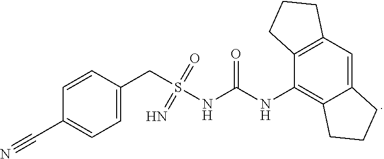 [SMILES: N#Cc1ccc(CS(=N)(=O)NC(=O)Nc2c3c(cc4c2CCC4)CCC3)cc1]

In some embodiments the compound of any of the formulae herein, when
(i) Formula AA is Formula AA-2,
(ii) ring A is phenyl,
(iii) the sum of m and n is 1, and
(iv) whichever of R¹ and R² that is present is CN;
then the position of the phenyl group that is para to the point of the phenyl group's connection to the sulfur of the S(O)(NHR³)═N moiety is substituted with hydrogen.

In some embodiments the compound of any of the formulae herein is not a compound disclosed in WO 2018225018, which is incorporated herein by reference in its entirety.

It is understood that the combination of variables in the formulae herein is such that the compounds are stable.

In some embodiments, provided herein is a compound that is selected from the group consisting of the compounds in Table 1A-1:

TABLE 1A-1

| Com- | |
|---|---|
| pound | Structure |
| 101 | 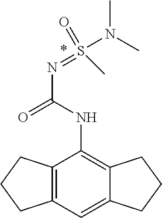 [SMILES: CN(C)S(C)(=O)=NC(=O)Nc1c2c(cc3c1CCC3)CCC2] |
| 102 | 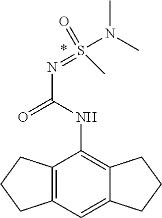 [SMILES: CN(C)S(C)(=O)=NC(=O)Nc1c2c(cc3c1CCC3)CCC2] |
| 103 | 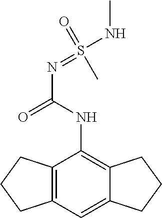 [SMILES: CNS(C)(=O)=NC(=O)Nc1c2c(cc3c1CCC3)CCC2] |
| 103a | 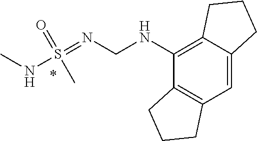 [SMILES: CNS(C)(=O)=NCNc1c2c(cc3c1CCC3)CCC2] |
| 103b | 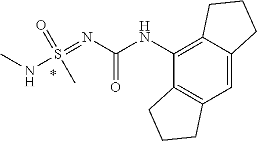 [SMILES: CNS(C)(=O)=NC(=O)Nc1c2c(cc3c1CCC3)CCC2] |
| 104 | 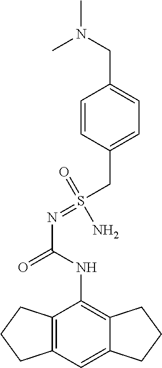 [SMILES: CN(C)Cc1ccc(CS(N)(=O)=NC(=O)Nc2c3c(cc4c2CCC4)CCC3)cc1] |
| 105 | 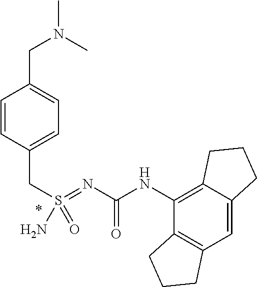 [SMILES: CN(C)Cc1ccc(CS(N)(=O)=NC(=O)Nc2c3c(cc4c2CCC4)CCC3)cc1] |
| 106 | 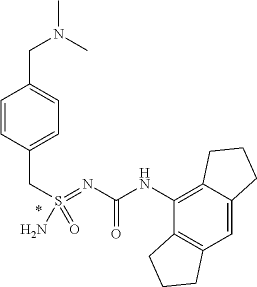 [SMILES: CN(C)Cc1ccc(CS(N)(=O)=NC(=O)Nc2c3c(cc4c2CCC4)CCC3)cc1] |
| 107 | 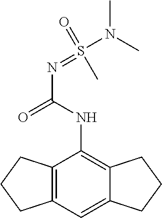 [SMILES: CN(C)S(C)(=O)=NC(=O)Nc1c2c(cc3c1CCC3)CCC2] |
| 108 | 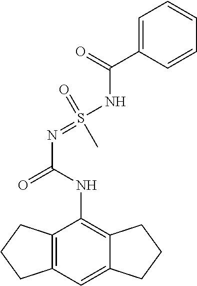 [SMILES: CS(=O)(=NC(=O)Nc1c2c(cc3c1CCC3)CCC2)NC(=O)c1ccccc1] |
| 109 | 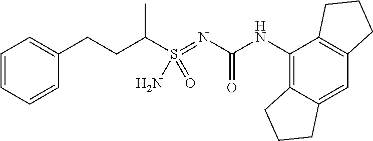 [SMILES: CC(CCc1ccccc1)S(N)(=O)=NC(=O)Nc1c2c(cc3c1CCC3)CCC2] |
| 110a | 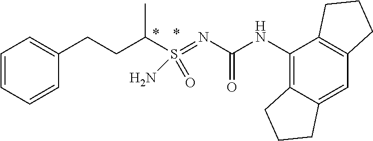 [SMILES: CC(CCc1ccccc1)S(N)(=O)=NC(=O)Nc1c2c(cc3c1CCC3)CCC2] |
| 110b | 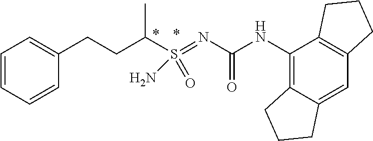 [SMILES: CC(CCc1ccccc1)S(N)(=O)=NC(=O)Nc1c2c(cc3c1CCC3)CCC2] |
| 110c | 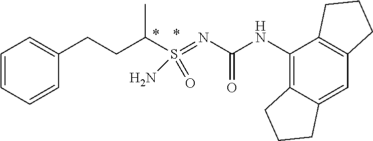 [SMILES: CC(CCc1ccccc1)S(N)(=O)=NC(=O)Nc1c2c(cc3c1CCC3)CCC2] |
| 110d | 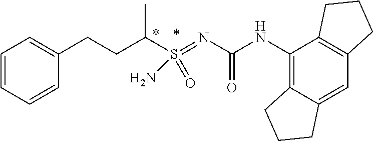 [SMILES: CC(CCc1ccccc1)S(N)(=O)=NC(=O)Nc1c2c(cc3c1CCC3)CCC2] |
| 111 | 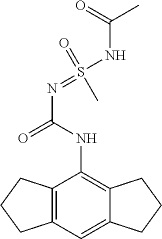 [SMILES: CC(=O)NS(C)(=O)=NC(=O)Nc1c2c(cc3c1CCC3)CCC2] |
| 112 | 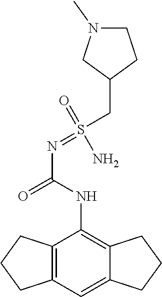 [SMILES: CN1CCC(CS(N)(=O)=NC(=O)Nc2c3c(cc4c2CCC4)CCC3)C1] |
| 113 | 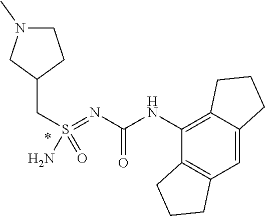 [SMILES: CN1CCC(CS(N)(=O)=NC(=O)Nc2c3c(cc4c2CCC4)CCC3)C1] |
| 114 | 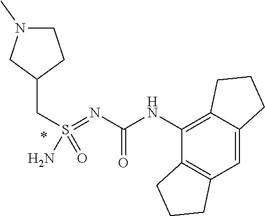 [SMILES: CN1CCC(CS(N)(=O)=NC(=O)Nc2c3c(cc4c2CCC4)CCC3)C1] |
| 115 | 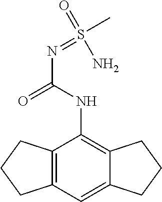 [SMILES: CS(N)(=O)=NC(=O)Nc1c2c(cc3c1CCC3)CCC2] |
| 116 | 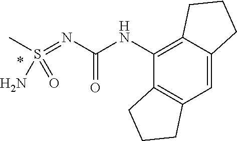 [SMILES: CS(N)(=O)=NC(=O)Nc1c2c(cc3c1CCC3)CCC2] |
| 117 | 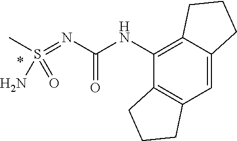 [SMILES: CS(N)(=O)=NC(=O)Nc1c2c(cc3c1CCC3)CCC2] |
| 118 | 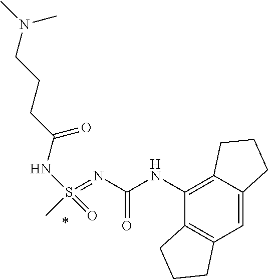 [SMILES: CN(C)CCCC(=O)NS(C)(=O)=NC(=O)Nc1c2c(cc3c1CCC3)CCC2] |
| 119 | 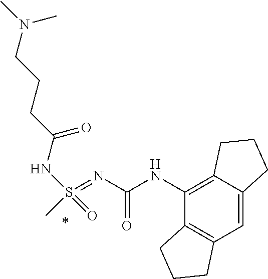 [SMILES: CN(C)CCCC(=O)NS(C)(=O)=NC(=O)Nc1c2c(cc3c1CCC3)CCC2] |
| 120 | 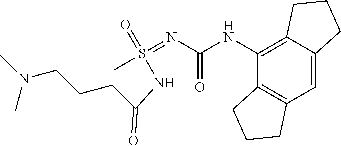 [SMILES: CN(C)CCCC(=O)NS(C)(=O)=NC(=O)Nc1c2c(cc3c1CCC3)CCC2] |
| 121 | 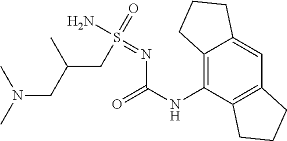 [SMILES: CC(CN(C)C)CS(N)(=O)=NC(=O)Nc1c2c(cc3c1CCC3)CCC2] |
| 122 | 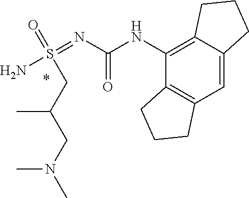 [SMILES: CC(CN(C)C)CS(N)(=O)=NC(=O)Nc1c2c(cc3c1CCC3)CCC2] |
| 123 | 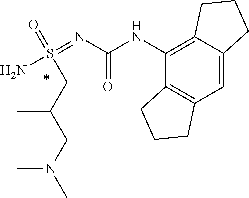 [SMILES: CC(CN(C)C)CS(N)(=O)=NC(=O)Nc1c2c(cc3c1CCC3)CCC2] |
| 124 | 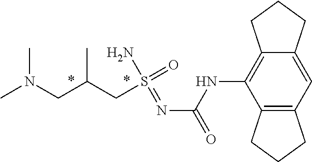 [SMILES: CC(CN(C)C)CS(N)(=O)=NC(=O)Nc1c2c(cc3c1CCC3)CCC2] |
| 125 | 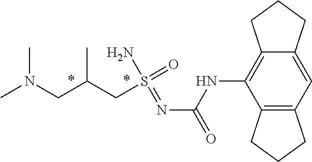 [SMILES: CC(CN(C)C)CS(N)(=O)=NC(=O)Nc1c2c(cc3c1CCC3)CCC2] |
| 126 | 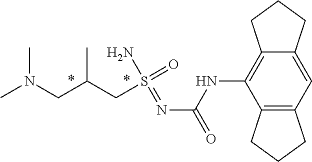 [SMILES: CC(CN(C)C)CS(N)(=O)=NC(=O)Nc1c2c(cc3c1CCC3)CCC2] |
| 127 | 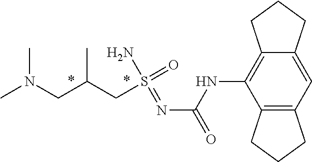 [SMILES: CC(CN(C)C)CS(N)(=O)=NC(=O)Nc1c2c(cc3c1CCC3)CCC2] |
| 129 | 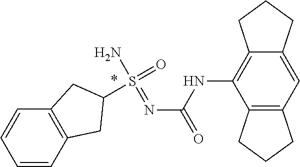 [SMILES: NS(=O)(=NC(=O)Nc1c2c(cc3c1CCC3)CCC2)C1Cc2ccccc2C1] |
| 130 | 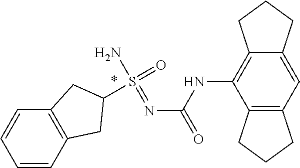 [SMILES: NS(=O)(=NC(=O)Nc1c2c(cc3c1CCC3)CCC2)C1Cc2ccccc2C1] |
| 131 | 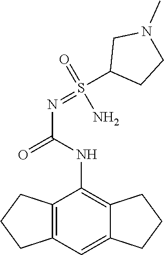 [SMILES: CN1CCC(S(N)(=O)=NC(=O)Nc2c3c(cc4c2CCC4)CCC3)C1] |
| 132 | 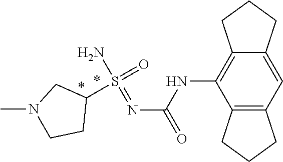 [SMILES: CN1CCC(S(N)(=O)=NC(=O)Nc2c3c(cc4c2CCC4)CCC3)C1] |
| 133 | 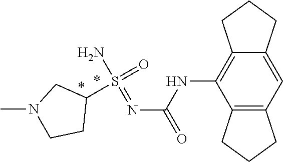 [SMILES: CN1CCC(S(N)(=O)=NC(=O)Nc2c3c(cc4c2CCC4)CCC3)C1] |
| 132 | 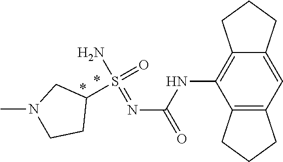 [SMILES: CN1CCC(S(N)(=O)=NC(=O)Nc2c3c(cc4c2CCC4)CCC3)C1] |
| 133 | 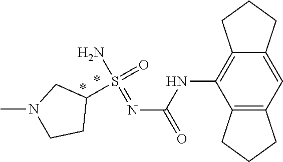 [SMILES: CN1CCC(S(N)(=O)=NC(=O)Nc2c3c(cc4c2CCC4)CCC3)C1] |
| 134 | 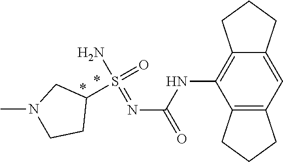 [SMILES: CN1CCC(S(N)(=O)=NC(=O)Nc2c3c(cc4c2CCC4)CCC3)C1] |
| 135 | 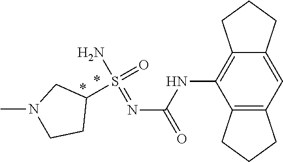 [SMILES: CN1CCC(S(N)(=O)=NC(=O)Nc2c3c(cc4c2CCC4)CCC3)C1] |
| 136 | 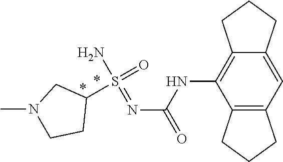 [SMILES: CN1CCC(S(N)(=O)=NC(=O)Nc2c3c(cc4c2CCC4)CCC3)C1] |
| 137 | 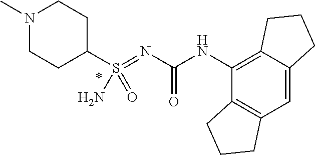 [SMILES: CN1CCC(S(N)(=O)=NC(=O)Nc2c3c(cc4c2CCC4)CCC3)CC1] |
| 138 | 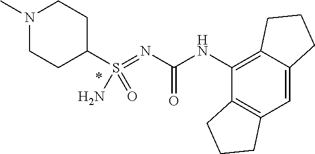 [SMILES: CN1CCC(S(N)(=O)=NC(=O)Nc2c3c(cc4c2CCC4)CCC3)CC1] |
| 139 | 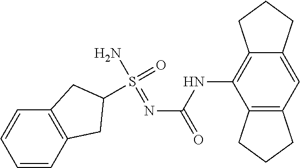 [SMILES: NS(=O)(=NC(=O)Nc1c2c(cc3c1CCC3)CCC2)C1Cc2ccccc2C1] |
| 140 | 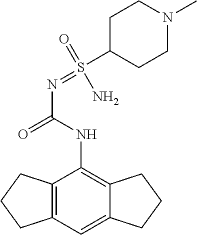 [SMILES: CN1CCC(S(N)(=O)=NC(=O)Nc2c3c(cc4c2CCC4)CCC3)CC1] |
| 141 | 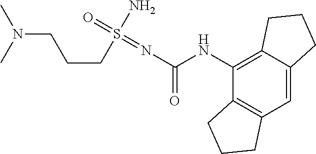 [SMILES: CN(C)CCCS(N)(=O)=NC(=O)Nc1c2c(cc3c1CCC3)CCC2] |
| 142 | 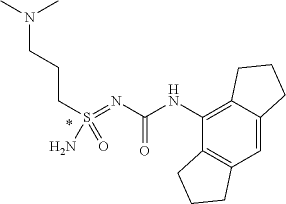 [SMILES: CN(C)CCCS(N)(=O)=NC(=O)Nc1c2c(cc3c1CCC3)CCC2] |
| 143 | 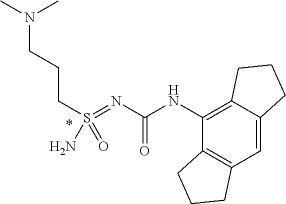 [SMILES: CN(C)CCCS(N)(=O)=NC(=O)Nc1c2c(cc3c1CCC3)CCC2] |
| 144 | 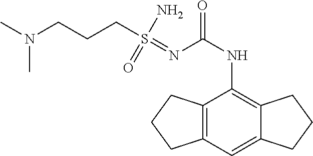 [SMILES: CN(C)CCCS(N)(=O)=NC(=O)Nc1c2c(cc3c1CCC3)CCC2] |
| 145 | 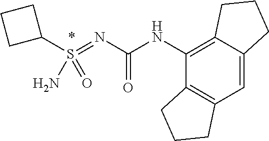 [SMILES: NS(=O)(=NC(=O)Nc1c2c(cc3c1CCC3)CCC2)C1CCC1] |
| 146 | 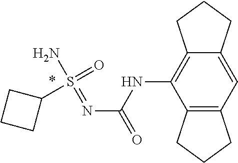 [SMILES: NS(=O)(=NC(=O)Nc1c2c(cc3c1CCC3)CCC2)C1CCC1] |
| 147 | 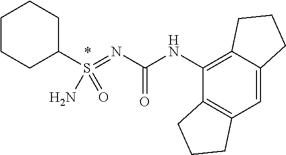 [SMILES: NS(=O)(=NC(=O)Nc1c2c(cc3c1CCC3)CCC2)C1CCCCC1] |
| 148 | 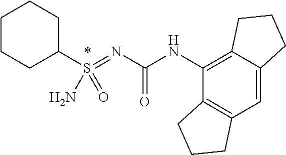 [SMILES: NS(=O)(=NC(=O)Nc1c2c(cc3c1CCC3)CCC2)C1CCCCC1] |
| 149 | 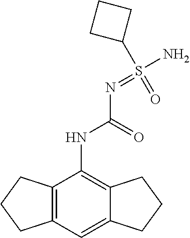 [SMILES: NS(=O)(=NC(=O)Nc1c2c(cc3c1CCC3)CCC2)C1CCC1] |
| 150 | 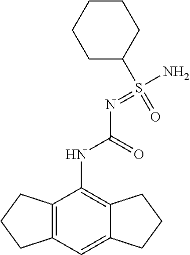 [SMILES: NS(=O)(=NC(=O)Nc1c2c(cc3c1CCC3)CCC2)C1CCCCC1] |
| 151 | 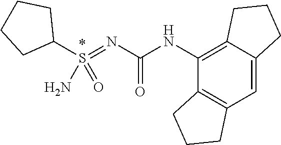 [SMILES: NS(=O)(=NC(=O)Nc1c2c(cc3c1CCC3)CCC2)C1CCCC1] |
| 152 | 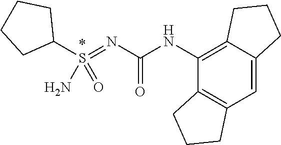 [SMILES: NS(=O)(=NC(=O)Nc1c2c(cc3c1CCC3)CCC2)C1CCCC1] |
| 153 | 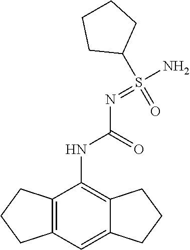 [SMILES: NS(=O)(=NC(=O)Nc1c2c(cc3c1CCC3)CCC2)C1CCCC1] |
| 154 | 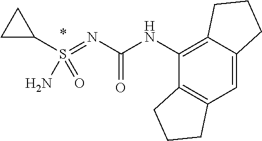 [SMILES: NS(=O)(=NC(=O)Nc1c2c(cc3c1CCC3)CCC2)C1CC1] |
| 155 | 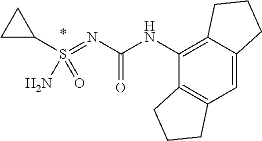 [SMILES: NS(=O)(=NC(=O)Nc1c2c(cc3c1CCC3)CCC2)C1CC1] |
| 156 | 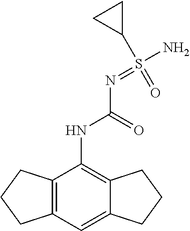 [SMILES: NS(=O)(=NC(=O)Nc1c2c(cc3c1CCC3)CCC2)C1CC1] |
| 157 | 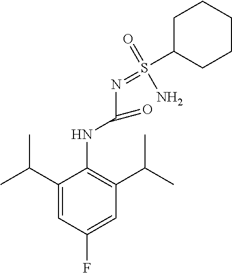 [SMILES: CC(C)c1cc(F)cc(C(C)C)c1NC(=O)N=S(N)(=O)C1CCCCC1] |
| 158 | 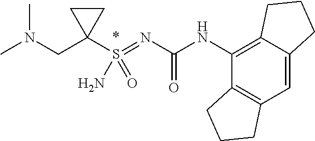 [SMILES: CN(C)CC1(S(N)(=O)=NC(=O)Nc2c3c(cc4c2CCC4)CCC3)CC1] |
| 159 | 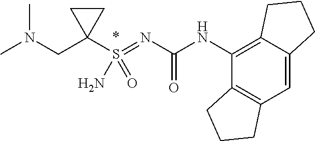 [SMILES: CN(C)CC1(S(N)(=O)=NC(=O)Nc2c3c(cc4c2CCC4)CCC3)CC1] |
| 160 | 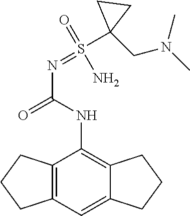 [SMILES: CN(C)CC1(S(N)(=O)=NC(=O)Nc2c3c(cc4c2CCC4)CCC3)CC1] |
| 161 | 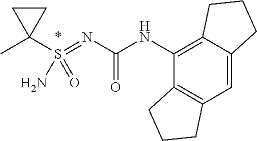 [SMILES: CC1(S(N)(=O)=NC(=O)Nc2c3c(cc4c2CCC4)CCC3)CC1] |
| 162 | 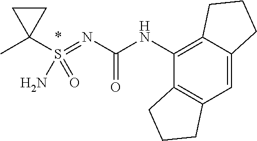 [SMILES: CC1(S(N)(=O)=NC(=O)Nc2c3c(cc4c2CCC4)CCC3)CC1] |
| 163 | 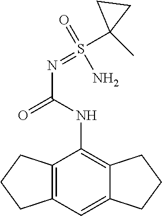 [SMILES: CC1(S(N)(=O)=NC(=O)Nc2c3c(cc4c2CCC4)CCC3)CC1] |
| 164 | 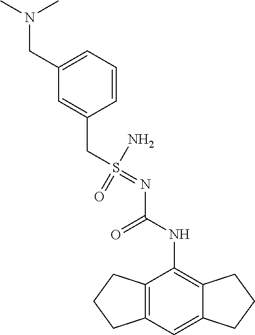 [SMILES: CN(C)Cc1cccc(CS(N)(=O)=NC(=O)Nc2c3c(cc4c2CCC4)CCC3)c1] |
| 165 | 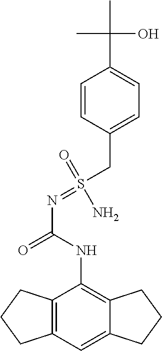 [SMILES: CC(C)(O)c1ccc(CS(N)(=O)=NC(=O)Nc2c3c(cc4c2CCC4)CCC3)cc1] |
| 165a | 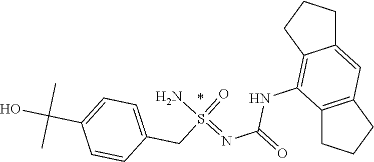 [SMILES: CC(C)(O)c1ccc(CS(N)(=O)=NC(=O)Nc2c3c(cc4c2CCC4)CCC3)cc1] |
| 165b | 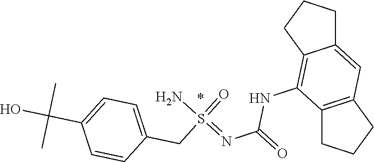 [SMILES: CC(C)(O)c1ccc(CS(N)(=O)=NC(=O)Nc2c3c(cc4c2CCC4)CCC3)cc1] |
| 166 | 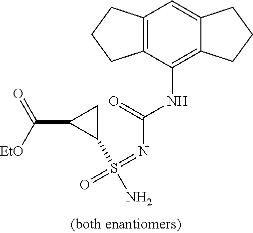 [SMILES: CCOC(=O)[C@H]1CC1[S@](N)(=O)=NC(=O)Nc1c2c(cc3c1CCC3)CCC2] |

In some embodiments, provided herein is a compound that is selected from the group consisting of the compounds in Table 1A-2:

TABLE 1A-2

| | |
|---|---|
| 201 | 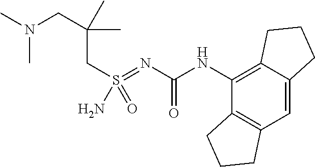 [SMILES: CN(C)CC(C)(C)CS(N)(=O)=NC(=O)Nc1c2c(cc3c1CCC3)CCC2] |
| 202 | 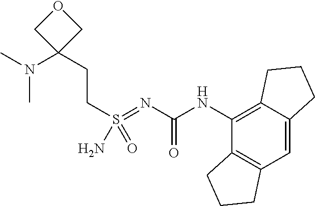 [SMILES: CN(C)C1(CCS(N)(=O)=NC(=O)Nc2c3c(cc4c2CCC4)CCC3)COC1] |
| 203 | 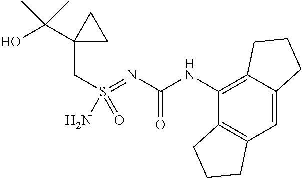 [SMILES: CC(C)(O)C1(CS(N)(=O)=NC(=O)Nc2c3c(cc4c2CCC4)CCC3)CC1] |
| 204 | 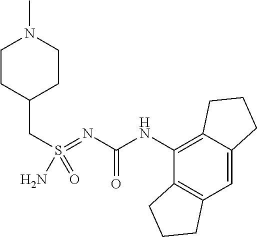 [SMILES: CN1CCC(CS(N)(=O)=NC(=O)Nc2c3c(cc4c2CCC4)CCC3)CC1] |
| 205 | 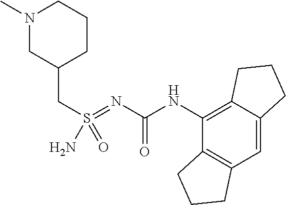 [SMILES: CN1CCCC(CS(N)(=O)=NC(=O)Nc2c3c(cc4c2CCC4)CCC3)C1] |
| 206 | 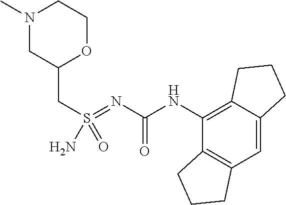 [SMILES: CN1CCOC(CS(N)(=O)=NC(=O)Nc2c3c(cc4c2CCC4)CCC3)C1] |
| 207 | 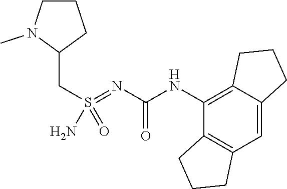 [SMILES: CN1CCCC1CS(N)(=O)=NC(=O)Nc1c2c(cc3c1CCC3)CCC2] |
| 208 | 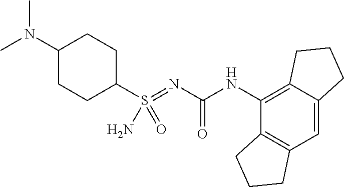 [SMILES: CN(C)C1CCC(S(N)(=O)=NC(=O)Nc2c3c(cc4c2CCC4)CCC3)CC1] |
| 209 | 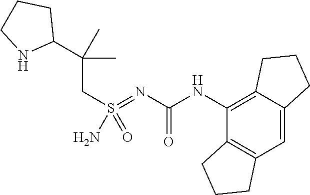 [SMILES: CC(C)(CS(N)(=O)=NC(=O)Nc1c2c(cc3c1CCC3)CCC2)C1CCCN1] |
| 210 | 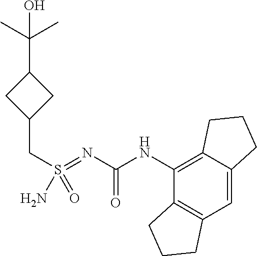 [SMILES: CC(C)(O)C1CC(CS(N)(=O)=NC(=O)Nc2c3c(cc4c2CCC4)CCC3)C1] |
| 211 | 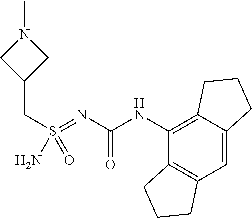 [SMILES: CN1CC(CS(N)(=O)=NC(=O)Nc2c3c(cc4c2CCC4)CCC3)C1] |
| 212 | 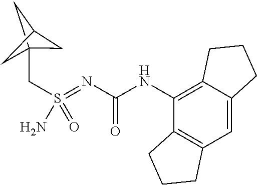 [SMILES: NS(=O)(CC12CC(C1)C2)=NC(=O)Nc1c2c(cc3c1CCC3)CCC2] |
| 213 | 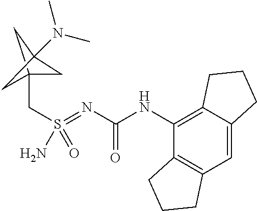 [SMILES: CN(C)C12CC(CS(N)(=O)=NC(=O)Nc3c4c(cc5c3CCC5)CCC4)(C1)C2] |
| 214 | 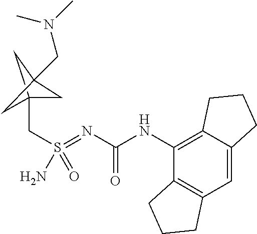 [SMILES: CN(C)CC12CC(CS(N)(=O)=NC(=O)Nc3c4c(cc5c3CCC5)CCC4)(C1)C2] |
| 215 | 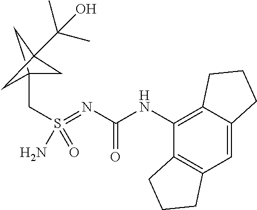 [SMILES: CC(C)(O)C12CC(CS(N)(=O)=NC(=O)Nc3c4c(cc5c3CCC5)CCC4)(C1)C2] |
| 216 | 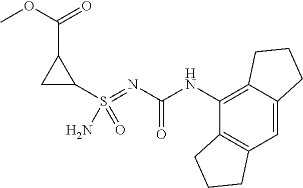 [SMILES: COC(=O)C1CC1S(N)(=O)=NC(=O)Nc1c2c(cc3c1CCC3)CCC2] |
| 217 | 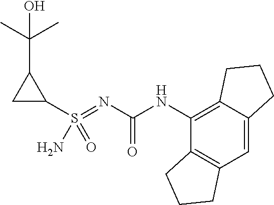 [SMILES: CC(C)(O)C1CC1S(N)(=O)=NC(=O)Nc1c2c(cc3c1CCC3)CCC2] |
| 218 | 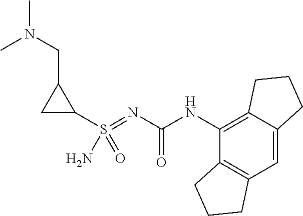 [SMILES: CN(C)CC1CC1S(N)(=O)=NC(=O)Nc1c2c(cc3c1CCC3)CCC2] |
| 219 | 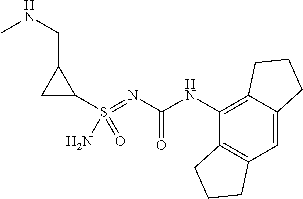 [SMILES: CNCC1CC1S(N)(=O)=NC(=O)Nc1c2c(cc3c1CCC3)CCC2] |
| 220 | 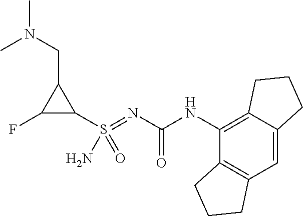 [SMILES: CN(C)CC1C(F)C1S(N)(=O)=NC(=O)Nc1c2c(cc3c1CCC3)CCC2] |
| 221 | 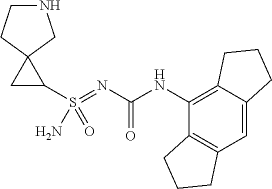 [SMILES: NS(=O)(=NC(=O)Nc1c2c(cc3c1CCC3)CCC2)C1CC12CCNC2] |
| 222 | 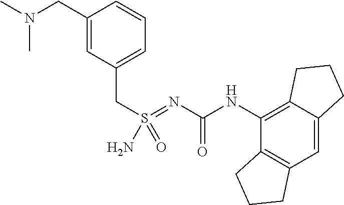 [SMILES: CN(C)Cc1cccc(CS(N)(=O)=NC(=O)Nc2c3c(cc4c2CCC4)CCC3)c1] |
| 223 | 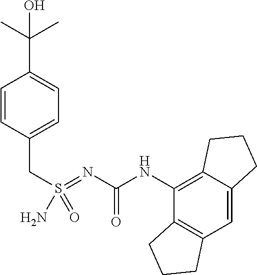 [SMILES: CC(C)(O)c1ccc(CS(N)(=O)=NC(=O)Nc2c3c(cc4c2CCC4)CCC3)cc1] |
| 224 | 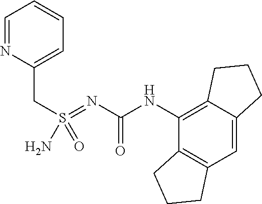 [SMILES: NS(=O)(Cc1ccccn1)=NC(=O)Nc1c2c(cc3c1CCC3)CCC2] |
| 225 | 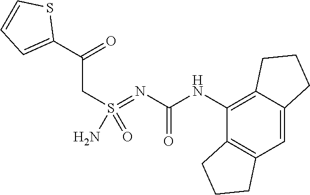 [SMILES: NS(=O)(CC(=O)c1cccs1)=NC(=O)Nc1c2c(cc3c1CCC3)CCC2] |
| 226 | 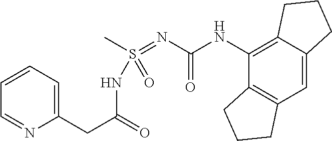 [SMILES: CS(=O)(=NC(=O)Nc1c2c(cc3c1CCC3)CCC2)NC(=O)Cc1ccccn1] |
| 227 | 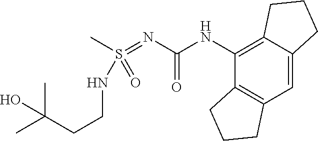 [SMILES: CC(C)(O)CCNS(C)(=O)=NC(=O)Nc1c2c(cc3c1CCC3)CCC2] |
| 228 | 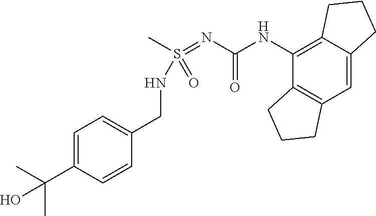 [SMILES: CC(C)(O)c1ccc(CNS(C)(=O)=NC(=O)Nc2c3c(cc4c2CCC4)CCC3)cc1] |
| 229 | 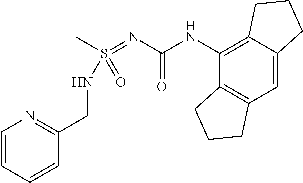 [SMILES: CS(=O)(=NC(=O)Nc1c2c(cc3c1CCC3)CCC2)NCc1ccccn1] |
| 230 | 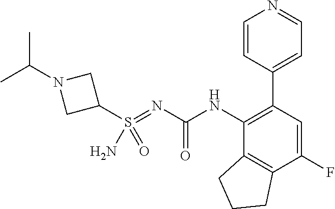 [SMILES: CC(C)N1CC(S(N)(=O)=NC(=O)Nc2c(-c3ccncc3)cc(F)c3c2CCC3)C1] |
| 231 | 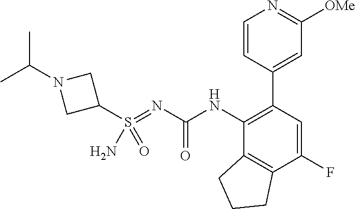 [SMILES: COc1cc(-c2cc(F)c3c(c2NC(=O)N=S(N)(=O)C2CN(C(C)C)C2)CCC3)ccn1] |
| 232 | 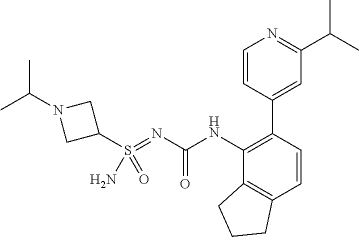 [SMILES: CC(C)c1cc(-c2ccc3c(c2NC(=O)N=S(N)(=O)C2CN(C(C)C)C2)CCC3)ccn1] |
| 233 | 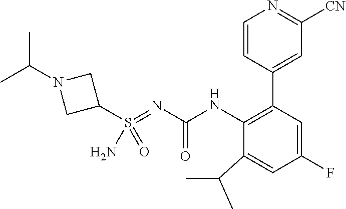 [SMILES: CC(C)c1cc(F)cc(-c2ccnc(C#N)c2)c1NC(=O)N=S(N)(=O)C1CN(C(C)C)C1] |
| 234 | 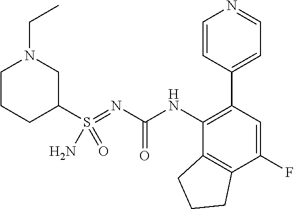 [SMILES: CCN1CCCC(S(N)(=O)=NC(=O)Nc2c(-c3ccncc3)cc(F)c3c2CCC3)C1] |
| 235 | 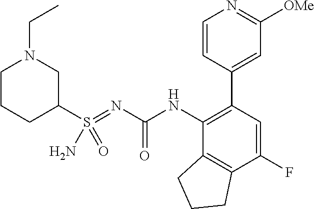 [SMILES: CCN1CCCC(S(N)(=O)=NC(=O)Nc2c(-c3ccnc(OC)c3)cc(F)c3c2CCC3)C1] |
| 236 | 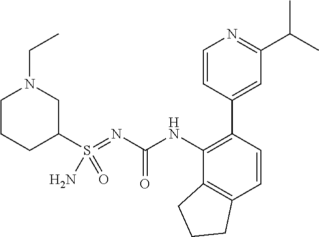 [SMILES: CCN1CCCC(S(N)(=O)=NC(=O)Nc2c(-c3ccnc(C(C)C)c3)ccc3c2CCC3)C1] |
| 237 | 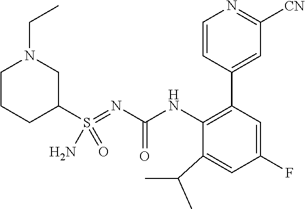 [SMILES: CCN1CCCC(S(N)(=O)=NC(=O)Nc2c(-c3ccnc(C#N)c3)cc(F)cc2C(C)C)C1] |
| 238 | 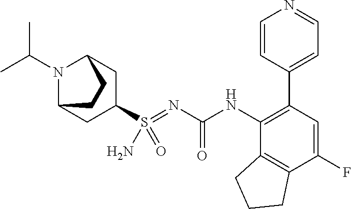 |
| 239 | 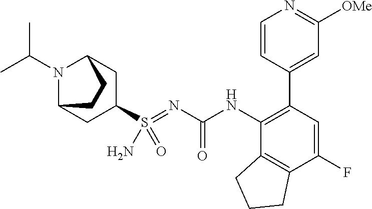 |
| 240 | 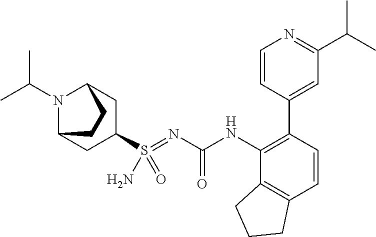 |
| 241 | 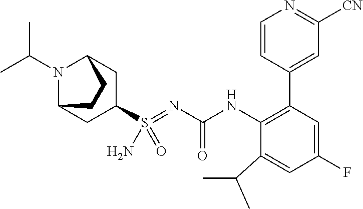 |
| 242 | 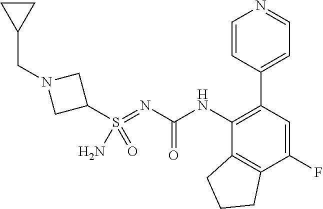 [SMILES: NS(=O)(=NC(=O)Nc1c(-c2ccncc2)cc(F)c2c1CCC2)C1CN(CC2CC2)C1] |
| 243 | 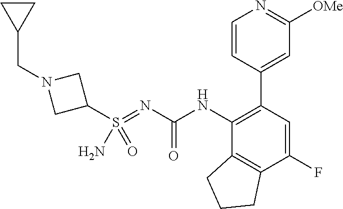 [SMILES: COc1cc(-c2cc(F)c3c(c2NC(=O)N=S(N)(=O)C2CN(CC4CC4)C2)CCC3)ccn1] |
| 244 | 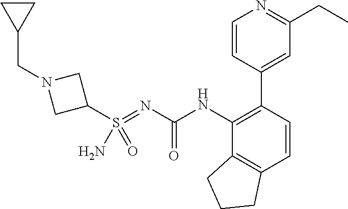 [SMILES: CCc1cc(-c2ccc3c(c2NC(=O)N=S(N)(=O)C2CN(CC4CC4)C2)CCC3)ccn1] |
| 245 | 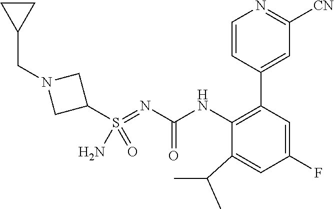 [SMILES: CC(C)c1cc(F)cc(-c2ccnc(C#N)c2)c1NC(=O)N=S(N)(=O)C1CN(CC2CC2)C1] |

TABLE 1B

| Compound | Structure |
|---|---|
| 301 | 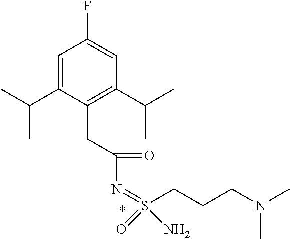 [SMILES: CC(C)c1cc(F)cc(C(C)C)c1CC(=O)N=S(N)(=O)CCCN(C)C] |
| 302 | 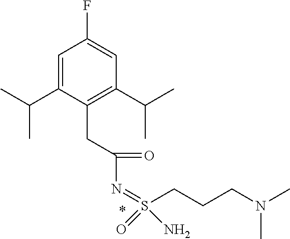 [SMILES: CC(C)c1cc(F)cc(C(C)C)c1CC(=O)N=S(N)(=O)CCCN(C)C] |
| 303 | 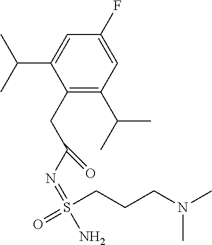 [SMILES: CC(C)c1cc(F)cc(C(C)C)c1CC(=O)N=S(N)(=O)CCCN(C)C] |
| 304 | 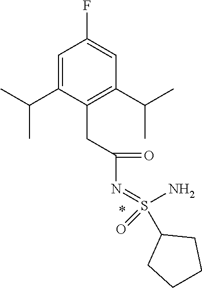 [SMILES: CC(C)c1cc(F)cc(C(C)C)c1CC(=O)N=S(N)(=O)C1CCCC1] |
| 305 | 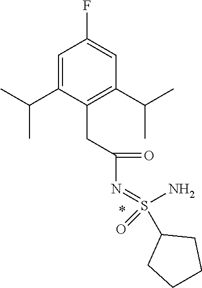 [SMILES: CC(C)c1cc(F)cc(C(C)C)c1CC(=O)N=S(N)(=O)C1CCCC1] |
| 306 | 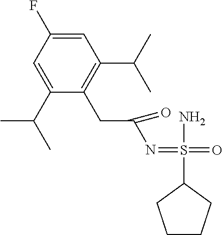 [SMILES: CC(C)c1cc(F)cc(C(C)C)c1CC(=O)N=S(N)(=O)C1CCCC1] |
| 307 | 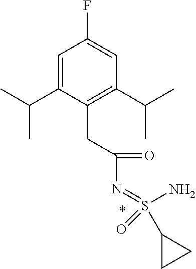 [SMILES: CC(C)c1cc(F)cc(C(C)C)c1CC(=O)N=S(N)(=O)C1CC1] |
| 308 | 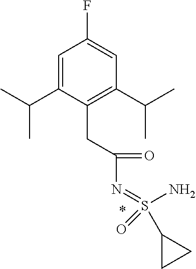 [SMILES: CC(C)c1cc(F)cc(C(C)C)c1CC(=O)N=S(N)(=O)C1CC1] |
| 309 | 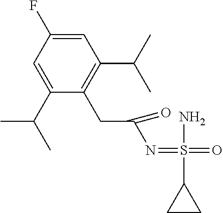 [SMILES: CC(C)c1cc(F)cc(C(C)C)c1CC(=O)N=S(N)(=O)C1CC1] |

TABLE 1C

| Compound | Structure |
|---|---|
| 401 | 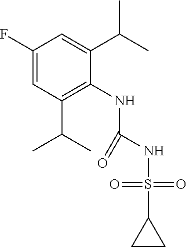 [SMILES: CC(C)c1cc(F)cc(C(C)C)c1NC(=O)NS(=O)(=O)C1CC1] |
| 402 | 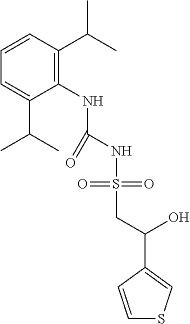 [SMILES: CC(C)c1cccc(C(C)C)c1NC(=O)NS(=O)(=O)CC(O)c1ccsc1] |
| 403 | 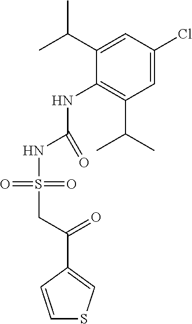 [SMILES: CC(C)c1cc(Cl)cc(C(C)C)c1NC(=O)NS(=O)(=O)CC(=O)c1ccsc1] |
| 404 | 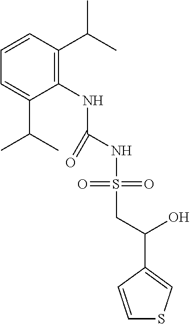 [SMILES: CC(C)c1cccc(C(C)C)c1NC(=O)NS(=O)(=O)CC(O)c1ccsc1] |
| 405 | 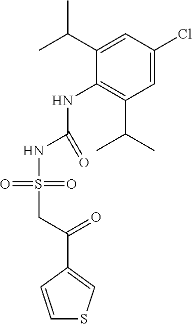 [SMILES: CC(C)c1cc(Cl)cc(C(C)C)c1NC(=O)NS(=O)(=O)CC(=O)c1ccsc1] |

TABLE 1D

| Compound | Structure |
|---|---|
| 501 | 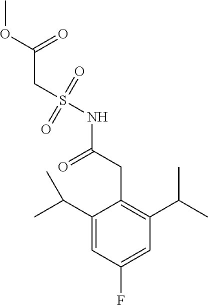 [SMILES: COC(=O)CS(=O)(=O)NC(=O)Cc1c(C(C)C)cc(F)cc1C(C)C] |
| 502 | 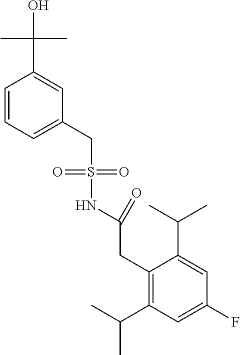 [SMILES: CC(C)c1cc(F)cc(C(C)C)c1CC(=O)NS(=O)(=O)Cc1cccc(C(C)(C)O)c1] |
| 503 | 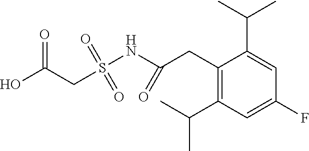 [SMILES: CC(C)c1cc(F)cc(C(C)C)c1CC(=O)NS(=O)(=O)CC(=O)O] |
| 504 | 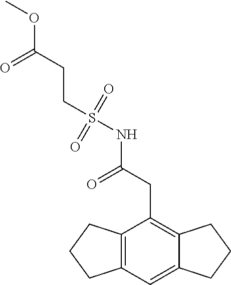 [SMILES: COC(=O)CCS(=O)(=O)NC(=O)Cc1c2c(cc3c1CCC3)CCC2] |
| 505 | 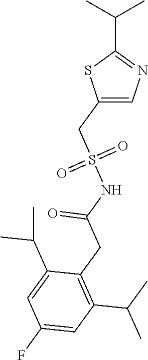 [SMILES: CC(C)c1ncc(CS(=O)(=O)NC(=O)Cc2c(C(C)C)cc(F)cc2C(C)C)s1] |
| 506 | 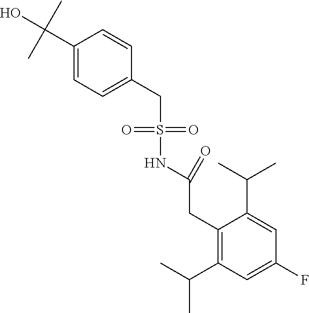 [SMILES: CC(C)c1cc(F)cc(C(C)C)c1CC(=O)NS(=O)(=O)Cc1ccc(C(C)(C)O)cc1] |
| 507 | 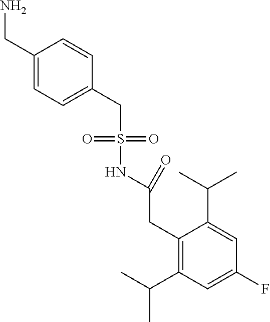 [SMILES: CC(C)c1cc(F)cc(C(C)C)c1CC(=O)NS(=O)(=O)Cc1ccc(CN)cc1] |
| 508 | 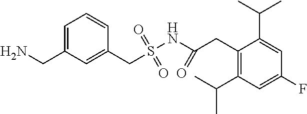 [SMILES: CC(C)c1cc(F)cc(C(C)C)c1CC(=O)NS(=O)(=O)Cc1cccc(CN)c1] |
| 509 | 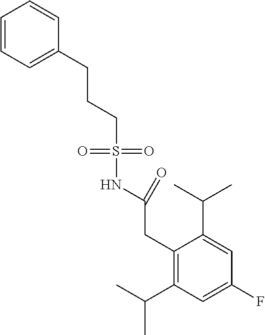 [SMILES: CC(C)c1cc(F)cc(C(C)C)c1CC(=O)NS(=O)(=O)CCCc1ccccc1] |
| 510 | 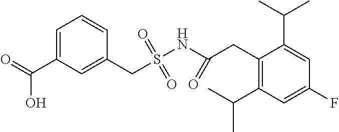 [SMILES: CC(C)c1cc(F)cc(C(C)C)c1CC(=O)NS(=O)(=O)Cc1cccc(C(=O)O)c1] |
| 511 | 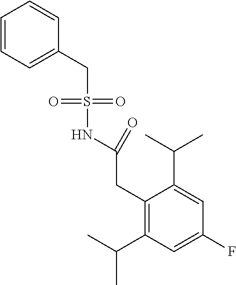 [SMILES: CC(C)c1cc(F)cc(C(C)C)c1CC(=O)NS(=O)(=O)Cc1ccccc1] |
| 512 | 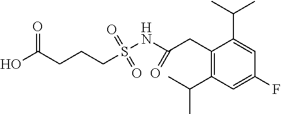 [SMILES: CC(C)c1cc(F)cc(C(C)C)c1CC(=O)NS(=O)(=O)CCCC(=O)O] |
| 513 | 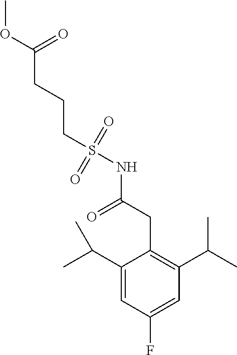 [SMILES: COC(=O)CCCS(=O)(=O)NC(=O)Cc1c(C(C)C)cc(F)cc1C(C)C] |
| 514 | 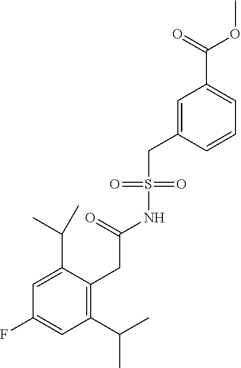 [SMILES: COC(=O)c1cccc(CS(=O)(=O)NC(=O)Cc2c(C(C)C)cc(F)cc2C(C)C)c1] |
| 515 | 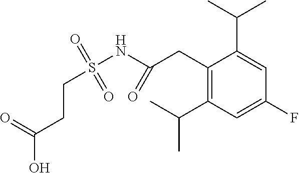 [SMILES: CC(C)c1cc(F)cc(C(C)C)c1CC(=O)NS(=O)(=O)CCC(=O)O] |
| 516 | 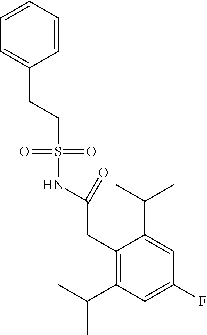 [SMILES: CC(C)c1cc(F)cc(C(C)C)c1CC(=O)NS(=O)(=O)CCc1ccccc1] |
| 517 | 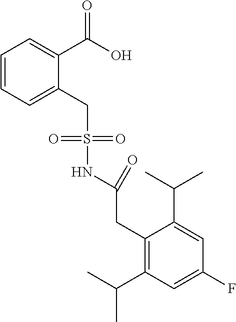 [SMILES: CC(C)c1cc(F)cc(C(C)C)c1CC(=O)NS(=O)(=O)Cc1ccccc1C(=O)O] |
| 518 | 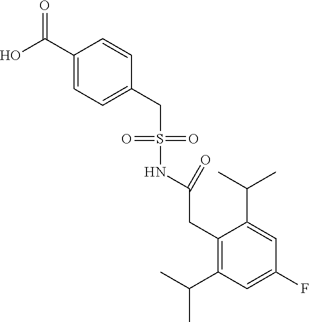 [SMILES: CC(C)c1cc(F)cc(C(C)C)c1CC(=O)NS(=O)(=O)Cc1ccc(C(=O)O)cc1] |
| 519 | 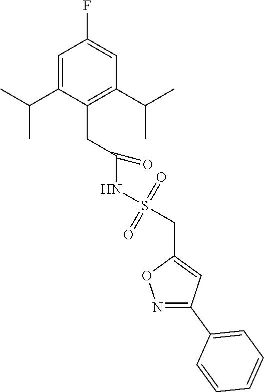 [SMILES: CC(C)c1cc(F)cc(C(C)C)c1CC(=O)NS(=O)(=O)Cc1cc(-c2ccccc2)no1] |
| 520 | 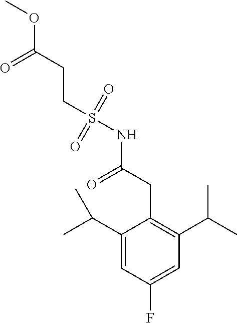 [SMILES: COC(=O)CCS(=O)(=O)NC(=O)Cc1c(C(C)C)cc(F)cc1C(C)C] |
| 521 | 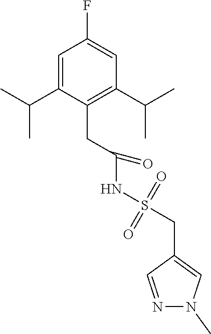 [SMILES: CC(C)c1cc(F)cc(C(C)C)c1CC(=O)NS(=O)(=O)Cc1cnn(C)c1] |
| 522 | 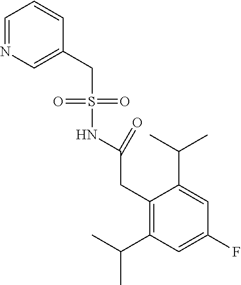 [SMILES: CC(C)c1cc(F)cc(C(C)C)c1CC(=O)NS(=O)(=O)Cc1cccnc1] |
| 523 | 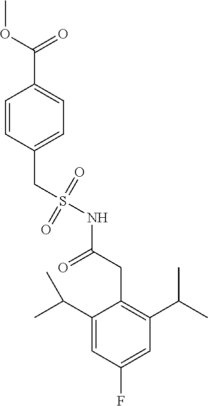 [SMILES: COC(=O)c1ccc(CS(=O)(=O)NC(=O)Cc2c(C(C)C)cc(F)cc2C(C)C)cc1] |
| 524 | 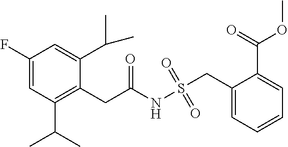 [SMILES: COC(=O)c1ccccc1CS(=O)(=O)NC(=O)Cc1c(C(C)C)cc(F)cc1C(C)C] |
| 525 | 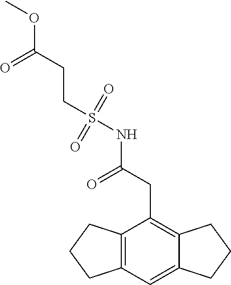 [SMILES: COC(=O)CCS(=O)(=O)NC(=O)Cc1c2c(cc3c1CCC3)CCC2] |
| 526 | 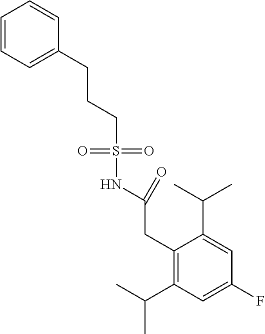 [SMILES: CC(C)c1cc(F)cc(C(C)C)c1CC(=O)NS(=O)(=O)CCCc1ccccc1] |
| 527 | 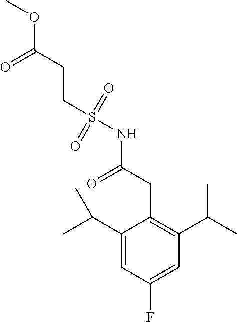 [SMILES: COC(=O)CCS(=O)(=O)NC(=O)Cc1c(C(C)C)cc(F)cc1C(C)C] |
| 528 | 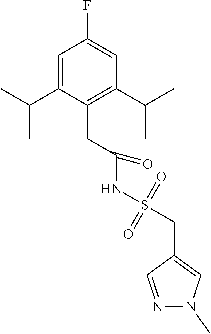 [SMILES: CC(C)c1cc(F)cc(C(C)C)c1CC(=O)NS(=O)(=O)Cc1cnn(C)c1] |

Pharmaceutical Compositions and Administration

General

In some embodiments, a chemical entity (e.g., a compound that modulates (e.g., antagonizes) NLRP3, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%400% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, an NLRP3 antagonist and/or an anti-TNFα agent disclosed herein is administered as a pharmaceutical composition that includes the NLRP3 antagonist and/or anti-TNFα agent and one or more pharmaceutically acceptable excipients, and optionally one or more additional therapeutic agents as described herein. Preferably the pharmaceutical composition that includes an NLRP3 antagonist and an anti-TNFα agent.

Preferably the above pharmaceutical composition embodiments comprise an NLRP3 antagonist disclosed herein. More preferably the above pharmaceutical composition embodiments comprise an NLRP3 antagonist and an anti-TNFα agent disclosed herein.

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acidmethyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phospahate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phospahate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a compound of Formula AA, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phospahate dehydrate, disodium phosphate dodecahydrate, crospovidone, lactose monohydrate, magnesium stearate, and talc. In certain of these embodiments, the chemical entity is a compound of Formula AA, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity (e.g., a compound of Formula AA, or a pharmaceutically acceptable salt and/or hydrate and/or cocrystal thereof; e.g., a compound of Formula AA) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a compound of Formula AA, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table 2.

TABLE 2

| Ingredient | Weight Percent |
|---|---|
| A compound of | 40 weight percent to about 80 weight |
| Formula AA | percent (e.g., from about 50 weight percent |
| | to about 70 weight percent, from about 55 |
| | weight percent to about 70 weight percent; |
| | from about 60 weight percent to about 65 |
| | weight percent; e.g., about 62.1 weight |
| | percent) |
| Crospovidone | 0.5 weight percent to about 5 weight |
| (Kollidon CL) | percent (e.g., from about 0.5 weight |
| | percent to about 3 weight percent, from |
| | about 1 weight percent to about 3 weight |
| | percent; about 1.93 weight percent |
| lactose monohydrate | about 10 weight percent to about 50 weight |
| (Pharmatose 200M) | percent (e.g., from about 20 weight percent |
| | to about 40 weight percent, from about 25 |
| | weight percent to about 35 weight percent; |
| | e.g., about 31.03 weight percent |
| Povidone | about 0.5 weight percent to about 5 weight |
| (Kollidon K30) | percent (e.g., from about 1.5 weight |
| | percent to about 4.5 weight percent, from |
| | about 2 weight percent to about 3.5 weight |
| | percent; e.g., about 2.76 weight percent |
| talc | 0.5 weight percent to about 5 weight |
| | percent (e.g., from about 0.5 weight |
| | percent to about 3 weight percent, from |
| | about 1 weight percent to about 3 weight |
| | percent; from about 1.5 weight percent to |
| | about 2.5 weight percent; from about 1.8 |
| | weight percent to about 2.2 weight |
| | percent; e.g., about 1.93 weight percent |
| Magnesium | about 0.05 weight percent to about 1 |
| stearate | weight percent (e.g., from about 0.05 |
| | weight percent to about 1 weight percent; |
| | from about 0.1 weight percent to about 1 |
| | weight percent; from about 0.1 weight |
| | percent to about 0.5 weight percent; e.g., |
| | about 0.27 weight percent |

In certain embodiments, component (i) includes the ingredients and amounts as shown in Table 3.

TABLE 3

| Ingredient | Weight Percent |
|---|---|
| A compound of Formula AA | About 62.1 weight percent) |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phospahate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phospahate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′″) is present.

In certain embodiments, component (ii) includes water (up to 100%) and the ingredients and amounts as shown in Table 4.

TABLE 4

| Ingredient | Weight Percent |
|---|---|
| methyl cellulose | 0.05 weight percent to about 5 weight |
| (Methocel A15C | percent (e.g., from about 0.05 weight |
| premium) | percent to about 3 weight percent, from |
| | about 0.1 weight percent to about 3 weight |
| | percent; e.g., about 1.4 weight percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 weight |
| | percent; e.g., about 1.0 weight percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight percent; |
| | e.g., about 0.20 weight percent) |
| disodium phosphate | about 0.05 weight percent to about 1 |
| dodecahydrate | weight percent (e.g., from about 0.05 |
| | weight percent to about 0.5 weight percent; |
| | e.g., about 0.15 weight percent) |
| sodium dihydrogen | about 0.005 weight percent to about 0.5 |
| phospahate dihydrate | weight percent (e.g., from about 0.005 |
| | weight percent to about 0.3 weight percent; |
| | e.g., about 0.15 weight percent) |

In certain embodiments, component (ii) includes water (up to 100%) and the ingredients and amounts as shown in Table 5.

TABLE 5

| Ingredient | Weight Percent |
|---|---|
| methyl cellulose (Methocel A15C | about 1.4 weight percent |
| premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 mL to about 500 mL, from about 1 mL to about 250 mL, from about 1 mL to about 100 mL, from about 10 mL to about 1000 mL, from about 10 mL to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid carrier.

In certain embodiments, enema formulations include from about 50 mg to about 250 mg (e.g., from about 100 mg to about 200; e.g., about 150 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 150 mg of a compound of Formula AA in about 60 mL of the liquid carrier.

In certain embodiments, enema formulations include from about 350 mg to about 550 mg (e.g., from about 400 mg to about 500; e.g., about 450 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 450 mg of a compound of Formula AA in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL; from about 0.01 mg/mL to about 5 mg/mL; from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the chemical entity in liquid carrier. In certain of these embodiments, the chemical entity is a compound of Formula AA, or a pharmaceutically acceptable salt and/or hydrate and/or cocrystal thereof. For example, enema formulations can include about 2.5 mg/mL or about 7.5 mg/mL of a compound of Formula AA in liquid carrier.

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which a decrease or increase in NLRP3 activity (e.g., an increase, e.g., NLRP3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same).

Indications

In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; auto-immune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, the condition, disease or disorder is selected from major adverse cardiovascular events such as carbiovascular death, non-fatal myocardial infarction and non-fatal stroke in patients with a prior hear attack and inflammatory atherosclerosis (see for example, NCT01327846).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity.

In some embodiments, the condition, disease or disorder is Type 2 Diabetes.

In some embodiments, the condition, disease or disorder is NASH.

In some embodiments, the condition, disease or disorder is Alzheimer's disease.

In some embodiments, the condition, disease or disorder is gout.

In some embodiments, the condition, disease or disorder is SLE.

In some embodiments, the condition, disease or disorder is rheumatoid arthritis.

In some embodiments, the condition, disease or disorder is IBD.

In some embodiments, the condition, disease or disorder is multiple sclerosis.

In some embodiments, the condition, disease or disorder is COPD.

In some embodiments, the condition, disease or disorder is asthma.

In some embodiments, the condition, disease or disorder is scleroderma.

In some embodiments, the condition, disease or disorder is pulmonary fibrosis.

In some embodiments, the condition, disease or disorder is age related macular degeneration (AMD).

In some embodiments, the condition, disease or disorder is cystic fibrosis.

In some embodiments, the condition, disease or disorder is Muckle Wells syndrome.

In some embodiments, the condition, disease or disorder is familial cold autoinflammatory syndrome (FCAS).

In some embodiments, the condition, disease or disorder is chronic neurologic cutaneous and articular syndrome.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis.

In some embodiments, the condition, disease or disorder is selected from:

myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis.

In some embodiments, the indication is MDS.

In some embodiments, the indication is non-small lung cancer in patients carrying mutation or overexpression of NLRP3.

In some embodiments, the indication is ALL in patients resistant to glucocorticoids treatment.

In some embodiments, the indication is LCH.

In some embodiments, the indication is multiple myeloma.

In some embodiments, the indication is promyelocytic leukemia.

In some embodiments, the indication is gastric cancer.

In some embodiments, the indication is lung cancer metastasis.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where polymorphism is a gain of function

In some embodiments, the methods described herein further include the step of identifying a subject (e.g., a patient) in need of treatment for an indication related to NLRP3 activity, such as an indication related to NLRP3 polymorphism found in CAPS syndromes.

In some embodiments, the methods described herein further include the step of identifying a subject (e.g., a patient) in need of treatment for an indication related to NLRP3 activity, such as an indication related NLRP3 polymorphism where the polymorphism is VAR_014104 (R262W)

In some embodiments, the methods described herein further include the step of identifying a subject (e.g., a patient) in need of treatment for an indication related to NLRP3 activity, such as an indication related NLRP3 polymorphism where the polymorphism is a natural variant reported in http://www.uniprot.org/uniprot/Q96P20.

In some embodiments, the methods described herein further include the step of identifying a subject (e.g., a patient) in need of treatment for an indication related to NLRP3 activity, such as an indication related to point mutation of NLRP3 signaling.

Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily 1B (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2) and/or. Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibit reduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (JNK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSF1A associated via death domain (TRADD), and TNF receptor associated factor 2 (TRAF2), in a cell), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et al., Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB.

In other embodiments, anti-TNFα agents that can indirectly block, down-regulate, impair, or reduce one or more components in a cell (e.g., a cell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), JNK, lipopolysaccharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP). For example, such indirect anti-TNFα agents can be an inhibitory nucleic acid that targets (decreases the expression) of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, LBP, MEK1/2, MEK3/6, MEK4/7, MK2, MyD88, NF-κB, NIK, IRAK, lipopolysaccharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, lipopolysaccharide binding protein (LBP), MEK1/2, MEK3/6, MEK4/7, MK2, MyD88, NF-κB, NIK, IRAK, lipopolysaccharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα. In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)2, a minibody, or a BiTE.

In some embodiments, an antibody can be a crossmab, a diabody, a scDiabody, a scDiabody-CH₃, a Diabody-CH₃, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kλ-body, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)—IgG, an IgG(L)-V, a V(L)-IgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fc, a scFv-HAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH₃ KIH, a Fab-scFv, a scFv-CH-CL-scFv, a F(ab′)2-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFv1-PEG-scFv2.

Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et al., Autoimmunity Rev. 13(1):24-30, 2014; Bongartz et al., JAMA 295(19):2275-2285, 2006; Butler et al., Eur. Cytokine Network 6(4):225-230, 1994; Cohen et al., Canadian J. Gastroenterol. Hepatol. 15(6):376-384, 2001; Elliott et al., Lancet 1994; 344: 1125-1127, 1994; Feldmann et al., Ann. Rev. Immunol. 19(1):163-196, 2001; Rankin et al., Br. J. Rheumatol. 2:334-342, 1995; Knight et al., Molecular Immunol. 30(16):1443-1453, 1993; Lorenz et al., J. Immunol. 156(4):1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic Dis. 72(12):1947-1955, 2013; Palladino et al., Nature Rev. Drug Discovery 2(9):736-746, 2003; Sandborn et al., Inflammatory Bowel Diseases 5(2):119-133, 1999; Atzeni et al., Autoimmunity Reviews 12(7):703-708, 2013; Maini et al., Immunol. Rev. 144(1):195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab (Golimumab™), adalimumab (Humira™), infliximab (Remicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pfizer, GS071 from Aprogen, Remsima™, PF-06438179 from Pfizer/Sandoz, NI-071 from Nichi-Iko Pharmaceutical Co., and ABP 710 from Amgen; adalimumab biosimilars such as Amgevita® (ABP 501) from Amgen and Exemptia™ from Zydus Cadila, BMO-2 or MYL-1401-A from Biocon/Mylan, CHS-1420 from Coherus, FKB327 from Kyowa Kirin, and BI 695501 from Boehringer Ingelheim; Solymbic®, SB5 from Samsung Bioepis, GP-2017 from Sandoz, ONS-3010 from Oncobiologics, M923 from Momenta, PF-06410293 from Pfizer, and etanercept biosimilars such as Erelzi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximabm, CDP571, and CDP 870.

In some embodiments, any of the antibodies or antigen-binding fragments described herein has a dissociation constant (K_{D}) of less than 1×10⁻⁵M (e.g., less than 0.5×10⁻⁵ M, less than 1×10⁻⁶ M, less than 0.5×10⁻⁶ M, less than 1×10⁻⁷ M, less than 0.5×10⁻⁷M, less than 1×10⁻⁸ M, less than 0.5×10⁻⁸ M, less than 1×10⁻⁹ M, less than 0.5×10⁻⁹M, less than 1×10⁻¹⁰ M, less than 0.5×10⁻¹⁰ M, less than 1×10⁻¹¹M, less than 0.5×10⁻¹¹M, or less than 1×10⁻¹² M), e.g., as measured in phosphate buffered saline using surface plasmon resonance (SPR).

In some embodiments, any of the antibodies or antigen-binding fragments described herein has a K_{D} of about 1×10⁻¹² M to about 1×10⁻⁵M, about 0.5×10⁻⁵ M, about 1×10⁻⁶ M, about 0.5×10⁻⁶ M, about 1×10⁻⁷M, about 0.5×10⁻⁷ M, about 1×10⁻⁸ M, about 0.5×10⁻⁸ M, about 1×10⁻⁹M, about 0.5×10⁻⁹ M, about 1×10⁻¹⁰ M, about 0.5×10⁻¹⁰ M, about 1×10⁻¹¹ M, or about 0.5×10⁻¹¹M (inclusive); about 0.5×10⁻¹¹M to about 1×10⁻⁵M, about 0.5×10⁻⁵ M, about 1×10⁻⁶ M, about 0.5×10⁻⁶ M, about 1×10⁻⁷M, about 0.5×10⁻⁷M, about 1×10⁻⁸ M, about 0.5×10⁻⁸ M, about 1×10⁻⁹M, about 0.5×10⁻⁹ M, about 1×10⁻¹⁰ M, about 0.5×10⁻¹⁰ M, or about 1×10⁻¹¹M (inclusive); about 1×10⁻¹¹M to about 1×10⁻⁵M, about 0.5×10⁻⁵ M, about 1×10⁻⁶ M, about 0.5×10⁻⁶ M, about 1×10⁻⁷M, about 0.5×10⁻⁷M, about 1×10⁻⁸ M, about 0.5×10⁻⁸ M, about 1×10⁻⁹M, about 0.5×10⁻⁹M, about 1×10⁻¹⁰ M, or about 0.5×10⁻¹⁰ M (inclusive); about 0.5×10⁻¹⁰ M to about 1×10⁻⁵M, about 0.5×10⁻⁵ M, about 1×10⁻⁵ M, about 0.5×10⁻⁶ M, about 1×10⁻⁶ M, about 0.5×10⁻⁷M, about 1×10⁻⁸ M, about 0.5×10⁻⁸ M, about 1×10⁻⁹M, about 0.5×10⁻⁹M, or about 1×10⁻¹⁰ M (inclusive); about 1×10⁻¹⁰ M to about 1×10⁻⁵M, about 0.5×10⁻⁵M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, or about 0.5×10⁻⁹ M (inclusive); about 0.5×10⁻⁹M to about 1×10⁻⁵M, about 0.5×10⁻⁵ M, about 1×10⁻⁶ M, about 0.5×10⁻⁶ M, about 1×10⁻⁷M, about 0.5×10⁻⁷ M, about 1×10⁻⁸ M, about 0.5×10⁻⁸ M, or about 1×10⁻⁹M (inclusive); about 1×10⁻⁹ M to about 1×10⁻⁵M, about 0.5×10⁻⁵ M, about 1×10⁻⁶ M, about 0.5×10⁻⁶ M, about 1×10⁻⁷M, about 0.5×10⁻⁷ M, about 1×10⁻⁸ M, or about 0.5×10⁻⁸ M (inclusive); about 0.5×10⁻⁸ M to about 1×10⁻⁵ M, about 0.5×10⁻⁵ M, about 1×10⁻⁶ M, about 0.5×10⁻⁶ M, about 1×10⁻⁷M, about 0.5×10⁻⁷ M, or about 1×10⁻⁸ M (inclusive); about 1×10⁻⁸ M to about 1×10⁻⁵M, about 0.5×10⁻⁵ M, about 1×10⁻⁶ M, about 0.5×10⁻⁶ M, about 1×10⁻⁷M, or about 0.5×10⁻⁷M (inclusive); about 0.5×10⁻⁷ M to about 1×10⁻⁵M, about 0.5×10⁻⁵ M, about 1×10⁻⁶ M, about 0.5×10⁻⁶ M, or about 1×10⁻⁷M (inclusive); about 1×10⁻⁷ M to about 1×10⁻⁵M, about 0.5×10⁻⁵ M, about 1×10⁻⁶ M, or about 0.5×10⁻⁶ M (inclusive); about 0.5×10⁻⁶ M to about 1×10⁻⁵M, about 0.5×10⁻⁵ M, or about 1×10⁻⁶ M (inclusive); about 1×10⁻⁶ M to about 1×10⁻⁵M or about 0.5×10⁻⁵ M (inclusive); or about 0.5×10⁻⁵M to about 1×10⁻⁵M (inclusive), e.g., as measured in phosphate buffered saline using surface plasmon resonance (SPR).

In some embodiments, any of the antibodies or antigen-binding fragments described herein has a K_{off} of about 1×10⁻⁶ s⁻¹ to about 1×10⁻³ s⁻¹, about 0.5×10⁻³ s⁻¹, about 1×10⁻⁴ s⁻¹, about 0.5×10⁻⁴ s⁻¹, about 1×10⁻⁵ s⁻¹, or about 0.5×10⁻⁵ s⁻¹ (inclusive); about 0.5×10⁻⁵ s⁻¹ to about 1×10⁻³ s⁻¹, about 0.5×10⁻³ s⁻¹, about 1×10⁻⁴ s⁻¹, about 0.5×10⁻⁴ s⁻¹, or about 1×10⁻⁵ s⁻¹ (inclusive); about 1×10⁻⁵ s⁻¹ to about 1×10⁻³ s⁻¹, about 0.5×10⁻³ s⁻¹, about 1×10⁻⁴ s⁻¹, or about 0.5×10⁻⁴ s⁻¹ (inclusive); about 0.5×10⁻⁴ s⁻¹ to about 1×10⁻³ s⁻¹, about 0.5×10⁻³ s⁻¹, or about 1×10⁻⁴ s⁻¹ (inclusive); about 1×10⁻⁴ s⁻¹ to about 1×10⁻³ s⁻¹, or about 0.5×10⁻³ s⁻¹ (inclusive); or about 0.5×10⁻⁵ s⁻¹ to about 1×10⁻³ s⁻¹ (inclusive), e.g., as measured in phosphate buffered saline using surface plasmon resonance (SPR).

In some embodiments, any of the antibodies or antigen-binding fragments described herein has a Kₒₙ of about 1×10² M⁻¹ s⁻¹ to about 1×10⁶M⁻¹ s⁻¹, about 0.5×10⁶ M⁻¹ s⁻¹, about 1×10⁵M⁻¹ s⁻¹, about 0.5×10⁵M⁻¹ s⁻¹, about 1×10⁴ M⁻¹ s about 0.5×10⁴ M⁻¹ s⁻¹, about 1×10³ M⁻¹ s⁻¹, or about 0.5×10³ M⁻¹ s⁻¹ (inclusive); about 0.5×10⁻³ M⁻¹ s⁻¹ to about 1×10⁶ M⁻¹ s⁻¹, about 0.5×10⁶ M⁻¹ s⁻¹, about 1×10⁵M⁻¹ s⁻¹, about 0.5×10⁵M⁻¹ s⁻¹, about 1×10⁴M⁻¹ s⁻¹, about 0.5×10⁴ M⁻¹ s⁻¹, or about 1×10³ M⁻¹ s⁻¹ (inclusive); about 1×10³ M⁻¹ s⁻¹ to about 1×10⁶M⁻¹ s⁻¹, about 0.5×10⁶M⁻¹ s⁻¹ about 1×10⁵M⁻¹ s⁻¹, about 0.5×10⁵M⁻¹ s⁻¹, about 1×10⁴ M⁻¹ s⁻¹, or about 0.5×10⁴ M⁻¹ s⁻¹ (inclusive); about 0.5×10⁴ M⁻¹ s⁻¹ to about 1×10⁶M⁻¹ s⁻¹, about 0.5×10⁶ M⁻¹ s⁻¹, about 1×10⁵M⁻¹ s⁻¹, about 0.5×10⁵ M⁻¹ s⁻¹, or about 1×10⁴ M⁻¹ s⁻¹ (inclusive); about 1×10⁴ M⁻¹ s⁻¹ to about 1×10⁶ M⁻¹ s⁻¹, about 0.5×10⁶M⁻¹ s⁻¹, about 1×10⁵M⁻¹ s⁻¹, or about 0.5×10⁵ M⁻¹ s⁻¹ (inclusive); about 0.5×10⁵ M⁻¹ s⁻¹ to about 1×10⁶ M⁻¹ s⁻¹, about 0.5×10⁶ M⁻¹ s⁻¹, or about 1×10⁵M⁻¹ s⁻¹ (inclusive); about 1×10⁵ M⁻¹ s⁻¹ to about 1×10⁶ M⁻¹ s⁻¹, or about 0.5×10⁶ M⁻¹ s⁻¹ (inclusive); or about 0.5×10⁶ M⁻¹ s⁻¹ to about 1×10⁶ M⁻¹ s⁻¹ (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2):162, 2002; Peppel et al., J. Exp. Med. 174(6):1483-1489, 1991) or a soluble TNFR (e.g., TNFR1 or TNFR2) that binds specifically to TNFα. In some embodiments, the anti-TNFα agent includes or is a soluble TNFα receptor (e.g., Bjornberg et al., Lymphokine Cytokine Res. 13(3):203-211, 1994; Kozak et al., Am. Physiol. Reg. Integrative Comparative Physiol. 269(1):R23-R29, 1995; Tsao et al., Eur Respir J. 14(3):490-495, 1999; Watt et al., J Leukoc Biol. 66(6):1005-1013, 1999; Mohler et al., J. Immunol. 151(3):1548-1561, 1993; Nophar et al., EMBO J. 9(10):3269, 1990; Piguet et al., Eur. Respiratory J. 7(3):515-518, 1994; and Gray et al., Proc. Natl. Acad. Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J. Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is complementary to all or part of a AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g., fully or partially complementary to all or a part of any one of the sequences presented in table E).

| | mRNA GenBank |
|---|---|
| Human gene | accession number(s) |
| Tumor necrosis factor (TNF, a.k.a. TNF- | NM_000594 |
| alpha) | |
| TNF receptor superfamily member 1A | NM_001065 |
| (TNFRSF1A) (a. k. a. TNFR1) | NM_001346091 |
| | NM_001346092 |
| TNF receptor superfamily member 1B | NM_001066 |
| (TNFRSF1B) (a.k.a. TNFR2) | XM_011542060 |
| | XM_011542063 |
| | XM_017002214 |
| | XM_017002215 |
| | XM_017002211 |
| TNFRSF1A associated via death domain | NM_003789 |
| (TRADD) | NM_001323552 |
| | XM_005256213 |
| | XM_017023815 |
| TNF receptor associated factor 2 (TRAF2) | NM_021138 |
| | XM_011518976 |
| | XM_011518977 |
| | XM_011518974 |
| JunD proto-oncogene, AP-1 transcription | NM_001286968 |
| factor subunit (JUND) | NM_005354 |
| Mitogen-activated protein kinase kinase | NM_005923 |
| kinase 5 (MAP3K5) (a.k.a. ASK1) | XM_017010875 |
| | XM_017010872 |
| | XM_017010873 |
| | XM_017010877 |
| | XM_017010874 |
| | XM_017010871 |
| | XM_017010870 |
| | XM_017010876 |
| | XM_011535839 |
| CD14 | NM_000591 |
| | NM_001040021 |
| | NM_001174104 |
| | NM_001174105 |
| Mitogen-activated protein kinase 3 | NM_001040056 |
| (MAPK3) (a.k.a. ERK1) | NM_001109891 |
| | NM_002746 |
| Mitogen-activated protein kinase 1 | NM_002745 |
| (MAPK1) (a.k.a. ERK2) | NM_138957 |
| Inhibitor of nuclear factor kappa B kinase | NM_001190720 |
| subunit beta (IKBKB) | NM_001242778 |
| | NM_001556 |
| | XM_005273491 |
| | XM_005273496 |
| | XM_005273493 |
| | XM_005273498 |
| | XM_011544518 |
| | XM_005273492 |
| | XM_005273490 |
| | XM_005273494 |
| | 12XM_017013396 |
| | XM_011544521 |
| | XM_011544522 |
| | XM_005273495 |
| | XM_011544517 |
| | XM_011544520 |
| | XM_011544519 |
| NFKB inhibitor alpha (NFKBIA) | NM_020529 |
| Interleukin 1 receptor associated kinase 1 | NM_001025242 |
| (IRAK1) | NM_001025243 |
| | NM_001569 |
| | XM_005274668 |
| Mitogen-activated protein kinase 8 | NM_001278547 |
| (MAPK8) (a.k.a. JNK) | NM_001278548 |
| | NM_001323302 |
| | NM_001323320 |
| | NM_001323321 |
| | NM_001323322 |
| | NM_001323323 |
| | NM_001323324 |
| | NM_001323325 |
| | NM_001323326 |
| | NM_001323327 |
| | NM_001323328 |
| | NM_001323329 |
| | NM_001323330 |
| | NM_001323331 |
| | NM_139046 |
| | NM_139049 |
| | XM_024448079 |
| | XM_024448080 |
| Lipopolysaccharide binding protein (LBP) | NM_004139 |
| Mitogen-activated protein kinase kinase 1 | NM_002755 |
| (MAP2K1) (a.k.a. MEK1) | XM_017022411 |
| | XM_011521783 |
| | XM_017022412 |
| | XM_017022413 |
| Mitogen-activated protein kinase kinase 2 | NM_030662 |
| (MAP2K2) (a.k.a. MEK2) | XM_006722799 |
| | XM_017026990 |
| | XM_017026989 |
| | XM_017026991 |
| Mitogen-activated protein kinase kinase 3 | NM_001316332 |
| (MAP2K3) (a.k.a. MEK3) | NM_002756 |
| | NM_145109 |
| | XM_017024859 |
| | XM_005256723 |
| | XM_017024857 |
| | XM_011523959 |
| | XM_017024858 |
| | XM_011523958 |
| Mitogen-activated protein kinase kinase 6 | NM_001330450 |
| (MAP2K6) (a.k.a. MEK6) | NM_002758 |
| | XM_005257516 |
| | XM_011525027 |
| | XM_011525026 |
| | XM_006721975 |
| Mitogen-activated protein kinase kinase | NM_005921 |
| kinase 1 (MAP3K1) (a.k.a. MEKK1) | XM_017009485 |
| | XM_017009484 |
| Mitogen-activated protein kinase kinase | NM_001330431 |
| kinase 3 (MAP3K3) (a.k.a. MEKK3) | NM_001363768 |
| | NM_002401 |
| | NM_203351 |
| | XM_005257378 |
| Mitogen-activated protein kinase kinase | NM_001291958 |
| kinase 4 (MAP3K4) (a.k.a. MEKK4) | NM_001301072 |
| | NM_001363582 |
| | NM_005922 |
| | NM_006724 |
| | XM_017010869 |
| Mitogen-activated protein kinase kinase | NM_001297609 |
| kinase 6 (MAP3K6) (a.k.a. MEKK6) | NM_004672 |
| | XM_017002771 |
| | XM_017002772 |
| Mitogen-activated protein kinase kinase | NM_003188 |
| kinase 7 (MAP3K7) (a.k.a. MEKK7) | NM_145331 |
| | NM_145332 |
| | NM_145333 |
| | XM_006715553 |
| | XM_017011226 |
| MAPK activated protein kinase 2 | NM_004759 |
| (MAPKAPK2) (a.k.a. MK2) | NM_032960 |
| | XM_005273353 |
| | XM_017002810 |
| MYD88, innate immune signal | NM_001172566 |
| transduction adaptor (MYD88) | NM_001172567 |
| | NM_001172568 |
| | NM_001172569 |
| | NM_001365876 |
| | NM_001365877 |
| | NM_002468 |
| Nuclear factor kappa B subunit 1 (NFKB1) | NM_001165412 |
| | NM_001319226 |
| | NM_003998 |
| | XM_024454069 |
| | XM_024454067 |
| | XM_011532006 |
| | XM_024454068 |
| Mitogen-activated protein kinase kinase | NM_003954 |
| kinase 14 (MAP3K14) (a.k.a. NIK) | XM_011525441 |
| Mitogen-activated protein kinase 14 | NM_001315 |
| (MAPK14) (a.k.a. p38) | NM_139012 |
| | NM_139013 |
| | NM_139014 |
| | XM_011514310 |
| | XM_017010300 |
| | XM_017010299 |
| | XM_017010301 |
| | XM_017010304 |
| | XM_017010303 |
| | XM_017010302 |
| | XM_006714998 |
| Eukaryotic translation initiation factor 2 | NM_001135651 |
| alpha kinase 2 (EIF2AK2) (a.k.a. PKR) | NM_001135652 |
| | NM_002759 |
| | XM_011532987 |
| | XM_017004503 |
| AKT serine/threonine kinase 1 (AKT1) | NM_001014431 |
| (a.k.a. RAC) | NM_001014432 |
| | NM_005163 |
| Zinc fingers and homeoboxes 2 (ZHX2) | NM_001362797 |
| (a.k.a. RAF) | NM_014943 |
| | XM_011516932 |
| | XM_005250836 |
| KRAS proto-oncogene, GTPase (KRAS) | NM_001369786 |
| | NM_001369787 |
| | NM_004985 |
| | NM_033360 |
| NRAS proto-oncogene, GTPase (NRAS) | NM_002524 |
| Receptor interacting serine/threonine | NM_001317061 |
| kinase 1 (RIPK1) (a.k.a. RIP) | NM_001354930 |
| | NM_001354931 |
| | NM_001354932 |
| | NM_001354933 |
| | NM_001354934 |
| | NM_003804 |
| | XM_017011405 |
| | XM_006715237 |
| | XM_017011403 |
| | XM_017011404 |
| TNF receptor associated factor 6 (TRAF6) | NM_004620 |
| | NM_145803 |
| | XM_017018220 |
| ZFP36 ring finger protein (ZFP36) (a.k.a. | NM_003407 |
| TTP) | |

An antisense nucleic acid molecule can be complementary to all or part of a non-coding region of the coding strand of a nucleotide sequence encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA, e.g., specificity for any one of SEQ ID NOs: 1-37). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein. For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g., U.S. Pat. Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB p38, PKR, rac, ras, raf, RIP, TNFα, TNFR1, TNFR2, TRADD, TRAF2, TRAF6, or TTP polypeptide can be inhibited by targeting nucleotide sequences complementary to the regulatory region of the gene encoding the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C87 (Ma et al., J. Biol. Chem. 289(18):12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750):1022-1025, 2005.

In some examples, the anti-TNFα agent is a small molecule that inhibits the activity of one of AP-1, ASK1, IKK, JNK, MAPK, MEKK 1/4, MEKK4/7, MEKK 3/6, NIK, TRADD, RIP, NF-κB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vemurafenib (PLX4032, RG7204), sorafenib tosylate, PLX-4720, dabrafenib (GSK2118436), GDC-0879, RAF265 (CHIR-265), AZ 628, NVP-BHG712, 5B590885, ZM 336372, sorafenib, GW5074, TAK-632, CEP-32496, encorafenib (LGX818), CCT196969, LY3009120, R05126766 (CH5126766), PLX7904, and MLN2480).

In some examples, the anti-TNFα agent TNFα inhibitor is a small molecule that inhibits the activity of one of MK2 (PF 3644022 and PHA 767491), JNK (e.g., AEG 3482, BI 78D3, CEP 1347, c-JUN peptide, IQ 15, JIP-1 (153-163), SP600125, SU 3327, and TCS JNK6o), c-jun (e.g., AEG 3482, BI 78D3, CEP 1347, c-JUN peptide, IQ 1S, JIP-1 (153-163), SP600125, SU 3327, and TCS JNK6o), MEK3/6 (e.g., Akinleye et al., J. Hematol. Oncol. 6:27, 2013), p38 (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX 011, TA 01, TA 02, TAK 715, VX 702, and VX 745), PKR (e.g., 2-aminopurine or CAS 608512-97-6), TTP (e.g., CAS 329907-28-0), MEK1/2 (e.g., Facciorusso et al., Expert Review Gastroentrol. Hepatol. 9:993-1003, 2015), ERK1/2 (e.g., Mandal et al., Oncogene 35:2547-2561, 2016), NIK (e.g., Mortier et al., Bioorg. Med. Chem. Lett. 20:4515-4520, 2010), IKK (e.g., Reilly et al., Nature Med. 19:313-321, 2013), Iid3 (e.g., Suzuki et al., Expert. Opin. Invest. Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med. Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 28 nucleotides, about 15 nucleotides to about 26 nucleotides, about 15 nucleotides to about 25 nucleotides, about 15 nucleotides to about 24 nucleotides, about 15 nucleotides to about 22 nucleotides, about 15 nucleotides to about 20 nucleotides, about 15 nucleotides to about 18 nucleotides, about 15 nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

In one embodiment, provided herein is a combination of a compound of any preceding embodiment, for use in the treatment or the prevention of a condition mediated by TNF-α, in a patient in need thereof, wherein the compound is administered to said patient at a therapeutically effective amount. Preferably, the subject is resistant to treatment with an anti-TNFα agent. Preferably, the condition is a gut disease or disorder.

In one embodiment, provided herein is a pharmaceutical composition of comprising a compound of any preceding embodiment, and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.

In one embodiment, provided herein is a pharmaceutical combination of a compound of any preceding embodiment, and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

In one embodiment, the present invention relates to an NLRP3 antagonist for use in the treatment or the prevention of a condition mediated by TNF-α, in particular a gut disease or disorder, in a patient in need thereof, wherein the NLRP3 antagonist is administered to said patient at a therapeutically effective amount.

In one embodiment, the present invention relates to an NLRP3 antagonist for use in the treatment or the prevention of a condition, in particular a gut disease or disorder, in a patient in need thereof wherein the NLRP3 antagonist is administered to said patient at a therapeutically effective amount.

In one embodiment, the present invention relates to an NLRP3 antagonist for use in the treatment, stabilization or lessening the severity or progression of gut disease or disorder, in a patient in need thereof wherein the NLRP3 antagonist is administered to said patient at a therapeutically effective amount.

In one embodiment, the present invention relates to an NLRP3 antagonist for use in the slowing, arresting, or reducing the development of a gut disease or disorder, in a patient in need thereof wherein the NLRP3 antagonist is administered to said patient at a therapeutically effective amount.

In one embodiment, the present invention relates to an NLRP3 antagonist for use according to above listed embodiments wherein the NLRP3 antagonist is a gut-targeted NLRP3 antagonist.

In one embodiment, the present invention relates to NLRP3 antagonist for use according to any of the above embodiments, wherein the gut disease is IBD.

In one embodiment, the present invention relates to an NLRP3 antagonist for use according to any of the above embodiments, wherein the gut disease is UC or CD.

In one embodiment, the present invention relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

In one embodiment, the present invention relates to a method for the treatment or the prevention of a condition, in particular a gut disease or disorder, in a patient in need thereof, comprising administering to said patient a therapeutically effective amount of a gut-targeted NLRP3 antagonist.

In one embodiment, the present invention relates to a method for the treatment, stabilization or lessening the severity or progression of gut disease or disorder, in a patient in need thereof comprising administering to said patient a therapeutically effective amount of a gut-targeted NLRP3 antagonist.

In one embodiment, the present invention relates to a method for slowing, arresting, or reducing the development of a gut disease or disorder, in a patient in need thereof comprising administering to said patient a therapeutically effective amount of a gut-targeted NLRP3 antagonist.

In one embodiment, the present invention relates to a method according to any of the above embodiments, wherein the gut disease is IBD.

In one embodiment, the present invention relates to a method according to any of the above embodiments x to xx, wherein the gut disease is UC or CD.

In one embodiment, the present invention relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### PREPARATIVE EXAMPLES

Racemic compounds of this invention can be resolved to give individual enantiomers using a variety of known methods. For example, chiral stationary phases can used and the elution conditions can include normal phase or super-critical fluid with or without acidic or basic additives. Enantiomerically pure acids or bases can be used to form diatereomeric salts with the racemic compounds whereby pure enantiomers can be obtained by fractional crystallization. The racemates can also be derivatized with enantiomerically pure auxiliary reagents to form diastereomeric mixtures that can be separated. The auxiliary is then removed to give pure enantiomers.

The following abbreviations have the indicated meanings:

ACN=acetonitrile
BTC=trichloromethyl chloroformate
Boc=t-butyloxy carbonyl

### Davephos=2-Dicyclohexylphosphino-2′-(N,N-dimethylamino)biphenyl

DCM=dichloromethane
DEA=diethylamine

### DMF=N,N-dimethylformamide

DMSO=dimethyl sulfoxide

### DIEA=N,N-diisopropylethylamine

DPPA=diphenylphosphoryl azide
dppf=1,1′-Bis(diphenylphosphino)ferrocene
EtOH=ethanol
HATU=1-[Bis(dimethylamino)methylene]-1H-1,2,3-triazolo[4,5-b]pyridinium 3-oxid hexafluorophosphate
Hex=hexane
HPLC=high performance liquid chromatography
LC-MS=liquid chromatography mass spectrometry
LiHMDS=lithium bis(trimethylsilyl)amide
LDA=lithium diisopropylamide

### M=mol/L

Me=methyl
MeOH=methanol
MSA=methanesulfonic acid

### NBS=N-bromosuccinimide

### NCS=N-chlorosuccinimide

NMR=nuclear magnetic resonance
Pd(dppf)Cl₂=dichloro[1,1′-bis(diphenylphosphino)ferrocene]palladium
Ph=phenyl
PPh₃Cl₂=dichlorotriphenylphosphorane
Py=pyridine
RT=room temperature
Rt=Retention time
R_{f}=Retardation factor
Sat.=saturated
TBAF=tetrabutylammonium fluoride
TBS=tert-butyldimethylsilyl
TBSCl=tert-butyldimethylsilyl chloride
TBDPSCl=tert-butyldiphenylsilyl chloride
TEA=triethylamine
TFA=trifluoroacetic acid
THF=tetrahydrofuran
TLC=thin layer chromatography
TsOH=4-methylbenzenesulfonic acid
UV=ultraviolet

### General

The progress of reactions was often monitored by TLC or LC-MS. The identity of the products was often confirmed by LC-MS. The LC-MS was recorded using one of the following methods.

Method A: Shim-pack XR-ODS, C18, 3×50 mm, 2.5 um column, 1.0 uL injection, 1.5 mL/min flow rate, 90-900 amu scan range, 190-400 nm UV range, 5-100% (1.1 min), 100% (0.6 min) gradient with ACN (0.05% TFA) and water (0.05% TFA), 2 minute total run time.

Method B: Kinetex EVO, C18, 3×50 mm, 2.2 um column, 1.0 uL injection, 1.5 mL/min flow rate, 90-900 amu scan range, 190-400 nm UV range, 10-95% (1.1 min), 95% (0.6 min) gradient with ACN and water (0.5% NH₄HCO₃), 2 minute total run time.

Method C: Shim-pack XR-ODS, C18, 3×50 mm, 2.5 um column, 1.0 uL injection, 1.5 mL/min flow rate, 90-900 amu scan range, 190-400 nm UV range, 5-100% (2.1 min), 100% (0.6 min) gradient with ACN (0.05% TFA) and water (0.05% TFA), 3 minute total run time.

Method D: Kinetex EVO, C18, 3×50 mm, 2.2 um column, 1.0 uL injection, 1.5 mL/min flow rate, 90-900 amu scan range, 190-400 nm UV range, 10-95% (2.1 min), 95% (0.6 min) gradient with ACN and water (0.5% NH₄HCO₃), 3 minute total run time.

Method F: Phenomenex, CHO-7644, Onyx Monolithic C18, 50×4.6 mm, 10.0 uL injection, 1.5 mL/min flow rate, 100-1500 amu scan range, 220 and 254 nm UV detection, 5% with ACN (0.1% TFA) to 100% water (0.1% TFA) over 9.5 min, with a stay at 100% (ACN, 0.1% TFA) for 1 min, then equilibration to 5% (ACN, 0.1% TFA) over 1.5 min.

The final targets were purified by Prep-HPLC. The Prep-HPLC was carried out using the following method.

Method E: Prep-HPLC: Column, XBridge Shield RP18 OBD (19×250 mm, 10 um); mobile phase, Water (10 mmol/L NH₄HCO₃) and ACN, UV detection 254/210 nm.

Method G: Prep-HPLC: Higgins Analytical Proto 200, C18 Column, 250×20 mm, 10 um; mobile phase, Water (0.1% TFA) and ACN (0.1% TFA), UV detection 254/210 nm.

NMR was recorded on BRUKER NMR 300.03 MHz, DUL-C-H, ULTRASHIELD™ 300, AVANCE II 300 B-ACS™ 120 or BRUKER NMR 400.13 MHz, BBFO, ULTRASHIELD™ 400, AVANCE III 400, B-ACS™ 120 or BRUKER AC 250 NMR instrument with TMS as reference measured in ppm (part per million).

Racemic compounds of this invention can be resolved to give individual enantiomers using a variety of known methods. For example, chiral stationary phases can used and the elution conditions can include normal phase or super-critical fluid with or without acidic or basic additives. Enantiomerically pure acids or bases can be used to form diatereomeric salts with the racemic compounds whereby pure enantiomers can be obtained by fractional crystallization. The racemates can also be derivatized with enantiomerically pure auxiliary reagents to form diastereomeric mixtures that can be separated. The auxiliary is then removed to give pure enantiomers.

General method for the preparation of PPh₃Cl₂-DCE solution (0.5 M): Into a 500 mL round-bottom flask purged and maintained with an inert atmosphere of nitrogen, was placed a solution of triphenylphosphane (30.00 g, 114.4 mmol, 1.0 equiv.) in DCE (230 mL). To the solution was added C₂Cl₆ (27.00 g, 114.4 mmol, 1.0 equiv.). The resulting solution was stirred for overnight at ambient temperature. The crude was used directly without workup.

### Schemes for the Preparation of Final Targets:

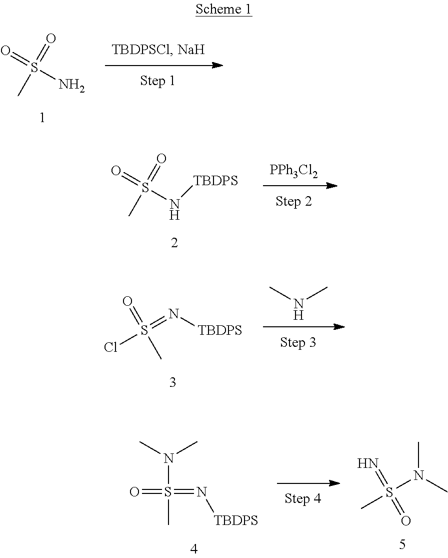

### Step 1: N-(tert-butyldiphenylsilyl)methanesulfonamide

Into a 250 mL round-bottom flask, was placed a solution of methanesulfonamide (10.00 g, 105.1 mmol, 1.0 equiv.) in THF (80.0 mL). To the solution were added NaH (60% wt in mineral oil, 8.41 g, 210.3 mmol, 2.0 equiv.) and this was followed by the addition of TBDPSCl (23.12 g, 84.1 mmol, 0.8 equiv.). The solution was stirred for overnight at ambient temperature and then quenched by the addition of water (80 mL). The resulting solution was extracted with ethyl acetate and the combined organic layers were concentrated under vacuum to give 11.2 g of crude N-(tert-butyldiphenylsilyl)methanesulfonamide as a brown solid. MS-ESI: 334.1 (M+1).

### Step 2: N-(tert-butyldiphenylsilyl)methanesulfonimidoyl chloride

Into a 250 mL 3-necked round-bottom flask purged and maintained with an inert atmosphere of nitrogen, was placed a solution of PPh₃Cl₂ in DCE (0.5 M, 60.0 mL, 30.0 mmol, 2.0 equiv.). To the solution was added 2,6-lutidine (6.43 g, 60.0 mmol, 4.0 equiv.) at 0° C., and this was followed by the addition of a solution of N-(tert-butyldiphenylsilyl)methanesulfonamide (5.00 g, 15.0 mmol, 1.0 equiv.) in DCE (10.0 mL) dropwise at 0° C. The resulting mixture was stirred for 40 mins at ambient temperature. The resulting mixture was used in the next step directly without further purification.

### Step 3: N′-(tert-butyldiphenylsilyl)-N,N-dimethylmethanesulfonimidamide

To the above solution from step 2, was added dimethylamine (2 M in THF, 30 mL, 60.0 mmol, 4.0 equiv.). The solution was stirred overnight at ambient temperature and then concentrated under reduced pressure. The residue was purified by Prep-TLC (petroleum ether/ethyl acetate=5:1) to give 4.5 g of N′-(tert-butyldiphenylsilyl)-N,N-dimethylmethanesulfonimidamide as a light yellow oil. MS-ESI: 361.2 (M+1).

### Step 4: N,N-dimethylmethanesulfonimidamide

Into a 50 mL round-bottom flask, was placed a solution of N′-(tert-butyldiphenylsilyl)-N,N-dimethylmethanesulfonimidamide (2.30 g, 6.4 mmol, 1.0 equiv.) in THF (10.0 mL). To the solution was added HF/Pyridine (1.00 mL, w/t 70%, 55.1 mmol, 8.6 equiv.). The solution was stirred for 1 h at ambient temperature and concentrated under reduced pressure to give 800 mg of crude N,N-dimethylmethanesulfonimidamide which was used in the next step directly without further purification. MS-ESI: 123.0 (M+1).

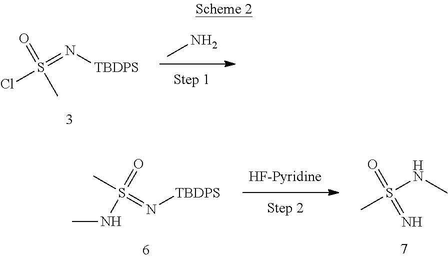

### Step 1: N′-(tert-butyldiphenylsilyl)-N-methylmethanesulfonimidamide

Into a 100 mL round-bottom flask, was placed a solution of N-(tert-butyldiphenylsilyl)methanesulfonimidoyl chloride (1.00 g, 2.8 mmol, 1.0 equiv.) in DCE (20.00 mL). To the solution was added methylamine (2 M in THF, 5.7 mL, 11.4 mmol, 4.0 equiv.). The solution was stirred overnight at ambient temperature. The resulting mixture was concentrated under reduced pressure and the residue was purified by Prep-TLC (petroleum ether/ethyl acetate=5:1) to afford 300 mg of N′-(tert-butyldiphenylsilyl)-N-methylmethanesulfonimidamide as a white solid. MS-ESI: 347.2 (M+1).

### Step 2: N-methylmethanesulfonimidamide

Into a 50 mL round-bottom flask, was placed a solution of N′-(tert-butyldiphenylsilyl)-N-methylmethanesulfonimidamide (100 mg, 0.29 mmol, 1.0 equiv.) in THF (5.0 mL). To the solution was added HF/Pyridine (0.70 mL, w/t 70%, 2.9 mmol, 10.0 equiv.). The solution was stirred for 1 h at ambient temperature and concentrated under reduced pressure to give 100 mg of crude N-methylmethanesulfonimidamide, which was used in the next step directly without further purification. MS-ESI: 109.0 (M+1).

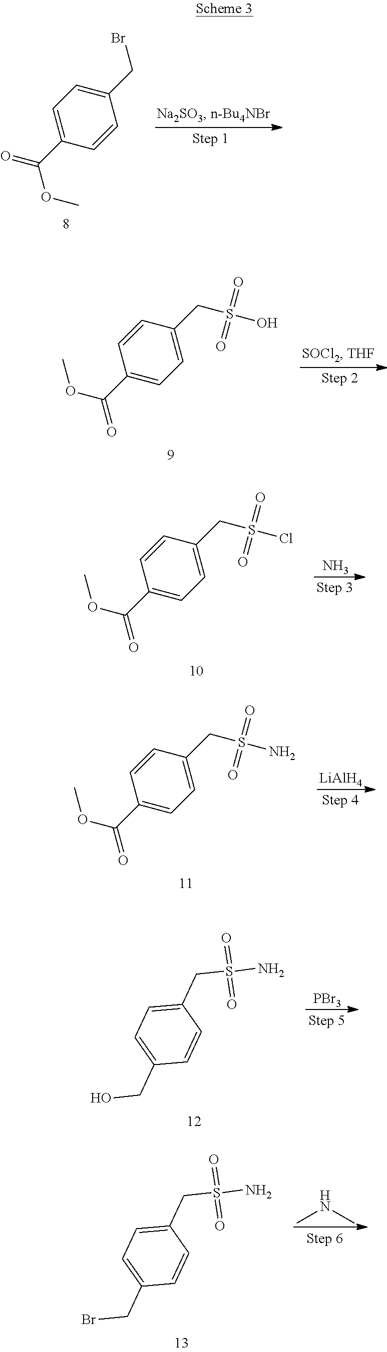

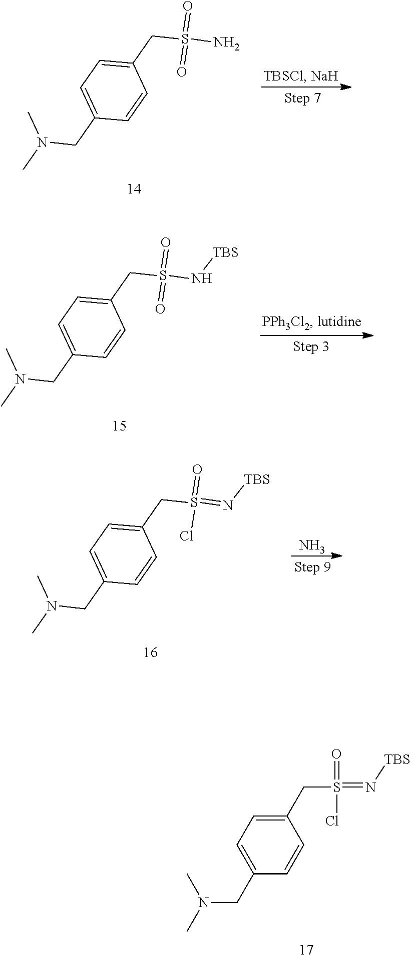

### Step 1: (4-(methoxycarbonyl)phenyl)methanesulfonic acid

Into a 500 mL round-bottom flask purged and maintained with an inert atmosphere of nitrogen, was placed a solution of methyl 4-(bromomethyl)benzoate (10.00 g, 43.6 mmol, 1.0 equiv.) in water (100.0 mL). To the solution were added Na₂SO₃ (7.15 g, 56.7 mmol, 1.3 equiv.) and tetrabutylammonium bromide (0.70 g, 2.2 mmol, 0.05 equiv.). The resulting solution was stirred for 12 hr at 80° C. in an oil bath. The resulting mixture was concentrated under vacuum to result in 8.85 g of (4-(methoxycarbonyl)phenyl)methanesulfonic acid as a white solid. MS-ESI: 229.0 (M−1).

### Step 2: methyl 4-[(chlorosulfonyl)methyl]benzoate

Into a 100 mL round-bottom flask purged and maintained with an inert atmosphere of nitrogen, was placed a solution of (4-(methoxycarbonyl)phenyl)methanesulfonic acid (4.00 g, 17.4 mmol, 1.0 equiv.) in THF (20.0 mL). To the solution was added SOCl₂ (3.10 g, 26.1 mmol, 1.5 equiv.). The resulting solution was stirred for 2 h at 0° C. in a water/ice bath and then quenched by the addition of ice water. The resulting solution was extracted with dichloromethane and the combined organic layers were dried over anhydrous sodium sulfate and concentrated under vacuum to give 3.75 g of methyl 4-[(chlorosulfonyl)methyl]benzoate as a white solid.

### Step 3: methyl 4-(sulfamoylmethyl)benzoate

Into a 100 mL round-bottom flask purged and maintained with an inert atmosphere of nitrogen, was placed a solution of methyl 4-[(chlorosulfonyl)methyl]benzoate (2.00 g, 8.0 mmol, 1.0 equiv.) in DCM (20.0 mL). To the above, NH_{3(g)} was bubbled at 0° C. for 30 min. The resulting solution was stirred for 2 h at 20° C. and then quenched by the addition of ice water (10 mL). The resulting solution was extracted with petroleum ether and the combined organic layers were dried over anhydrous sodium sulfate and concentrated under vacuum. The residue was purified by flash column chromatography on silica gel, eluting with ethyl acetate/petroleum ether (1:1) to give 65.0 g of methyl 4-(sulfamoylmethyl)benzoate as a white solid. MS-ESI: 230.0 (M+1).

### Step 4: (4-(hydroxymethyl)phenyl)methanesulfonamide

Into a 100 mL round-bottom flask purged and maintained with an inert atmosphere of nitrogen, was placed a solution of methyl 4-(sulfamoylmethyl)benzoate (1.50 g, 6.5 mmol, 1.0 equiv.) in THF (20.0 mL). To the solution was added LiAlH₄ (0.62 g, 16.4 mmol, 2.5 equiv.) at 0° C. The resulting solution was stirred for 5 min at 0° C. in a water/ice bath. The resulting solution was allowed to stir for an additional 2 h at 20° C. The reaction was then quenched by the addition of MeOH and concentrated under vacuum. The residue was purified by flash column chromatography on silica gel, eluting with dichloromethane/methanol (8:1) to give 850 mg of (4-(hydroxymethyl)phenyl)methanesulfonamide as a white solid. MS-ESI: 202.0 (M+1).

### Step 5: [4-(bromomethyl)phenyl]methanesulfonamide

Into a 50 mL round-bottom flask purged and maintained with an inert atmosphere of nitrogen, was placed a solution of [4-(hydroxymethyl)phenyl]methanesulfonamide (400 mg, 2.0 mmol, 1.0 equiv.) in THF (5.0 mL). To the solution was added PBr₃ (430 mg, 1.6 mmol, 0.8 equiv.) at 0° C. The resulting solution was stirred for 30 min at ambient temperature and then quenched by the addition of saturated aqueous NaHCO₃. The resulting solution was extracted with petroleum ether and the combined organic layers were dried over anhydrous sodium sulfate and concentrated under vacuum to give 455 mg of [4-(bromomethyl)phenyl]methanesulfonamide as a white solid. MS-ESI: 264.2 (M+1).

### Step 6: [4-[(dimethylamino)methyl]phenyl]methanesulfonamide

Into a 50 mL round-bottom flask purged and maintained with an inert atmosphere of nitrogen, was placed a solution of [4-(bromomethyl)phenyl]methanesulfonamide (450 mg, 1.7 mmol, 1.0 equiv.) in THF (10.0 mL). To the solution was added dimethylamine (2 M in THF, 4.3 mL, 8.6 mmol, 5.0 equiv.). The resulting solution was stirred for 12 h at ambient temperature and then concentrated under vacuum. The residue was purified by flash column chromatography on silica gel, with dichloromethane/methanol (10:1) to give 310 mg of [4-[(dimethylamino)methyl]phenyl]methanesulfonamide as a white solid. MS-ESI: 229.1 (M+1).

### Step 7: N-(tert-butyldimethylsilyl)-1-[4-[(dimethylamino)methyl]phenyl]methanesulfonamide

Into a 50 mL round-bottom flask purged and maintained with an inert atmosphere of nitrogen, was placed a solution of [4-[(dimethylamino)methyl]phenyl]methanesulfonamide (200 mg, 0.9 mmol, 1.0 equiv.) in THF (5.0 mL). To the solution was added NaH (60% wt in mineral oil, 72 mg, 1.8 mmol, 2.0 equiv.) under N_{2(g)} at 0° C. The resulting solution was stirred for 30 min at 0° C. and followed by the addition of TBSCl (200 mg, 1.3 mmol, 1.5 equiv.). The resulting solution was stirred for 30 min at 0° C. in a water/ice bath. The resulting solution was allowed to react, with stirring, for an additional 2 h at ambient temperature. The reaction was then quenched by the addition of ice water (1 mL). The resulting solution was extracted with petroleum ether and the combined organic layers were dried over anhydrous sodium sulfate and concentrated under vacuum to give 215 mg of N-(tert-butyldimethylsilyl)-1-[4-[(dimethylamino)methyl]phenyl]-methanesulfonamide as a white solid. MS-ESI: 343.2 (M+1).

### Step 8-9: N-(tert-butyldimethylsilyl)-1-[4-[(dimethylamino)methyl]phenyl]methane-sulfonimidamide

Into a 50 mL 3-necked round-bottom flask purged and maintained with an inert atmosphere of nitrogen, was placed a solution of PPh₃Cl₂ in DCE (0.5 M 2.5 mL, 1.75 mmol, 3.0 equiv.). This was followed by the addition of 2,6-lutidine (375 mg, 3.5 mmol, 6.0 equiv.) at 0° C. The resulting solution was stirred for 15 min at 0° C. and followed by the addition of N-(tert-butyldimethylsilyl)-1-[4-[(dimethylamino)methyl]phenyl]methanesulfonamide (200 mg, 0.6 mmol, 1.0 equiv.) at 0° C. The resulting mixture was stirred for additional 30 min at 0° C. To the above mixture, NH_{3(g)} was bubbled at 0° C. for 30 min. The resulting solution was allowed to react, with stirring, for an additional 15 h at ambient temperature. After concentration, the residue purified by flash column chromatography on silica gel, eluting with dichloromethane/methanol (8:1) to give 98 mg of N-(tert-butyldimethylsilyl)-1-[4-[(dimethylamino)methyl]phenyl]-methanesulfonimidamide as a solid. MS-ESI: 342.2 (M+1).

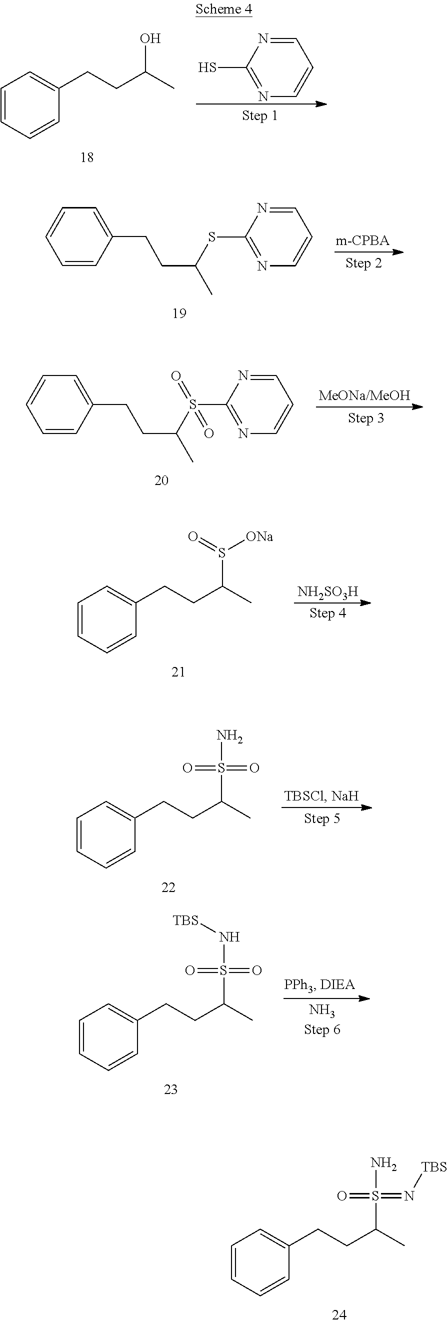

### Step 1: 2-((4-phenylbutan-2-yl)thio)pyrimidine

Into a 250 mL round-bottom flask purged and maintained with an inert atmosphere of nitrogen, was placed a solution of 2-hydroxy-4-phenylbutane (2.00 g, 13.3 mmol, 1.0 equiv.) in THF (50.0 mL). This was followed by the addition of PPh₃ (5.41 g, 20.6 mmol, 1.6 equiv.), DIEA (3.5 mL, 20.0 mmol, 1.5 equiv.) and 2-mercaptopyrimidine (2.31 g, 20.6 mmol, 1.6 equiv.) at 0° C. The resulting solution was stirred for 30 min at 0° C. The resulting solution was stirred for an additional 17 h at ambient temperature and then concentrated under vacuum. The residue was purified by flash column chromatography on silica gel, eluting with ethyl acetate/hexane (2:1) to give 2.2 g of 2-((4-phenylbutan-2-yl)thio)pyrimidine as a yellow solid. MS-ESI: 245.1 (M+1).

### Step 2: 2-((4-phenylbutan-2-yl)sulfonyl)pyrimidine

Into a 250 mL round-bottom flask, was placed a solution of 2-((4-phenylbutan-2-yl)thio)pyrimidine (4.00 g, 16.4 mmol, 1.0 equiv.) in DCM (40.0 mL). To the solution was added m-CPBA (5.93 g, 34.4 mmol, 2.1 equiv.) at 0° C. The resulting solution was stirred for additional 14 h at ambient temperature. After concentration, the residue was applied onto a silica gel column with ethyl acetate/hexane (1:1) to give 3.2 g of 2-((4-phenylbutan-2-yl)sulfonyl)pyrimidine as a yellow solid. MS-ESI: 277.1 (M+1).

### Step 3: sodium 4-phenylbutane-2-sulfinate

Into a 250 mL round-bottom flask, was placed a solution of 2-((4-phenylbutan-2-yl)sulfonyl)pyrimidine (3.00 g, 10.9 mmol, 1.0 equiv.) in MeOH (30.0 mL). To the solution was added MeONa (0.59 g, 10.9 mmol, 1.0 equiv.) at 0° C. The resulting solution was stirred for an additional 14 h at ambient temperature and then the resulting mixture was concentrated. The residue was washed with 5 mL of diethyl ether to give 1.5 g of sodium 4-phenylbutane-2-sulfinate as a white solid. MS-ESI: 197.1 (M−1).

### Step 4: 4-phenylbutane-2-sulfonamide

Into a 100 mL round-bottom flask, was placed a solution of sodium 4-phenylbutane-2-sulfinate (2.00 g, 9.1 mmol, 1.0 equiv.) in water (20.0 mL). To the solution were added CH₃COONa (0.93 g, 11.3 mmol, 1.3 equiv.) and NH₂SO₃H (1.10 g, 11.4 mmol, 1.3 equiv.). The resulting solution was stirred for 12 h at ambient temperature and then concentrated under vacuum. The residue was applied onto a silica gel column with ethyl acetate/hexane (2:1) to give 2 g of 4-phenylbutane-2-sulfonamide as a yellow solid. MS-ESI: 214.1 (M+1).

### Step 5: N-(tert-butyldimethylsilyl)-4-phenylbutane-2-sulfonamide

Into a 100 mL round-bottom flask, was placed a solution of 4-phenylbutane-2-sulfonamide (1.20 g, 5.6 mmol, 1.0 equiv.) in THF (20.0 mL). To the solution was added NaH (60% wt in mineral oil, 450 mg, 11.3 mmol, 2.0 equiv.) under N_{2(g)} at 0° C. The resulting solution was stirred for 30 min at 0° C. and followed by the addition of TBSCl (1.27 g, 8.4 mmol, 1.5 equiv.). The resulting solution stirred for additional 5 h at ambient temperature and then quenched by the addition of water (10 mL). The resulting solution was extracted with ethyl acetate and the combined organic layers were concentrated under vacuum to give 1.5 g of N-(tert-butyldimethylsilyl)-4-phenylbutane-2-sulfonamide as a yellow solid. MS-ESI: 328.2 (M+1).

### Step 6: N′-(tert-butyldimethylsilyl)-4-phenylbutane-2-sulfonimidamide

Into a 100 mL 3-necked round-bottom flask, was placed a solution of PPh₃Cl₂ in DCE (0.5 M, 45 mL, 22.5 mmol, 4.0 equiv.) under nitrogen atmosphere. This was followed by the addition of DIEA (7.83 mL, 44.9 mmol, 8.0 equiv.) at 0° C. The resulting solution was stirred for 15 min at 0° C. and followed by the addition of N-(tert-butyldimethylsilyl)-4-phenylbutane-2-sulfonamide (1.84 g, 5.6 mmol, 1.0 equiv.) at 0° C. The resulting mixture was stirred for additional 30 min at 0° C. To the above mixture, NH_{3(g)} was bubbled at 0° C. for 30 min. The resulting solution was allowed to stir for an additional 15 h at ambient temperature. After concentration, the residue was purified by flash column chromatography on silica gel, eluting with ethyl acetate/hexane (3:2) to give 225 mg of N′-(tert-butyldimethylsilyl)-4-phenylbutane-2-sulfonimidamide as a yellow solid. MS-ESI: 327.2 (M+1).

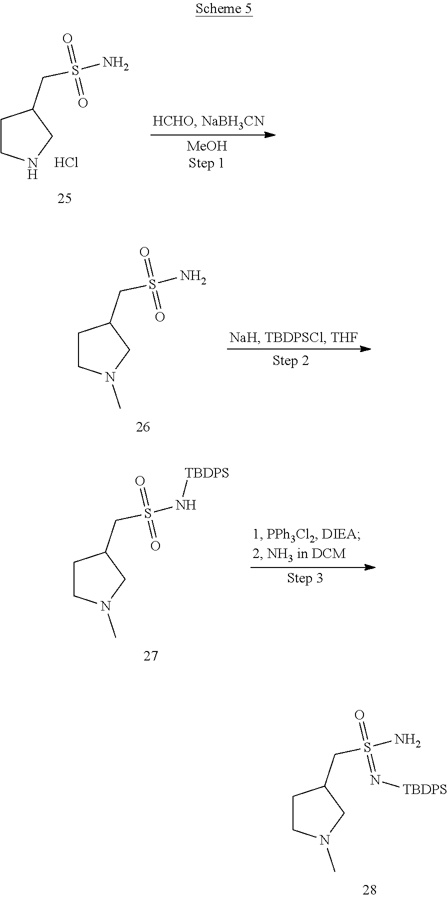

### Step 1: (1-methylpyrrolidin-3-yl)methanesulfonamide

To a solution of pyrrolidin-3-ylmethanesulfonamide hydrochloride (enamine, 1.00 g, 5.0 mmol, 1.0 equiv.) in MeOH (30.0 mL) was added HCHO (299 mg, 10.0 mmol, 2.0 equiv.). The solution was stirred for 2 h and then followed by the addition of NaBH₃CN (940 mg, 15.0 mmol, 3.0 equiv.) at ambient temperature. The resulting mixture was stirred overnight at ambient temperature and the resulting mixture was concentrated under vacuum to give the crude (1-methylpyrrolidin-3-yl)methanesulfonamide, which was used in the next step directly without further purification. MS-ESI: 179.1 (M+1).

### Step 2: N-(tert-butyldiphenylsilyl)-1-(1-methylpyrrolidin-3-yl)methanesulfonamide

To a solution of (1-methylpyrrolidin-3-yl)methanesulfonamide (1.30 g, 7.3 mmol, 1.0 equiv.) in THF (30.0 mL) was added NaH (60% wt in mineral oil, 585 mg, 14.6 mmol, 2.0 equiv.) at 0° C. under nitrogen atmosphere. The resulting mixture was stirred for 30 min at 0° C., then followed by the addition of TBDPSCl (3.01 g, 10.9 mmol, 1.5 equiv.) at 0° C. The resulting mixture was stirred for overnight at ambient temperature and then quenched by the addition of water (2 mL) at 0° C. The resulting mixture was extracted with DCM and the combined organic layers were concentrated under vacuum. The residue was purified by silica gel column chromatography, eluting with DCM/MeOH (12:1) to give 1.0 g of N-(tert-butyldiphenylsilyl)-1-(1-methylpyrrolidin-3-yl)methanesulfonamide as a yellow solid. MS-ESI: 417.2 (M+1).

### Step 3: N-(tert-butyldiphenylsilyl)-1-(1-methylpyrrolidin-3-yl)methanesulfonimidamide

To a solution of PPh₃Cl₂ in DCE (0.5 M, 25 mL, 12.5 mmol, 4.0 equiv.) was added DIEA (4.36 mL, 25.0 mmol, 8.0 equiv.) dropwise at 0° C. under nitrogen atmosphere. The resulting mixture was stirred for 15 min at 0° C. and followed by the addition of N-(tert-butyldiphenylsilyl)-1-(1-methylpyrrolidin-3-yl)methanesulfonamide (1.30 g, 3.1 mmol, 1.0 equiv.) in CHCl₃ (10.0 mL) dropwise at 0° C. The resulting mixture was stirred for additional 30 min at 0° C. To the above mixture, NH_{3(g)} was bubbled at 0° C. for 30 min. The resulting mixture was stirred for overnight at ambient temperature and the resulting mixture was concentrated under reduced pressure. The residue was purified by Prep-TLC (ethyl acetate/MeOH=10:1) to give 250 mg of N-(tert-butyldiphenylsilyl)-1-(1-methylpyrrolidin-3-yl)methanesulfonoimidamide as a yellow solid. MS-ESI: 416.2 (M+1).

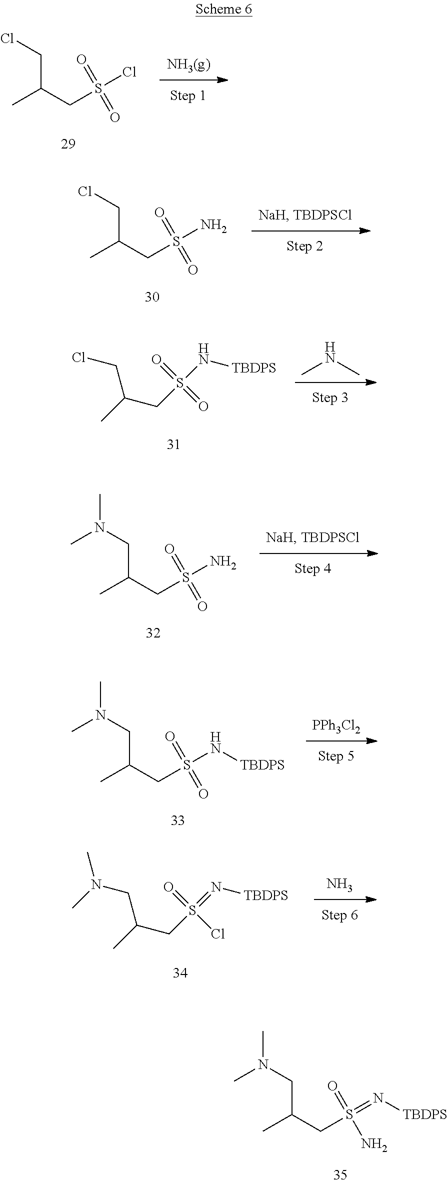

### Step 1: 3-chloro-2-methylpropane-1-sulfonamide

Into a 100 mL round-bottom flask, was placed a solution of 3-chloro-2-methylpropane-1-sulfonyl chloride (2.00 g, 10.5 mmol, 1.0 equiv.) in THF (50.0 mL). To the resulting solution, NH_{3(g)} was bubbled at 0° C. for 30 min. The resulting solution was stirred for 1 h at ambient temperature and then quenched by the addition of water. The resulting solution was extracted with ethyl acetate and the combined organic layers were dried over anhydrous sodium sulfate and concentrated under vacuum to give 1.6 g of 3-chloro-2-methylpropane-1-sulfonamide as yellow oil. MS-ESI: 172.1 (M+1).

### Step 2: N-(tert-butyldiphenylsilyl)-3-chloro-2-methylpropane-1-sulfonamide

Into a 100 mL round-bottom flask purged and maintained with an inert atmosphere of nitrogen, was placed a solution of 3-chloro-2-methylpropane-1-sulfonamide (1.60 g, 9.3 mmol, 1.0 equiv.) in THF (50.0 mL). To the solution was added NaH (60% wt in mineral oil, 745 mg, 18.6 mmol, 2.0 equiv.) in portions at 0° C. This was followed by the addition of TBDPSCl (3.84 g, 14.0 mmol, 1.5 equiv.). The resulting solution was stirred overnight at ambient temperature and then quenched by the addition of water. The resulting solution was extracted with ethyl acetate and the combined organic layers were dried over anhydrous sodium sulfate and concentrated under vacuum. The residue purified by flash column chromatography on silica gel, eluting with petroleum ether:ethyl acetate (10:1) to give 2.4 g of N-(tert-butyldiphenylsilyl)-3-chloro-2-methylpropane-1-sulfonamide as a yellow solid. MS-ESI: 408.1 (M−1).

### Step 3: 3-(dimethylamino)-2-methylpropane-1-sulfonamide

Into a 50 mL round-bottom flask, was placed a solution of N-(tert-butyldiphenylsilyl)-3-chloro-2-methylpropane-1-sulfonamide (800 mg, 2.0 mmol, 1.0 equiv.) in THF (20.0 mL). To the solution was added dimethylamine (2 M in THF, 2.0 mL, 4.0 mmol, 2.0 equiv.). The resulting solution was stirred for 2 days at 60° C. and then quenched by the addition of water. The resulting solution was extracted with ethyl acetate and the combined organic layers were dried over anhydrous sodium sulfate and concentrated under vacuum. The residue was purified by flash column chromatography on silica gel, eluting with petroleum ether:ethyl acetate (5:1) to give 200 mg of 3-(dimethylamino)-2-methylpropane-1-sulfonamide as a yellow solid. MS-ESI: 181.1 (M+1).

### Step 4: N-(tert-butyldiphenylsilyl)-3-(dimethylamino)-2-methylpropane-1-sulfonamide

Into a 100 mL round-bottom flask purged and maintained with an inert atmosphere of nitrogen, was placed a solution of 3-(dimethylamino)-2-methylpropane-1-sulfonamide (200 mg, 1.1 mmol, 1.0 equiv.) in THF (10 mL). To the solution was added NaH (60% wt in mineral oil, 90 mg, 2.2 mmol, 2.0 equiv.) in portions at 0° C. This was followed by the addition of TBDPSCl (305 mg, 1.1 mmol, 1.0 equiv.). The resulting solution was stirred for overnight at ambient temperature and then quenched by the addition of water. The resulting solution was extracted with ethyl acetate and the combined organic layers were dried over anhydrous sodium sulfate and concentrated under vacuum. The residue was purified by flash column chromatography on silica gel, eluting with petroleum ether:ethyl acetate (1:1) to give 200 mg of N-(tert-butyldiphenylsilyl)-3-(dimethylamino)-2-methylpropane-1-sulfonamide as a white solid. MS-ESI: 419.2 (M+1).

### Step 5-6: N-(tert-butyldiphenylsilyl)-3-(dimethylamino)-2-methyl propane-1-sulfonoimidamide

Into a 50-mL 3-necked round-bottom flask purged and maintained with an inert atmosphere of nitrogen, was placed a solution of PPh₃Cl₂ in DCE (0.5 M, 1.8 mL, 0.9 mmol, 3.0 equiv.). To the solution was added DIEA (0.3 mL, 1.7 mmol, 6.0 equiv.) dropwise at 0° C. The resulting mixture was stirred for 15 min at 0° C. and followed by the addition of N-(tert-butyldiphenylsilyl)-3-(dimethylamino)-2-methylpropane-1-sulfonamide (120 mg, 0.3 mmol, 1.0 equiv.) at 0° C. The resulting mixture was stirred for an additional 30 min at 0° C. To the above mixture, NH_{3(g)} was bubbled at 0° C. for 30 min. The resulting mixture was stirred overnight at ambient temperature and then quenched by the addition of water. The resulting mixture was extracted with ethyl acetate and the combined organic layers were dried over anhydrous sodium sulfate and concentrated under vacuum. The residue was purified by flash column chromatography on silica gel, eluting with petroleum ether:ethyl acetate (1:1) to give mg of N-(tert-butyldiphenylsilyl)-3-(dimethylamino)-2-methylpropane-1-sulfonoimidamide as a yellow solid. MS-ESI: 418.2 (M+1).

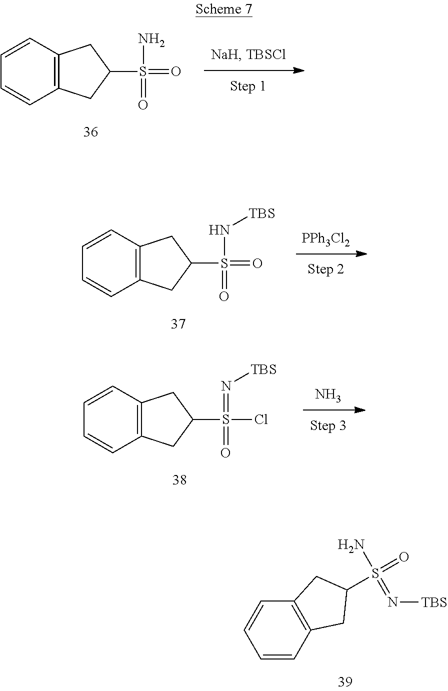

### Step 1: N-(tert-butyldimethylsilyl)-2,3-dihydro-1H-indene-2-sulfonamide

Into a 100 mL round-bottom flask purged and maintained with an inert atmosphere of nitrogen, was placed a solution of 2,3-dihydro-1H-indene-2-sulfonamide (500 mg, 2.5 mmol, 1.0 equiv.) in THF (20.0 mL). To the solution was added NaH (60% wt in mineral oil, 204 mg, 5.1 mmol, 2.0 equiv.). This was followed by the addition of TBSCl (764 mg, 5.1 mmol, 2.0 equiv.). The resulting solution was stirred for 3 h at ambient temperature. The reaction was then quenched by the addition of 20 mL of water. The resulting solution was extracted with ethyl acetate and the combined organic layers were concentrated to give 450 mg of N-(tert-butyldimethylsilyl)-2,3-dihydro-1H-indene-2-sulfonamide as a white solid. MS-ESI: 312.1 (M+1).

### Step 2-3: N′-(tert-butyldimethylsilyl)-2,3-dihydro-1H-indene-2-sulfonimidamide

Into a 50 mL round-bottom flask purged and maintained with an inert atmosphere of nitrogen, was placed a solution of PPh₃Cl₂ in DCE (0.5 M, 24 mL, 12.0 mmol, 2.5 equiv.). To the solution was added DIEA (2.1 mL, 12.1 mmol, 2.5 equiv.). The resulting solution was stirred for 10 min at 0° C. Then to the solution was added N-(tert-butyldimethylsilyl)-2,3-dihydro-1H-indene-2-sulfonamide (1.5 g, 4.8 mmol, 1.0 equiv.) and the solution was stirred for 30 min at 0° C. To the above, NH_{3(g)} was bubbled at 0° C. for 30 min. The resulting solution was stirred for additional 1 h at ambient temperature. The resulting mixture was concentrated, and the residue was purified by flash column chromatography on silica gel, eluting with ethyl acetate/petroleum ether (1:2) to give 50 mg of N′-(tert-butyldimethylsilyl)-2,3-dihydro-1H-indene-2-sulfonimidamide as a yellow solid. MS-ESI: 311.2 (M+1).

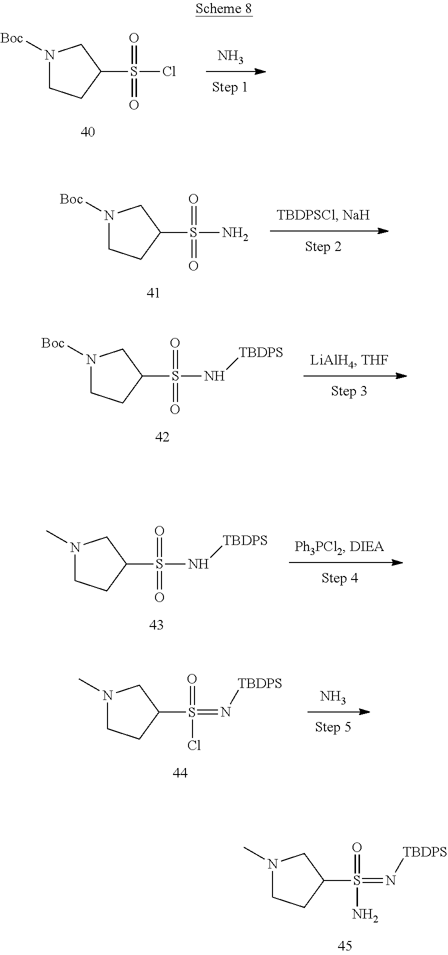

### Step 1: tert-butyl 3-sulfamoylpyrrolidine-1-carboxylate

Into a 100 mL 3-necked round-bottom flask was placed a solution of tert-butyl 3-(chlorosulfonyl)pyrrolidine-1-carboxylate (1.5 g, 5.6 mmol, 1.0 equiv.) in THF (30 mL). To the solution, NH_{3(g)} was bubbled at 0° C. for 30 min. The resulting solution was stirred for additional 1 h at 0° C. The solids were filtered out and the filtrate was concentrated under vacuum to give 1.31 g of tert-butyl 3-sulfamoylpyrrolidine-1-carboxylate as a yellow solid. MS-ESI: 248.9 (M−1).

### Step 2: tert-butyl 3-[(tert-butyldiphenylsilyl)sulfamoyl]pyrrolidine-1-carboxylate

Into a 250 mL round-bottom flask, was placed a solution of tert-butyl 3-sulfamoylpyrrolidine-1-carboxylate (1.87 g, 7.5 mmol, 1.0 equiv.) in THF (50 mL). To the solution was added NaH (60% wt in mineral oil, 800 mg, 20.0 mmol, 2.7 equiv.). This was followed by the addition of TBSCl (2.46 g, 9.0 mmol, 1.2 equiv.). The resulting solution was stirred for 4 h at ambient temperature and then quenched by the addition of 50 mL of ice water. The resulting solution was extracted with ethyl acetate and the combined organic layers were dried over anhydrous sodium sulfate and concentrated under vacuum. The residue was applied onto a silica gel column with dichloromethane/methanol (20:1) to give 1.2 g of tert-butyl 3-[(tert-butyldiphenylsilyl)sulfamoyl]pyrrolidine-1-carboxylate as a yellow solid. MS-ESI: 487.2 (M−1).

### Step 3: N-(tert-butyldiphenylsilyl)-1-methylpyrrolidine-3-sulfonamide

Into a 100 mL round-bottom flask, was placed a solution of tert-butyl 3-[(tert-butyldiphenylsilyl)sulfamoyl]pyrrolidine-1-carboxylate (1.2 g, 2.5 mmol, 1.0 equiv.) in THF (30 mL). This was followed by the addition of LiAlH₄ (0.38 g, 10.0 mmol, 4.1 equiv.) in portions over 10 min at 0° C. The resulting solution was stirred for 4 h at 80° C. and then quenched by the addition of 50 mL of ice water. The resulting solution was extracted with ethyl acetate and the combined organic layers were dried over anhydrous sodium sulfate and concentrated under vacuum. The residue was purified by flash column chromatography on silica gel, eluting with dichloromethane/methanol (19:1) to give 890 mg of N-(tert-butyldiphenylsilyl)-1-methylpyrrolidine-3-sulfonamide as a light yellow solid. MS-ESI: 403.2 (M+1).

### Step 4-5: N-(tert-butyldiphenylsilyl)-1-methylpyrrolidine-3-sulfonoimidamide

A 250 mL 3-necked round-bottom flask was charged with a solution of PPh₃Cl₂ in DCE (11 mL, 0.5 M, 5.5 mmol, 2.5 equiv.). To the solution, DIEA (1.93 mL, 11.1 mmol, 5.0 equiv.) was added dropwise at 0° C. The reaction mixture was stirred for 0.5 h at 0° C. This was followed by the addition of a solution of N-(tert-butyldiphenylsilyl)-1-methylpyrrolidine-3-sulfonamide (890 mg, 2.2 mmol, 1.0 equiv.) in DCM (10 ml) dropwise with stirring at 0° C. The reaction mixture was stirred for an additional 0.5 h, then NH_{3(g)} was bubbled into the reaction mixture for 10 min at 0° C. and then allowed to stir for an additional 2 h. The resulting mixture was concentrated, and the residue was purified by flash column chromatography on silica gel, eluting with dichloromethane/methanol (10:1) to give 720 mg of N-(tert-butyldiphenylsilyl)-1-methylpyrrolidine-3-sulfonoimidamide as a yellow solid. MS-ESI: 402.1 (M+1).

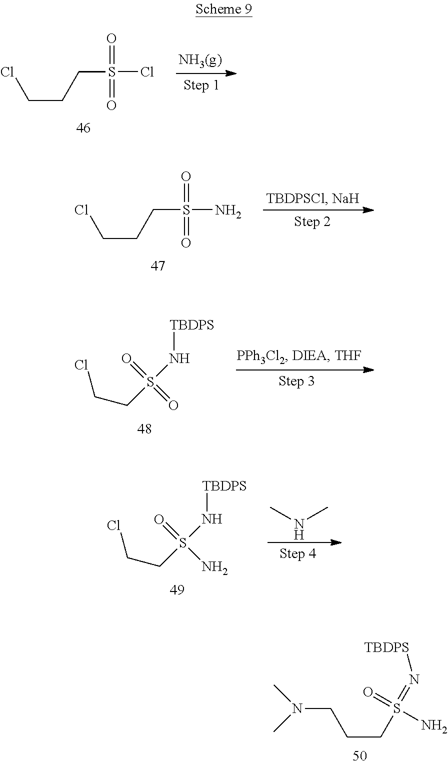

### Step 1: 3-chloropropane-1-sulfonamide

Into a 250 mL 3-necked round-bottom flask purged and maintained with an inert atmosphere of nitrogen, was placed a solution of 3-chloropropane-1-sulfonyl chloride (13.0 g, 73.4 mmol, 1.0 equiv.) in DCM (100 mL). To the above NH_{3(g)} was bubbled for 20 min at 0° C. The resulting solution was stirred for additional 1 h at 0° C. and then concentrated under vacuum. The crude product was re-crystallized from n-Hexane:DCM in the ratio of 1:20 to give 4.5 g of 3-chloropropane-1-sulfonamide as a yellow solid. MS-ESI: 158.0 (M+1).

### Step 2: N-(tert-butyldiphenylsilyl)-3-chloropropane-1-sulfonamide

Into a 250 mL round-bottom flask purged and maintained with an inert atmosphere of nitrogen, was placed a solution of 3-chloropropane-1-sulfonamide (4.5 g, 28.5 mmol, 1.0 equiv.) in THF (100 mL). To the solution was added NaH (60% wt in mineral oil, 2.28 g, 57.1 mmol, 2.0 equiv.). This was followed by the addition of TBSCl (9.42 g, 34.3 mmol, 1.2 equiv.). The resulting solution was stirred for 12 h at ambient temperature and then quenched by the addition of 50 mL of ice water. The resulting solution was extracted with ethyl acetate and the combined organic layers were dried over anhydrous sodium sulfate and concentrated under vacuum. The residue was purified by flash column chromatography on silica gel, eluting with ethyl acetate/petroleum ether (1:10) to give 8 g of N-(tert-butyldiphenylsilyl)-3-chloropropane-1-sulfonamide as yellow oil. MS-ESI: 396.0 (M+1).

### Step 3: N-(tert-butyldiphenylsilyl)-3-chloropropane-1-sulfonoimidamide

A 250 mL 3-necked round-bottom flask was charged with a solution of PPh₃Cl₂ (60.5 mL, 0.5 M in CHCl₃, 30.3 mmol, 3.0 equiv.) in CHCl₃ (10 mL) and then DIEA (10.6 mL, 60.6 mmol, 6.0 equiv.) was added dropwise at 0° C. The reaction mixture was stirred for 0.5 h at 0° C. This was followed by the addition of a solution of N-(tert-butyldiphenylsilyl)-3-chloropropane-1-sulfonamide (4 g, 10.1 mmol, 1.0 equiv.) in CHCl₃ (10 ml) dropwise with stirring at 0° C. The reaction mixture was stirred for an additional 0.5 h, then NH_{3(g)} was bubbled into the reaction mixture for 10 min at 0° C. and then allowed to stir for an additional 5 h. The resulting mixture was concentrated, and the residue was purified by flash column chromatography on silica gel, eluting with ethyl acetate/petroleum ether (1:2) to give 1 g of N-(tert-butyldiphenylsilyl)-3-chloropropane-1-sulfonoimidamide as a yellow solid. MS-ESI: 395.0 (M+1).

### Step 4: N-(tert-butyldiphenylsilyl)-3-(dimethylamino)propane-1-sulfonoimidamide

Into a 100 mL round-bottom flask was placed a solution of N-(tert-butyldiphenylsilyl)-3-chloropropane-1-sulfonoimidamide (2.00 g, 5.1 mmol, 1.0 equiv.) in dimethylamine (2 M in THF, 20 mL, 40 mmol, 8.0 equiv.). The resulting solution was stirred for 12 h at 50° C. and then concentrated under vacuum. The residue was purified by flash column chromatography on silica gel, eluting with dichloromethane/methanol (10:1) to give 600 mg of N-(tert-butyldiphenylsilyl)-3-(dimethylamino)propane-1-sulfonoimidamide as yellow oil. MS-ESI: 404.2 (M+1).

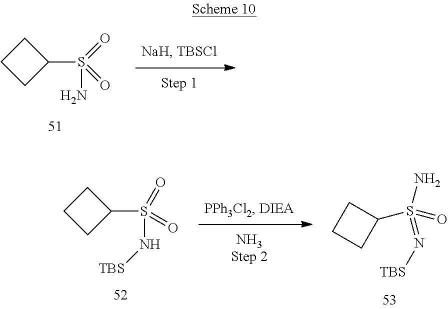

### Step 1: N-(tert-butyldimethylsilyl)cyclobutanesulfonamide

Into a 25 mL round-bottom flask purged and maintained with an inert atmosphere of nitrogen, was placed a solution of cyclobutanesulfonamide (900 mg, 6.7 mmol, 1.0 equiv.) in THF (10 mL). To the solution was added NaH (60% wt in mineral oil, 533 mg, 13.3 mmol, 2.0 equiv.). This was followed by the addition of TBSCl (1.51 g, 10.0 mmol, 1.5 equiv.). The resulting solution was stirred for 2 h at ambient temperature. The reaction was quenched by the addition of water (10 mL). The resulting solution was extracted with ethyl acetate and the combined organic layers were concentrated to give 1.2 g of crude N-(tert-butyldimethylsilyl)cyclobutanesulfonamide as yellow oil. MS-ESI: 250.1 (M+1).

### Step 2: N-(tert-butyldimethylsilyl)cyclobutanesulfonimidamide

Into a 50 mL round-bottom flask purged and maintained with an inert atmosphere of nitrogen, was placed a solution of PPh₃Cl₂ in DCE (0.5 M, 24 mL, 12.0 mmol, 2.5 equiv.) and DIEA (4.2 mL, 24.1 mmol, 5.00 equiv.) in CHCl₃ (10 mL). The resulting solution was stirred for 10 min at 0° C. Then to the solution was added N-(tert-butyldimethylsilyl)cyclobutanesulfonamide (1.2 g, 4.8 mmol, 1.0 equiv.) and the solution was stirred for 30 min at 0° C. To the above, NH_{3(g)} was bubbled at 0° C. for 30 min. The resulting solution was stirred for additional 1 h at ambient temperature. The resulting mixture was concentrated and the residue was purified by flash column chromatography on silica gel, eluting with ethyl acetate/petroleum ether (1:2) to give 50 mg of N-(tert-butyldimethylsilyl)cyclobutanesulfonoimidamide as a yellow solid. MS-ESI: 249.1 (M+1).

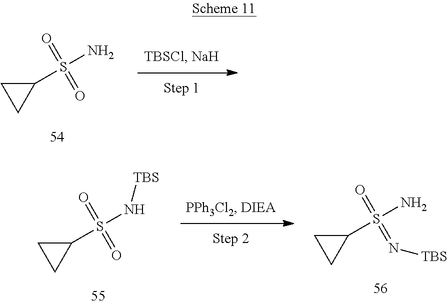

### Step 1: N-(tert-butyldimethylsilyl)cyclopropanesulfonamide

Into a 25 mL round-bottom flask purged and maintained with an inert atmosphere of nitrogen, was placed a solution of cyclopropanesulfonamide (500 mg, 4.1 mmol, 1.0 equiv.) in THF (10 mL). To the solution was added NaH (60% wt in mineral oil, 248 mg, 6.2 mmol, 1.5 equiv.) at 0° C. This was followed by the addition of TBSCl (933 mg, 6.2 mmol, 1.5 equiv.). The resulting solution was stirred for 12 h at ambient temperature. The reaction was then quenched by the addition of 10 mL of water. The resulting solution was extracted with ethyl acetate and the combined organic layers were concentrated. The residue was purified by flash column chromatography on silica gel, eluting with ethyl acetate/petroleum ether (1:1) to give 580 mg of N-(tert-butyldimethylsilyl)cyclopropanesulfonamide as yellow oil. MS-ESI: 236.1 (M+1).

### Step 2: N′-(tert-butyldimethylsilyl)cyclopropanesulfonimidamide

Into a 50 mL round-bottom flask purged and maintained with an inert atmosphere of nitrogen, was placed a solution of PPh₃Cl₂ in DCE (0.5 M, 25 mL, 12.5 mmol, 5.00 equiv.) and DIEA (4.44 mL, 25.5 mmol, 10.00 equiv.) in CHCl₃ (10 mL). The resulting solution was stirred for 10 min at 0° C. Then to the solution was added N-(tert-butyldimethylsilyl)cyclopropanesulfonamide (580 mg, 2.6 mmol, 1.0 equiv.) and the solution was stirred for 30 min at 0° C. To the above, NH_{3(g)} was bubbled at 0° C. for 30 min. The resulting solution was stirred for additional 1 h at ambient temperature. The resulting mixture was concentrated and the residue was purified by flash column chromatography on silica gel, eluting with ethyl acetate/petroleum ether (1:1) to give 300 mg of N′-(tert-butyldimethylsilyl)cyclopropanesulfonimidamide as a light yellow solid. MS-ESI: 235.1 (M+1).

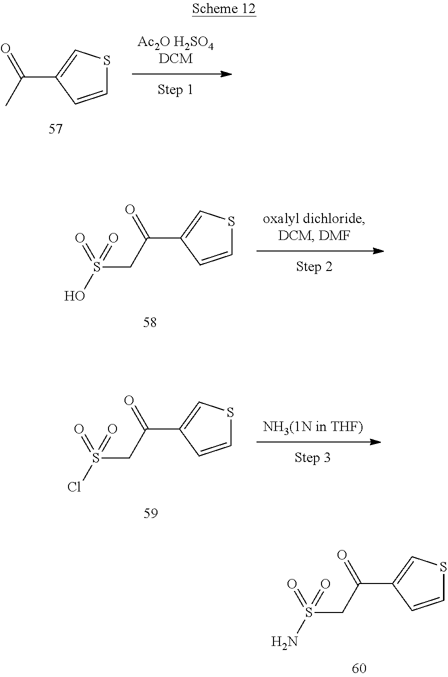

### Step 1: 2-oxo-2-(thiophen-3-yl)ethane-1-sulfonic acid

Into a 100 mL round-bottom flask, was placed a solution of 1-(thiophen-3-yl)ethan-1-one (2.00 g, 15.8 mmol, 1.0 equiv.) in DCM (40 mL) and acetic anhydride (5 mL). To the solution was added sulfuric acid (0.98 mL, 17.6 mmol, 1.1 equiv., 98% wt). The resulting solution was stirred for 2 h at 25° C. The resulting mixture was concentrated under vacuum to result in 3 g (crude) of 2-oxo-2-(thiophen-3-yl)ethane-1-sulfonic acid as yellow oil, which was used in the next step without purification. MS-ESI: 205.0 (M−1).

### Step 2: 2-oxo-2-(thiophen-3-yl)ethane-1-sulfonyl chloride

Into a 100 mL round-bottom flask, was placed a solution of 2-oxo-2-(thiophen-3-yl)ethane-1-sulfonic acid (3.00 g, 14.6 mmol, 1.0 equiv.) in DCM (50 mL). To the solution were added DMF (0.1 mL, 1.2 mmol, 0.08 equiv.) and oxalic dichloride (3.71 g, 29.2 mmol, 2.0 equiv.). The resulting solution was stirred for 3 h at 25° C. The resulting mixture was concentrated under vacuum to result in 3.1 g (crude) of 2-oxo-2-(thiophen-3-yl)ethane-1-sulfonyl chloride as yellow oil, which was used in the next step without purification.

### Step 3: 2-oxo-2-(thiophen-3-yl)ethane-1-sulfonamide

Into a 50 mL round-bottom flask, was placed a solution of 2-oxo-2-(thiophen-3-yl)ethane-1-sulfonyl chloride (3.00 g, 13.4 mmol, 1.0 equiv.) in THF (5 mL). To the solution was added NH₃/THF solution (1 M, 15 mL, 15 mmol, 1.1 equiv.). The resulting solution was stirred for 1 h at 25° C. The resulting mixture was concentrated under vacuum and the residue was purified by flash column chromatography on silica gel, eluting with ethyl acetate/petroleum ether (1:1) to give 540 mg of 2-oxo-2-(thiophen-3-yl)ethane-1-sulfonamide as a yellow solid. MS-ESI: 206.0 (M+1).

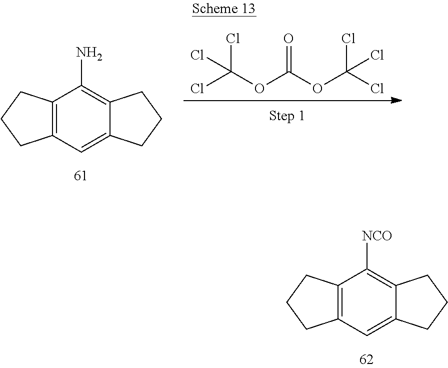

### Step 1: 4-isocyanato-1,2,3,5,6,7-hexahydro-s-indacene

Into a 500 mL round-bottom flask, was placed a solution of 1,2,3,5,6,7-hexahydro-s-indacen-4-amine (20.0 g, 115.4 mmol, 1.0 equiv.) in THF (250 mL). This was followed by the addition of ditrichloromethyl carbonate (13.70 g, 46.2 mmol, 0.4 equiv.) in portions. The resulting solution was stirred for 3 h at 70° C. (the reaction progress was monitored by quenching with MeOH, and the LCMS showed the corresponding signal of methyl (1,2,3,5,6,7-hexahydro-s-indacen-4-yl)carbamate. MS-ESI: 232.2 [M+H]⁺) and concentrated under vacuum to give in 22.5 g of crude 4-isocyanato-1,2,3,5,6,7-hexahydro-s-indacene as a yellow solid, which was used directly in the next step.

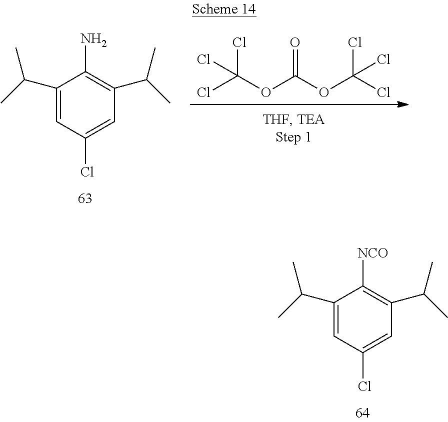

### Step 1: 5-chloro-2-isocyanato-1,3-diisopropylbenzene

Into a 40 mL sealed tube, was placed a solution of 4-chloro-2,6-bis(propan-2-yl)aniline (500 mg, 2.4 mmol, 1.0 equiv.) in THF (10 mL). To the solution were added ditrichloromethyl carbonate (232 mg, 0.8 mmol, 0.3 equiv.) and TEA (120 mg, 1.2 mmol, 0.5 equiv.). The resulting solution was stirred for 1 h at 70° C. and concentrated under vacuum. The residue was purified by flash column chromatography on silica gel, eluting with ethyl acetate/petroleum ether (1:1) to give 300 mg of 5-chloro-2-isocyanato-1,3-diisopropylbenzene as a yellow solid. ¹H NMR: (300 MHz, DMSO-d₆) δ: 7.18 (s, 2H), 3.22-3.19 (m, 2H), 1.26 (d, 12H).

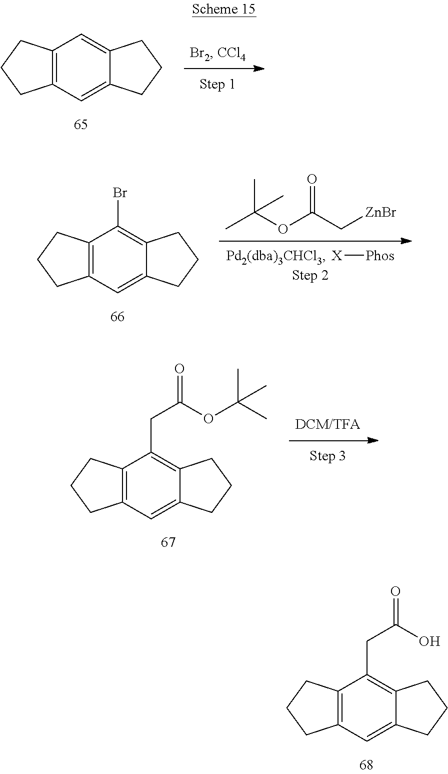

### Step 1: 4-bromo-1,2,3,5,6,7-hexahydro-s-indacene

Into a 500 mL 3-necked round-bottom flask purged and maintained with an inert atmosphere of nitrogen, was placed a solution of 1,2,3,5,6,7-hexahydro-s-indacene (15.00 g, 94.8 mmol, 1.0 equiv.) in CCl₄ (200 mL). To the solution was added 12 (1.2 g, 4.7 mmol, 0.05 equiv.). The solution was cooled to 0° C., then a solution of Br₂ (16.0 g, 100.1 mmol, 1.1 equiv.) in CCl₄ (50 mL) was added dropwise over 10 min. The resulting solution was stirred for additional 2 h at 0° C. and then quenched by the addition of 150 mL of saturated aqueous NH₄Cl solution. The resulting mixture was extracted with dichloromethane and the combined organic layers were dried over anhydrous sodium sulfate and concentrated under vacuum to give 23.3 g of 4-bromo-1,2,3,5,6,7-hexahydro-s-indacene as yellow oil. ¹H NMR: (300 MHz, DMSO-d₆) δ: 7.03 (s, 1H), 2.94-2.88 (m, 4H), 2.82-2.76 (m, 4H), 2.10-1.97 (m, 4H).

### Step 2: tert-butyl 2-(1,2,3,5,6,7-hexahydro-s-indacen-4-yl)acetate

Into a 40 mL sealed tube purged and maintained with an inert atmosphere of nitrogen, was placed a solution of 4-bromo-1,2,3,5,6,7-hexahydro-s-indacene (1.00 g, 4.2 mmol, 1.0 equiv.) in THF (20 mL). To the solution was added X-Phos (200 mg, 0.4 mmol, 0.1 equiv.) and Pd₂(dba)₃CHCl₃ (220 mg, 0.4 mmol, 0.1 equiv.) and the reaction mixture was stirred for 10 min at ambient temperature. This was followed by the addition of (2-(tert-butoxy)-2-oxoethyl)zinc(II) bromide (2.20 g, 8.5 mmol, 2.0 equiv.). The resulting solution heated to 80° C. for 4 h and then quenched by the addition of saturated NH₄C₁ solution (50 mL). The mixture was extracted with dichloromethane and the combined organic layers were dried over anhydrous sodium sulfate and concentrated under vacuum to give 1.4 g of tert-butyl 2-(1,2,3,5,6,7-hexahydro-s-indacen-4-yl)acetate as a brown oil. ¹H NMR: (400 MHz, DMSO-d₆) δ: 6.97 (s, 1H), 3.52 (s, 2H), 3.00-2.60 (m, 8H), 2.40-2.00 (m, 4H), 1.44 (s, 9H).

### Step 3: 2-(1,2,3,5,6,7-hexahydro-s-indacen-4-yl)acetic acid

Into a 50 mL round-bottom flask, was placed a solution of tert-butyl 2-(1,2,3,5,6,7-hexahydro-s-indacen-4-yl)acetate (530 mg, 1.9 mmol, 1.0 equiv.) in DCM (15 mL) and TFA (5 mL). The reaction mixture was stirred for 3 h at ambient temperature and then concentrated under vacuum. This resulted in 380 mg of 2-(1,2,3,5,6,7-hexahydro-s-indacen-4-yl)acetic acid as a yellow solid. MS-ESI: 215.1 (M−1). ¹H NMR: (400 MHz, DMSO-d₆) δ: 12.19 (s, 1H), 6.95 (s, 1H), 3.52-3.47 (m, 2H), 2.82-2.74 (m, 8H), 2.03-1.96 (m, 4H).

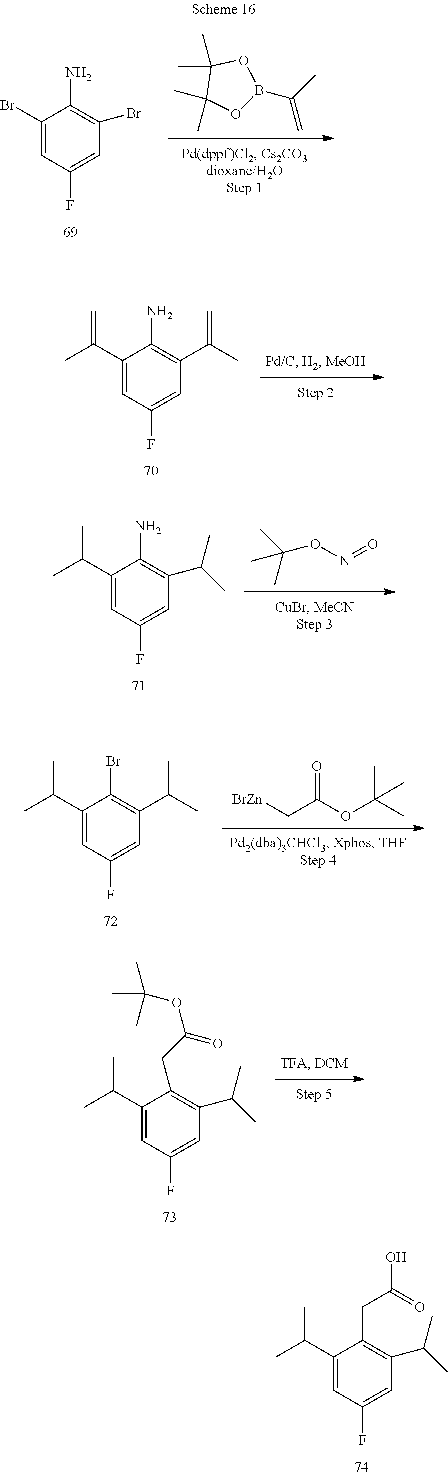

### Step 1: 4-Fluoro-2,6-bis(prop-1-en-2-yl)aniline

Into a 500 mL round-bottom flask purged with and maintained under nitrogen was placed a solution of 2,6-dibromo-4-fluoroaniline (15.0 g, 55.8 mmol, 1.0 equiv.) in dioxane (150 mL)/water (15 mL). To the solution was were added Cs₂CO₃ (55.0 g, 168.8 mmol, 3.0 equiv.), 4,4,5,5-tetramethyl-2-(prop-1-en-2-yl)-1,3,2-dioxaborolane (25.0 g, 148.8 mmol, 2.7 equiv.) and Pd(dppf)Cl₂ (4.00 g, 5.6 mmol, 0.1 equiv.). The resulting solution was stirred for 15 h at 100° C. under nitrogen. After concentration, the filtrate was concentrated under vacuum. The residue was diluted with 300 mL water, and the solution was extracted with ethyl acetate. The combined organic layers were dried over anhydrous Na₂SO₄ and concentrated under vacuum. The residue was purified by flash column chromatography on silica gel, eluting with ethyl acetate/petroleum ether (1:10 to 1:8) to give 9.2 g of 4-fluoro-2,6-bis(prop-1-en-2-yl)aniline as brown oil. MS-ESI: 192.1 (M+1).

### Step 2: 4-Fluoro-2,6-bis(propan-2-yl)aniline

Into a 500 mL round-bottom flask was placed a solution of 4-fluoro-2,6-bis(prop-1-en-2-yl)aniline (9.20 g, 48.1 mmol, 1.0 equiv.) in MeOH (200 mL). Then Pd/C (10% wt, 900 mg, 0.8 mmol, 0.2 equiv.) was added. The flask was evacuated and flushed three times with hydrogen. The resulting solution was stirred for 12 h at ambient temperature under an atmosphere of hydrogen with a balloon. The solids were filtered out. The resulting mixture was concentrated under vacuum and the residue was purified by flash column chromatography on silica gel, eluting with ethyl acetate/petroleum ether (1:10 to 1:8) to give 7.2 g of 4-fluoro-2,6-bis(propan-2-yl)aniline as brown oil. MS-ESI: 196.1 (M+1).

### Step 3: 2-Bromo-5-fluoro-1,3-bis(propan-2-yl)benzene

Into a 500 mL round-bottom flask purged with and maintained under nitrogen was placed a solution of 4-fluoro-2,6-bis(propan-2-yl)aniline (7.00 g, 35.9 mmol, 1.0 equiv.) in MeCN (300 mL). To the solution was added CuBr (7.71 g, 53.9 mmol, 1.5 equiv.) in portions. This was followed by the addition of tert-butyl nitrite (5.55 g, 53.8 mmol, 1.5 equiv.) dropwise at below 5° C. The resulting solution was stirred for 3 h at 60° C. and then concentrated under vacuum. The residue was purified by flash column chromatography on silica gel, eluting with petroleum ether to give 3.0 g of 2-bromo-5-fluoro-1,3-bis(propan-2-yl)benzene as yellow oil. ¹H NMR (400 MHz, DMSO-d₆): δ 7.09 (d, 2H), 3.40-3.36 (m, 2H), 1.20 (d, 12H).

### Step 4: Tert-butyl 2-[4-fluoro-2,6-bis(propan-2-yl)phenyl]acetate

Into a 250 mL 3-necked round-bottom flask purged with and maintained under nitrogen, was placed a solution of 2-bromo-5-fluoro-1,3-bis(propan-2-yl)benzene (3.00 g, 11.6 mmol, 1.0 equiv.) in THF (150 mL). To this solution were added X-Phos (553 mg, 1.2 mmol, 0.1 equiv.) and Pd₂(dba)₃CHCl₃ (600 mg, 0.58 mmol, 0.05 equiv.). The resulting solution was stirred for 0.5 h at ambient temperature. Then to the above was added (2-(tert-butoxy)-2-oxoethyl)zinc(II) bromide (6.00 g, 23.0 mmol, 2.0 equiv.). The resulting solution was stirred for 5 h at 70° C. and then quenched by the addition of 100 mL of NH₄C₁ aq. (sat.). The resulting solution was extracted with ethyl acetate and the organic layers were concentrated under vacuum. The residue was purified by flash column chromatography on silica gel, eluting with ethyl acetate/petroleum ether (1:100 to 3:97) to give 3.14 g of tert-butyl 2-[4-fluoro-2,6-bis(propan-2-yl)phenyl]acetate as yellow oil. ¹H NMR (400 MHz, DMSO-d₆) δ 6.93 (d, 2H), 3.67 (s, 2H), 3.19-3.07 (m, 2H), 1.39 (s, 9H), 1.15 (d, 12H).

### Step 5: 2-(4-Fluoro-2,6-diisopropylphenyl)acetic acid

Into a 50 mL round-bottom flask, was placed a solution of tert-butyl 2-[4-fluoro-2,6-bis(propan-2-yl)phenyl]acetate (1.56 g, 5.30 mmol) in DCM (10 mL). To the solution was added TFA (10 mL) dropwise at ambient temperature. The resulting solution was stirred for 3 h at ambient temperature and then concentrated under vacuum. The crude product was dissolved in 100 mL of NaOH aq. (4 N). The resulting solution was washed with DCM and the pH value of aqueous layer was adjusted to 2 with HCl aq. (4 N). The solution was extracted with DCM and the combined organic layers were dried over anhydrous Na₂SO₄ and concentrated under vacuum to give 1.09 g of 2-(4-fluoro-2,6-diisopropylphenyl)acetic acid as a light yellow solid. MS-ESI: 237.1 (M−1).

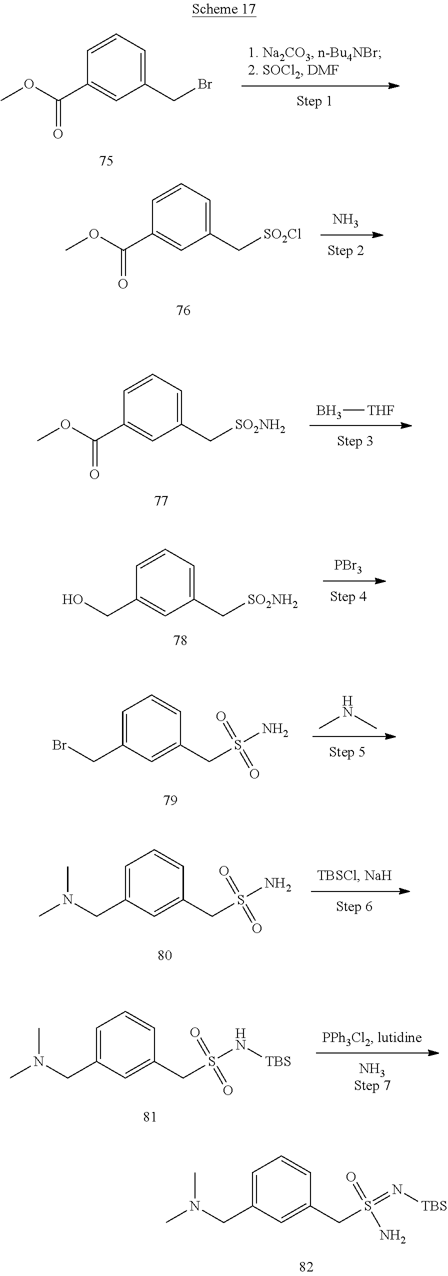

### Step 1: Methyl 3-[(chlorosulfonyl)methyl]benzoate

Into a 250 mL round-bottom flask purged and maintained with an inert atmosphere of nitrogen, was placed a solution of methyl 3-(bromomethyl)benzoate (5.00 g, 21.8 mmol, 1.0 equiv.) in water (50 mL). To the solution were added Na₂SO₃ (3.58 g, 28.4 mmol, 1.3 equiv.) and tetrabutylammonium bromide (0.30 g, 1.1 mmol, 0.05 equiv.). The resulting solution was stirred for 6 h at 80° C. and concentrated to give (3-(methoxycarbonyl)phenyl)methanesulfonic acid (MS-ESI: 229.0 (M−1)), which was used without additional purification. This was dissolved in DMF (50 mL). To the solution was added SOCl₂ (5.89 g, 43.6 mmol, 2.0 equiv.) and the reaction mixture was stirred for 30 min at 25° C. The reaction was then quenched by the addition of 100 mL of ice water and extracted with ethyl acetate. The organic layer was concentrated to give 3.5 g of methyl 3-[(chlorosulfonyl)methyl]benzoate as a yellow solid.

### Step 2: Methyl 3-(sulfamoylmethyl)benzoate

Into a 250 mL round-bottom flask purged and maintained with an inert atmosphere of nitrogen, was placed a solution of methyl 3-[(chlorosulfonyl)methyl]benzoate (3.50 g, 14.1 mmol, 1.0 equiv.) in DCM (50 mL). To the solution, NH_{3(g)} was bubbled at 0° C. for 30 min. The resulting solution was stirred for 2 h at ambient temperature and then concentrated under vacuum. The residue was purified by flash column chromatography on silica gel, eluting with dichloromethane/methanol (10:1) to give 3 g of methyl 3-(sulfamoylmethyl)benzoate as a yellow solid. MS-ESI: 230.0 (M+1).

### Step 3: [3-(hydroxymethyl)phenyl]methanesulfonamide

Into a 100 mL round-bottom flask purged and maintained with an inert atmosphere of nitrogen, was placed a solution of methyl 3-(sulfamoylmethyl)benzoate (3.00 g, 13.1 mmol, 1.0 equiv.) in THF (50 mL). To the solution was added BH₃.THF (0.9 M in THF, 29.1 mL, 26.2 mmol, 2.00 equiv). The resulting solution was stirred for 2 h at 50° C. and then quenched by the addition of MeOH (20 mL). The resulting mixture was concentrated to give 3 g of [3-(hydroxymethyl)phenyl]methanesulfonamide as yellow oil, which was used to next step without further purification. MS-ESI: 202.0 (M+1).

### Step 4: [3-(bromomethyl)phenyl]methanesulfonamide

Into a 250 mL round-bottom flask purged and maintained with an inert atmosphere of nitrogen, was placed a solution of [3-(hydroxymethyl)phenyl]methanesulfonamide (3.00 g, 14.9 mmol, 1.0 equiv.) in THF (50 mL). To the solution was added PBr₃ (3.23 g, 11.9 mmol, 0.8 equiv.). The resulting solution was stirred for 30 min at ambient temperature and then quenched by the addition of water (50 mL). The resulting solution was extracted with ethyl acetate and the organic layer was concentrated to give 3 g of [3-(bromomethyl)phenyl]methanesulfonamide as yellow oil, which was used to next step without purification. MS-ESI: 264.1 (M+1), 266.1 (M+1).

### Step 5: [3-[(dimethylamino)methyl]phenyl]methanesulfonamide

Into a 250 mL round-bottom flask purged and maintained with an inert atmosphere of nitrogen, was placed a solution of [3-(bromomethyl)phenyl]methanesulfonamide (3.00 g, 11.4 mmol, 1.0 equiv.) in THF (50 mL). To the solution was added dimethylamine (1.55 mL, 22.715 mmol, 2.00 equiv). The resulting solution was stirred for 2 h at ambient temperature and then concentrated under vacuum. The residue was purified by flash column chromatography on silica gel, eluting with dichloromethane/methanol (5:1) to give 2.5 g of [3-[(dimethylamino)methyl]phenyl]methanesulfonamide as yellow oil. MS-ESI: 229.1 (M+1).

### Step 6: N-(tert-butyldimethylsilyl)-1-[3-[(dimethylamino)methyl]phenyl]methanesulfonamide

Into a 250 mL round-bottom flask purged and maintained with an inert atmosphere of nitrogen, was placed a solution of [3-[(dimethylamino)methyl]phenyl]methanesulfonamide (2.50 g, 10.9 mmol, 1.0 equiv.) in THF (50 mL). To the solution was added NaH (60% wt in mineral oil, 0.88 g, 21.9 mmol, 2.0 equiv.). This was followed by the addition of TBSCl (2.48 g, 16.4 mmol, 1.5 equiv.). The resulting solution was stirred for 2 h at ambient temperature. The reaction was then quenched by the addition of 20 mL of water. The resulting solution was extracted with ethyl acetate and the combined organic layers were concentrated to give 3 g of N-(tert-butyldimethylsilyl)-1-[3-[(dimethylamino)methyl]phenyl]methanesulfonamide as yellow oil. MS-ESI: 343.2 (M+1).

### Step 7: N-(tert-butyldimethylsilyl)-1-[3-[(dimethylamino)methyl]phenyl]methanesulfonoimidamide

Into a 250 mL 3-necked round-bottom flask, was placed a solution of PPh₃Cl₂ (0.5 M in DCE, 44 mL, 22.0 mmol, 2.5 equiv.) under nitrogen atmosphere. This was followed by the addition of 2,6-Dimethylpyridine (5.10 mL, 43.8 mmol, 5.0 equiv.) at 0° C. The resulting solution was stirred for 15 min at 0° C. and followed by the addition of N-(tert-butyldimethylsilyl)-1-[3-[(dimethylamino)methyl]phenyl]methanesulfonamide (3.00 g, 8.8 mmol, 1.0 equiv.) at 0° C. The resulting mixture was stirred for additional 30 min at 0° C. To the above mixture, NH_{3(g)} was bubbled at 0° C. for 30 min. The resulting solution was allowed to stir for an additional 15 h at ambient temperature. After concentration, the residue was purified by flash column chromatography on silica gel, eluting with dichloromethane/methanol (10:1) to give 500 mg of N-(tert-butyldimethylsilyl)-1-[3-[(dimethylamino)methyl]phenyl]methanesulfonoimidamide as yellow oil. MS-ESI: 342.2 (M+1).

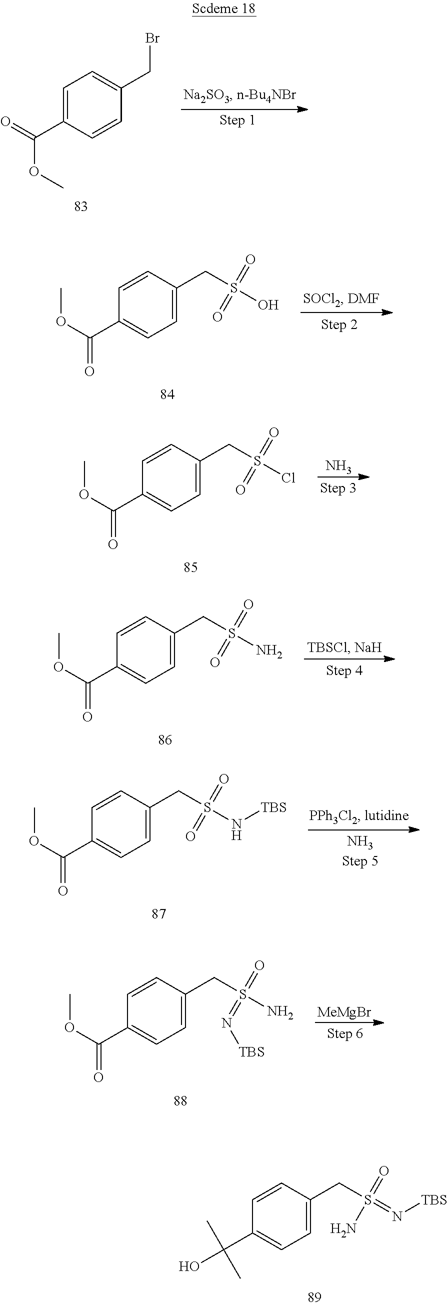

### Step 1: [4-(methoxycarbonyl)phenyl]methanesulfonic acid

Into a 250 mL round-bottom flask purged and maintained with an inert atmosphere of nitrogen, was placed a solution of methyl 4-(bromomethyl)benzoate (10.00 g, 43.7 mmol, 1.0 equiv.) in water (50 mL). To the solution were added Na₂SO₃ (7.15 g, 56.7 mmol, 1.3 equiv.) and tetrabutylammonium bromide (0.70 g, 2.2 mmol, 0.05 equiv.). The resulting solution was stirred for 15 h at 80° C. and concentrated under vacuum to give a crude solid, which was further washed with 50 mL of isopropyl alcohol to give 15 g of [4-(methoxy carbonyl)phenyl]methanesulfonic acid as a white solid. MS-ESI: 229.0 (M−1).

### Step 2: methyl 4-[(chlorosulfonyl)methyl]benzoate

Into a 250 mL round-bottom flask, was placed a solution of [4-(methoxycarbonyl)phenyl]methanesulfonic acid (15.0 g, 65.2 mmol, 1.0 equiv.) in DMF (20 mL). To the solution was added SOCl₂ (7.1 mL, 97.7 mmol, 1.5 equiv.). The resulting solution was stirred for 30 min at 0° C. The resulting solution was allowed to stir, for an additional 5 h at ambient temperature. The reaction was then quenched by the addition of water (20 mL). The resulting solution was extracted with dichloromethane and the combined organic layer were washed with water, dried over anhydrous Na₂SO₄ and concentrated under vacuum to give 12 g of methyl 4-[(chlorosulfonyl)methyl]benzoate as a yellow solid, which was used in the next step without purification.

### Step 3: methyl 4-(sulfamoylmethyl)benzoate

Into a 250 mL round-bottom flask, was placed a solution of methyl 4-[(chlorosulfonyl)methyl]benzoate (12.00 g, 48.3 mmol, 1.0 equiv.) in DCM (50 mL). To the solution, NH_{3(g)} was bubbled at 0° C. for 30 min. The resulting solution was stirred for 12 h at ambient temperature and then concentrated under vacuum. The residue was applied onto a silica gel column with ethyl acetate/hexane (40:60) to give 4.5 g of methyl 4-(sulfamoylmethyl)benzoate as a yellow solid. MS-ESI: 230.0 (M+1).

### Step 4: methyl 4-((N-(tert-butyldimethylsilyl)sulfamoyl)methyl)benzoate

Into a 100 mL round-bottom flask, was placed a solution of methyl 4-(sulfamoylmethyl)benzoate (1.40 g, 6.1 mmol, 1.0 equiv.) in THF (20 mL). To the solution was added NaH (60% wt in mineral oil, 0.49 g, 12.2 mmol, 2.0 equiv.). This was followed by the addition of TBSCl (1.38 g, 9.2 mmol, 1.5 equiv.). The resulting solution was stirred for 5 h at ambient temperature. The reaction was then quenched by the addition of 20 mL of water. The resulting solution was extracted with ethyl acetate and the combined organic layers were concentrated to give 2 g of methyl 4-4N-(tert-butyldimethylsilyl)sulfamoyl)methyl)benzoate as a yellow solid. MS-ESI: 344.1 (M+1).

### Step 5: methyl 4-((N′-(tert-butyldimethylsilyl)sulfamidimidoyl)methyl)benzoate

Into a 250 mL 3-necked round-bottom flask, was placed a solution of PPh₃Cl₂ (0.5 M in DCE, 46.6 mL, 23.3 mmol, 4.0 equiv.) under nitrogen atmosphere. This was followed by the addition of 2,6-dimethylpyridine (2.7 mL 7.83 mL, 23.3 mmol, 4.0 equiv.) at 0° C. The resulting solution was stirred for 15 min at 0° C. and followed by the addition of methyl 4-O-(tert-butyldimethylsilyl)sulfamoyl)methyl)benzoate (2.00 g, 5.8 mmol, 1.0 equiv.) at 0° C. The resulting mixture was stirred for additional 30 min at 0° C. To the above mixture, NH_{3(g)} was bubbled at 0° C. for 30 min. The resulting solution was allowed to stir for an additional 15 h at ambient temperature. After concentration, the residue was purified by flash column chromatography on silica gel, eluting with ethyl acetate/hexane (40:60) to give 1.67 g of methyl 4-((N′-(tert-butyldimethylsilyl) sulfamidimidoyl)methyl)benzoate as a yellow solid. MS-ESI: 343.2 (M+1).

### Step 6: N′-(tert-butyldimethylsilyl)-1-(4-(2-hydroxypropan-2-yl)phenyl)methanesulfonimidamide

Into a 100 mL 3-necked round-bottom flask, was placed a solution of methyl 4-0′-(tert-butyldimethylsilyl) sulfamidimidoyl)methyl)benzoate (1.40 g, 4.1 mmol, 1.0 equiv.) in THF (25 mL). To the solution was added MeMgBr (2 M in THF, 8.2 mL, 16.4 mmol, 4.0 equiv.) under N_{2(g)}. The resulting solution was stirred for 17 h at room temperature and then concentrated under vacuum. The residue was applied onto a silica gel column with ethyl acetate/hexane (44:56) to give 898 mg of N′-(tert-butyldimethylsilyl)-1-(4-(2-hydroxypropan-2-yl)phenyl)methane-sulfonimidamide as a yellow solid. MS-ESI: 343.2 (M+1).

### Examples 1-3

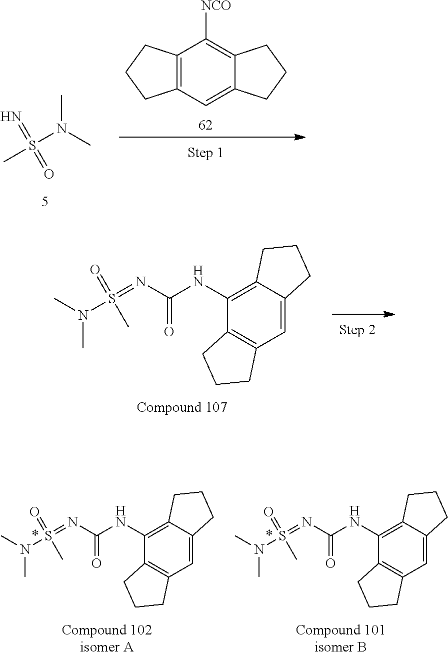

### Step 1: N′-((1,2,3,5,6,7-hexahydro-s-indacen-4-yl)carbamoyl)-N,N-dimethylmethanesulfonimidamide

Into a 50 mL round-bottom flask purged and maintained with an inert atmosphere of nitrogen, was placed a solution of N,N-dimethylmethanesulfonoimidamide (220 mg, 1.8 mmol, 1.0 equiv.) and TEA (0.75 mL, 5.4 mmol, 3.0 equiv.) in THF (15.0 mL). To the solution was added 4-isocyanato-1,2,3,5,6,7-hexahydro-s-indacene (286 mg, 1.4 mmol, 0.8 equiv.). The solution was stirred overnight at ambient temperature. The reaction mixture was concentrated under reduced pressure and the residue was purified by Prep-HPLC with the following conditions: Column: XBridge Prep OBD C18 Column, 30×150 mm, 5 um; Mobile Phase A: Water (10 mmol/L NH₄HCO₃+0.1% NH₄OH), Mobile Phase B: ACN; Flow rate: 60 mL/min; Gradient: 30% B to 50% B in 7 min; Detector, UV 220/254 nm. This resulted in 80 mg of N′-((1,2,3,5,6,7-hexahydro-s-indacen-4-yl(carbamoyl)-N,N-dimethylmethanesulfonimidamide as a white solid. MS-ESI: 322.2 (M+1). ¹H NMR: (400 MHz, DMSO-d₆) δ: 8.44 (br s, 1H), 6.89 (s, 1H), 3.11 (s, 3H), 2.80-2.68 (m, 14H), 1.99-1.91 (m, 4H)

### Step 2: Isomer a (Front Peak, Compound 102) and Isomer B (Second Peak, Compound 101)

The racemic N′-((1,2,3,5,6,7-hexahydro-s-indacen-4-yl)carbamoyl)-N,N-dimethylmethanesulfonimidamide (75 mg) was separated by Prep-Chiral-HPLC with the following conditions Column: Column: CHIRALPAK IC, 2*25 cm, 5 um; Mobile Phase A:Hex:DCM=3:1 (10 mmol/L NH₃-MeOH)-HPLC, Mobile Phase B:EtOH-HPLC; Flow rate: 20 mL/min; Gradient: 10% B to 10% B in 18 min; Detector: 220/254 nm; RT1:9.158; RT2:13.657. This resulted in 24.2 mg of isomer A (front peak, compound 102) as a white solid and 25.5 mg of isomer B (second peak, compound 101) as a white solid.

Compound 102: MS-ESI: 322.2 (M+1). ¹H NMR: (400 MHz, DMSO-d₆) δ: 8.44 (br s, 1H), 6.89 (s, 1H), 3.11 (s, 3H), 2.80-2.69 (m, 14H), 1.97-1.93 (m, 4H).

Compound 101: MS-ESI: 322.2 (M+1). ¹H NMR: (400 MHz, DMSO-d₆) δ: 8.44 (br s, 1H), 6.89 (s, 1H), 3.11 (s, 3H), 2.80-2.69 (m, 14H), 1.97-1.93 (m, 4H).

TABLE 6
Examples in the following table were prepared using similar conditions as described
in Examples 1-3, from common intermediate 62.

| | | | | | Exact |
|---|---|---|---|---|---|
| Example | Final Target | Sulfonimidamide | | | Mass |
| # | Number | Fragment | Structure | IUPAC Name | [M + H]⁺ |
| 4 | Compound 103 | 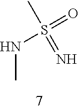 | 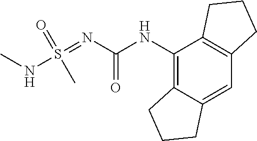 [SMILES: CNS(C)(=O)=NC(=O)Nc1c2c(cc3c1CCC3)CCC2] | N′-((1,2,3,5,6,7- hexahydro-s- indacen-4- yl)carbamoy1)-N- methylmethane- sulfonimidamide | 308.2 |
| 5 | Compound 103a | 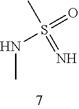 | 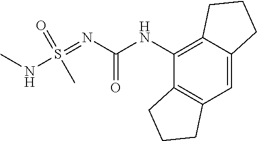 [SMILES: CNS(C)(=O)=NC(=O)Nc1c2c(cc3c1CCC3)CCC2] | (R)-N′- ((1,2,3,5,6,7- hexahydro-s- indacen-4- yl)carbamoy1)-N- methylmethane- sulfonimidamide (Isomer A) | 308.2 |
| 6 | Compound 103b | 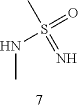 | 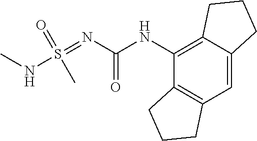 [SMILES: CNS(C)(=O)=NC(=O)Nc1c2c(cc3c1CCC3)CCC2] | (S)-N′- ((1,2,3,5,6,7- hexahydro-s- indacen-4- yl)carbamoy1)-N- methylmethane- sulfonimidamide (Isomer B) | 308.2 |
| 7 | Compound 115 | 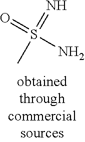 | 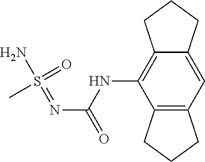 [SMILES: CS(N)(=O)=NC(=O)Nc1c2c(cc3c1CCC3)CCC2] | N′-((1,2,3,5,6,7- hexahydro-s- indacen-4- yl)carbamoyl) methane- sulfonimidamide | 294.1 |
| 8 | Compound 117 | 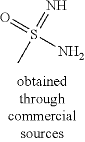 | 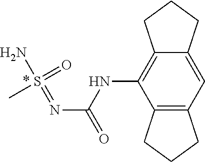 [SMILES: CS(N)(=O)=NC(=O)Nc1c2c(cc3c1CCC3)CCC2] | (S)-N′- ((1,2,3,5,6,7- hexahydro-s- indacen-4- yl)carbamoyl) methane- sulfonimidamide (Isomer A) | 294.1 |
| 9 | Compound 116 | 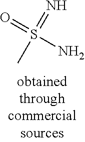 | 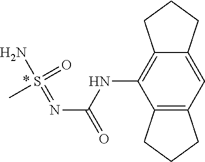 [SMILES: CS(N)(=O)=NC(=O)Nc1c2c(cc3c1CCC3)CCC2] | (R)-N′-((1,2,3,5,6,7- hexahydro-s- indacen-4- yl)carbamoyl) methane- sulfonimidamide (Isomer B) | 294.1 |

### Examples 10-12

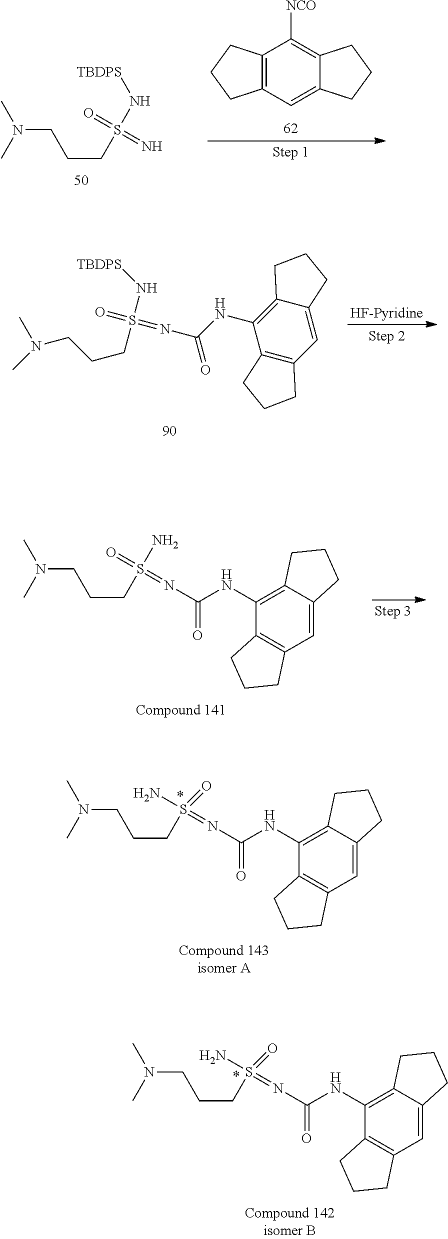

### Step 1: N-(tert-butyldiphenylsilyl)-3-(dimethylamino)-N′-((1,2,3,5,6,7-hexahydro-s-indacen-4-yl)carbamoyl)propane-1-sulfonimidamide

Into a 50 mL round-bottom flask purged and maintained with an inert atmosphere of nitrogen, was placed a solution of N-(tert-butyldiphenylsilyl)-3-(dimethylamino)propane-1-sulfonoimidamide (300 mg, 0.7 mmol, 1.0 equiv.) and NaH (60% wt in mineral oil, 44 mg, 1.1 mmol, 1.5 equiv.) in THF (10 mL). To the solution was added 4-isocyanato-1,2,3,5,6,7-hexahydro-s-indacene (148 mg, 0.7 mmol, 1.0 equiv.) at 0° C. The resulting solution was stirred for an additional 1 h at 0° C. in a water/ice bath and then quenched by the addition of water. The reaction mixture was extracted with ethyl acetate and the combined organic layers were concentrated to give 300 mg of N-(tert-butyldiphenylsilyl)-3-(dimethylamino)-N′-((1,2,3,5,6,7-hexahydro-s-indacen-4-yl)carbamoyl)propane-1-sulfonimidamide as a yellow solid. MS-ESI: 602.9 (M+1).

### Step 2: 3-(dimethylamino)-N′-((1,2,3,5,6,7-hexahydro-s-indacen-4-yl)carbamoyl)propane-1-sulfonimidamide

Into a 50 mL round-bottom flask purged and maintained with an inert atmosphere of nitrogen, was placed a solution of N-(tert-butyldiphenylsilyl)-3-(dimethylamino)-N′-((1,2,3,5,6,7-hexahydro-s-indacen-4-yl)carbamoyl)propane-1-sulfonimidamide (300 mg, 0.5 mmol, 1.0 equiv.) in THF (10 mL). To the solution was added HF-Pyridine (0.1 mL, w/t 70%, 5.5 mmol, 11.0 equiv.). The resulting solution was stirred for 12 h at room temperature then concentrated in vacuo. The residue was purified by Prep-HPLC with the following conditions: Column, XBridge Shield RP18 OBD Column, 19*250 mm, 10 um; mobile phase, Water (10 mmol/L NH₄HCO₃) and ACN (34% Phase B up to 44% in 8 min); Detector, UV 220/254 nm. This resulted in 120 mg of 3-(dimethylamino)-N′-((1,2,3,5,6,7-hexahydro-s-indacen-4-yl)carbamoyl)propane-1-sulfonimidamide as a white solid. MS-ESI: 365.2 (M+1). ¹H NMR: (300 MHz, DMSO-d₆) δ: 8.23 (s, 1H), 6.89 (s, 1H), 6.84-6.75 (m, 2H), 3.51-3.47 (m, 2H), 2.83-2.71 (m, 8H), 2.34-2.31 (m, 2H), 2.14 (s, 6H), 1.98-1.91 (m, 4H), 1.87-1.83 (m, 2H).

### Step 3: Isomer a (Front Peak, Compound 143) and Isomer B (Second Peak, Compound 142)

The racemic 3-(dimethylamino)-N′-((1,2,3,5,6,7-hexahydro-s-indacen-4-yl)carbamoyl)propane-1-sulfonimidamide (100 mg) was purified by Prep-Chiral-HPLC with the following conditions: Column: Column, CHIRALCEL OD-H, 2*25 mm, 5 um; mobile phase, Hex (8 mmol/L NH₃.MeOH) and EtOH (hold 15% EtOH in 13 min); Detector, UV 220/254 nm. This resulted in 30 mg of isomer A (front peak, compound 143) as a white solid and 30 mg of isomer B (second peak, compound 142) as a white solid.

Compound 143: MS-ESI: 365.2 (M+1). 1H NMR: (300 MHz, DMSO-d₆) δ: 8.21 (s, 1H), 6.93 (s, 1H), 6.87 (s, 2H), 3.49-3.41 (m, 2H), 2.81-2.71 (m, 8H), 2.34-2.30 (m, 2H), 2.13 (s, 6H), 1.98-1.94 (m, 4H), 1.89-1.83 (m, 2H).

Compound 142: MS-ESI: 365.2 (M+1). 1H NMR: (300 MHz, DMSO-d₆) δ: 8.22 (s, 1H), 6.93 (s, 1H), 6.87 (s, 2H), 3.51-3.42 (m, 2H), 2.81-2.71 (m, 8H), 2.35-2.31 (m, 2H), 2.14 (s, 6H), 1.99-1.94 (m, 4H), 1.89-1.86 (m, 2H).

TABLE 7
Examples in the following table were prepared using similar conditions as described
in Examples 10-12 from common intermediate 62.

| | | | | | Exact |
|---|---|---|---|---|---|
| Example | Final Target | Sulfonimidamide | | | Mass |
| # | Number | Fragment | Structure | IUPAC Name | [M + H]⁺ |
| 13 | Compound 104 | 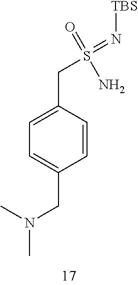 | 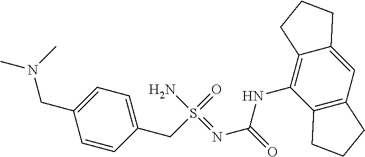 [SMILES: CN(C)Cc1ccc(CS(N)(=O)=NC(=O)Nc2c3c(cc4c2CCC4)CCC3)cc1] | 1-(4- ((dimethylamino) methyl)pheny1)- N′-((1,2,3,5,6,7- hexahydro-s- indacen-4- yl)carbamoyl) methane- sulfonimidamide | 427.2 |
| 14 | Compound 106 | 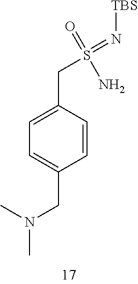 | 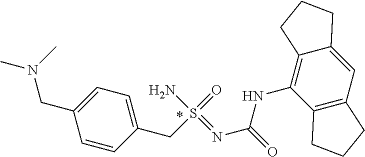 [SMILES: CN(C)Cc1ccc(CS(N)(=O)=NC(=O)Nc2c3c(cc4c2CCC4)CCC3)cc1] | (S)-1-(4- ((dimethylamino) methyl)pheny1)- N′-((1,2,3,5,6,7- hexahydro-s- indacen-4- yl)carbamoyl) methane- sulfonimidamide (Isomer A) | 427.2 |
| 15 | Compound 105 | 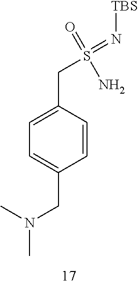 | 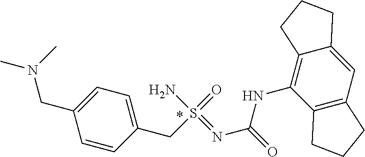 [SMILES: CN(C)Cc1ccc(CS(N)(=O)=NC(=O)Nc2c3c(cc4c2CCC4)CCC3)cc1] | (R)-1-(4- ((dimethylamino) methyl)pheny1)- N′-((1,2,3,5,6,7- hexahydro-s- indacen-4- yl)carbamoyl) methane- sulfonimidamide (Isomer B) | 427.2 |
| 16 | Compound 112 | 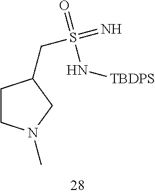 | 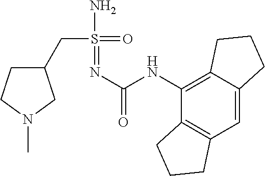 [SMILES: CN1CCC(CS(N)(=O)=NC(=O)Nc2c3c(cc4c2CCC4)CCC3)C1] | N′-((1,2,3,5,6,7- hexahydro-s- indacen-4- yl)carbamoy1)-1- (1- methylpyrrolidin- 3-yl) methane- sulfonimidamide | 377.2 |
| 17 | Compound 114 | 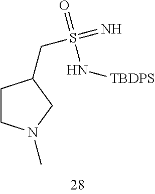 | 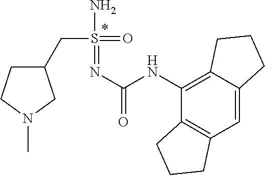 [SMILES: CN1CCC(CS(N)(=O)=NC(=O)Nc2c3c(cc4c2CCC4)CCC3)C1] | (S)-N′- ((1,2,3,5,6,7- hexahydro-s- indacen-4- yl)carbamoy1)-1- (1- methylpyrrolidin- 3-yl)methane- sulfonimidamide (Isomer A) | 377.2 |
| 18 | Compound 113 | 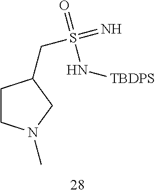 | 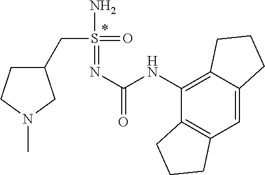 [SMILES: CN1CCC(CS(N)(=O)=NC(=O)Nc2c3c(cc4c2CCC4)CCC3)C1] | (R)-N′- ((1,2,3,5,6,7- hexahydro-s- indacen-4- yl)carbamoy1)-1- (1- methylpyrrolidin- 3-yl)methane- sulfonimidamide (Isomer B) | 377.2 |
| 19 | Compound 164 | 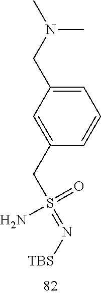 | 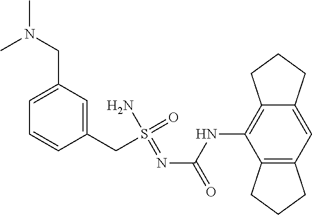 [SMILES: CN(C)Cc1cccc(CS(N)(=O)=NC(=O)Nc2c3c(cc4c2CCC4)CCC3)c1] | 1-(3- ((dimethylamino) methyl)pheny1)- N′-((1,2,3,5,6,7- hexahydro-s- indacen-4- yl)carbamoyl) methane- sulfonimidamide | 427.3 |
| 20 | Compound 139 | 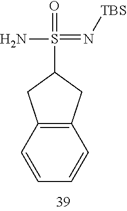 | 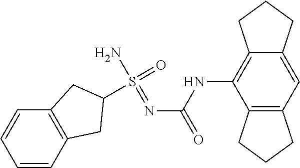 [SMILES: NS(=O)(=NC(=O)Nc1c2c(cc3c1CCC3)CCC2)C1Cc2ccccc2C1] | N′-((1,2,3,5,6,7- hexahydro-s- indacen-4- yl)carbamoy1)- 2,3-dihydro-1H- indene-2- sulfonimidamide | 395.2 |
| 21 | Compound 130 | 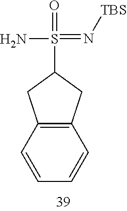 | 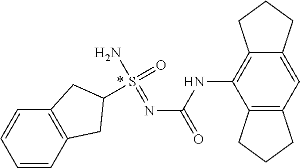 [SMILES: NS(=O)(=NC(=O)Nc1c2c(cc3c1CCC3)CCC2)C1Cc2ccccc2C1] | (S)-N′- ((1,2,3,5,6,7- hexahydro-s- indacen-4- yl)carbamoy1)- 2,3-dihydro-1H- indene-2- sulfonimidamide (Isomer A) | 395.2 |
| 22 | Compound 129 | 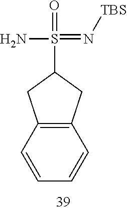 | 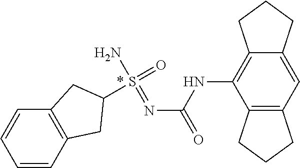 [SMILES: NS(=O)(=NC(=O)Nc1c2c(cc3c1CCC3)CCC2)C1Cc2ccccc2C1] | (R)-N′- ((1,2,3,5,6,7- hexahydro-s- indacen-4- yl)carbamoy1)- 2,3-dihydro-1H- indene-2- sulfonimidamide (Isomer B) | 395.2 |
| 23 | Compound 149 | 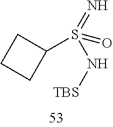 | 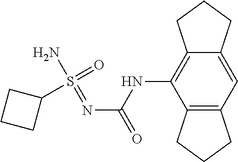 [SMILES: NS(=O)(=NC(=O)Nc1c2c(cc3c1CCC3)CCC2)C1CCC1] | N′-((1,2,3,5,6,7- hexahydro-s- indacen-4- yl)carbamoyl) cyclobutane- sulfonimidamide | 334.2 |
| 24 | Compound 146 | 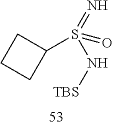 | 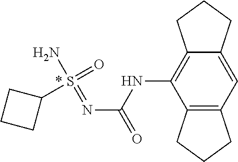 [SMILES: NS(=O)(=NC(=O)Nc1c2c(cc3c1CCC3)CCC2)C1CCC1] | (R)-N′- ((1,2,3,5,6,7- hexahydro-s- indacen-4- yl)carbamoyl) cyclobutane- sulfonimidamide (Isomer A) | 334.2 |
| 25 | Compound 145 | 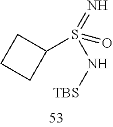 | 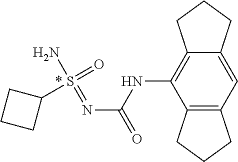 [SMILES: NS(=O)(=NC(=O)Nc1c2c(cc3c1CCC3)CCC2)C1CCC1] | (S)-N′- ((1,2,3,5,6,7- hexahydro-s- indacen-4- yl)carbamoyl) cyclobutane- sulfonimidamide (Isomer B) | 334.2 |
| 26 | Compound 156 | 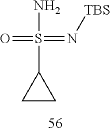 | 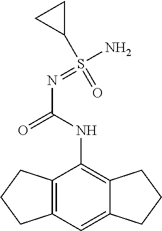 [SMILES: NS(=O)(=NC(=O)Nc1c2c(cc3c1CCC3)CCC2)C1CC1] | N′-((1,2,3,5,6,7- hexahydro-s- indacen-4- yl)carbamoyl) cyclopropane- sulfonimidamide | 320.2 |
| 27 | Compound 155 | 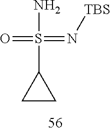 | 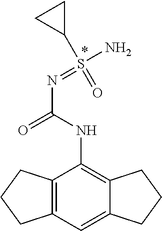 [SMILES: NS(=O)(=NC(=O)Nc1c2c(cc3c1CCC3)CCC2)C1CC1] | (R)-N′- ((1,2,3,5,6,7- hexahydro-s- indacen-4- yl)carbamoyl) cyclopropane- sulfonimidamide (Isomer A) | 320.2 |
| 28 | Compound 154 | 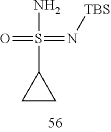 | 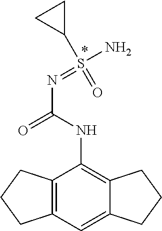 [SMILES: NS(=O)(=NC(=O)Nc1c2c(cc3c1CCC3)CCC2)C1CC1] | (S)-N′- ((1,2,3,5,6,7- hexahydro-s- indacen-4- yl)carbamoyl) cyclopropane- sulfonimidamide (Isomer B) | 320.2 |
| 29 | Compound 165 | 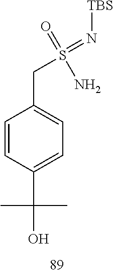 | 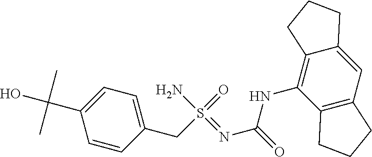 [SMILES: CC(C)(O)c1ccc(CS(N)(=O)=NC(=O)Nc2c3c(cc4c2CCC4)CCC3)cc1] | N′-((1,2,3,5,6,7- hexahydro-s- indacen-4- yl)carbamoyl)-1- (4-(2- hydroxypropan-2- yl)phenyl)methane- sulfonimidamide | 428.2 |
| 30 | Compound 165a | 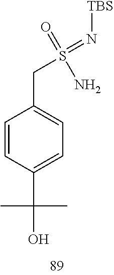 | 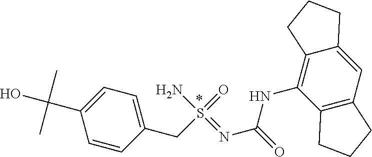 [SMILES: CC(C)(O)c1ccc(CS(N)(=O)=NC(=O)Nc2c3c(cc4c2CCC4)CCC3)cc1] | (S)-N′- ((1,2,3,5,6,7- hexahydro-s- indacen-4- yl)carbamoy1)-1- (4-(2- hydroxypropan-2- yl)phenyl)methane- sulfonimidamide (isomer A) | 428.2 |
| 31 | Compound 165b | 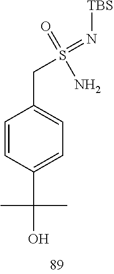 | 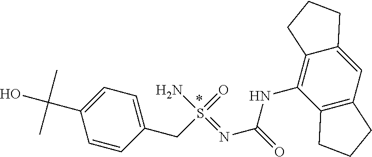 [SMILES: CC(C)(O)c1ccc(CS(N)(=O)=NC(=O)Nc2c3c(cc4c2CCC4)CCC3)cc1] | (R)-N- ((1,2,3,5,6,7- hexahydro-s- indacen-4- yl)carbamoy1)-1- (4-(2- hydroxypropan-2- yl)phenyl)methane- sulfonimidamide (isomer B) | 428.2 |

### Examples 32-36

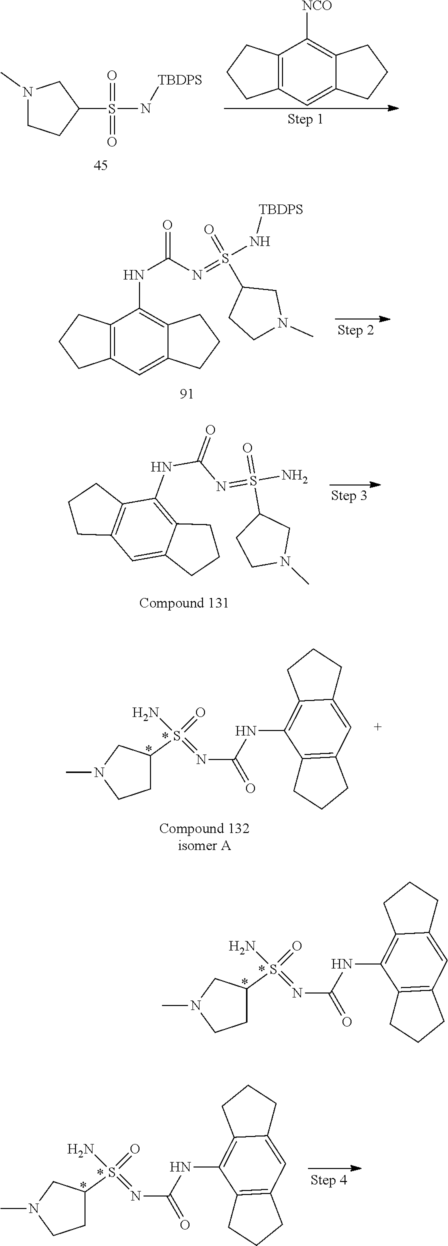

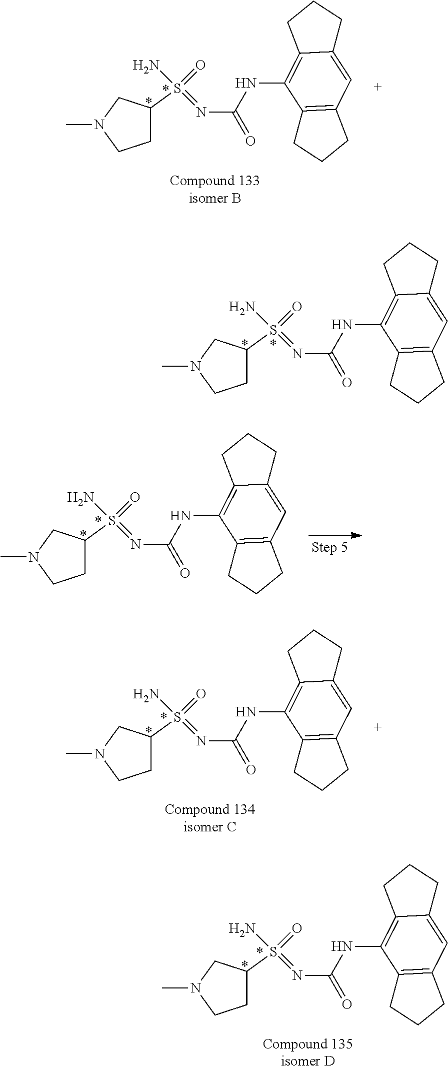 [SMILES: CN1CCC(S(N)(=O)=NC(=O)Nc2c3c(cc4c2CCC4)CCC3)C1 ; CN1CCC(S(N)(=O)=NC(=O)Nc2c3c(cc4c2CCC4)CCC3)C1 ; CN1CCC(S(N)(=O)=NC(=O)Nc2c3c(cc4c2CCC4)CCC3)C1 ; CN1CCC(S(N)(=O)=NC(=O)Nc2c3c(cc4c2CCC4)CCC3)C1 ; CN1CCC(S(N)(=O)=NC(=O)Nc2c3c(cc4c2CCC4)CCC3)C1]

### Step 1: N-(tert-butyldiphenylsilyl)-N′-((1,2,3,5,6,7-hexahydro-s-indacen-4-yl)carbamoyl)-1-methylpyrrolidine-3-sulfonimidamide

Into a 50 mL round-bottom flask, was placed a solution of N-(tert-butyldiphenylsilyl)-1-methylpyrrolidine-3-sulfonoimidamide (400 mg, 1.0 mmol, 1.0 equiv.) in THF (10 mL). To the solution was added NaH (60% wt in mineral oil, 100 mg, 2.5 mmol, 2.5 equiv.) and this was followed by the addition of 4-isocyanato-1,2,3,5,6,7-hexahydro-s-indacene (260 mg, 1.3 mmol, 1.3 equiv.). The resulting solution was stirred for 1 h at ambient temperature and then quenched by the addition of ice water (15 mL). The resulting solution was extracted with ethyl acetate and the combined organic layers were dried over anhydrous sodium sulfate and concentrated under vacuum to give 700 mg of N-(tert-butyldiphenylsilyl)-N′-((1,2,3,5,6,7-hexahydro-s-indacen-4-yl)carbamoyl)-1-methylpyrrolidine-3-sulfonimidamide as a yellow crude solid. MS-ESI: 601.4 (M+1).

### Step 2: N′-((1,2,3,5,6,7-hexahydro-s-indacen-4-yl)carbamoyl)-1-methylpyrrolidine-3-sulfonimidamide

Into a 100 mL round-bottom flask, was placed a solution of N-(tert-butyldiphenylsilyl)-N′-((1,2,3,5,6,7-hexahydro-s-indacen-4-yl)carbamoyl)-1-methylpyrrolidine-3-sulfonimidamide (700 mg, 1.2 mmol, 1.0 equiv.) in THF (20 mL). To the solution was added HF/Pyridine (0.2 mL, w/t 70%, 11.1 mmol, 10 equiv.) was added. The resulting solution was stirred for 1 h at ambient temperature and concentrated under vacuum. The crude product was purified by Prep-HPLC with the following conditions: Column: XBridge Prep OBD C18 Column 30×150 mm, 5 um; Mobile Phase A: Water (10 mmol/L NH₄HCO₃+0.1% NH₄OH), Mobile Phase B: ACN; Flow rate: 60 mL/min; Gradient: 25% B to 45% B in 7 min; Detector: 254/210 nm; Rt: 5.65 min. This resulted in 260 mg of N′-((1,2,3,5,6,7-hexahydro-s-indacen-4-yl(carbamoyl)-1-methylpyrrolidine-3-sulfonimidamide as a white solid. MS-ESI: 363.2 (M+1). ¹H NMR: (300 MHz, DMSO-d₆) δ: 8.21 (brs, 1H), 7.01 (brs, 2H), 6.87 (s, 1H), 4.33-4.22 (m, 1H), 2.84-2.68 (m, 10H), 2.56-2.51 (m, 2H), 2.25 (s, 3H), 2.18-2.08 (m, 2H), 1.98-1.92 (m, 4H).

### Step 3: Isomer A (Compound 132)

The isomers mixture (240 mg) was separated by Prep-SFC with the following conditions: Column: CHIRALPAK IG UL001, 20*250 mm, 5 um; Mobile Phase A: CO₂: 60, Mobile Phase B: MeOH (2 mM NH₃-MeOH): 40; Flow rate: 40 mL/min; Detector: 220 nm; RT1: 5.65; RT2: 7.63. This resulted in 38.5 mg of isomer A (front peak, compound 132) as a white solid and 160 mg of mixture with the other three isomers. MS-ESI: 363.2 (M+1). ¹H NMR: (300 MHz, DMSO-d₆) δ: 8.21 (brs, 1H), 7.01 (brs, 2H), 6.87 (s, 1H), 4.37-4.22 (m, 1H), 2.83-2.68 (m, 10H), 2.60-2.50 (m, 2H), 2.24 (s, 3H), 2.19-2.00 (m, 2H), 1.98-1.89 (m, 4H).

### Step 4: Isomer B (Compound 133)

The mixture of the other three isomers (150 mg) was separated by Prep-SFC with the following conditions: Column: CHIRALPAK AS-H, 2.0 cm*25 cm, 5 um; Mobile Phase A: CO₂: 80, Mobile Phase B: EtOH (2 mM NH₃-MeOH)-HPLC: 20; Flow rate: 40 mL/min; Detector: 220 nm; RT1: 4.91; RT2: 6.58. This resulted in 25.3 mg of isomer B (front peak, compound 133) as a white solid and 60.1 mg of the mixture of remaining two isomers. MS-ESI: 363.2 (M+1). ¹H NMR: (300 MHz, DMSO-d₆) δ: 8.21 (brs, 1H), 7.00 (brs, 2H), 6.87 (s, 1H), 4.32-4.22 (m, 1H), 2.80-2.68 (m, 10H), 2.61-2.50 (m, 2H), 2.24 (s, 3H), 2.18-2.02 (m, 2H), 1.99-1.88 (m, 4H).

### Step 5: Isomer A (Front Peak, Compound 134) and Isomer B (Second Peak, Compound 135)

The mixture of the rest two isomers (50 mg) was separated by Prep-SFC with the following conditions: Column: CHIRALPAK IE, 2*25 cm, 5 um; Mobile Phase A: Hex (8 mmol/L NH₃.MeOH)-HPLC, Mobile Phase B: EtOH-HPLC; Flow rate: 17 mL/min; Gradient: 50% B to 50% B in 18 min; Detector: 220/254 nm; RT1: 13.151; RT2: 14.919. This resulted in 17.8 mg of isomer A (front peak, compound 134) as a white solid and 17.9 mg of isomer B (second peak, compound 135) as a white solid.

Compound 134: MS-ESI: 363.2 (M+1). ¹H NMR: (300 MHz, DMSO-d₆) δ: 8.21 (brs, 1H), 7.00 (brs, 2H), 6.91 (s, 1H), 4.32-4.22 (m, 1H), 2.88-2.71 (m, 10H), 2.58-2.52 (m, 2H), 2.24 (s, 3H), 2.18-2.05 (m, 2H), 1.98-1.91 (m, 4H).

Compound 135: MS-ESI: 363.2 (M+1). ¹H NMR: (300 MHz, DMSO-d₆) δ: 8.20 (brs, 1H), 7.01 (brs, 2H), 6.87 (s, 1H), 4.34-4.25 (m, 1H), 2.83-2.71 (m, 10H), 2.59-2.51 (m, 2H), 2.24 (s, 3H), 2.18-2.07 (m, 2H), 1.99-1.92 (m, 4H).

### Examples 37-41

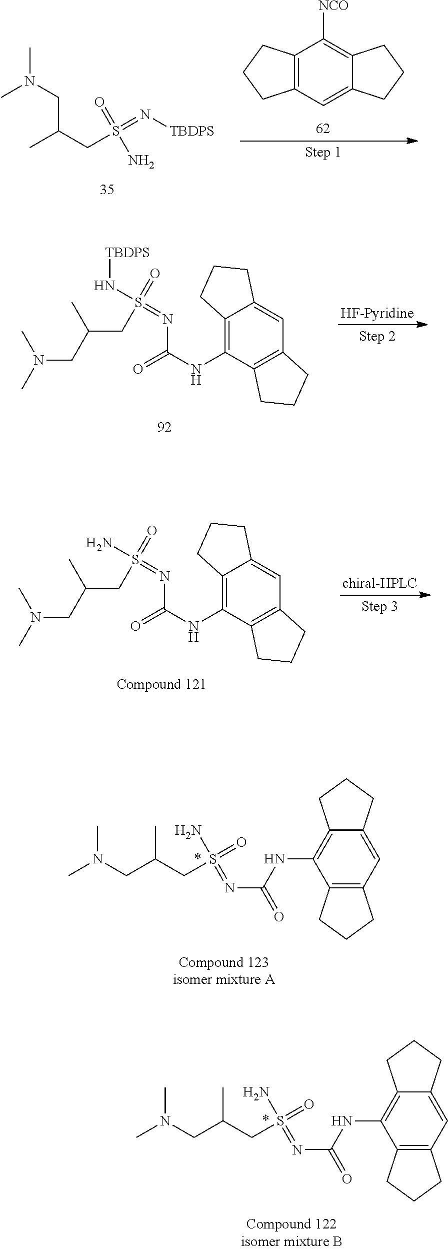

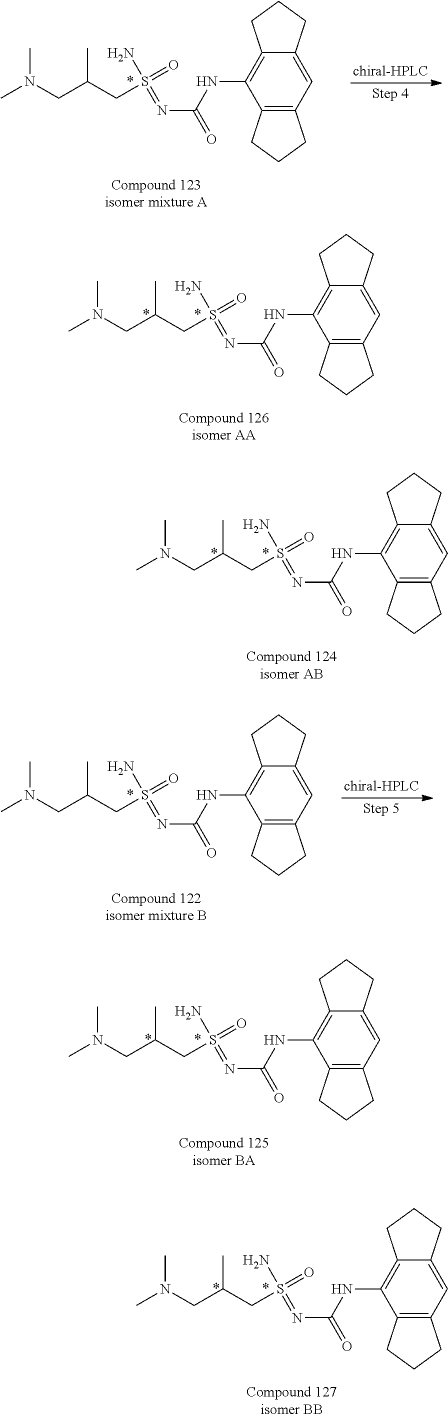 [SMILES: CC(CN(C)C)CS(N)(=O)=NC(=O)Nc1c2c(cc3c1CCC3)CCC2 ; CC(CN(C)C)CS(N)(=O)=NC(=O)Nc1c2c(cc3c1CCC3)CCC2 ; CC(CN(C)C)CS(N)(=O)=NC(=O)Nc1c2c(cc3c1CCC3)CCC2 ; CC(CN(C)C)CS(N)(=O)=NC(=O)Nc1c2c(cc3c1CCC3)CCC2 ; CC(CN(C)C)CS(N)(=O)=NC(=O)Nc1c2c(cc3c1CCC3)CCC2 ; CC(CN(C)C)CS(N)(=O)=NC(=O)Nc1c2c(cc3c1CCC3)CCC2]

### Step 1: N-(tert-butyldiphenylsilyl)-3-(dimethylamino)-N′-((1,2,3,5,6,7-hexahydro-s-indacen-4-yl)carbamoyl)-2-methylpropane-1-sulfonimidamide

Into a 250 mL round-bottom flask purged and maintained with an inert atmosphere of nitrogen, was placed a solution of N-(tert-butyldiphenylsilyl)-3-(dimethylamino)-2-methylpropane-1-sulfonoimidamide (1.25 g, 3.0 mmol, 1.0 equiv.) in THF (50 mL). This was followed by the addition of DBU (685 mg, 4.5 mmol, 1.5 equiv.) and 4-isocyanato-1,2,3,5,6,7-hexahydro-s-indacene (600 mg, 3.0 mmol, 1.0 equiv.). The resulting solution was stirred for 2 days at ambient temperature and then concentrated under vacuum to give 750 mg of crude N-(tert-butyldiphenylsilyl)-3-(dimethylamino)-N′-((1,2,3,5,6,7-hexahydro-s-indacen-4-yl)carbamoyl)-2-methylpropane-1-sulfonimidamide as a yellow crude solid, which was used to next step directly. MS-ESI: 617.3 (M+1).

### Step 2: 3-(dimethylamino)-N′-((1,2,3,5,6,7-hexahydro-s-indacen-4-yl)carbamoyl)-2-methylpropane-1-sulfonimidamide

Into a 100 mL round-bottom flask, was placed a solution of N-(tert-butyldiphenylsilyl)-3-(dimethylamino)-N′-((1,2,3,5,6,7-hexahydro-s-indacen-4-yl)carbamoyl)-2-methylpropane-1-sulfonimidamide (750 mg, 1.2 mmol, 1.0 equiv.) in THF (50 mL). To the solution was added HF/Pyridine (0.2 mL, w/t 70%, 11.1 mmol, 9.3 equiv.). The resulting solution was stirred for 1 h at ambient temperature and then quenched by the addition of water. The resulting solution was extracted with ethyl acetate and the combined organic layers were dried over anhydrous sodium sulfate and concentrated under vacuum. The crude product was purified by Flash-Prep-HPLC with the following conditions: Column, C18 silica gel; mobile phase, water/MeCN=90:10 increasing to water/MeCN=10:90 within 30 min; Detector, 220 nm. This resulted in 310 mg of 3-(dimethylamino)-N′-((1,2,3,5,6,7-hexahydro-s-indacen-4-yl)carbamoyl)-2-methylpropane-1-sulfonimidamide as a white solid. MS-ESI: 379.2 (M+1). ¹H NMR: (400 MHz, DMSO-d₆) δ 8.21 (d, 1H), 7.06 (s, 2H), 6.87 (s, 1H), 3.66-3.47 (m, 1H), 2.77-2.73 (m, 9H), 2.19 (d, 2H), 2.13 (d, 6H), 1.97-1.95 (m, 5H), 1.04 (d, 3H).

### Step 3: Isomer Mixture A (Front Peak, Compound 123) as a White Solid and 85 mg of Isomer Mixture B (Second Peak, Compound 122)

The racemic 3-(dimethylamino)-N′-((1,2,3,5,6,7-hexahydro-s-indacen-4-yl)carbamoyl)-2-methylpropane-1-sulfonimidamide (250.00 mg) was purified by Chiral-Prep-HPLC with the following conditions: Column: CHIRALPAK IC, 2*25 cm, 5 um; Mobile Phase A: MTBE (10 mM NH₃-MEOH)-HPLC-hotkey, Mobile Phase B: EtOH-HPLC; Flow rate: 20 mL/min; Gradient: 10% B to 10% B in 11 min; Detector, 254/220 nm; RT1: 6.028; RT2: 6.6. This resulted in 90 mg of isomer mixture A (front peak, compound 123) as a white solid and 85 mg of isomer mixture B (second peak, compound 122) as a white solid.

Compound 123: MS-ESI: 379.2 (M+1).

Compound 122: MS-ESI: 379.2 (M+1).

### Step 4: Isomer AA (Front Peak, Compound 126) and Isomer AB (Second Peak, Compound 124)

Isomer mixture A (front peak, compound 123) (85 mg) was separated by Chiral-Prep-HPLC with the following conditions: Column: CHIRALPAK ID, 2*25 cm, 5 um; Mobile Phase A: Hex:DCM=5:1 (10 mM NH₃-MEOH)-HPLC, Mobile Phase B: IPA-HPLC; Flow rate: 20 mL/min; Gradient: 5% B to 5% B in 30 min; Detector, 254/220 nm; RT1: 14.582; RT2: 24.937; This resulted in 17 mg of isomer AA (front peak, compound 126) as a white solid and 25 mg of isomer AB (second peak, compound 124) as a white solid.

Compound 126: MS-ESI: 379.2 (M+1). ¹H NMR: (400 MHz, DMSO-d₆) δ: 8.21 (d, 1H), 7.06 (s, 2H), 6.87 (s, 1H), 3.66-3.47 (m, 1H), 2.78-2.73 (m, 9H), 2.19 (d, 2H), 2.13 (d, 6H), 1.97-1.94 (m, 5H), 1.04 (d, 3H).

Compound 124: MS-ESI: 379.2 (M+1). ¹H NMR: (400 MHz, DMSO-d₆) δ: 8.21 (d, 1H), 7.06 (s, 2H), 6.87 (s, 1H), 3.66-3.47 (m, 1H), 2.78-2.73 (m, 9H), 2.19 (d, 2H), 2.13 (d, 6H), 1.97-1.94 (m, 5H), 1.04 (d, 3H).

### Step 5: Isomer BA (Front Peak, Compound 125) and Isomer BB (Second Peak, Compound 127)

Isomer mixture B (second peak, compound 122) (80 mg) was separated by Chiral-Prep-HPLC with the following conditions: Column: CHIRALPAK ID, 2*25 cm, 5 um; Mobile Phase A: Hex:DCM=5:1 (10 mM NH₃-MEOH)-HPLC, Mobile Phase B: IPA-HPLC; Flow rate: 20 mL/min; Gradient: 5% B to 5% B in 30 min; Detector, 254/220 nm; RT1: 14.582; RT2: 24.937. This resulted in 24 mg of isomer BA (front peak, compound 125) as a white solid and 24 mg of isomer BB (second peak, compound 127) as a white solid.

Compound 125: MS-ESI: 379.2 (M+1). ¹H NMR: (400 MHz, DMSO-d₆) δ: 8.21 (d, 1H), 7.06 (s, 2H), 6.87 (s, 1H), 3.66-3.47 (m, 1H), 2.78-2.73 (m, 9H), 2.19 (d, 2H), 2.13 (d, 6H), 1.97-1.94 (m, 5H), 1.04 (d, 3H).

Compound 127: MS-ESI: 379.2 (M+1). ¹H NMR: (400 MHz, DMSO-d₆) δ: 8.21 (d, 1H), 7.06 (s, 2H), 6.87 (s, 1H), 3.66-3.47 (m, 1H), 2.78-2.73 (m, 9H), 2.19 (d, 2H), 2.13 (d, 6H), 1.97-1.94 (m, 5H), 1.04 (d, 3H).

TABLE 8
Examples in the following table were prepared using similar conditions as described
25 in Examples 37-41 from common intermediate 62.

| | | | | | Exact |
|---|---|---|---|---|---|
| | Final Target | Sulfonimidamide | | | Mass |
| Example # | Number | Fragment | Structure | IUPAC Name | [M + H]⁺ |
| 42 | Compound 109 | 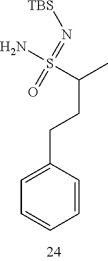 | 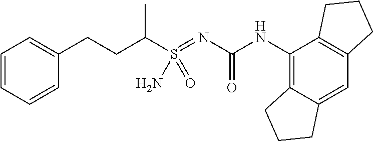 [SMILES: CC(CCc1ccccc1)S(N)(=O)=NC(=O)Nc1c2c(cc3c1CCC3)CCC2] | N′-((1,2,3,5,6,7- hexahydro-s- indacen-4- yl)carbamoy1)-3- phenylpropane-1- sulfonimidamide | 412.1 |
| 43 | Compound 110 | 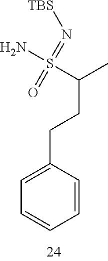 | 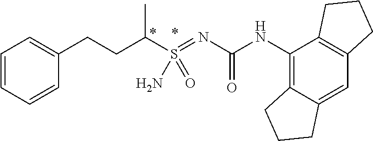 [SMILES: CC(CCc1ccccc1)S(N)(=O)=NC(=O)Nc1c2c(cc3c1CCC3)CCC2] | (R,2R)-N′- ((1,2,3,5,6,7- hexahydro-s- indacen-4- yl)carbamoy1)-4- phenylbutane-2- sulfonimidamide (Isomer A) | 412.1 |
| 44 | Compound 215 | 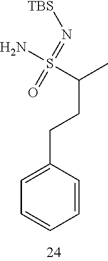 | 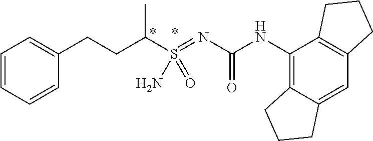 [SMILES: CC(CCc1ccccc1)S(N)(=O)=NC(=O)Nc1c2c(cc3c1CCC3)CCC2] | (R,2S)-N′- ((1,2,3,5,6,7- hexahydro-s- indacen-4- yl)carbamoy1)-4- phenylbutane-2- sulfonimidamide (Isomer B) | 412.1 |
| 45 | Compound 217 | 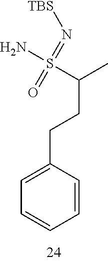 | 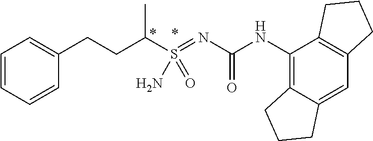 [SMILES: CC(CCc1ccccc1)S(N)(=O)=NC(=O)Nc1c2c(cc3c1CCC3)CCC2] | (S,2R)-N′- ((1,2,3,5,6,7- hexahydro-s- indacen-4- yl)carbamoy1)-4- phenylbutane-2- sulfonimidamide (Isomer C) | 412.1 |
| 46 | Compound 216 | 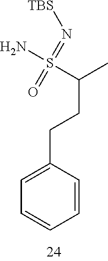 | 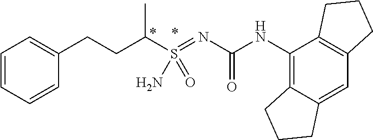 [SMILES: CC(CCc1ccccc1)S(N)(=O)=NC(=O)Nc1c2c(cc3c1CCC3)CCC2] | (S,2S)-N′- ((1,2,3,5,6,7- hexahydro-s- indacen-4- yl)carbamoy1)-4- phenylbutane-2- sulfonimidamide (Isomer D) | 412.1 |

### Example 47

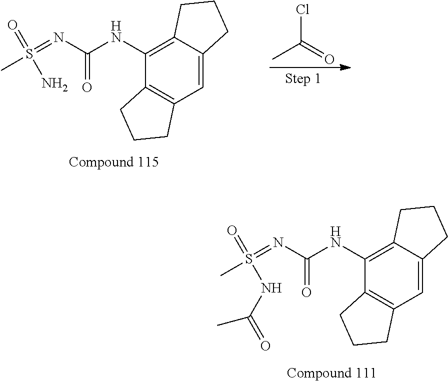

### Step 1: N—(N-((1,2,3,5,6,7-hexahydro-s-indacen-4-yl)carbamoyl)-S-methylsulfonimidoyl)acetamide

Into a 25 mL round-bottom flask purged and maintained with an inert atmosphere of nitrogen, was placed a solution of N′-((1,2,3,5,6,7-hexahydro-s-indacen-4-yl(carbamoyl)methanesulfonimidamide (100 mg, 0.34 mmol, 1.0 equiv.) and TEA (0.2 mL, 1.0 mmol, 3.0 equiv.) in DCM (10.0 mL). To the solution was added acetyl chloride (55 mg, 0.7 mmol, 2.0 equiv.). The resulting solution was stirred for overnight at ambient temperature and then concentrated under vacuum. The crude product was purified by Prep-HPLC with the following conditions: XSelect CSH Prep C18 OBD Column, 19*250 mm, 5 um; Mobile Phase A: Water (10 mmol/L NH₄HCO₃+0.1% NH₄OH), Mobile Phase B: ACN; Flow rate: 25 mL/min; Gradient: 16% B to 31% B in 7 min; Detector, 254/210 nm. This resulted in 5 mg of N—(N-((1,2,3,5,6,7-hexahydro-s-indacen-4-yl(carbamoyl)-S-methylsulfonimidoyl)acetamide as a white solid. MS-ESI: 336.2 (M+1). ¹H NMR: (400 MHz, DMSO-d₆) δ: 7.89 (br s, 1H), 7.07 (br s, 1H), 6.82 (s, 1H), 3.20 (s, 3H), 2.80-2.67 (m, 8H), 1.97-1.90 (m, 4H), 1.81 (s, 3H).

### Examples 48-50

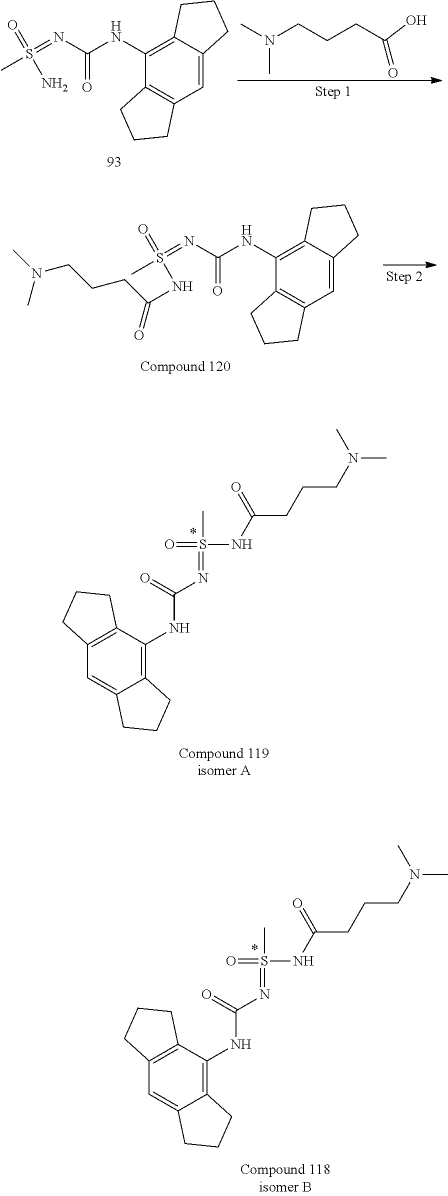

### Step 1: 4-(dimethylamino)-N—(N-((1,2,3,5,6,7-hexahydro-s-indacen-4-yl)carbamoyl)-S-methylsulfonimidoyl)butanamide

Into a 50 mL round-bottom flask purged and maintained with an inert atmosphere of nitrogen, was placed a solution of 4-(dimethylamino)butanoic acid (200 mg, 1.5 mmol, 1.5 equiv.), HATU (1.00 g, 2.6 mmol, 2.6 equiv.) and DIEA (500.00 mg, 3.9 mmol, 3.9 equiv.) in DMF (5.0 mL). To the solution was added N′-((1,2,3,5,6,7-hexahydro-s-indacen-4-yl(carbamoyl)methanesulfonimidamide (300 mg, 1.0 mmol, 1.0 equiv.). The resulting solution was stirred for overnight at ambient temperature and then concentrated under vacuum. The crude product was purified by Prep-HPLC with the following conditions: Column, XSelect CSH Prep C18 OBD Column, 5 um, 19*150 mm; mobile phase, Water (0.1% FA) and ACN (11% Phase B up to 30% in 7 min, hold 30% in 1 min); Detector, 220/254 nm. This resulted in 170 mg of 4-(dimethylamino)-N—(N-((1,2,3,5,6,7-hexahydro-s-indacen-4-yl(carbamoyl)-S-methylsulfonimidoyl)butanamide as a white solid. MS-ESI: 407.3 (M+1). ¹H NMR: (400 MHz, Methanol-d₄) δ: 8.19 (s, 1H), 6.94 (s, 1H), 3.40 (s, 3H), 3.17 (t, 2H), 2.91-2.72 (m, 14H), 2.48-2.45 (m, 2H), 2.13-2.10 (m, 4H), 2.01-1.98 (m, 2H).

### Step 2: Isomer A (Front Peak, Compound 119) and Isomer B (Second Peak, Compound 118)

The racemate (150 mg) was purified by Chiral-Prep-HPLC with the following conditions: Column: CHIRALPAK IC, 2*25 cm, 5 um; Mobile Phase A: MTBE (0.1% FA)-HPLC, Mobile Phase B: MeOH-HPLC; Flow rate: 20 mL/min; Gradient: 30% B to 30% B in 20 min; 254/220 nm; RT1:8.857; RT2:15.433. This resulted in 59.0 mg of isomer A (front peak, compound 119) as a white solid and 45.0 mg of isomer B (second peak, compound 118) as a white solid.

Compound 119: MS-ESI: 407.3 (M+1). ¹H NMR: (400 MHz, Methanol-d₄) δ: 8.19 (s, 1H), 6.94 (s, 1H), 3.40 (s, 3H), 3.18 (t, 2H), 2.89-2.78 (m, 14H), 2.48-2.44 (m, 2H), 2.12-2.08 (m, 4H), 2.01-1.95 (m, 2H).

Compound 118: MS-ESI: 407.3 (M+1). ¹H NMR: (400 MHz, Methanol-d₄) δ: 8.21 (s, 1H), 6.94 (s, 1H), 3.40 (s, 3H), 3.18 (t, 2H), 2.88-2.78 (m, 14H), 2.47-2.44 (m, 2H), 2.12-2.08 (m, 4H), 2.01-1.95 (m, 2H).

### Example 51

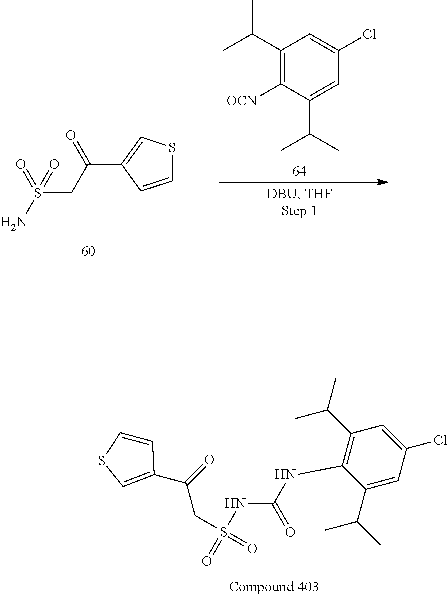

### Step 1: N-((4-chloro-2,6-diisopropylphenyl)carbamoyl)-2-oxo-2-(thiophen-3-yl)ethane-1-sulfonamide

Into a 25 mL round-bottom flask, was placed a solution of 2-oxo-2-(thiophen-3-yl)ethane-1-sulfonamide (80 mg, 0.4 mmol, 1.0 equiv.) and DBU (91 mg, 0.6 mmol, 1.5 equiv.) in tetrahydrofuran (5 mL). To the solution was added 5-chloro-2-isocyanato-1,3-bis(propan-2-yl)benzene (143 mg, 0.6 mmol, 1.5 equiv.). The resulting solution was stirred for 1 h at 25° C. and concentrated under vacuum. The crude product was purified by Prep-HPLC with the following conditions: Column: XBridge C18 OBD Prep Column, 19*250 mm, 10 um; Mobile Phase A: Water (0.05% TFA), Mobile Phase B: ACN; Flow rate: 20 mL/min; Gradient: 40% B to 70% B in 9 min; Detective: 254/220 nm. This resulted in 33.9 mg of N-((4-chloro-2,6-diisopropylphenyl)carbamoyl)-2-oxo-2-(thiophen-3-yl)ethane-1-sulfonamide as a light yellow solid. MS-ESI: 443.2 (M+1). ¹H NMR: (300 MHz, DMSO-d₆) δ 10.82 (s, 1H), 8.75-8.67 (m, 1H), 8.00 (s, 1H), 7.73-7.64 (m, 1H), 7.61-7.52 (m, 1H), 7.17 (s, 2H), 5.14 (s, 2H), 3.14-2.96 (m, 2H), 1.09 (d, 12H).

### Example 52

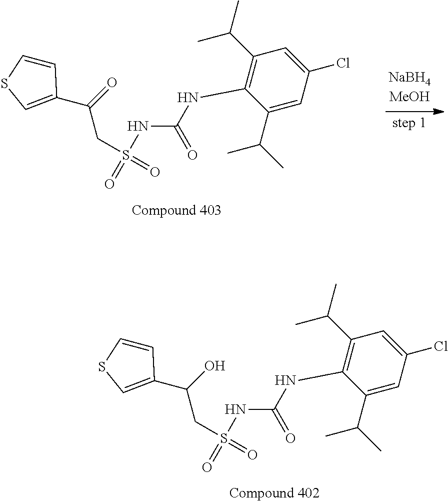 [SMILES: CC(C)c1cc(Cl)cc(C(C)C)c1NC(=O)NS(=O)(=O)CC(=O)c1ccsc1 ; CC(C)c1cc(Cl)cc(C(C)C)c1NC(=O)NS(=O)(=O)CC(O)c1ccsc1]

### Step 1: N-((4-chloro-2,6-diisopropylphenyl)carbamoyl)-2-hydroxy-2-(thiophen-3-yl)ethane-1-sulfonamide

Into a 50 mL round-bottom flask purged and maintained with an inert atmosphere of nitrogen, was placed a solution of N-((4-chloro-2,6-diisopropylphenyl)carbamoyl)-2-oxo-2-(thiophen-3-yl)ethane-1-sulfonamide (140 mg, 0.3 mmol, 1.0 equiv.) in methanol (10 mL). To the solution was added NaBH₄ (18 mg, 0.5 mmol, 1.5 equiv.). The resulting solution was stirred for 1 h at 25° C. and then concentrated under vacuum. The crude product was purified by Prep-HPLC with the following conditions: Column, XBridge C18 OBD Prep Column, 10 μm, 19 mm×250 mm; mobile phase, Water (10 mmol/L NH₄HCO₃) and ACN (10.0% ACN up to 70.0% in 8 min); Detector, 254 nm. This resulted in 32.8 mg of N-((4-chloro-2,6-diisopropylphenyl)carbamoyl)-2-hydroxy-2-(thiophen-3-yl)ethane-1-sulfonamide as a white solid. MS-ESI: 467.1 (M+Na). ¹H NMR: (400 MHz, DMSO-d₆) δ 10.24 (s, 1H), 8.00 (s, 1H), 7.53-7.45 (m, 1H), 7.41-7.35 (m, 1H), 7.16 (s, 2H), 7.09-7.06 (m, 1H), 5.81 (s, 1H), 5.18-5.10 (m, 1H), 3.81-3.70 (m, 1H), 3.68-3.58 (m, 1H), 3.10-3.05 (m, 2H), 1.17-1.05 (m, 12H).

### Example 53

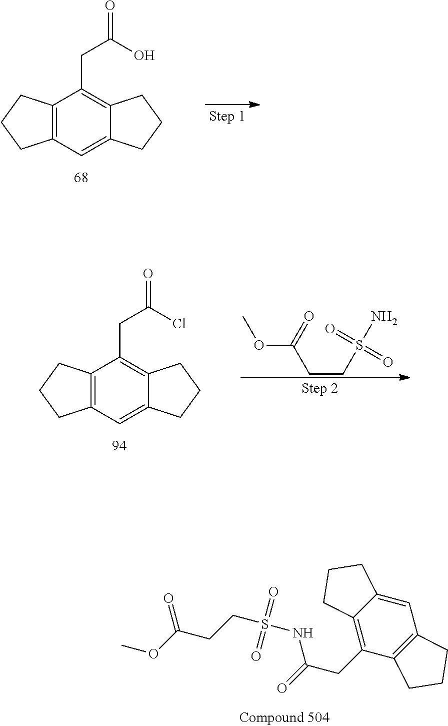

### Step 1: 2-(1,5-dihydro-s-indacen-4-yl)acetyl chloride

Into a 50 mL round-bottom flask, was placed a solution of 2-(1,5-dihydro-s-indacen-4-yl)acetic acid (175 mg, 0.8 mmol, 1.0 equiv.) in DCM (15 mL). To the solution was added oxalyl dichloride (2 mL, 23.5 mmol, 29.3 equiv.) and DMF (0.05 mL, 0.6 mmol, 0.75 equiv.). The resulting solution was stirred for 1 h at ambient temperature and then concentrated under vacuum to give 180 mg of crude 2-(1,5-dihydro-s-indacen-4-yl)acetyl chloride, which was used directly in the next step.

### Step 2: methyl 3-(N-(2-(1,2,3,5,6,7-hexahydro-s-indacen-4-yl)acetyl)sulfamoyl)propanoate

Into a 50 mL round-bottom flask, was placed a solution of methyl 3-sulfamoylpropanoate (140 mg, 0.8 mmol, 1.0 equiv.) and TEA (0.5 mL, 3.6 mmol, 4.5 equiv.) in DCM (20 mL). This was followed by the addition of a solution of 2-(1,5-dihydro-s-indacen-4-yl)acetyl chloride (180 mg, 0.8 mmol, 1 equiv) in DCM (5 mL) dropwise with stirring. The resulting solution was stirred for 1 h at ambient temperature and then concentrated under vacuum. The residue was purified by Prep-HPLC with the following conditions: Column, XBridge Shield RP18 OBD Column, 19*250 mm, 10 um; mobile phase, Water (10 mmol/L NH₄HCO₃) and ACN (13% Phase B up to 53% in 7 min); Detector, 220/254 nm. This resulted in 31.8 mg of methyl 3-(N-(2-(1,2,3,5,6,7-hexahydro-s-indacen-4-yl)acetyl)sulfamoyl)propanoate as a white solid. MS-ESI: 364.1 (M−1). ¹H NMR: (300 MHz, MeOH-d₄): 6.97 (s, 1H), 3.70-3.67 (m, 5H), 3.61 (s, 2H), 2.87-2.79 (m, 10H), 2.10-2.02 (m, 4H).

### Example 54

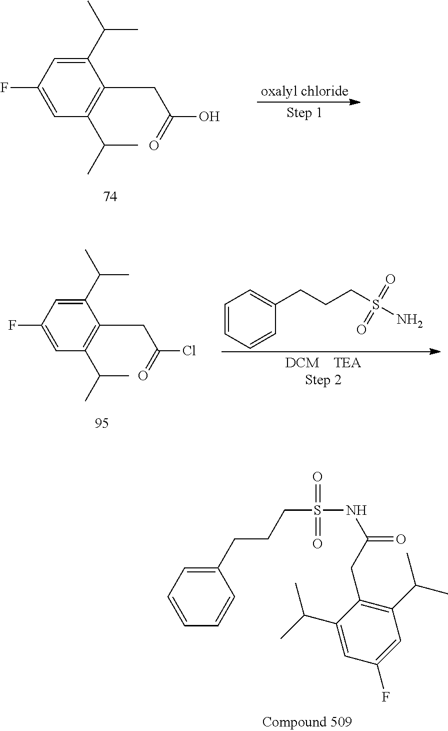

### Step 1: 2-(4-fluoro-2,6-diisopropylphenyl)acetyl chloride

Into a 50 mL round-bottom flask, was placed a solution of 2-(4-fluoro-2,6-diisopropylphenyl)acetic acid (60 mg, 0.25 mmol, 1.0 equiv.) in DCM (15 mL). To the solution was added oxalyl dichloride (1 mL, 11.8 mmol, 47.2 equiv.) and DMF (0.05 mL, 0.6 mmol, 2.4 equiv.). The resulting solution was stirred for 1 h at ambient temperature and then concentrated under vacuum to give 75 mg of crude 2-(4-fluoro-2,6-diisopropylphenyl)acetyl chloride, which was used directly in the next step.

### Step 2: 2-(4-fluoro-2,6-diisopropylphenyl)-N-((3-phenylpropyl)sulfonyl)acetamide

Into a 50 mL round-bottom flask, was placed a solution of 3-phenylpropane-1-sulfonamide (Enamine, 59 mg, 0.3 mmol, 1.0 equiv.) and TEA (0.2 mL, 1.4 mmol, 5.0 equiv.) in DCM (10 mL). This was followed by the addition of a solution of 2-(4-fluoro-2,6-diisopropylphenyl)acetyl chloride (75 mg, 0.3 mmol, 1.0 equiv.) in DCM (5 mL) dropwise with stirring. The resulting solution was stirred for 1 h at ambient temperature and then concentrated under vacuum. The residue was purified by Prep-HPLC with the following conditions: Column, XBridge Shield RP18 OBD Column, 19*250 mm, 10 um; mobile phase, Water (10 mmol/L NH₄HCO₃) and ACN (32% Phase B up to 60% in 8 min); Detector, UV220/254 nm. This resulted in 17.9 mg of 2-(4-fluoro-2,6-diisopropylphenyl)-N-((3-phenylpropyl)sulfonyl)acetamide as a white solid. MS-ESI: 418.2 (M−1). ¹H NMR: (300 MHz, MeOH-d₄): 7.27-7.22 (m, 2H), 7.17-7.13 (m, 3H), 6.80 (d, 2H), 3.74 (s, 2H), 3.40-3.20 (m, 2H), 3.04-3.00 (m, 2H), 2.72 (t, 2H), 2.08-2.03 (m, 2H), 1.15 (d, 12H).

TABLE 9
Examples in the following table were prepared using similar conditions as described
in Example 54 from common intermediate 74.

| | | | | | Exact |
|---|---|---|---|---|---|
| Example | Final Target | | | | Mass |
| # | Number | Sulfonamide | Structure | IUPAC Name | [M − H]⁺ |
| 55 | Compound 521 | 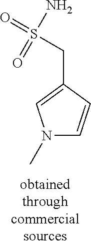 | 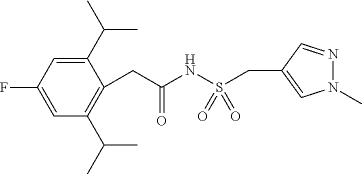 [SMILES: CC(C)c1cc(F)cc(C(C)C)c1CC(=O)NS(=O)(=O)Cc1cnn(C)c1] | 2-(4-fluoro-2,6- diisopropylphenyl)- N-(((1-methyl- 1H-pyrazol-4- yl)methyl) sulfony1) acetamide | 394.2 |
| 56 | Compound 520 | 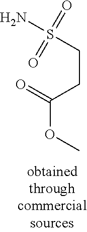 | 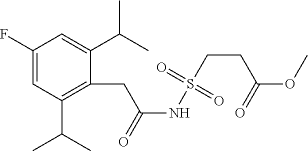 [SMILES: COC(=O)CCS(=O)(=O)NC(=O)Cc1c(C(C)C)cc(F)cc1C(C)C] | methyl 3-(N-(2- (4-fluoro-2,6- diisopropylphenyl) acetyl)sulfamoyl) propanoate | 386.2 |

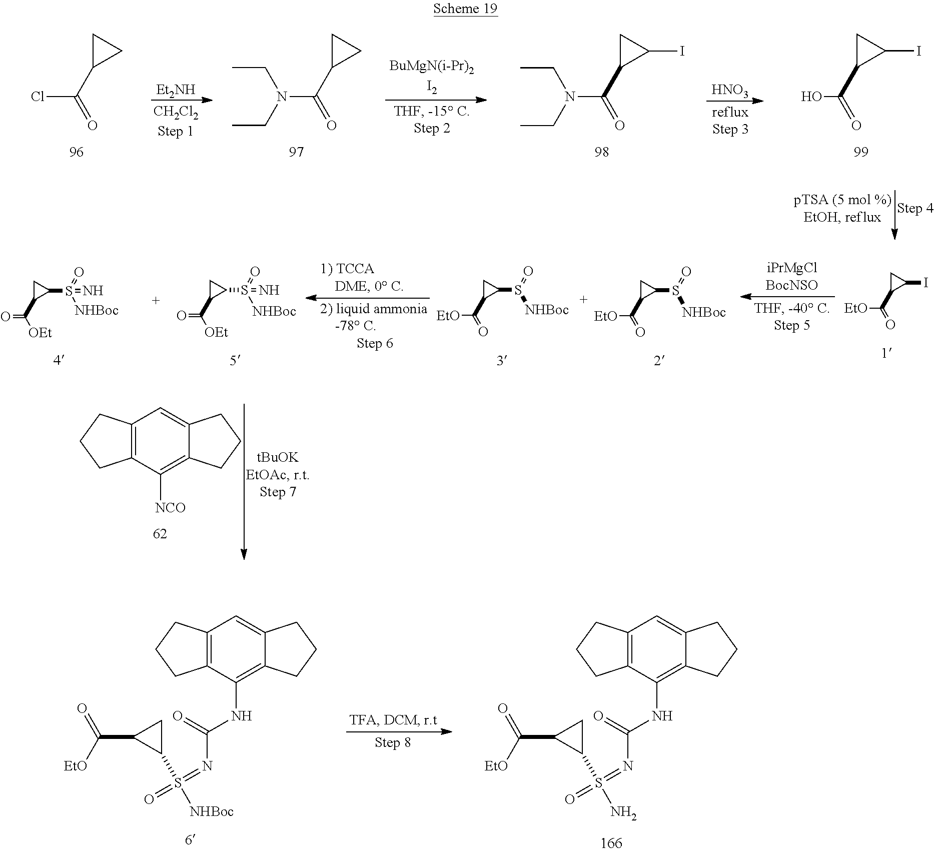

### ethyl trans-2-((R)—N′-((1,2,3,5,6,7-hexahydro-s-indacen-4-yl)carbamoyl)sulfamidimidoyl)cyclopropane-1-carboxylate (166)

### Step 1: N,N′-diethylcyclopropanecarboxamide, 97

To a 500 mL round bottom flask containing a 0° C. solution of diethylamine (42 g, 53.4 mL, 574 mmol, 3 equiv.) in 200 mL of dichloromethane, was added dropwise cyclopropyl carbonyl chloride (20 g, 17.4 mL, 191.3 mmol, 1 equiv.). The ice bath was removed, and the reaction mixture was stirred an additional hour at room temperature. The reaction was quenched by the addition of water (100 mL). The organic phase was washed with 1 M HCl (100 mL), saturated NaHCO₃ aq. (100 mL), and brine (100 mL). The solvent was removed in vacuo and the residue was purified by flash column chromatography on silica gel, using a gradient of 100% hexanes to 30% EtOAc in hexanes to yield 26.0 g of the title compound as a pale yellow oil.

### Step 2: cis-N,N-diethyl-2-iodocyclopropane-1-carboxamide, 98

To a flame dried 250 mL round bottom flask charged with dibutylmagnesium (1.0 M solution in heptane, 100 mL, 100 mmol, 1.05 equiv.) was added dropwise diisopropylamine (14.1 mL, 100 mmol, 1.05 equiv.), keeping the internal temperature below 40° C. The reaction mixture was stirred without external heating for 1 h then heated to reflux for 15 min and allowed to cool to room temperature. A solution of cis-cyclopropyl-N,N-diethylcarboxamide (13.5 g, 95.6 mmol, 1 equiv.) in anhydrous THF (50 mL) was added to the above mixture via a cannula, and the mixture was heated to 50° C. for 12 h.

A flame dried 500 mL round bottom flask was charged with a solution of 12 (48 g, 189 mmol, 2 equiv.) in anhydrous THF (140 mL), and the solution was cooled to 15° C. The reaction mixture containing the cyclopropane derivative was cooled to 0° C. before being added dropwise to the 12 solution via a cannula. After complete addition, the reaction mixture was stirred at 0° C. for 30 min. The reaction was quenched with concentrated H₂SO₄ (40 mL), and the THF was removed in vacuo. The crude material was dissolved in 200 mL of dichloromethane and 150 mL of water was added. The two phases were separated, and the aqueous phase was extracted with 2×200 mL of dichloromethane. The combined organic extracts were washed with saturated Na₂S₂O₃ aq. (3×200 mL), brine (200 mL) and dried over anhydrous MgSO₄. The filtrate was concentrated under reduced pressure to yield 27.88 g of the title compound as an oil that was used in the next step without any further purification.

### Step 3: cis-2-iodocyclopropane-1-carboxylic acid, 99

In a 500 mL round bottom flask was introduced cis-N,N-diethyl-2-iodocyclopropane-1-carboxamide (27.88 g, 104.4 mmol) followed by a solution of nitric acid (46.5 mL) in water (50 mL). The reactor was equipped with a condenser and the reaction was refluxed overnight. Upon reaction completion, the reaction mixture was cooled to ambient temperature and extracted with ethyl acetate (3×200 mL). The combined ethyl acetate extracts were washed with brine (2×100 mL), dried over MgSO4, and the solvent was removed in vacuo to give 16.4 g of the title compound as a pale orange solid that was used in the next step without any further purification. MS(ESI): 212.9 (M+1).

### Step 4: ethyl cis-2-iodocyclopropane-1-carboxylate, 1′

A 500 mL round bottom flask equipped with a reflux condenser was charged with cis-2-iodocyclopropane-1-carboxylic acid (6 g, 28.303), p-toluenesulfonic acid, (270 mg, 1.4 mmol, 0.05 equiv), and absolute ethanol (250 mL). The reaction mixture was stirred and heated to reflux for 12 h. The reaction mixture was allowed to cool to ambient temperature and the solvent removed under reduced pressure. The resulting liquid residue was taken up in EtOAc (200 mL) and water (100 mL), washed with saturated aqueous NaHCO₃ (2×150 mL) and brine (1×150 mL), dried over MgSO₄, filtered, and concentrated. The residue was purified by flash column chromatography on silica gel, using a gradient of 100% hexanes to 20% EtOAc in hexanes, 20 CV) to give 4.5 g of the title compound as a pale yellow oil.

### Step 5: ethyl cis-2-((S)-((tert-butoxycarbonyl)amino)sulfinyl)cyclopropane-1-carboxylate, 3′ and ethyl cis-2-((R)-((tert-butoxycarbonyl)amino)sulfinyl)cyclopropane-1-carboxylate, 2′

In a flame dried 250 mL round bottom flask was introduced ethyl cis-2-iodocyclopropane-1-carboxylate (5.0 g, 20.8 mmol, 1 equiv.) and dry THF (80 mL). The reaction mixture was cooled to −40° C. and i-PrMgCl solution (2M in THF, 10.9 mL, 21.9 mmol, 1.05 equiv.) was added. The reaction was stirred for 10 min at 40° C. and a solution of N-sulfinyl-tert-butylcarbamate (3.57 g, 2.97 mL, 21.9 mmol, 1.05 equiv., prepared according to J. Am. Chem Soc, 2004, 126(40), 12740-12741) in 20 mL of dry THF was added dropwise. The reaction was stirred at −40° C. for 1 h. At this point the reaction was quenched with 1M formic acid solution in THF (22 mL, 21.9 mL, 1.05 equiv.) and the reaction mixture was allowed to warm to ambient temperature. The reaction mixture was filtrated through a pad of silica gel and the solvent was evaporated in vacuo. The reaction was purified by flash column chromatography on silica gel, using a gradient of 100% hexanes to 100% EtOAc in hexanes to give two product diastereomers (ethyl cis-2-((S)-((tert-butoxycarbonyl)amino)sulfinyl)cyclopropane-1-carboxylate, 0.7 g, and ethyl cis-2-((R)-((tert-butoxy carbonyl)amino)sulfinyl)cyclopropane-1-carboxylate, 2.1 g) as white solids. MS(ESI): 300.1 (M+Na).

### Step 6: ethyl trans-2-(N-(tert-butoxycarbonyl)sulfamidimidoyl)cyclopropane-1-carboxylate, 4′, and ethyl cis-2-(N-(tert-butoxycarbonyl)sulfamidimidoyl)cyclopropane-1-carboxylate, 5′

In a flame dried 100 mL round bottom flask (flask 1) was introduced ethyl cis-2-((R)-((tert-butoxycarbonyl)amino)sulfinyl)cyclopropane-1-carboxylate, NC-65B (660 mg, 2.380 mmol, 1 equiv.) and dry DME (20 mL). The reaction mixture was cooled to 0° C. and trichlorocyanuric acid (276.5, 1.91 mmol, 005 equiv.) in 3 mL of DME was added dropwise. The reaction was stirred 1 h at 0° C. A second flame dried 100 mL round bottom flask (flask 2) was cooled to −78° C. and 4.5 mL of ammonia (gas) (4 g, 238 mmol, 100 equiv.) was condensed in the flask. At this point, 7 mL of dry DME was added and the solution was kept at −78° C. Flask 1 was cooled to −50° C. and added dropwise to the solution containing liquid ammonia (flask 2) via cannula. After 1 hour, the excess of ammonia was removed in vacuo at 78° C. and the reaction mixture was filtered through a pad of silica (1 cm). The solvent was evaporated in vacuo and the residue was purified by flash column chromatography on silica gel, using a gradient of 100% hexanes to 100% EtOAc in hexanes to yield 300 mg of ethyl trans-2-(N-(tert-butoxy carbonyl)sulfamidimidoyl)cyclopropane-1-carboxylate and 200 mg of ethyl cis-2-(N-(tert-butoxycarbonyl)sulfamidimidoyl)cyclopropane-1-carboxylate as white solids. MS(ESI): 315.1 (M+Na)

### Step 7: potassium (Z)—N—((S)-((tert-butoxycarbonyl)amino)(trans-2-(ethoxycarbonyl) cyclopropyl)(oxo)-λ⁶-sulfanylidene)-N′-(1,2,3,5,6,7-hexahydro-s-indacen-4-yl)carbamimidate, 6′

In a flame dried 50 ml round bottom flask under argon was introduced ethyl trans-2-(N-(tert-butoxycarbonyl)sulfamidimidoyl)cyclopropane-1-carboxylate (150 mg, 0.513 mmol, 1 equiv.) and 5 ml of EtOAc. A solution of 4-isocyanato-1,2,3,5,6,7-hexahydro-s-indacene (107.3 mg, 0.539 mmol, 1.05 equiv.) in 1 mL of EtOAc was added, followed by the dropwise addition of potassium tert-butoxide (1 M in THF, 0.539 mL, 539 mmol, 1.05 equiv.). The reaction mixture was stirred for 30 min at ambient temperature. The reaction mixture was quenched by the addition of 100 mL of hexanes and the solvents were removed in vacuo. The resulting salt was dried under vacuum to remove traces of tert-butanol, resulting in a white powder. The powder was suspended in 30 mL of heptane and sonicated for 5 min. The milky suspension was filtered and the resulting powder was placed under vacuum for 1 h, to give 215 mg of the title compound as a white powder. MS(ESI): 492.2 (M+1)

### Step 8: ethyl trans-2-((R)-N′-((1,2,3,5,6,7-hexahydro-s-indacen-4-yl)carbamoyl) sulfamidimidoyl)cyclopropane-1-carboxylate, Compound 166

In a flame dried 50 mL round bottom flask was introduced potassium (Z)—N—((S)-((tert-butoxycarbonyl)amino)(trans-2-(ethoxycarbonyl) cyclopropyl)(oxo)-λ⁶-sulfanylidene)-N-(1,2,3,5,6,7-hexahydro-s-indacen-4-yl)carbamimidate (200 mg, 0.41 mmol, 1 equiv.) and 20 mL of DCM. TFA was added dropwise (6 mL, 81 mmol, 200 equiv.) and the reaction was stirred for 30 min at ambient temperature. At this point the reaction was diluted with 200 mL of heptane and the solvents were removed in vacuo. Residual TFA was removed by co-evaporation with 200 mL of a 1:1 mixture of DCM and heptane. The resulting material was dissolved in 200 mL of EtOAc and the precipitate was removed by filtration on a paper filter. The filtrate was passed through a 1 cm silica pad and rinsed with another 100 mL portion of EtOAc. The EtOAc fraction was discarded and the expected product was eluted from the silica pad using 200 mL of DCM:MeOH (1:1). The solvents were evaporated and the white solid was dissolved in 100 mL of DCM:heptane (1:1) and sonicated for 15 min. Sonication was stopped and the product was allowed to crystalize over 1 h. Filtration yielded 20 mg of the title compound as a white powder (20 mg, d.r. 11:1). ¹H NMR (DMSO-d₆, 400 MHz): 8.33 (br s, 1H), 7.13 (s, 2H), 6.87 (s, 1H), 4.12-4.07 (m, 2H), 3.48 (ddd, 1H), 2.78 (t, 4H), 2.70-2.65 (m, 4H), 2.34-2.30 (m, 1H), 1.93 (ddd, 4H), 1.68-1.64 (m, 1H), 1.51 (dt, 1H), 1.20 (t, 3H), MS(ESI): 392.2 (M+1).

### Scheme for the Preparation of Key Intermediates of Formula AB

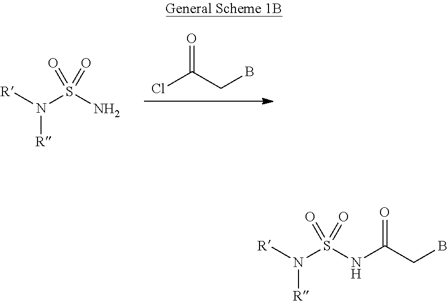

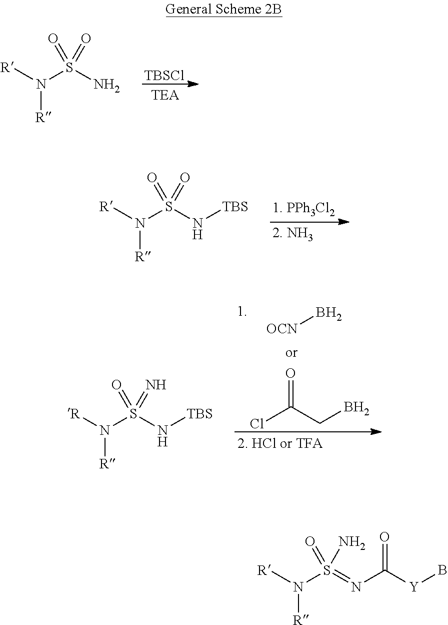

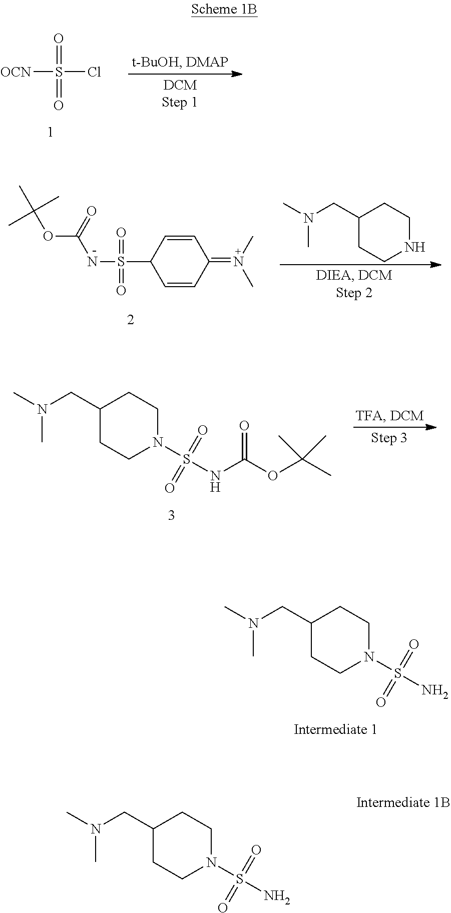

### 4-((dimethylamino)methyl)piperidine-1-sulfonamide

### Step 1: (tert-butoxycarbonyl)((4-(dimethyliminio)cyclohexa-2,5-dien-1-yl)sulfonyl)amide

Into a 50 mL round-bottom flask purged and maintained with an inert atmosphere of nitrogen, was placed a solution of t-BuOH (2.10 g, 28.3 mmol, 1.0 equiv.) in DCM (20 mL). To this was added sulfurisocyanatidic chloride (4.0 g, 28.2 mmol, 1.0 equiv.) dropwise with stirring. That was followed by the addition of DMAP (6.91 g, 56.6 mmol, 2.0 equiv.). The resulting solution was stirred for 1 h at ambient temperature. The resulting mixture was washed with brine, dried over anhydrous sodium sulfate and concentrated to result in 8 g of (tert-butoxycarbonyl)((4-(dimethylamino)pyridin-1-ium-1-yl)sulfonyl)amide as a white solid. MS-ESI: 301.1 (M+1).

### Step 2: tert-butyl ((4-((dimethylamino)methyl)piperidin-1-yl)sulfonyl)carbamate

Into a 25-mL round-bottom flask, was placed a solution of dimethyl(piperidin-4-ylmethyl)amine (284 mg, 2.0 mmol, 1.0 equiv.) in DCM (10 mL). This was followed by the addition of (tert-butoxycarbonyl)((4-(dimethyliminio)cyclohexa-2,5-dien-1-yl)sulfonyl)amide (600 mg, 2.0 mmol, 1.0 equiv.). The resulting solution was stirred for 2 h at ambient temperature. The resulting mixture was concentrated and the residue was purified by flash column chromatography on silica gel, eluting with dichloromethane/methanol (4:1) to result in 220 mg of tert-butyl ((4-((dimethylamino)methyl)piperidin-1-yl)sulfonyl)carbamate as a yellow solid. MS-ESI: 322.2 (M+1).

### Step 3: 4-((dimethylamino)methyl)piperidine-1-sulfonamide

Into a 25-mL round-bottom flask, was placed a solution of tert-butyl ((4-((dimethylamino)methyl)piperidin-1-yl)sulfonyl)carbamate (220 mg, 0.7 mmol, 1.0 equiv.) in DCM (2 mL). This was followed by the addition of TFA (2 mL, 26.9 mmol, 38.4 equiv.) dropwise with stirring. The resulting solution was stirred for 1 h at ambient temperature. The resulting mixture was concentrated to result in 145 mg of crude 4-((dimethylamino)methyl)piperidine-1-sulfonamide as a yellow solid, which was used to next step without further purification. MS-ESI: 222.1 (M+1).

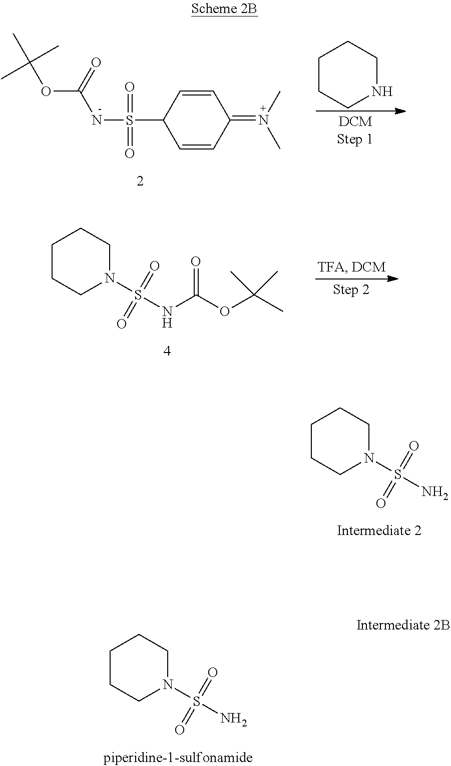

### Step 1: tert-butyl (piperidin-1-ylsulfonyl)carbamate

Into a 25 mL round-bottom flask, was placed a solution of (tert-butoxycarbonyl)((4-(dimethyliminio)cyclohexa-2,5-dien-1-yl)sulfonyl)amide (300 mg, 1.0 mmol, 1.0 equiv.) in DCM (10 mL). This was followed by the addition of piperidine (85 mg, 1.0 mmol, 1.0 equiv.). The resulting solution was stirred overnight at ambient temperature. The resulting mixture was concentrated and the residue was purified by flash column chromatography on silica gel, eluting with ethyl acetate/petroleum ether (1:1) to give 150 mg of tert-butyl (piperidin-1-ylsulfonyl)carbamate as a white solid. MS-ESI: 265.1 (M+1).

### Step 2: piperidine-1-sulfonamide

Into a 25 mL round-bottom flask, was placed a solution of tert-butyl (piperidin-1-ylsulfonyl)carbamate (100 mg, 0.38 mmol, 1.0 equiv.) in DCM (2 mL). This was followed by the addition of TFA (2 mL, 26.9 mmol, 70.8 equiv.) dropwise with stirring at 0° C. The resulting solution was stirred for 30 min at ambient temperature. The resulting mixture was concentrated, and the residue was applied onto a silica gel column with ethyl acetate/petroleum ether (1:2) to give 50 mg of piperidine-1-sulfonamide as a yellow solid. MS-ESI: 165.1 (M+1).

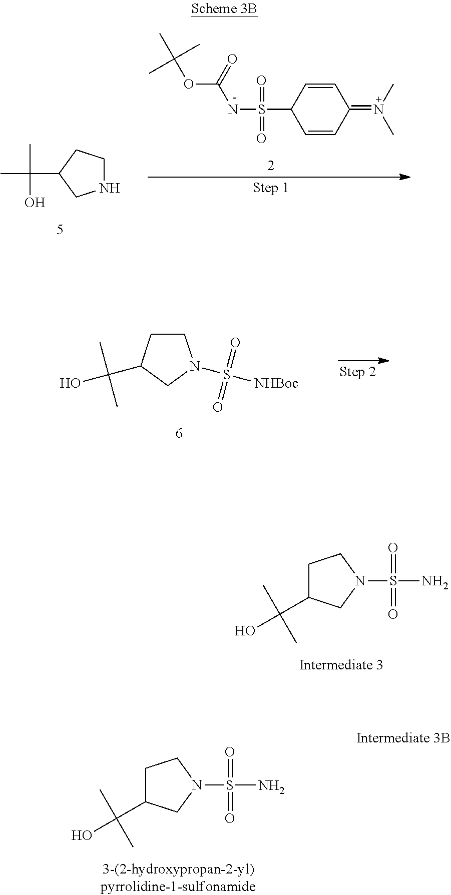

### Step 1: tert-butyl ((3-(2-hydroxypropan-2-yl)pyrrolidin-1-yl)sulfonyl)carbamate

To a solution of 2-(pyrrolidin-3-yl)propan-2-ol (100 mg, 0.77 mmol, 1.0 equiv.) in DCM(10 mL), was added (tert-butoxycarbonyl)((4-(dimethyliminio)cyclohexa-2,5-dien-1-yl)sulfonyl)amide (233 mg, 0.77 mmol, 1.0 equiv.). The solution was stirred for overnight at ambient temperature. The resulting mixture was concentrated under reduced pressure and the residue was purified by flash column chromatography on silica gel, eluting with petroleum ether/ethyl acetate (1:1) to give 140 mg of tert-butyl ((3-(2-hydroxypropan-2-yl)pyrrolidin-1-yl)sulfonyl)carbamate as a white solid. MS-ESI: 309.1 (M+1).

### Step 2: 3-(2-hydroxypropan-2-yl)pyrrolidine-1-sulfonamide

tert-butyl 43-(2-hydroxypropan-2-yl)pyrrolidin-1-yl)sulfonyl)carbamate (140 mg, 0.45 mmol, 1 equiv.) was dissolved into concentrated hydrogen chloride aq. (5 mL). The solution was stirred for 3 h at ambient temperature. The resulting mixture was concentrated under vacuum and the residue purified by flash column chromatography on silica gel, eluting with petroleum ether/ethyl acetate (1:1) to give 75 mg of 3-(2-hydroxypropan-2-yl)pyrrolidine-1-sulfonamide as a white solid. MS-ESI: 209.1 (M+1).

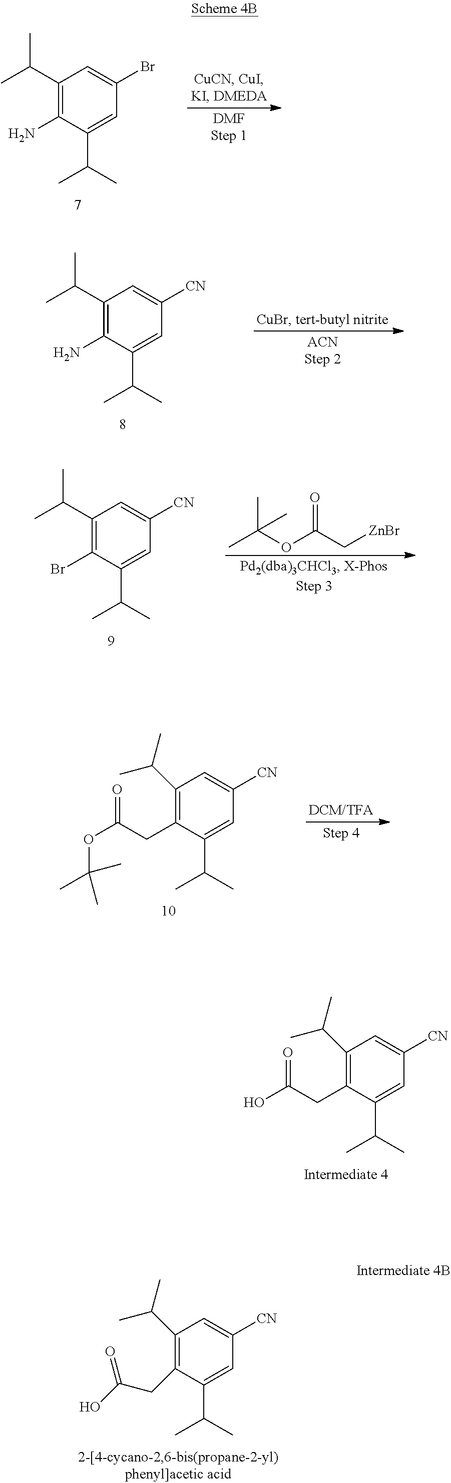

### Step 1: 4-amino-3,5-bis(propan-2-yl)benzonitrile

Into a 100 mL round-bottom flask purged and maintained with an inert atmosphere of nitrogen, was placed a solution of 4-bromo-2,6-bis(propan-2-yl)aniline (5.1 g, 19.9 mmol, 1.0 equiv.) in DMF (30 mL). To the solution were added CuCN (2.16 g, 24.1 mmol, 1.20 equiv), Cul (380 mg, 2.0 mmol, 0.1 equiv.), KI (664 mg, 4.0 mmol, 0.20 equiv) and TMEDA (2.0 mL, 2.0 mmol, 1.00 equiv). The resulting solution was stirred for 24 h at 100° C. in an oil bath and then quenched with water (20 mL). The resulting solution was extracted with ethyl acetate and the combined organic layers were washed with brine, dried over anhydrous sodium sulfate and concentrated under vacuum. The residue was purified by flash column chromatography on silica gel, eluting with ethyl acetate/petroleum ether (1:20) to give 1.2 g of 4-amino-3,5-bis(propan-2-yl)benzonitrile as a yellow solid. MS-ESI: 203.2 (M+1).

### Step 2: 4-bromo-3,5-bis(propan-2-yl)benzonitrile

Into a 250 mL round-bottom flask, was placed a solution of 4-amino-3,5-bis(propan-2-yl)benzonitrile (7.00 g, 34.6 mmol, 1.0 equiv.) and CuBr (9.90 g, 69.2 mmol, 2.0 equiv.) in ACN (150 mL). This was followed by the addition of tert-butyl nitrite (7.1 g, 69.2 mmol, 2.0 equiv.) dropwise with stirring at 0° C. The resulting solution was stirred for 3 h at 60° C. in an oil bath and then concentrated under vacuum. The residue was purified by flash column chromatography on silica gel, eluting with ethyl acetate/petroleum ether (1:100) to give 4.1 g of 4-bromo-3,5-bis(propan-2-yl)benzonitrile as a yellow solid.

### Step 3: tert-butyl 2-[4-cyano-2,6-bis(propan-2-yl)phenyl]acetate

Into a 40 mL sealed tube purged and maintained with an inert atmosphere of nitrogen, was placed a solution of 4-bromo-3,5-bis(propan-2-yl)benzonitrile (470 mg, 1.8 mmol, 1.0 equiv.) in THF (15 mL). To the solution were added Pd₂(dba)₃CHCl₃ (182 mg, 0.2 mmol, 0.1 equiv.), X-Phos (84 mg, 0.2 mmol, 0.1 equiv.), and tert-butyl 2-(bromozincio)acetate (1.37 g, 5.3 mmol, 3.0 equiv.). The resulting solution was stirred for 16 h at 70° C. and then quenched by the addition of water (30 mL). The resulting solution was extracted with dichloromethane and the combined organic layers were dried over anhydrous sodium sulfate and concentrated under vacuum to give 300 mg of tert-butyl 2-[4-cyano-2,6-bis(propan-2-yl)phenyl]acetate as a yellow solid.

### Step 4: 2-[4-cyano-2,6-bis(propan-2-yl)phenyl]acetic acid

Into a 50 mL round-bottom flask, was placed a solution of tert-butyl 2-[4-cyano-2,6-bis(propan-2-yl)phenyl]acetate (300 mg, 1.0 mmol, 1.0 equiv.) in DCM (10 mL) and TFA (3 mL). The resulting solution was stirred for 6 h at ambient temperature and concentrated under vacuum to give 260 mg (crude) of 2-[4-cyano-2,6-bis(propan-2-yl)phenyl]acetic acid as a yellow solid, which was used directly without additional purification. MS-ESI: 244.1 (M−1). ¹H NMR: (400 MHz, DMSO-d₆) δ: 12.56 (s, 1H), 7.58 (s, 2H), 3.80 (s, 2H), 6.87 (s, 1H), 3.17-3.11 (m, 2H), 1.18-1.16 (m, 12H).

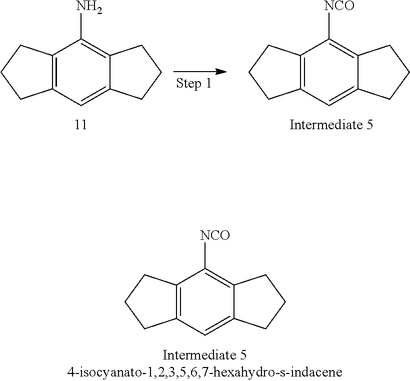

### Step 1: 4-isocyanato-1,2,3,5,6,7-hexahydro-s-indacene

Into a 500 mL round-bottom flask, was placed a solution of 1,2,3,5,6,7-hexahydro-s-indacen-4-amine (20.0 g, 115.4 mmol, 1.0 equiv.) in THF (250 mL). This was followed by the addition of ditrichloromethyl carbonate (13.70 g, 46.2 mmol, 0.4 equiv.) in portions. The resulting solution was stirred for 3 h at 70° C. and concentrated under vacuum to give in 22.5 g of crude 4-isocyanato-1,2,3,5,6,7-hexahydro-s-indacene as a yellow solid, which was used directly in the next step.

### Example 1B (Compound 201B)

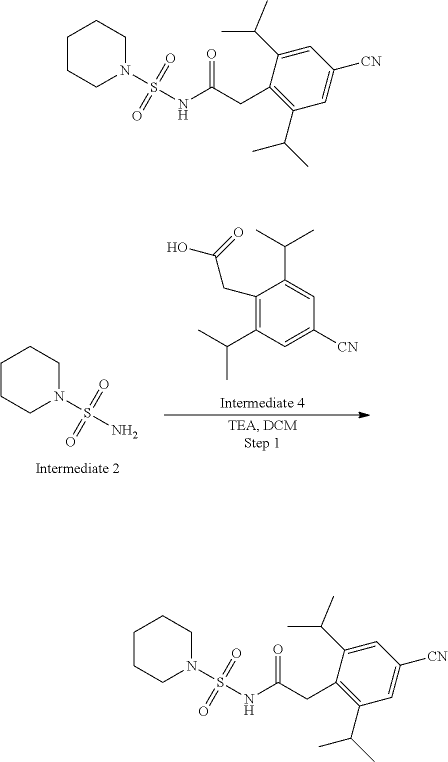

### Step 1: 2-(4-cyano-2,6-diisopropylphenyl)-N-(piperidin-1-ylsulfonyl)acetamide

Into a 50 mL round-bottom flask, was placed a solution of 2-(4-cyano-2,6-diisopropylphenyl)acetic acid (147 mg, 0.6 mmol, 2.0 equiv.) in DCM (5 mL). To the solution were added oxalyl dichloride (2 mL, 23.5 mmol, 29.3 equiv.) and DMF (0.05 mL, 0.6 mmol, 2.0 equiv.). The resulting solution was stirred for 1 h at ambient temperature and then concentrated under vacuum. The residue was dissolved in DCM (3 mL) and the resulting solution was added to a solution of piperidine-1-sulfonamide (50 mg, 0.3 mmol, 1.0 equiv.) and TEA (92 mg, 0.9 mmol, 3.0 equiv.) in DCM (5 mL) dropwise at 0° C. The resulting solution was stirred for 30 min at ambient temperature and then concentrated under vacuum. The crude product was purified by Prep-HPLC with the following conditions (Prep-HPLC-018): Column, XBridge Prep OBD C18 Column, 19*250 mm, 5 um; mobile phase, Water (10 mmol/L NH₄HCO₃) and ACN (22% Phase B up to 45% in 10 min); Detector, UV 220/254 nm. This resulted in 43.3 mg of 2-(4-cyano-2,6-diisopropylphenyl)-N-(piperidin-1-ylsulfonyl)acetamide as a white solid. MS-ESI: 390.2 (M−1). ¹H NMR: (400 MHz, Methanol-d₄) δ: 7.51 (s, 2H), 3.90 (s, 2H), 2.33-2.38 (m, 4H), 3.19-3.14 (m, 2H), 1.63-1.62 (m, 4H), 1.56-1.54 (m, 2H), 1.28-1.26 (m, 12H).

TABLE 2
Examples in the following table were prepared using similar conditions as described
in Example 1 from common intermediate 4.

| | | | | | Exact |
|---|---|---|---|---|---|
| Example | Compound | | | | Mass |
| # | Number | Sulfonamide | Structure | IUPAC Name | [M + H]⁺ |
| 2B | 202B | 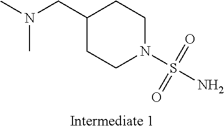 | 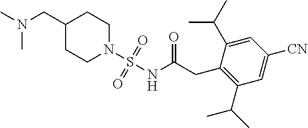 [SMILES: CC(C)c1cc(C#N)cc(C(C)C)c1CC(=O)NS(=O)(=O)N1CCC(CN(C)C)CC1] | 2-(4-cyano-2,6- diisopropylphenyl)- N-((4- ((dimethylamino) methyl)piperidin- 1-yl)sulfonyl) acetamide | 449.3 |
| 3B | 203B | 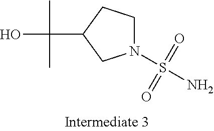 | 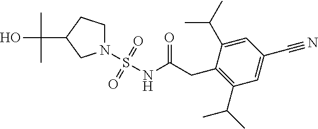 [SMILES: CC(C)c1cc(C#N)cc(C(C)C)c1CC(=O)NS(=O)(=O)N1CCC(C(C)(C)O)C1] | N′-((1,2,3,5,6,7- hexahydro-s- indacen-4- yl)carbamoy1)-1- (1- methylpyrrolidin- 3- yl)methane- sulfonimidamide | 434.2 ([M − H]−) |

### Example 4B (Compound 101B)

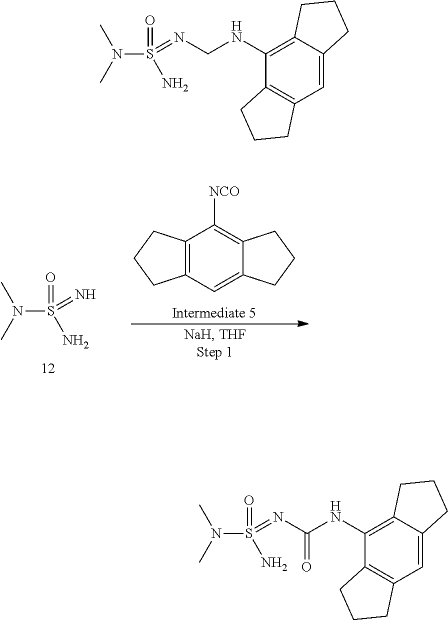

### Step 1: 3-[amino(dimethylamino)sulfinylidene]-1-(1,2,3,5,6,7-hexahydro-s-indacen-4-yl)urea

Into a 50 mL round-bottom flask purged and maintained with an inert atmosphere of nitrogen, was placed a solution of S-aminosulfonimidoyldimethylamine (Enamine, 300 mg, 2.4 mmol, 1.0 equiv.) in THF (20 mL). To the solution were added TEA (1.00 mL, 7.2 mmol, 3.0 equiv.) and 4-isocyanato-1,2,3,5,6,7-hexahydro-s-indacene (480 mg, 2.4 mmol, 1.0 equiv.). The resulting solution was stirred for overnight at ambient temperature and concentrated under vacuum. The crude product was purified by Prep-HPLC with the following conditions: Column, XSelect CSH Prep C18 OBD Column, 5 um, 19*150 mm; mobile phase, Water (10 mmol/L NH₄HCO₃+0.1% NH₄OH) and ACN (22% Phase B up to 52% in 7 min); Detector, 254/220 nm. This resulted in 60 mg of 3-[amino(dimethylamino)oxo-lambda6-sulfanylidene]-1-(1,2,3,5,6,7-hexahydro-s-indacen-4-yl)urea as a white solid. MS-ESI: 323.2 (M+1). ¹H NMR: (400 MHz, DMSO-d₆): 8.20 (br s, 1H), 7.16 (s, 2H), 6.88 (s, 1H), 2.81-2.78 (m, 4H), 2.72-2.68 (m, 4H), 2.70 (s, 6H), 1.97-1.93 (m, 4H).

Racemic compounds of this invention can be resolved to give individual enantiomers using a variety of known methods. For example, chiral stationary phases can used and the elution conditions can include normal phase or super-critical fluid with or without acidic or basic additives. Enantiomerically pure acids or bases can be used to form diatereomeric salts with the racemic compounds whereby pure enantiomers can be obtained by fractional crystallization. The racemates can also be derivatized with enantiomerically pure auxiliary reagents to form diastereomeric mixtures that can be separated. The auxiliary is then removed to give pure enantiomers.

In one embodiment, provided herein is a pharmaceutical composition comprising any NLRP3 antagonist species defined here (for example, a compound of any of the compound tables, for examples of tables 10 or 3B), and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.

In one embodiment, provided herein is a pharmaceutical combination of a compound of any NLRP3 antagonist species defined here (for example, a compound or example of any of the compound tables, for examples of tables 10 or 3B), and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

### The Following Protocol is Suitable for Testing the Activity of the Compounds Disclosed Herein.

### Procedure 1: IL-1β Production in PMA-Differentiated THP-1 Cells Stimulated with Gramicidin

THP-1 cells were purchased from the American Type Culture Collection and sub-cultured according to instructions from the supplier. Cells were cultured in complete RPMI 1640 (containing 10% heat inactivated FBS, penicillin (100 units/ml) and streptomycin (100 μg/ml)), and maintained in log phase prior to experimental setup. Prior to the experiment, compounds were dissolved in dimethyl sulfoxide (DMSO) to generate a 30 mM stock. The compound stock was first pre-diluted in DMSO to 3, 0.34, 0.042 and 0.0083 mM intermediate concentrations and subsequently spotted using Echo550 liquid handler into an empty 384-well assay plate to achieve desired final concentration (e.g. 100, 33, 11, 3.7, 1.2, 0.41, 0.14, 0.046, 0.015, 0.0051, 0.0017 μM). DMSO was backfilled in the plate to achieve a final DMSO assay concentration of 0.37%. The plate was then sealed and stored at room temperature until required.

THP-1 cells were treated with PMA (Phorbol 12-myristate 13-acetate) (20 ng/ml) for 16-18 hours. On the day of the experiment the media was removed and adherent cells were detached with trypsin for 5 minutes. Cells were then harvested, washed with complete RPMI 1640, spun down, and resuspended in RPMI 1640 (containing 2% heat inactivated FBS, penicillin (100 units/ml) and streptomycin (100 μg/ml). The cells were plated in the 384-well assay plate containing the spotted compounds at a density of 50,000 cells/well (final assay volume 50 μl). Cells were incubated with compounds for 1 hour and then stimulated with gramicidin (5 μM) (Enzo) for 2 hours. Plates were then centrifuged at 340 g for 5 min. Cell free supernatant (404) was collected using a 96-channel PlateMaster (Gilson) and the production of IL-1β was evaluated by HTRF (cisbio). The plates were incubated for 18 h at 4° C. and read using the preset HTRF program (donor emission at 620 nm, acceptor emission at 668 nm) of the SpectraMax i3x spectrophotometer (Molecular Devices, software SoftMax 6). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-113 production in PMA-differentiated THP-1 cells.

### Procedure 2: IL-1R Production in PMA-Differentiated THP-1 Cells Stimulated with Gramicidin

THP-1 cells were purchased from the American Type Culture Collection and sub-cultured according to instructions from the supplier. Prior to experiments, cells were cultured in complete RPMI 1640 (containing 10% heat inactivated FBS, penicillin (100 units/ml) and streptomycin (100 μg/ml)), and maintained in log phase prior to experimental setup. Prior to the experiment THP-1 were treated with PMA (Phorbol 12-myristate 13-acetate) (20 ng/ml) for 16-18 hours. Compounds were dissolved in dimethyl sulfoxide (DMSO) to generate a 30 mM stock. On the day of the experiment the media was removed and adherent cells were detached with trypsin for 5 minutes. Cells were then harvested, washed with complete RPMI 1640, spun down, resuspended in RPMI 1640 (containing 2% heat inactivated FBS, penicillin (100 units/ml) and streptomycin (100 μg/ml). The cells were plated in a 384-well plate at a density of 50,000 cells/well (final assay volume 50 μl). Compounds were first dissolved in assay medium to obtain a 5× top concentration of 500 μM. 10 step dilutions (1:3) were then undertaken in assay medium containing 1.67% DMSO. 5× compound solutions were added to the culture medium to achieve desired final concentration (e.g. 100, 33, 11, 3.7, 1.2, 0.41, 0.14, 0.046, 0.015, 0.0051, 0.0017 μM). Final DMSO concentration was at 0.37%. Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

### Procedure 3

### 1. Experimental Procedure

1.1 Cell Culture
1) Culture THP-1 cells in the complete RPMI-1640 medium with 10% FBS at 37° C., 5% CO₂.
2) Passage the cells every 3 days by inoculating 3×10⁵ cells per ml.

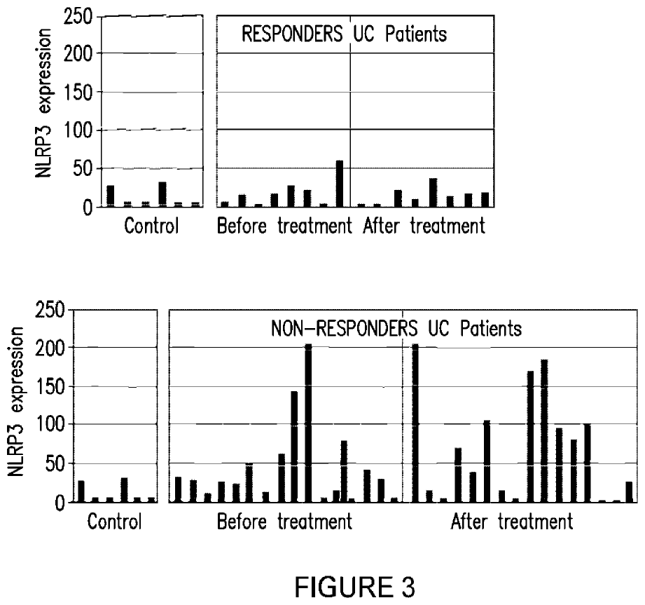

1.2 Compound Preparation
Prepare the 3-fold serial dilution of the compounds with DMSO in a 384-well LDV Microplate using TECAN EVO system to generate the compound source plate with 10 concentrations. Top concentration is 30 mM. FIG. 3 depicts the layout of the microplate.

1.3 Cell Preparation
1) Centrifuge THP-1 cells at 350 g for 5 min.
2) Re-suspend cells with complete RMPI-1640 medium, and count cells.
3) Seed cells in T225 flask, about 2.5×10⁷ per flask, treat cells with 20 ng/ml PMA (final DMSO concentration <1%).
4) Incubate overnight.

1.4 THP-1 Stimulation
1) Wash adherent THP-1 cells with PBS, and detach cells with 4 ml trypsin for T225 flask.
2) Centrifuge cells at 350 g for 5 min, re-suspend cells with RPMI-1640 containing 2% FBS and count cells with trypan blue.
3) Transfer 50 nl/well the serial dilution of test compound to 384-well plate by Echo; For the high control and first point of CRID3 (MCC950), transfer 165 nl, then backfill to make the DMSO concentration is consistent in all wells, the plate layout is as below.
4) Seed 50 k cells in 40ul RPMI-1640 with 2% FBS per well in 384-well plate.
5) Incubate for 1 h at 37° C., 5% CO₂.
6) Prepare 5× gramicidin, add 10 μl per well, the final concentration is 5 μM, incubate for 2 hrs at 37° C., 5% CO₂.
7) Centrifuge at 350 g for 1 min.
8) Pipet 16 μl supernatant by apricot, and transfer into white 384 proxiplate. FIG. 3 depicts the layout of the plates: HC: 100 μM CRID3 (MCC950)+5 μM gramicidin LC: 5 μM Gramicidin.

1.5 IL-1β Detection
1) Homogenize the 5× diluent #5 with a vortex and add 1 volume of stock solution in 4 volumes of distilled water.
2) Thaw 20× stock solution of anti-IL1β-Cryptate-antibody and anti-IL1β XL-antibody. Dilute these two antibodies to 1× with detection buffer #3.
3) Pre-mix the two ready-to-use antibody solutions just prior to use.
4) Dispense 4 μl of pre-mixed Anti-IL1β antibodies working solution into all wells.
5) Seal the plate and incubate overnight at 4° C.
6) Read the cell plate using EnVison and plot Readout vs. the test compound concentration to calculate the IC₅₀.

### 2. Data Analysis

1. IC₅₀ of compounds can be calculated using the following formulas Formula for IC₅₀

% inhibition=100−100×[HCₐᵥₑ−Readout/(HCₐᵥₑ−LCₐᵥₑ)]
2. Fit the normalized data in a dose-response manner using XLfit, and calculate the compound concentration.
   Tables 10 and 3B shows the biological activity of compounds in hTHP-1 assay containing 2% fetal bovine serum: <0.008 μM=“++++++”; >0.008 and <0.04 μM=“+++++”; >0.04 and <0.2 μM=“++++”; >0.2 and <1 μM=“+++”; >1 and <5 μM=“++”; >5 and <30 μM=

TABLE 10
Average IC₅₀ of compounds in hTHP-1 assay

| Example | | |
|---|---|---|
| # | Compound | hTHP-1 IC₅₀ |
| | 101 | >30.0000 |
| | 102 | >30.0000 |
| | 103 | >30.0000 |
| | 104 | ++ |
| | 105 | >30.0000 |
| | 106 | ++ |
| | 107 | >30.0000 |
| | 108 | >30.0000 |
| | 109 | + |
| | 110a | + |
| | 111 | + |
| | 112 | ++ |
| | 113 | >30.0000 |
| | 114 | + |
| | 115 | + |
| | 116 | >30.0000 |
| | 117 | + |
| | 118 | >30.0000 |
| | 119 | + |
| | 120 | |
| | 121 | + |
| | 122 | ++ |
| | 123 | + |
| | 124 | >30.0000 |
| | 125 | ++ |
| | 126 | >30.0000 |
| | 127 | >30.0000 |
| | 129 | + |
| | 130 | >30.0000 |
| | 131 | >30.0000 |
| | 132 | >30.0000 |
| | 133 | >30.0000 |
| | 134 | >30.0000 |
| | 135 | >30.0000 |
| | 137 | >30.0000 |
| | 138 | >30.0000 |
| | 139 | + |
| | 140 | >30.0000 |
| | 141 | + |
| | 142 | ++ |
| | 143 | + |
| | 144 | + |
| | 145 | >30.0000 |
| | 146 | >30.0000 |
| | 147 | >30.0000 |
| | 148 | >30.0000 |
| | 149 | >30.0000 |
| | 150 | >30.0000 |
| | 151 | >30.0000 |
| | 152 | >30.0000 |
| | 153 | + |
| | 154 | + |
| | 155 | + |
| | 156 | >30.0000 |
| | 157 | |
| | 158 | >30.0000 |
| | 159 | >30.0000 |
| | 160 | >30.0000 |
| | 161 | >30.0000 |
| | 162 | +++ |
| | 163 | >30.0000 |
| | 164 | + |
| | 165 | +++ |
| | 165a | ++ |
| | 165b | ++++ |
| | 301 | >30.0000 |
| | 302 | >30.0000 |
| | 303 | >30.0000 |
| | 304 | >30.0000 |
| | 305 | >30.0000 |
| | 306 | >30.0000 |
| | 307 | +++++ |
| | 308 | >30.0000 |
| | 309 | >30.0000 |
| | 401 | 31.494 |
| | 402 | + |
| | 403 | + |
| | 501 | >100.0000 |
| | 502 | >100.0000 |
| | 503 | >100.0000 |
| | 504 | ++ |
| | 505 | >97.1235 |
| | 506 | + |
| | 507 | >100.0000 |
| | 508 | >100.0000 |
| | 509 | >30.0000 |
| | 510 | >100.0000 |
| | 511 | >100.0000 |
| | 512 | >100.0000 |
| | 513 | >30.0000 |
| | 514 | >100.0000 |
| | 515 | >100.0000 |
| | 516 | >30.0000 |
| | 517 | >100.0000 |
| | 518 | >100.0000 |
| | 519 | >30.0000 |
| | 520 | ++ |
| | 528 | >30.0000 |
| | 522 | >100.0000 |

TABLE 3B
Average IC₅₀ of compounds in hTHP-1 assay

| Example # | Compound | hTHP-1 IC₅₀ |
|---|---|---|
| 1B | 201B | + |
| 2B | 202B | + |
| 3B | 203B | + |
| 4B | 101B | + |

A number of embodiments of the invention have been described. Nevertheless, it will be understood that various modifications may be made without departing from the spirit and scope of the invention. Accordingly, other embodiments are within the scope of the following claims.

### Study Example 1

The CARD8 gene is located within the inflammatory bowel disease (IBD) 6 linkage region on chromosome 19. CARD8 interacts with NLRP3, and Apoptosis-associated Speck-like protein to form a caspase-1 activating complex termed the NLRP3 inflammasome. The NLRP3 inflammasome mediates the production and secretion of interleukin-1β, by processing pro-IL-1P into mature secreted IL-1β. In addition to its role in the inflammasome, CARD8 is also a potent inhibitor of nuclear factor NF-κB. NF-κB activation is essential for the production of pro-IL-14. Since over-production of IL-1β and dysregulation of NF-κB are hallmarks of Crohn's disease, CARD8 is herein considered to be a risk gene for inflammatory bowel disease. A significant association of CARD8 with Crohn's disease was detected in two British studies with a risk effect for the minor allele of the non-synonymous single-nucleotide polymorphism (SNP) of a C allele at rs2043211. This SNP introduces a premature stop codon, resulting in the expression of a severely truncated protein. This variant CARD8 protein is unable to suppress NF-κB activity, leading to constitutive production of pro-IL-1β, which is a substrate for the NLRP3 inflammasome. It is believed that a gain-of-function mutation in an NLRP3 gene (e.g., any of the gain-of-function mutations described herein, e.g., any of the gain-of-function mutations in an NLRP3 gene described herein) combined with a loss-of-function mutation in a CARD8 gene (e.g., a C allele at rs2043211) results in the development of diseases related to increased NLRP3 inflammasome expression and/or activity. Patients having, e.g., a gain-of-function mutation in an NLRP3 gene and/or a loss-of-function mutation in a CARD8 gene are predicted to show improved therapeutic response to treatment with an NLRP3 antagonist.

A study is designed to determine: whether NLRP3 antagonists inhibit inflammasome function and inflammatory activity in cells and biopsy specimens from patients with Crohn's disease or ulcerative colitis; and whether the specific genetic variants identify patients with Crohn's disease or ulcerative colitis who are most likely to respond to treatment with an NLRP3 antagonist.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if baseline (no ex vivo treatment) RNA levels of NLRP3, ASC, and IL-1β are greater in biopsy samples from patients with anti-TNFα agent resistance status; and stratify the results according to presence of specific genetic mutations in genes encoding ATG16L1, NLRP3, and CARD8 (e.g., any of the mutations in the ATG16L1 gene, NLRP3 gene, and CARD8 gene described herein).

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.
Sequence patient DNA to detect specific genetic mutations in the ATG16L1 gene, NLRP3 gene, and CARD8 gene (e.g., any of the exemplary mutations in these genes described herein) and then stratify the results of functional assays according to the presence of these genetic mutations.

### Experimental Design

Human subjects and tissue:
Endoscopic or surgical biopsies from areas of disease in patients with Crohn's disease and ulcerative colitis who are either anti-TNFα treatment nave or resistant to anti-TNFα treatment; additionally biopsies from control patients (surveillance colonoscopy or inflammation-free areas from patients with colorectal cancer) are studied.

Ex vivo Treatment Model:
Organ or LPMC culture as determined appropriate

Endpoints to be measured:
Before ex vivo treatment—NLRP3 RNA level
After ex vivo treatment—inflammasome activity (either processed IL-1β, processed caspase-1, or secreted IL-1β); RNA for inflammatory cytokines (Nanostring); viable T cell number and/or T cell apoptosis.

Data Analysis Plan:
Determine if NLRP3 antagonist treatment decreases processed IL-1β, processed caspase-1 or secreted IL-1β, and inflammatory cytokine RNA levels.
Stratify response data according to treatment status at biopsy and the presence of genetic mutations in the NLRP3 gene, CARD8 gene, and ATG16L1 gene (e.g., any of the exemplary genetic mutations of these genes described herein).

### Study Example 2. Treatment of Anti-TNFα Resistant Patients with NLRP3 Antagonists

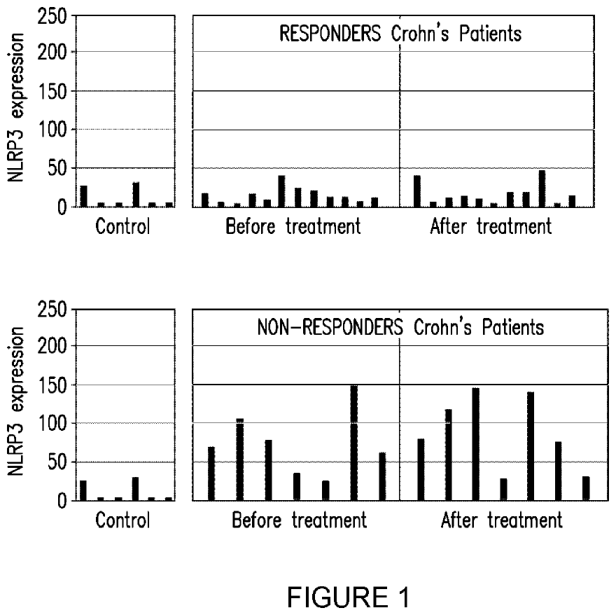

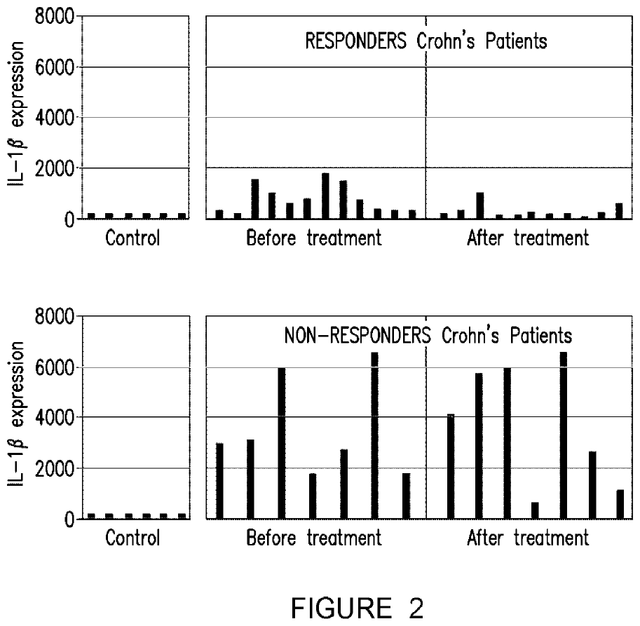

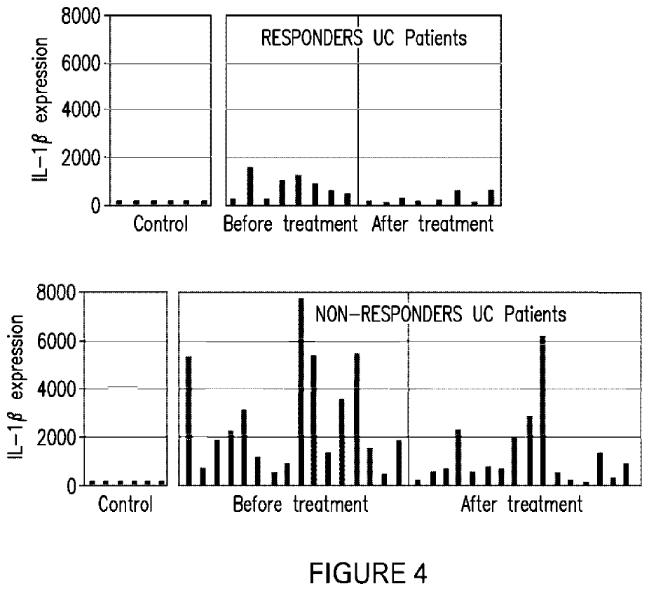

PLoS One 2009 Nov. 24; 4(11):e7984, describes that mucosal biopsies were obtained at endoscopy in actively inflamed mucosa from patients with Ulcerative Colitis, refractory to corticosteroids and/or immunosuppression, before and 4-6 weeks after their first infliximab (an anti-TNFα agent) infusion and in normal mucosa from control patients. The patients in this study were classified for response to infliximab based on endoscopic and histologic findings at 4-6 weeks after first infliximab treatment as responder or non-responder. Transcriptomic RNA expression levels of these biopsies were accessed by the inventors of the invention disclosed herein from GSE 16879, the publically available Gene Expression Omnibus (https://www.ncbi.nlm.nih.gov/geo2r/%acc=GSE16879). Expression levels of RNA encoding NLRP3 and IL-1β were determined using GEO2R (a tool available on the same website), based on probe sets 207075_at and 205067_at, respectively. It was surprisingly found that in Crohn's disease patients that are non-responsive to the infliximab (an anti-TNFα agent) have higher expression of NLRP3 and IL-1β RNA than responsive patients (FIGS. 1 and 2). Similar surprising results of higher expression of NLRP3 and IL-1p RNA in UC patients that are non-responsive to infliximab (an anti-TNFα agent) compared to infliximab (an anti-TNFα agent) responsive patients (FIGS. 3 and 4) were found.

Said higher levels of NLRP3 and IL-1β RNA expression levels in anti-TNFα agent non-responders, is hypothesised herein to lead to NLRP3 activation which in turns leads of release of IL-1β that induces IL-23 production, leading to said resistance to anti-TNFα agents. Therefore, treatment of Crohn's and UC anti-TNFα non-responders with an NLRP3 antagonist would prevent this cascade, and thus prevent development of non-responsiveness to anti-TNFα agents. Indeed, resistance to anti-TNFα agents is common in other inflammatory or autoimmune diseases. Therefore, use of an NLRP3 antagonist for the treatment of inflammatory or autoimmune diseases will block the mechanism leading to non-responsiveness to anti-TNFα agents. Consequently, use of NLRP3 antagonists will increase the sensitivity of patients with inflammatory or autoimmune diseases to anti-TNFα agents, resulting in a reduced dose of anti-TNFα agents for the treatment of these diseases. Therefore, a combination of an NLRP3 antagonist and an anti-TNFα agent can be used in the treatment of diseases wherein TNFα is overexpressed, such as inflammatory or autoimmune diseases, to avoid such non-responsive development of patients to anti-TNFα agents. Preferably, this combination treatment can be used in the treatment of IBD, for example Crohn's disease and UC.

Further, use of NLRP3 antagonists offers an alternative to anti-TNFα agents for the treatment of diseases wherein TNFα is overexpressed. Therefore, NLRP3 antagonists offers an alternative to anti-TNFα agents inflammatory or autoimmune diseases, such as IBD (e.g. Crohn's disease and UC).

Systemtic anti-TNFα agents are also known to increase the risk of infection. Gut restricted NLRP3 antagonists, however, offers a gut targeted treatment (i.e. non-systemic treatment), preventing such infections. Therefore, treatment of TNFα gut diseases, such as IBD (i.e. Crohn's disease and UC), with gut restricted NLRP3 antagonists has the additional advantage of reducing the risk of infection compared to anti-TNFα agents.

### Proposed Experiment

Determine the expression of NLRP3 and caspase-1 in LPMCs and epithelial cells in patients with non-active disease, in patients with active disease, in patients with active disease resistant to corticosteroids, patients with active disease resistant to TNF-blocking agents. The expression of NLRP3 and caspase-1 in LPMCs and epithelial cells will be analyzed by RNAScope technology. The expression of active NLRP3 signature genes will be analyzed by Nanostring technology. A pilot analysis to determine feasibility will be performed with 5 samples from control, 5 samples from active CD lesions, and 5 samples from active UC lesions.

### Study Example 3

It is presented that NLRP3 antagonists reverse resistance to anti-TNF induced T cell depletion/apoptosis in biopsy samples from IBD patients whose disease is clinically considered resistant or unresponsive to anti-TNF therapy.

A study is designed to determine: whether NLRP3 antagonists inhibit inflammasome function and inflammatory activity in cells and biopsy specimens from patients with Crohn's disease or ulcerative colitis; and whether an NLRP3 antagonist will synergize with anti-TNFα therapy in patients with Crohn's disease or ulcerative colitis.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if an NLRP3 antagonist in the absence of co-treatment with anti-TNFα antibody induces T cell depletion in Crohn's disease and ulcerative colitis biopsy samples; and determine if baseline (no ex vivo treatment) RNA levels of NLRP3, ASC, and IL-1β are greater in biopsy samples from patients with anti-TNFα agent resistance status.

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if there is synergy between an NLRP3 antagonist and anti-TNF antibody with respect to effects on T cell depletion/apoptosis in biopsies of disease from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.

### Experimental Design

Human subjects and tissue:
Endoscopic or surgical biopsies from areas of disease in patients with Crohn's disease and ulcerative colitis who are either anti-TNFα treatment nave or resistant to anti-TNFα treatment; additionally biopsies from control patients (surveillance colonoscopy or inflammation-free areas from patients with colorectal cancer) are studied.

Ex vivo Treatment Model:
Organ or LPMC culture as determined appropriate

Ex vivo Treatments:
NLRP3 antagonist (2 concentrations), negative control (vehicle), positive control (caspase-1 inhibitor) each in the presence or absence of anti-TNF antibody at a concentration appropriate to distinguish differences in the T cell apoptotic between biopsies from anti-TNF resistant and anti-TNF-sensitive Crohn's disease patients. Each treatment condition is evaluated in a minimum in duplicate samples.

Endpoints to be measured:
Before ex vivo treatment—NLRP3 RNA level
After ex vivo treatment—inflammasome activity (either processed IL-1β, processed caspase-1, or secreted IL-1β); RNA for inflammatory cytokines (Nanostring); viable T cell number and/or T cell apoptosis.

Data Analysis Plan:
Determine if NLRP3 antagonist co-treatment increases T cell apoptosis/deletion in response to anti-TNF.
Determine if the level of NLRP3 RNA expression is greater in TNF-resistant Crohn's disease and ulcerative colitis samples compared to anti-TNF treatment-nave samples.
Determine if NLRP3 antagonist treatment decreases processed IL-1β, processed caspase-1 or secreted IL-1β, and inflammatory cytokine RNA levels.

### Biological Assay—Nigericin-Stimulated IL-1β Secretion Assay in THP-1 Cells

Monocytic THP-1 cells (ATCC: TIB-202) were maintained according to providers' instructions in RPMI media (RPMI/Hepes+10% fetal bovine serum+Sodium Pyruvate+0.05 mM Beta-mercaptoethanol (1000× stock)+Pen-Strep). Cells were differentiated in bulk with 0.5 μM phorbol 12-myristate 13-acetate (PMA; Sigma #P8139) for 3 hours, media was exchanged, and cells were plated at 50,000 cells per well in a 384-well flat-bottom cell culture plates (Greiner, #781986), and allowed to differentiate overnight. Compound in a 1:3.16 serial dilution series in DMSO was added 1:100 to the cells and incubated for 1 hour. The NLRP3 inflammasome was activated with the addition of 15 μM (final concentration) Nigericin (Enzo Life Sciences, #BML-CA421-0005), and cells were incubated for 3 hours. 10 μL supernatant was removed, and IL-1β levels were monitored using an HTRF assay (CisBio, #62IL1PEC) according to manufacturers' instructions. Viability and pyroptosis was monitored with the addition of PrestoBlue cell viability reagent (Life Technologies, #A13261) directly to the cell culture plate.

Further, enumerated, embodiments of the invention are defined below, these embodiments may be combined (as practical) with any features of other embodiments disclosed herein.

Embodiment 1 refers to either embodiment 1A, or embodiment 1B, as defined below
1A. A compound of Formula AA

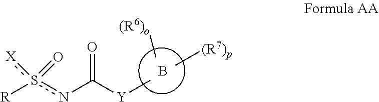

wherein

### R is Z-Q;

### Q is:

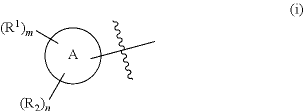

wherein ring A is selected from the group consisting of 5- to 10-membered heteroaryl, C₆-C₁₀ aryl, C₃-C₁₀ cycloalkyl, and 3-10-membered heterocycloalkyl; or

### (ii) H

### Z is:

(i) C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
(ii) 3-10-membered heterocycloalkylene optionally substituted by one or more R¹ and/or R²; or
(iii) C₃-C₁₀ cycloalkyl optionally substituted by one or more R¹ and/or R²; [symbol]
represents a single or double bond;
wherein one of the following apply:
(iii) When X is NHR³, a single bond is present between X and S, a double bond is present between S and N, and Y is selected from NH and CR⁴R⁵; or
(iv) When X is 0, a double bond is present between X and S, a single bond is present between S and N, the N that is bonded to S is further substituted with an H, and Y is CR⁴R⁵;
   B is selected from the group consisting of 5-membered heteroaryl, 7-10 membered heteroaryl, and C₆-C₁₀ aryl;
   m=0, 1, or 2;
   n=0, 1, or 2;
   R¹ and R² are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
   wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R¹⁶ and R¹⁷ are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹²,
   wherein the C₁-C₆ alkyl and C₁-C₆ haloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   R³ is selected from hydrogen, hydroxy, C₁-C₆ alkoxy, C₁-C₆ alkyl, and

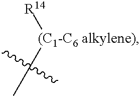

wherein the C₁-C₆ alkylene group is optionally substituted by oxo;
each of R⁴ and R⁵ is independently selected from hydrogen and C₁-C₆ alkyl;
o=1 or 2;
p=0, 1, 2, or 3;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl, wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
   each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹⁰ is C₁-C₆ alkyl;
   each of R¹¹ and R¹² at each occurrence is independently selected from hydrogen and C₁-C₆ alkyl;
   R¹³ is C₁-C₆ alkyl, C₆-C₁₀ aryl, or 5- to 10-membered heteroaryl;
   R¹⁴ is hydrogen, hydroxy, C₁-C₆ alkyl, NR⁸R⁹, 5- to 10-membered monocyclic or bicyclic heteroaryl, or C₆-C₁₀ monocyclic or bicyclic aryl, wherein each C₁-C₆ alkyl, aryl or heteroaryl is optionally independently substituted with 1 or 2 R⁶;
   each R¹⁵ at each occurrence are each independently selected from the group consisting of C₁-C₆ alkyl, C₁-C₆ haloalkyl, C₁-C₆ alkoxy, C₁-C₆ haloalkoxy, CN, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
   wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹⁵ C₃-C₇ cycloalkyl or of the R¹⁵ 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   with the proviso that the compound of Formula AA is not the following structure:

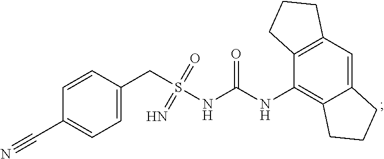 [SMILES: N#Cc1ccc(CS(=N)(=O)NC(=O)Nc2c3c(cc4c2CCC4)CCC3)cc1]

or a pharmaceutically acceptable salt thereof
1B. A compound of Formula AB

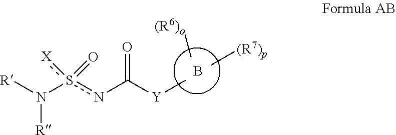

wherein
R′ and R″ are each independently selected from:

### (i) H;

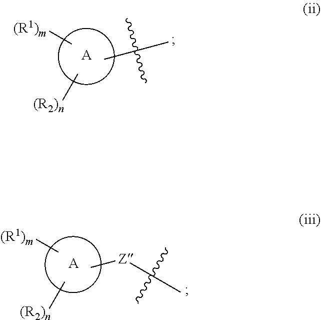

and

### (iv) Z″—H; or

alternatively, R′ and R″ are taken together with the N to which they are attached to form a 5-10-membered heterocycloalkyl ring optionally substituted with one or more R¹ and/or R²; wherein ring A is selected from the group consisting of 5- to 10-membered heteroaryl, C₆-C₁₀ aryl, C₃-C₁₀ cycloalkyl, and 3-10-membered heterocycloalkyl;
Z″ is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O); [symbol]
represents a single or double bond;
wherein one of the following apply:
(i) When X is NHR³, a single bond is present between X and S, a double bond is present between S and N, and Y is selected from NH and CR⁴R⁵; or
(ii) When X is 0, a double bond is present between X and S, a single bond is present between S and N, the N that is bonded to S is further substituted with an H, and Y is CR⁴R⁵;
   B is selected from the group consisting of 5-membered heteroaryl, 7-10 membered heteroaryl, and C₆-C₁₀ aryl;
   m=0, 1, or 2;
   n=0, 1, or 2;
   R¹ and R² are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
   wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo; wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
   R¹⁶ and R¹⁷ are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹²,
   wherein the C₁-C₆ alkyl and C₁-C₆ haloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
   R³ is selected from hydrogen, hydroxy, C₁-C₆ alkoxy, C₁-C₆ alkyl, and

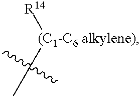

wherein the C₁-C₆ alkylene group is optionally substituted by oxo;
each of R⁴ and R⁵ is independently selected from hydrogen and C₁-C₆ alkyl;
o=1 or 2;
p=0, 1, 2, or 3;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
   R¹⁰ is C₁-C₆ alkyl;
   each of R¹¹ and R¹² at each occurrence is independently selected from hydrogen and C₁-C₆ alkyl;
   R¹³ is C₁-C₆ alkyl, C₆-C₁₀ aryl, or 5- to 10-membered heteroaryl;
   R¹⁴ is hydrogen, hydroxy, C₁-C₆ alkyl, NR⁸R⁹, 5- to 10-membered monocyclic or bicyclic heteroaryl, or C₆-C₁₀ monocyclic or bicyclic aryl, wherein each C₁-C₆ alkyl, aryl or heteroaryl is optionally independently substituted with 1 or 2 R⁶;
   each R¹⁵ at each occurrence are each independently selected from the group consisting of C₁-C₆ alkyl, C₁-C₆ haloalkyl, C₁-C₆ alkoxy, C₁-C₆ haloalkoxy, CN, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
   wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹⁵ C₃-C₇ cycloalkyl or of the R¹⁵ 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
   or a pharmaceutically acceptable salt thereof
2. The compound of embodiment 1, wherein X is O, a double bond is present between X and S, a single bond is present between S and N, the N that is bonded to S is further substituted with an H, and Y is CR⁴R⁵.
3. The compound of embodiment 1, wherein X is NHR³, a single bond is present between X and S, and a double bond is present between S and N.
4. The compound of embodiment 3, wherein Y is NH.
5. The compound of embodiment 3, wherein Y is CR⁴R⁵.
6. The compound of any one of embodiments 1-5, wherein Z is (i) C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O).
7. The compound of embodiment 6, wherein Z is C₁₋₆alkylene having from 1-6 carbon atoms independently selected from the group consisting of CH₂, CH, C, CR¹⁶, CR¹⁷, CHR¹⁶, CHR¹⁷, CR¹⁶C¹⁶, CR¹⁷R¹⁷, CR¹⁶R¹⁷, and C(O).
8. The compound of embodiment 6, wherein Z is C₁₋₂alkylene having from 1-2 carbon atoms independently selected from the group consisting of CH₂, CHR¹⁶, CHR¹⁷, CR¹⁶R¹⁶, CR¹⁷R¹⁷, CR¹⁶R¹⁷, and C(O).
9. The compound of embodiment 6, wherein Z is C₁ alkylene having 1 carbon atom selected from the group consisting of CH₂, CHR¹⁶, CHR¹⁷, CR¹⁶R¹⁶, CR¹⁷R¹⁷, CR¹⁶R¹⁷, and C(O).
10. The compound of any one of embodiments 6-9, wherein the alkylene is CH₂.
11. The compound of any one of embodiments 6-9, wherein the alkylene comprises C(O).
12. The compound of any one of embodiments 6-9, wherein the alkylene is C(O).
13. The compound of any one of embodiments 6-7, wherein the alkylene is 1-methyl-1-propyl.
14. The compound of any one of embodiments 6-7, wherein the alkylene is 2-methyl-1-propyl.
15. The compound of any one of embodiments 6-7, wherein the alkylene is 2,2-dimethyl-1-propyl.
16. The compound of any one of embodiments 6-8, wherein the alkylene is ethyl.
17. The compound of any one of embodiments 6-7, wherein the alkylene is n-propyl.
18. The compound of any one of embodiments 6-7, wherein the alkylene is n-butyl.
19. The compound of any one of embodiments 6-7, wherein the alkylene is branched.
20. The compound of any one of embodiments 6-7, wherein the alkylene is linear.
21. The compound of any one of embodiments 1-5, wherein Z is (ii) 3-10-membered heterocycloalkylene optionally substituted by one or more R¹ and/or R².
22. The compound of any one of embodiments 1-5 and 21, wherein Z is a 5-6-membered heterocycloalkylene optionally substituted by one or more R¹ and/or R².
23. The compound of any one of embodiments 1-5 and 21, wherein Z is a 5-membered heterocycloalkylene optionally substituted by one or more R¹ and/or R².
24. The compound of any one of embodiments 1-5 and 21, wherein Z is a 6-membered heterocycloalkylene optionally substituted by one or more R¹ and/or R².
25. The compound of any one of embodiments 1-5 and 21-23, wherein Z is pyrrolidinylene (e.g., 3-pyrrolidinylene) optionally substituted by one or more R¹ and/or R².
26. The compound of any one of embodiments 1-5, 21-22, and 24, wherein Z is piperidinylene (e.g., 4-piperidinylene) optionally substituted by one or more R¹ and/or R².
27. The compound of any one of embodiments 1-5, wherein Z is (iii) C₃-C₁₀ cycloalkyl optionally substituted by one or more R¹ and/or R².
28. The compound of any one of embodiments 1-5 and 27, wherein Z is cyclohexyl optionally substituted by one or more R¹ and/or R²
29. The compound of any one of embodiments 1-5 and 27, wherein Z is cyclopentyl optionally substituted by one or more R¹ and/or R².
30. The compound of any one of embodiments 1-5 and 27, wherein Z is cyclobutyl optionally substituted by one or more R¹ and/or R².
31. The compound of any one of embodiments 1-5 and 27, wherein Z is cyclopropyl optionally substituted by one or more R¹ and/or R².
32. The compound of any one of embodiments 1-31, wherein Q is

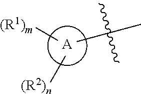

wherein ring A is selected from the group consisting of 5- to 10-membered heteroaryl, C₆-C₁₀ aryl, C₃-C₁₀ cycloalkyl, and 3-10-membered heterocycloalkyl.
33. The compound of any one of embodiments 1-31, wherein Q is H.
34. The compound of any one of embodiments 1-32, wherein ring A is selected from the group consisting of 5- to 10-membered heteroaryl, C₆-C₁₀ aryl, and 3-10-membered heterocycloalkyl.
35. The compound of any one of embodiments 1-32 and 34, wherein ring A is 5- to 10-membered heteroaryl.
36. The compound of any one of embodiments 1-32 and 34, wherein ring A is C₆-C₁₀ aryl.
37. The compound of any one of embodiments 1-32 and 34, wherein ring A is 3-10-membered heterocycloalkyl.
38. The compound of any one of embodiments 1-32 and 34-35, wherein ring A is thiophenyl (e.g., 3-thiophenyl).
39. The compound of any one of embodiments 1-32 and 34-35, wherein ring A is thiazolyl (e.g., 5-thiazolyl).
40. The compound of any one of embodiments 1-32 and 34-35, wherein ring A is pyrazolyl (e.g., 4-pyrazolyl).
41. The compound of any one of embodiments 1-32 and 34-35, wherein ring A is isoxazolyl (e.g., 5-isoxazolyl).
42. The compound of any one of embodiments 1-32, 34, and 36, wherein ring A is isoxazolyl (e.g., 5-isoxazolyl).
43. The compound of any one of embodiments 1-32, 34, and 37, wherein ring A is pyrrolidinyl (e.g., 2-pyrrolidinyl or 3-pyrrolidinyl).
44. The compound of any one of embodiments 1-32, 34, and 37, wherein ring A is piperidinyl (e.g., 3-piperidinyl or 4-piperidinyl).
45. The compound of any one of embodiments 1-32, 34, and 37, wherein ring A is azetidinyl (e.g., 2-azetidinyl).
46. The compound of any one of embodiments 1-32, 34, and 37, wherein ring A is morpholinyl (e.g., 2-morpholinyl).
47. The compound of any one of embodiments 1-32, 34, and 37, wherein ring A is pyrrolidinyl (e.g., 2-pyrrolidinyl or 3-pyrrolidinyl).
48. The compound of any one of embodiments 1-20 and 32-47, wherein R¹⁶ and R¹⁷ are each independently selected from C₁-C₆ alkyl, C₁-C₆ alkoxy, C₁-C₆ haloalkoxy, halo, CN, CO₂H, COC₁-C₆ alkyl, CO—C₆-C₁₀ aryl; CO(5- to 10-membered heteroaryl); CO₂C₁-C₆ alkyl, OCOC₁-C₆ alkyl, NH₂, NHC₁-C₆ alkyl, N(C₁-C₆ alkyl)₂, NHCOC₁-C₆ alkyl, NHCOC₆-C₁₀ aryl, NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹², wherein the C₁-C₆ alkyl is optionally substituted with one or more substituents each independently selected from hydroxy, halo, CN, oxo, C₁-C₆ alkoxy, NR⁸R⁹, ═NR¹⁰, COOC₁-C₆ alkyl, CONR⁸R⁹, OCOC₁-C₆ alkyl, NHCOC₁-C₆ alkyl, NHCOC₆-C₁₀ aryl, and NHCOC₂-C₆ alkynyl.
49. The compound of any one of embodiments 1-20, 32-47, and 48, wherein R¹⁶ and R¹⁷ are each independently selected from C₁-C₆ alkyl, C₁-C₆ alkoxy, halo, CN, CO₂H, COC₁-C₆ alkyl, CO₂C₁-C₆ alkyl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, and S(O₂)NR¹¹R¹², wherein the C₁-C₆ alkyl is optionally substituted with one or more substituents each independently selected from hydroxy, halo, CN, oxo, C₁-C₆ alkoxy, NR⁸R⁹, CONR⁸R⁹, and NHCOC₁-C₆ alkyl.
50. The compound of any one of embodiments 1-49, wherein R¹ and R² are each independently selected from C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents each independently selected from hydroxy, halo, oxo, C₁-C₆ alkoxy, and NR⁸R⁹.
51. The compound of any one of embodiments 1-50, wherein R¹ and R² are each independently selected from methyl, ethyl, isopropyl, 2-hydroxy-2-propyl, dimethylamino, aminomethyl, methylaminomethyl, dimethylaminomethyl, methoxycarbonyl, and carboxyl.
52. The compound of embodiment 1, wherein X is NHR³, a single bond is present between X and S, and a double bond is present between S and N; and the compound of Formula AA is a compound of Formula AA-1, Formula AA-2, or Formula AA-3:

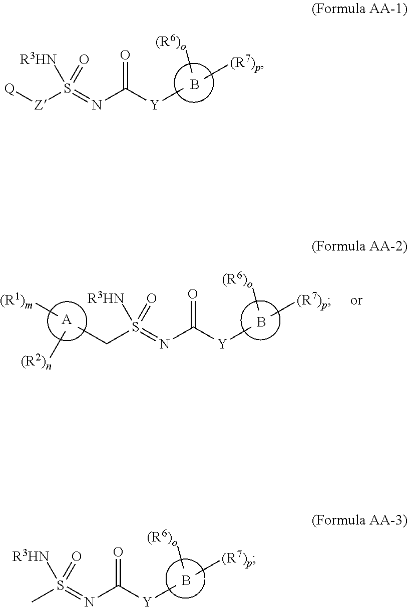

wherein

### Z′ is:

(i) C₂-C₈ alkylene having from 2-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
(ii) CHR¹⁶, CHR¹⁷, CR¹⁶R¹⁶, CR¹⁷R¹⁷, CR¹⁶R¹⁷, or C(O);
(ii) 3-10-membered heterocycloalkylene optionally substituted by one or more R¹ and/or R²; or
(iii) C₃-C₁₀ cycloalkyl optionally substituted by one or more R¹ and/or R²; and
wherein when

### (i) Formula AA is Formula AA-2,

(ii) ring A is phenyl,
(iii) the sum of m and n is 1, and
(iv) whichever of R¹ and R² that is present is CN;
then the position of the phenyl group that is para to the point of the phenyl group's connection to the sulfur of the S(O)(NHR³)═N moiety is substituted with hydrogen.
53. The compound of embodiment 52, wherein the compound is a compound of Formula AA-1:

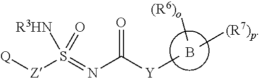

54. The compound of embodiment 52, wherein the compound is a compound of Formula AA-2:

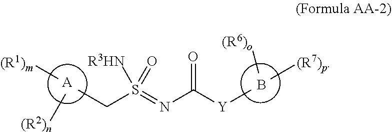

55. The compound of embodiment 52, wherein the compound is a compound of Formula AA-3:

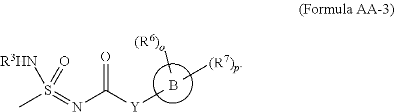

56. The compound of any one of embodiments 52-55, wherein Y is NH.
57. The compound of any one of embodiments 52-55, wherein Y is CR⁴R⁵.
58. The compound of any one of embodiments 52-53, wherein Z′ is (i) C₂-C₆ alkylene having from 2-6 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O).
59. The compound of any one of embodiments 52-53, wherein Z′ is C₂-C₄ alkylene having from 2-4 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O).
60. The compound of any one of embodiments 52-53, wherein Z′ is C₂ alkylene having 2 carbon atoms independently selected from the group consisting of CH₂, CHR¹⁶, CHR¹⁷, CR¹⁶R¹⁶, CR¹⁷R¹⁷, CR¹⁶R¹⁷, and C(O).
61. The compound of any one of embodiments 58-60, wherein the alkylene comprises C(O).
62. The compound of any one of embodiments 58-61, wherein the alkylene is C(O).
63. The compound of any one of embodiments 58-59, wherein the alkylene is 1-methyl-1-propyl.
64. The compound of any one of embodiments 58-59, wherein the alkylene is 2-methyl-1-propyl.
65. The compound of embodiment 58, wherein the alkylene is 2,2-dimethyl-1-propyl.
66. The compound of any one of embodiments 58-60, wherein the alkylene is ethyl.
67. The compound of any one of embodiments 58-59, wherein the alkylene is n-propyl.
68. The compound of any one of embodiments 58-59, wherein the alkylene is n-butyl.
69. The compound of any one of embodiments 58-59, wherein the alkylene is branched.
70. The compound of any one of embodiments 58-59, wherein the alkylene is linear.
71. The compound of any one of embodiments 52-53, wherein Z′ is (ii) 3-10-membered heterocycloalkylene optionally substituted by one or more R¹ and/or R².
72. The compound of any one of embodiments 52-53 and 69, wherein Z′ is a 5-6-membered heterocycloalkylene optionally substituted by one or more R¹ and/or R².
73. The compound of any one of embodiments 52-53 and 69, wherein Z′ is a 5-membered heterocycloalkylene optionally substituted by one or more R¹ and/or R².
74. The compound of any one of embodiments 52-53 and 69, wherein Z′ is a 6-membered heterocycloalkylene optionally substituted by one or more R¹ and/or R².
75. The compound of any one of embodiments 52-53 and 69-73, wherein Z′ is pyrrolidinylene (e.g., 3-pyrrolidinylene) optionally substituted by one or more R¹ and/or R².
76. The compound of any one of embodiments 52-53, 69-72, and 74, wherein Z′ is piperidinylene (e.g., 4-piperidinylene) optionally substituted by one or more R¹ and/or R².
77. The compound of any one of embodiments 52-53, wherein Z′ is (iii) C₃-C₁₀ cycloalkyl optionally substituted by one or more R¹ and/or R².
78. The compound of any one of embodiments 52-53 and 77, wherein Z′ is cyclohexyl optionally substituted by one or more R¹ and/or R²
79. The compound of any one of embodiments 52-53 and 77, wherein Z′ is cyclopentyl optionally substituted by one or more R¹ and/or R².
80. The compound of any one of embodiments 52-53 and 77, wherein Z′ is cyclobutyl optionally substituted by one or more R¹ and/or R².
81. The compound of any one of embodiments 52-53 and 77, wherein Z′ is cyclopropyl optionally substituted by one or more R¹ and/or R².
82. The compound of any one of embodiments 52-54 and 56-81, wherein Q is

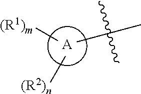

wherein ring A is selected from the group consisting of 5- to 10-membered heteroaryl, C₆-C₁₀ aryl, C₃-C₁₀ cycloalkyl, and 3-10-membered heterocycloalkyl.
83. The compound of any one of embodiments 52-54 and 56-81, wherein Q is H.
84. The compound of any one of embodiments 52-54 and 56-82, wherein ring A is selected from the group consisting of 5- to 10-membered heteroaryl, C₆-C₁₀ aryl, and 3-10-membered heterocycloalkyl.
85. The compound of any one of embodiments 52-54, 56-82, and 84, wherein ring A is selected from the group consisting of 5- to 10-membered heteroaryl.
86. The compound of any one of embodiments 52-54, 56-82, and 84, wherein ring A is selected from the group consisting of C₆-C₁₀ aryl.
87. The compound of any one of embodiments 52-54, 56-82, and 84, wherein ring A is selected from the group consisting of 3-10-membered heterocycloalkyl.
88. The compound of any one of embodiments 52-54, 56-82, and 84-85, wherein ring A is thiophenyl (e.g., 3-thiophenyl).
89. The compound of any one of embodiments 50-52, 54-80, and 82-83, wherein ring A is thiazolyl (e.g., 5-thiazolyl).
90. The compound of any one of embodiments 50-52, 54-80, and 82-83, wherein ring A is pyrazolyl (e.g., 4-pyrazolyl).
91. The compound of any one of embodiments 50-52, 54-80, and 82-83, wherein ring A is isoxazolyl (e.g., 5-isoxazolyl).
92. The compound of any one of embodiments 50-52, 54-80, and 82-83, wherein ring A is isoxazolyl (e.g., 5-isoxazolyl).
93. The compound of any one of embodiments 52-54, 56-82, 84, and 86, wherein ring A is phenyl.
94. The compound of any one of embodiments 52-54, 56-82, 84, and 87, wherein ring A is pyrrolidinyl (e.g., 2-pyrrolidinyl or 3-pyrrolidinyl).
95. The compound of any one of embodiments 52-54, 56-82, 84, and 87, wherein ring A is piperidinyl (e.g., 3-piperidinyl or 4-piperidinyl).
96. The compound of any one of embodiments 52-54, 56-82, 84, and 87, wherein ring A is azetidinyl (e.g., 2-azetidinyl).
97. The compound of any one of embodiments 52-54, 56-82, 84, and 87, wherein ring A is morpholinyl (e.g., 2-morpholinyl).
98. The compound of any one of embodiments 52-54, 56-82, 84, and 87, wherein ring A is pyrrolidinyl (e.g., 2-pyrrolidinyl or 3-pyrrolidinyl).
99. The compound of any one of embodiments 53 and 56-98, wherein R¹⁶ and R¹⁷ are each independently selected from C₁-C₆ alkyl, C₁-C₆ alkoxy, C₁-C₆ haloalkoxy, halo, CN, CO₂H, COC₁-C₆ alkyl, CO—C₆-C₁₀ aryl; CO(5- to 10-membered heteroaryl); CO₂C₁-C₆ alkyl, OCOC₁-C₆ alkyl, NH₂, NHC₁-C₆ alkyl, N(C₁-C₆ alkyl)₂, NHCOC₁-C₆ alkyl, NHCOC₆-C₁₀ aryl, NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹²,
   wherein the C₁-C₆ alkyl is optionally substituted with one or more substituents each independently selected from hydroxy, halo, CN, oxo, C₁-C₆ alkoxy, NR⁸R⁹, ═NR¹⁰, COOC₁-C₆ alkyl, CONR⁸R⁹, OCOC₁-C₆ alkyl, NHCOC₁-C₆ alkyl, NHCOC₆-C₁₀ aryl, and NHCOC₂-C₆ alkynyl.
100. The compound of any one of embodiments 53 and 56-98, wherein R¹⁶ and R¹⁷ are each independently selected from C₁-C₆ alkyl, C₁-C₆ alkoxy, C₁-C₆ haloalkoxy, halo, CN, CO₂H, COC₁-C₆ alkyl, CO—C₆-C₁₀ aryl; CO(5- to 10-membered heteroaryl); CO₂C₁-C₆ alkyl, OCOC₁-C₆ alkyl, NH₂, NHC₁-C₆ alkyl, N(C₁-C₆ alkyl)₂, NHCOC₁-C₆ alkyl, NHCOC₆-C₁₀ aryl, NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹²,
   wherein the C₁-C₆ alkyl is optionally substituted with one or more substituents each independently selected from hydroxy, halo, CN, oxo, C₁-C₆ alkoxy, NR⁸R⁹, ═NR¹⁰, COOC₁-C₆ alkyl, CONR⁸R⁹, OCOC₁-C₆ alkyl, NHCOC₁-C₆ alkyl, NHCOC₆-C₁₀ aryl, and NHCOC₂-C₆ alkynyl.
101. The compound of any one of embodiments 53-100, wherein R¹ and R² are each independently selected C₁-C₆ alkyl, C₁-C₆ alkoxy, halo, CN, CO₂H, COC₁-C₆ alkyl, CO₂C₁-C₆ alkyl, C₆-C₁₀ aryl, 5- to 10-membered heteroaryl, NH₂, NHC₁-C₆ alkyl, N(C₁-C₆ alkyl)₂, NHCOC₁-C₆ alkyl, CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
   wherein the C₁-C₆ alkyl is optionally substituted with one or more substituents
   each independently selected from hydroxy, halo, oxo, C₁-C₆ alkoxy, and NR⁸R⁹.
102. The compound of any one of embodiments 53-100, wherein R¹ and R² are each independently selected from methyl, ethyl, isopropyl, 2-hydroxy-2-propyl, dimethylamino, aminomethyl, methylaminomethyl, dimethylaminomethyl, methoxycarbonyl, and carboxyl.
103. The compound of any one of embodiments 1-102, wherein B is phenyl substituted with 1 or 2 R⁶ and optionally substituted with 1, 2, or 3 R⁷.
104. The compound of embodiment 103, wherein o=2 and p=0.
105. The compound of any one of embodiments 103-104, wherein B is

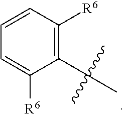

106. The compound of embodiment 105, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
107. The compound of any one of embodiments 105-106, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.
108. The compound of embodiment 103, wherein o=1 and p=1.
109. The compound of embodiment 103, wherein o=2 and p=1.
110. The compound of embodiment 109, wherein B is

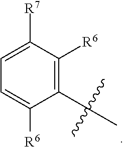

111. The compound of embodiment 110, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
   wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
112. The compound of embodiment 109, wherein B is

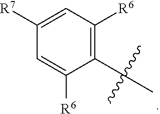

113. The compound of embodiment 112, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
   wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.
114. The compound of embodiment 103, wherein o=2 and p=2.
115. The compound of embodiment 114, wherein B is

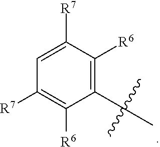

116. The compound of embodiment 115, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
   wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
117. The compound of any one of embodiments 115-116, wherein B is

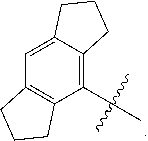 [SMILES: *c1c2c(cc3c1CCC3)CCC2]

118. The compound of embodiment 114, wherein B is

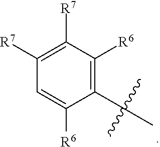

119. The compound of embodiment 118, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
   wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
120. The compound of embodiment 103, wherein o=2 and p=3
121. The compound of embodiment 120, wherein B is

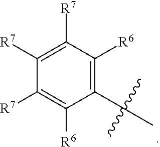

122. The compound of embodiment 121, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,
   wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
123. The compound of any one of the preceding embodiments, wherein R³ is hydrogen.
124. The compound of embodiment 1, wherein the compound of Formula AA is a compound of Formula BB:

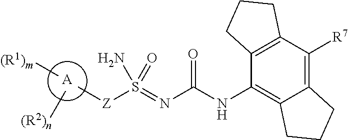

wherein:
Z is C₁₋₄alkylene having from 1-4 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O); and
when
   (i) ring A is phenyl,
   (ii) the sum of m and n is 1, and
   (iii) whichever of R¹ and R² that is present is CN;
then the position of the phenyl group that is para to the point of the phenyl group's connection to the sulfur of the S(O)(NHR³)═N moiety is substituted with hydrogen.
125. The compound of embodiment 1, wherein the compound of Formula AA is a compound of Formula CC:

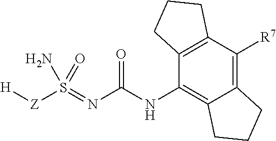

wherein Z is:
(ii) 3-10-membered heterocycloalkylene optionally substituted by one or more R¹ and/or R²; or
(iii) C₃-C₁₀ cycloalkyl optionally substituted by one or more R¹ and/or R².
126. A compound selected from the group consisting of the compounds in Table 1A-1, and pharmaceutically acceptable salts thereof
127. A compound selected from the group consisting of the compounds in Table 1A-2, and pharmaceutically acceptable salts thereof
128. A compound selected from the group consisting of the compounds in Table 1B, and pharmaceutically acceptable salts thereof
129. A compound selected from the group consisting of the compounds in Table 1C, and pharmaceutically acceptable salts thereof
130. A compound selected from the group consisting of the compounds in Table 1D, and pharmaceutically acceptable salts thereof
131. The compound of any one of the preceding embodiments, wherein the sulfur in the moiety S(═O)(NHR³)═N— has (S) stereochemistry.
132. The compound of any one of embodiments 1 to 130, wherein the sulfur in the moiety S(═O)(NHR³)═N— has (R) stereochemistry.
133. A pharmaceutical composition comprising a compound or salt as defined in any one of embodiments 1-132 and one or more pharmaceutically acceptable excipients.
134. A method for modulating NLRP3 activity, the method comprising contacting NLRP3 with an effective amount of a compound as defined in any one of embodiments 1-132 or a pharmaceutical composition as defined in embodiment 133.
135. The method of embodiment 134, wherein the modulating comprises antagonizing NLRP3.
136. The method of any one of embodiments 134 or 135, which is carried out in vitro.
137. The method of embodiment 134-136, wherein the method comprises contacting a sample comprising one or more cells comprising NLRP3 with the compound.
138. The method of any one of embodiments 134, 135, and 137, which is carried out in vivo.
139. The method of embodiment 138, wherein the method comprises administering the compound to a subject having a disease in which NLRP3 signaling contributes to the pathology and/or symptoms and/or progression of the disease.
140. The method of embodiment 139, wherein the subject is a human.
141. A method of treating a disease, disorder or condition that is a metabolic disorder, comprising administering to a subject in need of such treatment an effective amount of a compound as defined in any one of embodiments 1-132 or a pharmaceutical composition as defined in embodiment 133.
142. The method of embodiment 141, wherein the metabolic disorder is Type 2 diabetes, atherosclerosis, obesity or gout.
143. A method of treating a disease, disorder or condition that is a disease of the central nervous system, comprising administering to a subject in need of such treatment an effective amount of a compound as defined in any one of embodiments 1-132 or a pharmaceutical composition as defined in embodiment 133.
144. The method of embodiment 143, wherein the disease of the central nervous system is Alzheimer's disease, multiple sclerosis, Amyotrophic Lateral Sclerosis, or Parkinson's disease.
145. A method of treating a disease, disorder or condition that is lung disease, comprising administering to a subject in need of such treatment an effective amount of a compound as defined in any one of embodiments 1-132 or a pharmaceutical composition as defined in embodiment 133.
146. The method of embodiment 145, wherein the lung disease is asthma, COPD or pulmonary idiopathic fibrosis.
147. A method of treating a disease, disorder or condition that is liver disease, comprising administering to a subject in need of such treatment an effective amount of a compound as defined in any one of embodiments 1-132 or a pharmaceutical composition as defined in embodiment 133.
148. The method of embodiment 147, wherein the liver disease is NASH syndrome, viral hepatitis or cirrhosis.
149. A method of treating a disease, disorder or condition that is pancreatic disease, comprising administering to a subject in need of such treatment an effective amount of a compound as defined in any one of embodiments 1-132 or a pharmaceutical composition as defined in embodiment 133.
150. The method of embodiment 149, wherein the pancreatic disease is acute pancreatitis or chronic pancreatitis.
151. A method of treating a disease, disorder or condition that is kidney disease, comprising administering to a subject in need of such treatment an effective amount of a compound as defined in any one of embodiments 1-132 or a pharmaceutical composition as defined in embodiment 133.
152. The method of embodiment 151, wherein the kidney disease is acute kidney injury or chronic kidney injury.
153. A method of treating a disease, disorder or condition that is intestinal disease, comprising administering to a subject in need of such treatment an effective amount of a compound as defined in any one of embodiments 1-132 or a pharmaceutical composition as defined in embodiment 133.
154. The method of embodiment 153, wherein the intestinal disease is Crohn's disease or Ulcerative Colitis.
155. A method of treating a disease, disorder or condition that is skin disease, comprising administering to a subject in need of such treatment an effective amount of a compound as defined in any one of embodiments 1-132 or a pharmaceutical composition as defined in embodiment 133.
156. The method of embodiment 155, wherein the skin disease is psoriasis.
157. A method of treating a disease, disorder or condition that is musculoskeletal disease, comprising administering to a subject in need of such treatment an effective amount of a compound as defined in any one of embodiments 1-132 or a pharmaceutical composition as defined in embodiment 133.
158. The method of embodiment 157, wherein the musculoskeletal disease is scleroderma.
159. A method of treating a disease, disorder or condition that is a vessel disorder, comprising administering to a subject in need of such treatment an effective amount of a compound as defined in any one of embodiments 1-132 or a pharmaceutical composition as defined in embodiment 133.
160. The method of embodiment 159 wherein the vessel disorder is giant cell arteritis.
161. A method of treating a disease, disorder or condition that is a disorder of the bones, comprising administering to a subject in need of such treatment an effective amount of a compound as defined in any one of embodiments 1-132 or a pharmaceutical composition as defined in embodiment 133.
162. The method of embodiment 161, wherein the disorder of the bones is osteoarthritis, osteoporosis or osteopetrosis disorders.
163. A method of treating a disease, disorder or condition that is eye disease, comprising administering to a subject in need of such treatment an effective amount of a compound as defined in any one of embodiments 1-132 or a pharmaceutical composition as defined in embodiment 133.
164. The method of embodiment 163, wherein the eye disease is glaucoma or macular degeneration.

165. A method of treating a disease, disorder or condition that is a disease caused by viral infection, comprising administering to a subject in need of such treatment an effective amount of a compound as defined in any one of embodiments 1-132 or a pharmaceutical composition as defined in embodiment 133.
166. The method of embodiment 165, wherein the diseases caused by viral infection is HIV or AIDS.
167. A method of treating a disease, disorder or condition that is an autoimmune disease, comprising administering to a subject in need of such treatment an effective amount of a compound as defined in any one of embodiments 1-132 or a pharmaceutical composition as defined in embodiment 133.
168. The method of embodiment 167, wherein the autoimmune disease is Rheumatoid Arthritis, Systemic Lupus Erythematosus, Autoimmune Thyroiditis.
169. A method of treating a disease, disorder or condition that is cancer or aging, comprising administering to a subject in need of such treatment an effective amount of a compound as defined in any one of embodiments 1-132 or a pharmaceutical composition as defined in embodiment 133.
170. A method of treating a disease, disorder or condition that is a cancer selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML); chronic myeloid leukemia (CML); gastric cancer; and lung cancer metastasis, comprising administering to a subject in need of such treatment an effective amount of a compound as defined in any one of embodiments 1-132 or a pharmaceutical composition as defined in embodiment 133.
171. The method of embodiment 170, wherein the cancer is MDS.
172. The method of embodiment 170, wherein the cancer is non-small lung cancer.
173. The method of embodiment 170, wherein the cancer is acute lymphoblastic leukemia.
174. The method of embodiment 170, wherein the cancer is LCH.
175. The method of embodiment 170, wherein the cancer is multiple myeloma.
176. The method of embodiment 170, wherein the cancer is promyelocytic leukemia.
177. The method of embodiment 170, wherein the cancer is acute myeloid leukemia (AML).
178. The method of embodiment 170, wherein the cancer is chronic myeloid leukemia (CML).
179. The method of embodiment 170, wherein the cancer is gastric cancer.
180. The method of embodiment 170, wherein the cancer is lung cancer metastasis.
181. The method of any one of embodiments 139-180, further comprising administering a therapeutically effective amount of an anti-TNFα agent to the subject.
182. The method of embodiment 181, wherein the NLRP3 antagonist is administered to the subject prior to administration of the anti-TNFα agent to the subject.
183. The method of embodiment 181, wherein the anti-TNFα agent is administered to the subject prior to the administration of the NLRP3 antagonist to the subject.
184. The method of embodiment 181, wherein the NLRP3 antagonist and the anti-TNFα agent are administered to the subject at substantially the same time.
185. The method of embodiment 181, wherein the NLRP3 antagonist and the anti-TNFα agent are formulated together in a single dosage form.

A number of embodiments of the invention have been described. Nevertheless, it will be understood that various modifications may be made without departing from the spirit and scope of the invention. Accordingly, other embodiments are within the scope of the following claims.

1. A compound of Formula AA 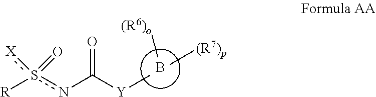
wherein
R is: Z-Q, or NR′R″;
Q is: 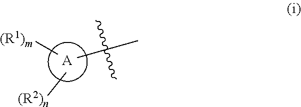
wherein ring A is selected from the group consisting of 5- to 10-membered heteroaryl, C₆-C₁₀ aryl, C₃-C₁₀ cycloalkyl, and 3-10-membered heterocycloalkyl; or
(ii) H
Z is:
(i) C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
(ii) 3-10-membered heterocycloalkylene optionally substituted by one or more R¹ and/or R²; or
(iii) C₃-C₁₀ cycloalkyl optionally substituted by one or more R¹ and/or R²;
R′ and R″ are each independently selected from:
(i) Q; or
(ii) Z″-Q, wherein Z″ is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
or alternatively, wherein R′ and R″ are taken together with the N to which they are attached to form a 5-10-membered heterocycloalkyl ring optionally substituted with one or more R¹ and/or R²; [symbol]
represents a single or double bond;
wherein one of the following apply:
(i) When X is NHR³, a single bond is present between X and S, a double bond is present between S and N, and Y is selected from NH and CR⁴R⁵; or
(ii) When X is O, a double bond is present between X and S, a single bond is present between S and N, the N that is bonded to S is further substituted with an H, and Y is CR⁴R⁵;
B is selected from the group consisting of 5-membered heteroaryl, 7-10 membered heteroaryl, and C₆-C₁₀ aryl;
m=0, 1, or 2;
n=0, 1, or 2;
R¹ and R² are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R¹⁶ and R¹⁷ are each independently selected from C₁-C₆ alkyl, C₁-C₆ haloalkyl, C₁-C₆ alkoxy, C₁-C₆ haloalkoxy, halo, CN, NO₂, CO₂H, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, and S(O₂)NR¹¹R¹²,
wherein the C₁-C₆ alkyl and C₁-C₆ haloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
R³ is selected from hydrogen, hydroxy, C₁-C₆ alkoxy, C₁-C₆ alkyl, and 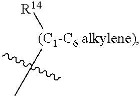
wherein the C₁-C₆ alkylene group is optionally substituted by oxo;
each of R⁴ and R⁵ is independently selected from hydrogen and C₁-C₆ alkyl;
o=1 or 2;
p=0, 1, 2, or 3;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
R¹⁰ is C₁-C₆ alkyl;
each of R¹¹ and R¹² at each occurrence is independently selected from hydrogen and C₁-C₆ alkyl;
R¹³ is C₁-C₆ alkyl, C₆-C₁₀ aryl, or 5- to 10-membered heteroaryl;
R¹⁴ is hydrogen, hydroxy, C₁-C₆ alkyl, NR⁸R⁹, 5- to 10-membered monocyclic or bicyclic heteroaryl, or C₆-C₁₀ monocyclic or bicyclic aryl, wherein each C₁-C₆ alkyl, aryl or heteroaryl is optionally independently substituted with 1 or 2 R⁶;
each R¹⁵ at each occurrence are each independently selected from the group consisting of C₁-C₆ alkyl, C₁-C₆ haloalkyl, C₁-C₆ alkoxy, C₁-C₆ haloalkoxy, CN, COC₁-C₆ alkyl, CO—C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, =pNR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹⁵ C₃-C₇ cycloalkyl or of the R¹⁵ 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
with the proviso that the compound of Formula AA is not the following structure: 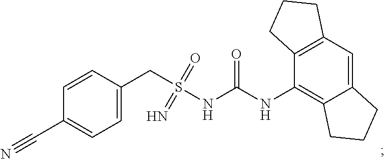 [SMILES: N#Cc1ccc(CS(=N)(=O)NC(=O)Nc2c3c(cc4c2CCC4)CCC3)cc1]
or a pharmaceutically acceptable salt thereof.

2. The compound of claim 1, wherein R is —Z-Q.

3. The compound of claim 1, wherein R is NR′R″.

4. The compound of claim 3, wherein R′ and R″ are:
(i) each independently selected from —Z″-Q, wherein Z″ is C₁-C₈ alkylene having from 1-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O); or
(ii) wherein R′ and R″ are taken together with the N to which they are attached to form a 5-10-membered heterocycloalkyl ring optionally substituted with one or more R¹ and/or R².

5. The compound according to claim 1, wherein X is O, a double bond is present between X and S, a single bond is present between S and N, the N that is bonded to S is further substituted with an H, and Y is CR⁴R⁵.

6. The compound according to claim 1, wherein X is NHR³, a single bond is present between X and S, and a double bond is present between S and N.

7. The compound of claim 1, wherein X is NHR³, a single bond is present between X and S, and a double bond is present between S and N; and the compound of Formula AA is a compound of Formula AA-1, Formula AA-2, or Formula AA-3: 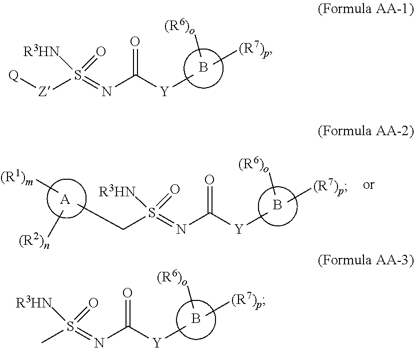
wherein
Z′ is:
(i) C₂-C₈ alkylene having from 2-8 carbon atoms independently selected from the group consisting of CH₂, CH, C, CR¹⁶, CR¹⁷, CHR¹⁶, CHR¹⁷, CR¹⁶R¹⁶, CR¹⁷R¹⁷, CR¹⁶R¹⁷, and C(O);
(ii) CHR¹⁶, CHR¹⁷, CR¹⁶R¹⁶, CR¹⁷R¹⁷, CR¹⁶R¹⁷, or C(O);
(ii) 3-10-membered heterocycloalkylene optionally substituted by one or more R¹ and/or R²; or
(iii) C₃-C₁₀ cycloalkyl optionally substituted by one or more R¹ and/or R²; and
wherein when
(i) Formula AA is Formula AA-2,
(ii) ring A is phenyl,
(iii) the sum of m and n is 1, and
(iv) whichever of R¹ and R² that is present is CN;
then the position of the phenyl group that is para to the point of the phenyl group's connection to the sulfur of the S(O)(NHR³)═N moiety is substituted with hydrogen.

8. The compound according to claim 1, wherein B is phenyl substituted with 1 or 2 R⁶ and optionally substituted with 1, 2, or 3 R⁷.

9. The compound according to claim 1, wherein B is

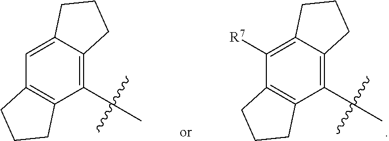

10. The compound according to claim 1, wherein o=2, p=1.

11. The compound according to claim 1, wherein the sulfur in the moiety S(═O)(NHR³)═N— has (S) stereochemistry, or (R) stereochemistry.

12. A pharmaceutical composition comprising a compound or salt as claimed in claim 1 and one or more pharmaceutically acceptable excipients.

13-14. (canceled)

15. A method of treating a disease modulated by NLPR3, comprising administering to a subject in need thereof an effective amount of a compound according to claim 1.

16. A method of treating a disease modulated by TNFα, comprising administering to a subject in need thereof an effective amount of a compound according to claim 1.
